SEQUENCE LISTING (252 sequences)

SEQ ID NO: 1 - protein, 9 aa, synthetic construct
FLDKGTYTL

SEQ ID NO: 2 - protein, 9 aa, synthetic construct
YLSGANLNL

SEQ ID NO: 3 - protein, 9 aa, synthetic construct
NLVPMVATV

SEQ ID NO: 4 - protein, 9 aa, synthetic construct
GILGFVFTL

SEQ ID NO: 5 - protein, 9 aa, synthetic construct
KTWGQYWQV

SEQ ID NO: 6 - protein, 10 aa, synthetic construct
FLPSDFFPSV

SEQ ID NO: 7 - protein, 9 aa, synthetic construct
YMLDLQPET

SEQ ID NO: 8 - protein, 9 aa, synthetic construct
LLMGTLGIV

SEQ ID NO: 9 - protein, 9 aa, synthetic construct
KIFGSLAFL

SEQ ID NO: 10 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 12 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 17 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 23 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 27 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 29 - protein, 10 aa, synthetic construct
YMLDLQPETT

SEQ ID NO: 30 - protein, 9 aa, synthetic construct
FLGYLILGV

SEQ ID NO: 31 - protein, 9 aa, synthetic construct
YLLEMLWRL

SEQ ID NO: 32 - protein, 9 aa, synthetic construct
KVAELVHFL

SEQ ID NO: 33 - protein, 10 aa, synthetic construct
GVYDGREHTV

SEQ ID NO: 34 - protein, 9 aa, synthetic construct
KVLEHVVRV

SEQ ID NO: 35 - protein, 9 aa, synthetic construct
SLLMWITQV

SEQ ID NO: 36 - protein, 9 aa, synthetic construct
SLLMWITQA

SEQ ID NO: 37 - protein, 9 aa, synthetic construct
LLGRNSFEV

SEQ ID NO: 38 - protein, 9 aa, synthetic construct
LLHETDSAV

SEQ ID NO: 39 - protein, 9 aa, synthetic construct
LTLGEFLKL

SEQ ID NO: 40 - protein, 9 aa, synthetic construct
YMDGTMSQV

SEQ ID NO: 41 - protein, 9 aa, synthetic construct
RMFPNAPYL

SEQ ID NO: 42 - protein, 11 aa, synthetic construct
RPHAIRRPLAL

SEQ ID NO: 43 - protein, 9 aa, synthetic construct
FLDEFMEGV

SEQ ID NO: 44 - protein, 10 aa, synthetic construct
LTDDRLFTCY

SEQ ID NO: 45 - protein, 9 aa, synthetic construct
VVMSWAPPV

SEQ ID NO: 46 - protein, 10 aa, synthetic construct
LLLDDLLVSI

SEQ ID NO: 47 - protein, 9 aa, synthetic construct
KTLTSVFQK

SEQ ID NO: 48 - protein, 9 aa, synthetic construct
EAFIQPITR

SEQ ID NO: 49 - protein, 9 aa, synthetic construct
RPHVPESAF

SEQ ID NO: 50 - protein, 10 aa, synthetic construct
SLADEAEVYL

SEQ ID NO: 51 - protein, 9 aa, synthetic construct
KLYEEPLLK

SEQ ID NO: 52 - protein, 9 aa, synthetic construct
KIFSEVTLK

SEQ ID NO: 53 - protein, 9 aa, synthetic construct
ETVSEQSNV

SEQ ID NO: 54 - protein, 9 aa, synthetic construct
VLHDDLLEA

SEQ ID NO: 55 - protein, 10 aa, synthetic construct
GIVEGLITTV

SEQ ID NO: 56 - protein, 9 aa, synthetic construct
DYLQYVLQI

SEQ ID NO: 57 - protein, 10 aa, synthetic construct
SLFEGIDIYT

SEQ ID NO: 58 - protein, 10 aa, synthetic construct
FIASNGVKLV

SEQ ID NO: 59 - protein, 9 aa, synthetic construct
CILGKLFTK

SEQ ID NO: 60 - protein, 9 aa, synthetic construct
QTACEVLDY

SEQ ID NO: 61 - protein, 9 aa, synthetic construct
ILNAMIAKI

SEQ ID NO: 62 - protein, 9 aa, synthetic construct
MVGPWPRPV

SEQ ID NO: 63 - protein, 10 aa, synthetic construct
KEFEDDIINW

SEQ ID NO: 64 - protein, 9 aa, synthetic construct
YTDFHCQYV

SEQ ID NO: 65 - protein, 10 aa, synthetic construct
EEKRGSLHVW

SEQ ID NO: 66 - protein, 11 aa, synthetic construct
SELFRSGLDSY

SEQ ID NO: 67 - protein, 9 aa, synthetic construct
AEPIDIQTW

SEQ ID NO: 68 - protein, 10 aa, synthetic construct
TLDWLLQTPK

SEQ ID NO: 69 - protein, 10 aa, synthetic construct
GLFGDIYLAI

SEQ ID NO: 70 - protein, 9 aa, synthetic construct
ILDKVLVHL

SEQ ID NO: 71 - protein, 9 aa, synthetic construct
SYLDSGIHF

SEQ ID NO: 72 - protein, 10 aa, synthetic construct
KILDAVVAQK

SEQ ID NO: 73 - protein, 9 aa, synthetic construct
KELEGILLL

SEQ ID NO: 74 - protein, 9 aa, synthetic construct
KINKNPKYK

SEQ ID NO: 75 - protein, 11 aa, synthetic construct
FLEGNEVGKTY

SEQ ID NO: 76 - protein, 9 aa, synthetic construct
AQQITKTEV

SEQ ID NO: 77 - protein, 10 aa, synthetic construct
ACDPHSGHFV

SEQ ID NO: 78 - protein, 9 aa, synthetic construct
AVCPWTWLR

SEQ ID NO: 79 - protein, 11 aa, synthetic construct
MEIFIEVFSHF

SEQ ID NO: 80 - protein, 10 aa, synthetic construct
RVWDLPGVLK

SEQ ID NO: 81 - protein, 9 aa, synthetic construct
EEKLIVVLF

SEQ ID NO: 82 - protein, 9 aa, synthetic construct
TPNQRQNVC

SEQ ID NO: 83 - protein, 9 aa, synthetic construct
LVVVGAVGV

SEQ ID NO: 84 - protein, 10 aa, synthetic construct
KLVVVGADGV

SEQ ID NO: 85 - protein, 10 aa, synthetic construct
KLVVVGAVGV

SEQ ID NO: 86 - protein, 9 aa, synthetic construct
GADGVGKSA

SEQ ID NO: 87 - protein, 10 aa, synthetic construct
GADGVGKSAL

SEQ ID NO: 88 - protein, 9 aa, synthetic construct
VVGADGVGK

SEQ ID NO: 89 - protein, 9 aa, synthetic construct
VVGAVGVGK

SEQ ID NO: 90 - protein, 10 aa, synthetic construct
VVVGADGVGK

SEQ ID NO: 91 - protein, 10 aa, synthetic construct
VVVGAVGVGK

SEQ ID NO: 92 - protein, 9 aa, synthetic construct
EVDPIGHLY

SEQ ID NO: 93 - protein, 9 aa, synthetic construct
FLWGPRALV

SEQ ID NO: 94 - protein, 9 aa, synthetic construct
ALSRKVAEL

SEQ ID NO: 95 - protein, 9 aa, synthetic construct
KVTDLVQFL

SEQ ID NO: 96 - protein, 9 aa, synthetic construct
GLYDGMEHL

SEQ ID NO: 97 - protein, 9 aa, synthetic construct
SLLKFLAKV

SEQ ID NO: 98 - protein, 9 aa, synthetic construct
ALLPAVPSL

SEQ ID NO: 99 - protein, 9 aa, synthetic construct
RLDQLLRHV

SEQ ID NO: 100 - protein, 9 aa, synthetic construct
SLLQHLIGL

SEQ ID NO: 101 - protein, 11 aa, synthetic construct
SLFHPEDTGQV

SEQ ID NO: 102 - protein, 11 aa, synthetic construct
SIEPEEFLTPK

SEQ ID NO: 103 - protein, 9 aa, synthetic construct
SLYTKVVHY

SEQ ID NO: 104 - protein, 9 aa, synthetic construct
FMVFLQTHI

SEQ ID NO: 105 - protein, 9 aa, synthetic construct
CLGGLLTMV

SEQ ID NO: 106 - protein, 9 aa, synthetic construct
ALYDKTKRI

SEQ ID NO: 107 - protein, 9 aa, synthetic construct
KLFTSVFGV

SEQ ID NO: 108 - protein, 9 aa, synthetic construct
SLWRRPFNV

SEQ ID NO: 109 - protein, 9 aa, synthetic construct
GLMATPAKL

SEQ ID NO: 110 - protein, 9 aa, synthetic construct
RLFEQVMRI

SEQ ID NO: 111 - protein, 11 aa, synthetic construct
ATYKEVSKMVK

SEQ ID NO: 112 - protein, 9 aa, synthetic construct
LYAPTILLW

SEQ ID NO: 113 - protein, 9 aa, synthetic construct
KIQEILTQV

SEQ ID NO: 114 - protein, 9 aa, synthetic construct
GYLQGLVSF

SEQ ID NO: 115 - protein, 9 aa, synthetic construct
HPEYNRPLL

SEQ ID NO: 116 - protein, 9 aa, synthetic construct
TLMSAMTNL

SEQ ID NO: 117 - protein, 9 aa, synthetic construct
HPEDTGQVF

SEQ ID NO: 118 - protein, 9 aa, synthetic construct
SLYNTVATL

SEQ ID NO: 119 - protein, 9 aa, synthetic construct
ILKEPVHGV

SEQ ID NO: 120 - protein, 9 aa, synthetic construct
AISPRTLNA

SEQ ID NO: 121 - protein, 9 aa, synthetic construct
FLLTRILTI

SEQ ID NO: 122 - protein, 11 aa, synthetic construct
GTLPQDHIVQK

SEQ ID NO: 123 - protein, 9 aa, synthetic construct
YATISLLTV

SEQ ID NO: 124 - protein, 9 aa, synthetic construct
PFPQPELPY

SEQ ID NO: 125 - protein, 9 aa, synthetic construct
PQPELPYPQ

SEQ ID NO: 126 - protein, 10 aa, synthetic construct
ALWGPDPAAA

SEQ ID NO: 127 - protein, 15 aa, synthetic construct
GGGGSGGGGSGGGGS

SEQ ID NO: 128 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 131 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 132 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 133 - protein, 4 aa, synthetic construct
CPPC

SEQ ID NO: 134 - protein, 4 aa, synthetic construct
CPSC

SEQ ID NO: 135 - protein, 20 aa, synthetic construct
EPKSCDKTHTCPPCPAPPVA

SEQ ID NO: 136 - protein, 17 aa, synthetic construct
ESKYGPPCPPCPAPPVA

SEQ ID NO: 137 - protein, 15 aa, synthetic construct
CPPCPAPGGGGPSVF

SEQ ID NO: 138 - protein, 14 aa, synthetic construct
CPPCPAPGGGPSVF

SEQ ID NO: 139 - protein, 13 aa, synthetic construct
CPPCPAPGGPSVF

SEQ ID NO: 140 - protein, 12 aa, synthetic construct
CPPCPAPGPSVF

SEQ ID NO: 141 - protein, 11 aa, synthetic construct
  VARIANT 1..10: note = This region may encompass 1-10 residues
GGGGGGGGGGS

SEQ ID NO: 142 - protein, 11 aa, synthetic construct
  VARIANT 2..11: note = This region may encompass 1-10 residues
SGGGGGGGGGG

SEQ ID NO: 143 - protein, 5 aa, synthetic construct
GGGGS

SEQ ID NO: 144 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 145 - protein, 4 aa, synthetic construct
GGGG

SEQ ID NO: 146 - protein, 5 aa, synthetic construct
GGGGG

SEQ ID NO: 147 - protein, 6 aa, synthetic construct
GGGGGG

SEQ ID NO: 148 - protein, 7 aa, synthetic construct
GGGGGGG

SEQ ID NO: 149 - protein, 8 aa, synthetic construct
GGGGGGGG

SEQ ID NO: 150 - protein, 9 aa, synthetic construct
GGGGGGGGG

SEQ ID NO: 151 - protein, 10 aa, synthetic construct
GGGGSGGGGS

SEQ ID NO: 152 - protein, 4 aa, synthetic construct
GGGS

SEQ ID NO: 153 - protein, 8 aa, synthetic construct
GGGSGGGS

SEQ ID NO: 154 - protein, 6 aa, synthetic construct
GGSGGS

SEQ ID NO: 155 - protein, 9 aa, synthetic construct
GGSGGSGGS

SEQ ID NO: 156 - protein, 4 aa, synthetic construct
GSGS

SEQ ID NO: 157 - protein, 6 aa, synthetic construct
GSGSGS

SEQ ID NO: 158 - protein, 5 aa, synthetic construct
GSGGS

SEQ ID NO: 159 - protein, 5 aa, synthetic construct
AAAGG

SEQ ID NO: 160 - protein, 5 aa, synthetic construct
ADAAP

SEQ ID NO: 161 - protein, 11 aa, synthetic construct
ADAAPTVSIFP

SEQ ID NO: 162 - protein, 12 aa, synthetic construct
ADAAPTVSIFPP

SEQ ID NO: 163 - protein, 6 aa, synthetic construct
AKTTAP

SEQ ID NO: 164 - protein, 13 aa, synthetic construct
AKTTAPSVYPLAP

SEQ ID NO: 165 - protein, 17 aa, synthetic construct
AKTTPKLEEGEFSEARV

SEQ ID NO: 166 - protein, 9 aa, synthetic construct
AKTTPKLGG

SEQ ID NO: 167 - protein, 6 aa, synthetic construct
AKTTPP

SEQ ID NO: 168 - protein, 13 aa, synthetic construct
AKTTPPSVTPLAP

SEQ ID NO: 169 - protein, 6 aa, synthetic construct
ASTKGP

SEQ ID NO: 170 - protein, 26 aa, synthetic construct
ASTKGPSVFPLAPASTKGPSVFPLAP

SEQ ID NO: 171 - protein, 14 aa, synthetic construct
EGKSSGSGSESKST

SEQ ID NO: 172 - protein, 15 aa, synthetic construct
GEGESGEGESGEGES

SEQ ID NO: 173 - protein, 20 aa, synthetic construct
GEGESGEGESGEGESGEGES

SEQ ID NO: 174 - protein, 15 aa, synthetic construct
GEGGSGEGGSGEGGS

SEQ ID NO: 175 - protein, 15 aa, synthetic construct
GENKVEYAPALMALS

SEQ ID NO: 176 - protein, 15 aa, synthetic construct
GGEGSGGEGSGGEGS

SEQ ID NO: 177 - protein, 15 aa, synthetic construct
GGGESGGEGSGEGGS

SEQ ID NO: 178 - protein, 15 aa, synthetic construct
GGGESGGGESGGGES

SEQ ID NO: 179 - protein, 20 aa, synthetic construct
GGGGSGGGGSGGGGSGGGGS

SEQ ID NO: 180 - protein, 15 aa, synthetic construct
GGGKSGGGKSGGGKS

SEQ ID NO: 181 - protein, 15 aa, synthetic construct
GGGKSGGKGSGKGGS

SEQ ID NO: 182 - protein, 5 aa, synthetic construct
GGGSG

SEQ ID NO: 183 - protein, 15 aa, synthetic construct
GGKGSGGKGSGGKGS

SEQ ID NO: 184 - protein, 4 aa, synthetic construct
GGSG

SEQ ID NO: 185 - protein, 5 aa, synthetic construct
GGSGG

SEQ ID NO: 186 - protein, 9 aa, synthetic construct
GGSGGGGSG

SEQ ID NO: 187 - protein, 13 aa, synthetic construct
GGSGGGGSGGGGS

SEQ ID NO: 188 - protein, 15 aa, synthetic construct
GHEAAAVMQVQYPAS

SEQ ID NO: 189 - protein, 15 aa, synthetic construct
GKGGSGKGGSGKGGS

SEQ ID NO: 190 - protein, 15 aa, synthetic construct
GKGKSGKGKSGKGKS

SEQ ID NO: 191 - protein, 20 aa, synthetic construct
GKGKSGKGKSGKGKSGKGKS

SEQ ID NO: 192 - protein, 15 aa, synthetic construct
GKPGSGKPGSGKPGS

SEQ ID NO: 193 - protein, 21 aa, synthetic construct
GKPGSGKPGSGKPGSGKPSGS

SEQ ID NO: 194 - protein, 15 aa, synthetic construct
GPAKELTPLKEAKVS

SEQ ID NO: 195 - protein, 12 aa, synthetic construct
GSAGSAAGSGEF

SEQ ID NO: 196 - protein, 5 aa, synthetic construct
GSGGG

SEQ ID NO: 197 - protein, 5 aa, synthetic construct
GSGSG

SEQ ID NO: 198 - protein, 4 aa, synthetic construct
GSSG

SEQ ID NO: 199 - protein, 10 aa, synthetic construct
GSSGGSGGSG

SEQ ID NO: 200 - protein, 11 aa, synthetic construct
GSSGGSGGSGG

SEQ ID NO: 201 - protein, 12 aa, synthetic construct
GSSGGSGGSGGS

SEQ ID NO: 202 - protein, 13 aa, synthetic construct
GSSGGSGGSGGSG

SEQ ID NO: 203 - protein, 16 aa, synthetic construct
GSSGGSGGSGGSGGGS

SEQ ID NO: 204 - protein, 11 aa, synthetic construct
GSSGGSGGSGS

SEQ ID NO: 205 - protein, 5 aa, synthetic construct
GSSGT

SEQ ID NO: 206 - protein, 5 aa, synthetic construct
GSSSG

SEQ ID NO: 207 - protein, 18 aa, synthetic construct
GSTSGSGKPGSGEGSTKG

SEQ ID NO: 208 - protein, 19 aa, synthetic construct
GTAAAGAGAAGGAAAGAAG

SEQ ID NO: 209 - protein, 19 aa, synthetic construct
GTSGSSGSGSGGSGSGGGG

SEQ ID NO: 210 - protein, 14 aa, synthetic construct
IRPRAIGGSKPRVA

SEQ ID NO: 211 - protein, 18 aa, synthetic construct
KESGSVSSEQLAQFRSLD

SEQ ID NO: 212 - protein, 15 aa, synthetic construct
KTTPKLEEGEFSEAR

SEQ ID NO: 213 - protein, 19 aa, synthetic construct
PRGASKSGSASQTGSAPGS

SEQ ID NO: 214 - protein, 6 aa, synthetic construct
QPKAAP

SEQ ID NO: 215 - protein, 13 aa, synthetic construct
QPKAAPSVTLFPP

SEQ ID NO: 216 - protein, 27 aa, synthetic construct
RADAAAAGGGGSGGGGSGGGGSGGGGS

SEQ ID NO: 217 - protein, 12 aa, synthetic construct
RADAAAAGGPGS

SEQ ID NO: 218 - protein, 6 aa, synthetic construct
RADAAP

SEQ ID NO: 219 - protein, 9 aa, synthetic construct
RADAAPTVS

SEQ ID NO: 220 - protein, 6 aa, synthetic construct
SAKTTP

SEQ ID NO: 221 - protein, 18 aa, synthetic construct
SAKTTPKLEEGEFSEARV

SEQ ID NO: 222 - protein, 10 aa, synthetic construct
SAKTTPKLGG

SEQ ID NO: 223 - protein, 14 aa, synthetic construct
STAGDTHLGGEDFD

SEQ ID NO: 224 - protein, 5 aa, synthetic construct
TVAAP

SEQ ID NO: 225 - protein, 12 aa, synthetic construct
TVAAPSVFIFPP

SEQ ID NO: 226 - protein, 24 aa, synthetic construct
TVAAPSVFIFPPTVAAPSVFIFPP

SEQ ID NO: 227 - protein, 15 aa, synthetic construct
GCGASGGGGSGGGGS

SEQ ID NO: 228 - protein, 15 aa, synthetic construct
GGGASGGGGSGGGGS

SEQ ID NO: 229 - protein, 10 aa, synthetic construct
GGGASGGGGS

SEQ ID NO: 230 - protein, 5 aa, synthetic construct
GCGGS

SEQ ID NO: 231 - protein, 124 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 232 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 233 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 234 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 235 - protein, 123 aa, synthetic construct
EVQLVQSGAEVKKPGESLKISCSGSGYRFTNYWIAWVRQMPGKGLEWMGLIHPDDSDIRYSPSFQGQVTFSVDKSINTAYLQWSSLKASDTAMYYCTRQDGILWSHNAWFDPWGQGTLVTVSS

SEQ ID NO: 236 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 237 - protein, 118 aa, synthetic construct
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 238 - protein, 127 aa, synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 239 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 240 - protein, 119 aa, synthetic construct
QVHLVESGGGVVQPGRSLRLSCSVSGLTFNSYGMHWVRQAPGKGLEWVTMISYDGSDKYYLDSVKGRFTISRDNSKNILYLQINSLRPEDTAVYYCVNWDNWRGNFDYWGQGTLVTVSS

SEQ ID NO: 241 - protein, 419 aa, synthetic construct
YMLDLQPETGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 242 - protein, 9 aa, synthetic construct
YMLDLQPET

SEQ ID NO: 243 - protein, 419 aa, synthetic construct
SLLMWITQCGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 244 - protein, 9 aa, synthetic construct
SLLMWITQC

SEQ ID NO: 245 - protein, 420 aa, synthetic construct
GVYDGREHTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 246 - protein, 10 aa, synthetic construct
GVYDGREHTV

SEQ ID NO: 247 - protein, 419 aa, synthetic construct
NLVPMVATVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 248 - protein, 9 aa, synthetic construct
NLVPMVATV

SEQ ID NO: 249 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 250 - protein, 99 aa, synthetic construct [sequence omitted]

SEQ ID NO: 251 - protein, 276 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 252 - protein, 410 aa, synthetic construct
GCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

(19) United States
(12) Patent Application Publication
(10) Pub. No.: US 2024/0287186 A1
(43) Pub. Date: Aug. 29, 2024

(54) T CELL ACTIVATORS AND METHODS OF USE THEREOF

(71) Applicant: Regeneron Pharmaceuticals, Inc., Tarrytown, NY (US)

(72) Inventors: Aarthi PUTARJUNAN, Yonkers, NY (US); Kyle STAHMER, Wyckoff, NY (US); Lauren BOUCHER, Stamford, CT (US); Lianjie LI, Allendale, NY (US); Raquel DEERING, Ridgefield, CT (US); Chia-Yang LIN, Scarsdale, NY (US); George YANCOPOULOS, Yorktown Heights, NY (US); Johanna HANSEN, Greenwich, CT (US)

(73) Assignee: Regeneron Pharmaceuticals, Inc., Tarrytown, NY (US)

(21) Appl. No.: 18/590,725

(22) Filed: Feb. 28, 2024

Related U.S. Application Data
(60) Provisional application No. 63/487,386, filed on Feb. 28, 2023.
(60) Provisional application No. 63/494,604, filed on Apr. 6, 2023.

(51) Int. Cl. C07K 16/28; A61P 35/00; C07K 14/74; C07K 16/30

(52) U.S. Cl. CPC C07K 16/2818; A61P 35/00; C07K 14/70539; C07K 16/2803; C07K 16/2809; C07K 16/2887; C07K 16/3076; C07K 2317/31; C07K 2317/52; C07K 2317/56

(57) ABSTRACT

The present disclosure relates to multispecific molecules comprising a peptide-MHC complex and an immune cell antigen targeting moiety. Particular aspects relate to multimeric (e.g., dimeric) molecules comprising a peptide-MHC complex, an immune cell antigen targeting moiety, and a multimerization moiety. The disclosure further provides pharmaceutical compositions comprising the multispecific molecules, and methods of use of multispecific molecules in antigen-specific T cell activation, in inducing an antigen-specific immune response, and in therapeutic applications, as well as nucleic acids encoding the multispecific molecules, recombinant cells that express the multispecific molecules, and methods of producing the multispecific molecules.

### 1. CROSS-REFERENCE TO RELATED APPLICATIONS

This application claims the priority benefit of U.S. provisional application No. 63/487,386, filed Feb. 28, 2023, and U.S. provisional application No. 63/494,604, filed Apr. 6, 2023, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Feb. 28, 2024 is named RGN-033US_SL.xml and is 229,868 bytes in size.

### 3. BACKGROUND

Activation of T cells is key to enabling antigen recognition, which is essential for the immune system to develop an effective response to health threats. Hence, antigen-specific T cell activation can be used to prevent and/or treat several ailments, such as viral, bacterial, fungal, and/or protozoal infections, as well as various cancers.

Activation of T cells is considered to involve three signals: (1) antigen recognition, (2) co-stimulation, and (3) cytokine-mediated differentiation and expansion. The first signal, often referred to as signal 1, triggers the initial activation of T cells, which involves encountering antigenic peptides held by major histocompatibility complex (MHC) molecules, usually presented on the surfaces of antigen presenting cells (APCs). These peptide-MHC (pMHC) molecules bind to T cell receptors (TCRs) complexed with CD3 co-receptors. Signal 2 involves co-stimulation of T cells by secondary signals, such as molecules that bind to CD28 co-receptors. This co-stimulation of T cells regulates their proliferation and survival.

Treatment of T cells with antibodies against CD3 have been shown to cluster CD3 on T cells, causing T cell activation in a manner similar to the engagement of the TCR by peptide-loaded MHC molecules. However, such activation is not antigen specific. There is a need in the art for molecules that will trigger antigen-specific activation and expansion of T cells for prevention and/or treatment of various infectious diseases and cancers.

### 4. SUMMARY

The present disclosure provides multispecific molecules containing at least one peptide-MHC complex and at least one immune cell antigen targeting moiety (e.g., T cell antigen targeting moiety, B cell antigen targeting moiety, etc.), referred to herein for convenience as “T cell activators”. Particular T cell activators described herein include those comprising at least one T cell antigen (TCA) targeting moiety (e.g., CD3 targeting moiety, CD28 targeting moiety, etc.), in some cases further comprising one or more additional immune cell antigen targeting moieties (e.g., a B cell antigen targeting moiety such as a CD19 targeting moiety, CD20 targeting moiety, or CD22 targeting moiety). Without being bound by theory, it is believed that inclusion of a peptide-MHC complex and a TCA targeting moiety gives rise to a molecule which can cluster and/or activate T cell receptors (TCRs) specific for a particular antigen (e.g., viral antigen, tumor antigen, etc.) independent of antigen presenting cells. As further described herein, where the TCA targeting moiety is a CD3 targeting moiety, certain disclosed T cell activators are capable of activating TCR signaling independent of costimulatory signal 2. Additional T cell activators described herein include those comprising at least one B cell antigen (BCA) targeting moiety (e.g., CD19 targeting moiety, CD20 targeting moiety, CD22 targeting moiety, etc.), in some cases further comprising one or more additional immune cell antigen targeting moieties (e.g., a T cell antigen targeting moiety such as a CD28 targeting moiety or CD3 targeting moiety). T cell activators described herein are useful in various therapeutic and prophylactic methods where stimulation of the immune system of the host is beneficial.

Peptide-MHC complexes that can be used in the T cell activators of the disclosure are described in Section 6.3.

Immune cell antigen targeting moieties that can be used in the T cell activators of the disclosure are described in Section 6.4.

In certain cases, T cell activators also comprise a tumor antigen targeting moiety. Tumor antigen targeting moieties that can be used in the T cell activators of the disclosure are described in Section 6.5.

In some cases, T cell activators also comprise a multimerization moiety such as an Fc domain. Multimerization moieties that can be used in the T cell activators of the disclosure are described in Section 6.7.

Various exemplary configurations of the T cell activators of the disclosure are described in specific embodiments 1 to 566, infra.

Linkers that can be used to connect different components of the T cell activators of the disclosures are described in Section 6.8.

The disclosure further provides nucleic acids encoding the T cell activators of the disclosure. The nucleic acids encoding the T cell activators can be a single nucleic acid (e.g., a vector encoding all polypeptide chains of a T cell activator) or a plurality of nucleic acids (e.g., two or more vectors encoding the different polypeptide chains of a T cell activator). The disclosure further provides host cells and cell lines engineered to express the nucleic acids and T cell activators of the disclosure. The disclosure further provides methods of producing a T cell activator of the disclosure. Exemplary nucleic acids, host cells, and cell lines, and methods of producing a T cell activator are described in Section 6.9 and specific embodiments 567 to 570 infra.

The disclosure further provides pharmaceutical compositions comprising the T cell activators of the disclosure (or nucleic acids encoding the T cell activators), optionally in addition to one or more multispecific antigen binding molecules (e.g., as described in Section 6.6). Exemplary pharmaceutical compositions are described in Section 6.10 and specific embodiments 575 to 580, infra, with exemplary pharmaceutical compositions comprising T cell activator polypeptides described in Section 6.10.1 and exemplary pharmaceutical compositions comprising T cell activator encoding nucleic acids described in Section 6.10.2.

Further provided herein are methods of using the T cell activators and the pharmaceutical compositions of the disclosure, e.g., for treating or preventing cancer. Exemplary methods are described in Section 6.11 and include therapeutic methods (e.g., via delivery of T cell activator polypeptides) and prophylactic methods (e.g., via delivery of T cell activator encoding nucleic acids such as plasmids, DNA, mRNA, or viral vectors). The T cell activators of the disclosure are useful in combination therapy, for example in combination with a multispecific antigen binding molecule (e.g., as described in Section 6.6), cytokine therapeutic, or other cancer therapeutic. Exemplary combination therapy methods are disclosed in Section 6.12. Specific embodiments of the methods of treatment, prevention, and immune cell activation of the disclosure are described in specific embodiments 581 to 637, infra.

### 5. BRIEF DESCRIPTION OF THE FIGURES

FIGS. 1A-11 illustrate exemplary T cell activator constructs of the disclosure, where:

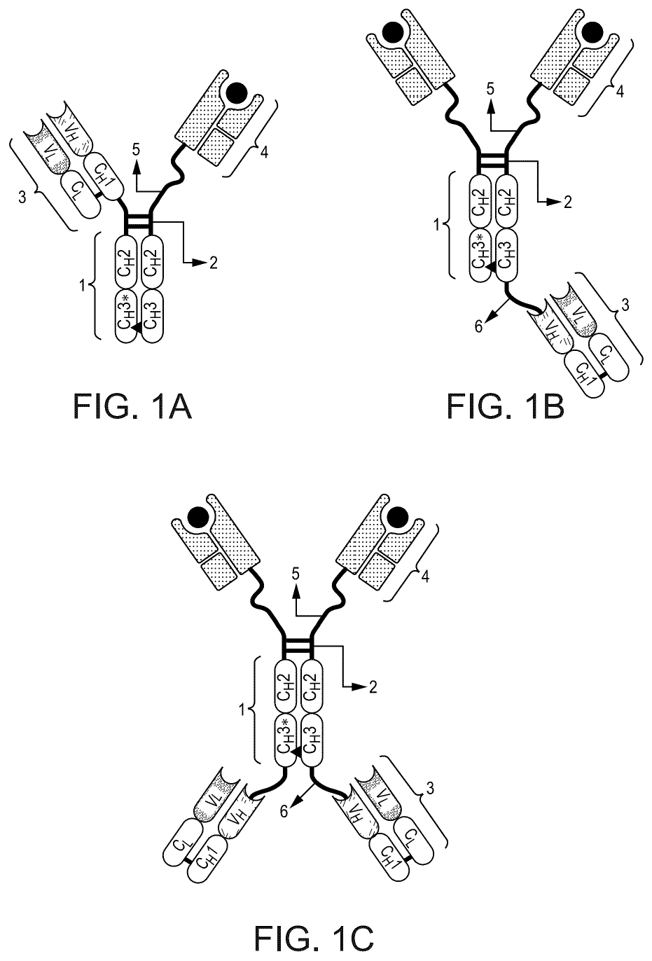

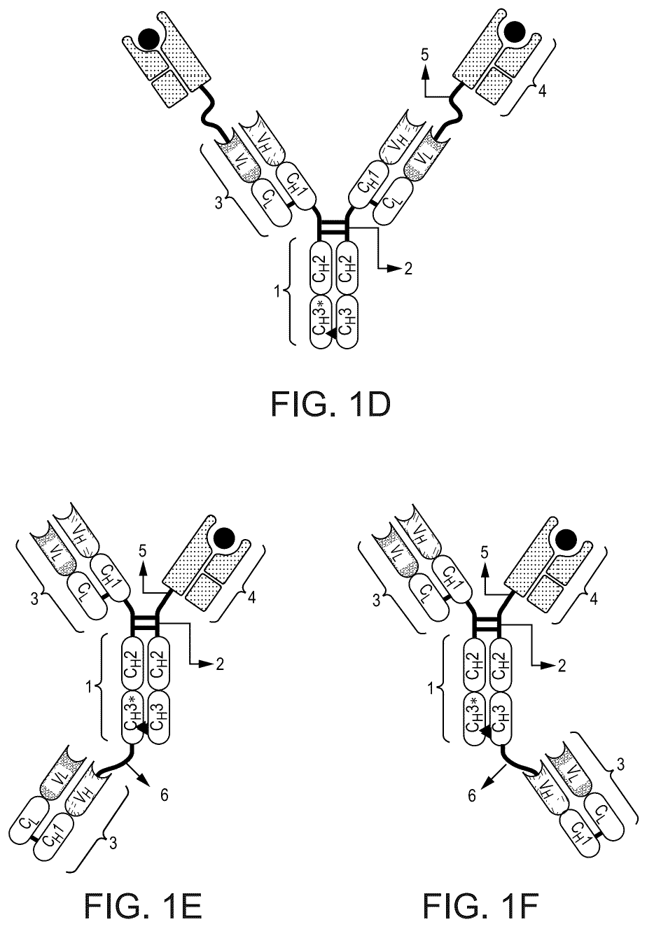

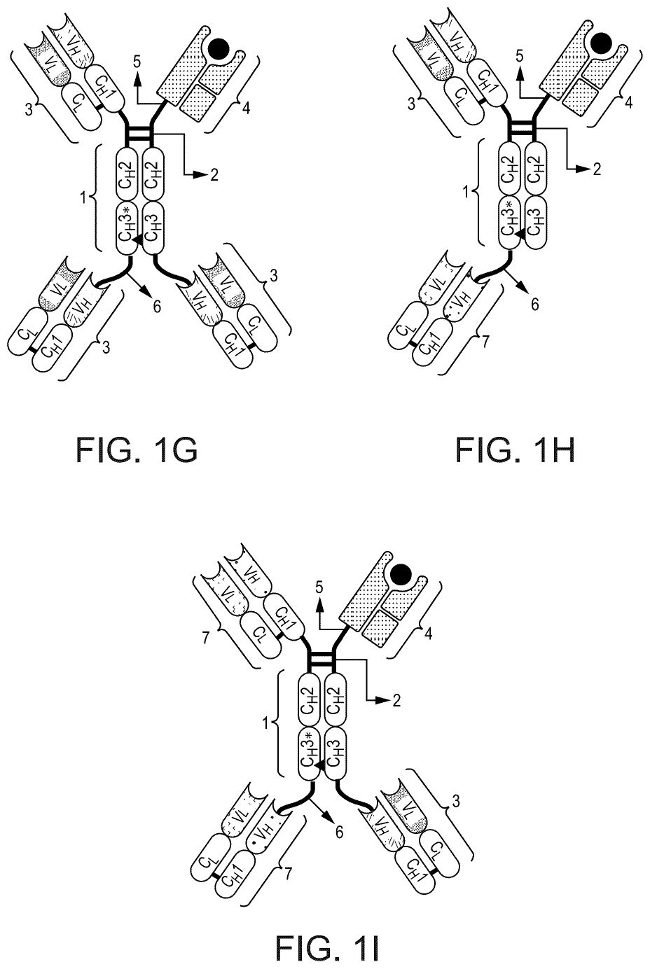

(1) represents an Fc domain of an immunoglobulin, as described in Section 6.7.1, e.g., an IgG such as an IgG1 or an IgG4, which typically includes a CH2 domain, CH3 domain, and optionally a hinge region (represented separately as (2) in FIG. 1), e.g., as set forth in Section 6.7.1.3;

(2) represents the hinge region of an immunoglobulin Fc domain, e.g., an IgG hinge domain, which in various embodiments is an IgG1 or an IgG4 hinge domain or a chimeric hinge domain, as described in Section 6.7.1.3;

(3) represents an immune cell antigen targeting moiety (depicted as a Fab domain but which may be any immune cell antigen targeting moiety described herein), for example a T cell targeting moiety (e.g., an antigen-binding domain of an anti-CD3 or anti-CD28 antibody) or a B cell targeting moiety (e.g., an antigen-binding domain of an anti-CD19, anti-CD20, or anti-CD22 antibody), as described in Section 6.4.

(4) represents a peptide-MHC (pMHC) complex, as described in Section 6.3;

(5-6) represent linkers, for example linkers as described in Section 6.8. Linkers may be used to connect a pMHC complex to the hinge region at the N-terminus of an Fc domain, or to connect an immune cell antigen targeting moiety or a tumor antigen targeting moiety to the C-terminus of an Fc domain, wherein:

(5) represents a linker between a pMHC complex and the hinge region at the N-terminus of an Fc domain;

(6) represents a linker between the C-terminus of an Fc domain and a targeting moiety.

(7) represents a tumor antigen targeting moiety (depicted as a Fab domain but which may be any tumor antigen targeting moiety).

Asymmetrical constructs are depicted as Fc heterodimers, with knob mutations (bulge) and hole mutations (cavity) to facilitate heterodimerization and “star” (star) mutations to facilitate purification. However, any alternative heterodimerization strategies may be used, or the knob and hole and/or star mutation chains may be reversed.

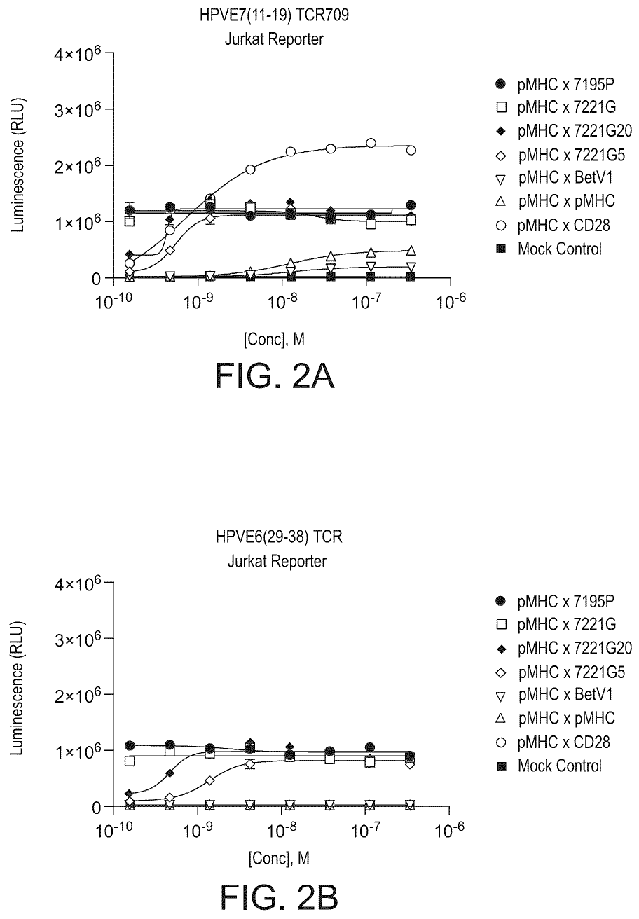

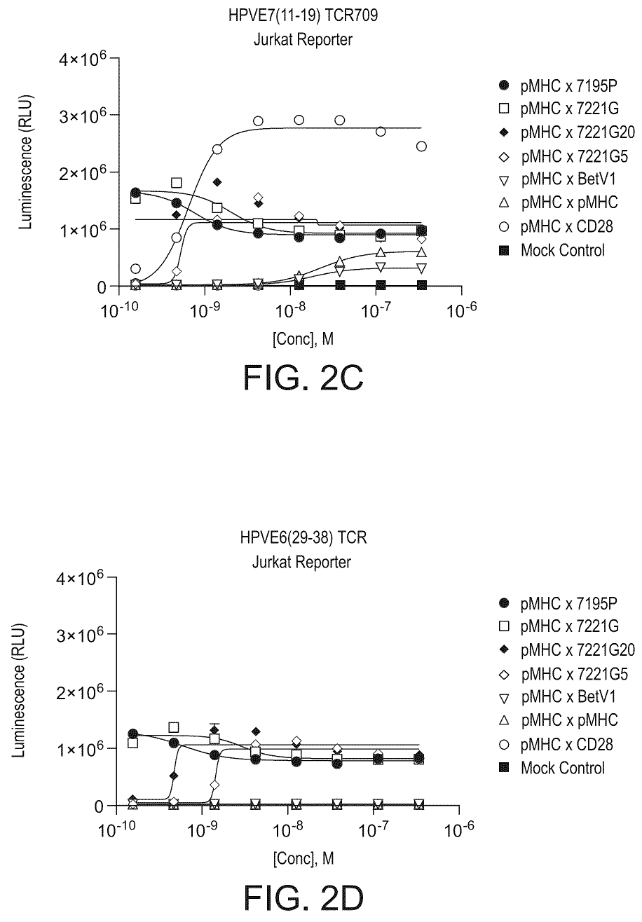

FIGS. 2A-2D show Jurkat reporter cell activation by plate-bound monovalent T cell activator constructs, each comprising a single pMHC complex. FIG. 2A shows activation of Jurkat/NFAT-Luc/Cl 3C7/HPV16E7(11-19) TCR709 cells by plate-bound T cell activator constructs, wherein the pMHC complex of each is connected to the hinge region of the Fc domain via a short linker. FIG. 2B shows activation of Jurkat/NFAT-Luc/Cl 3C7/HPV16E6(29-38) TCR cells by plate-bound T cell activator constructs, wherein the pMHC complex is connected to the hinge region of the Fc domain via a short linker. FIG. 2C shows activation of Jurkat/NFAT-Luc/Cl 3C7/HPV16E7(11-19) TCR709 cells by plate-bound T cell activator constructs, wherein the pMHC complex is connected to the hinge region of the Fc domain via a long linker. FIG. 2D shows activation of Jurkat/NFAT-Luc/Cl 3C7/HPV16E6(29-38) TCR cells by plate-bound T cell activator constructs, wherein the pMHC complex is connected to the hinge region of the Fc domain via a long linker.

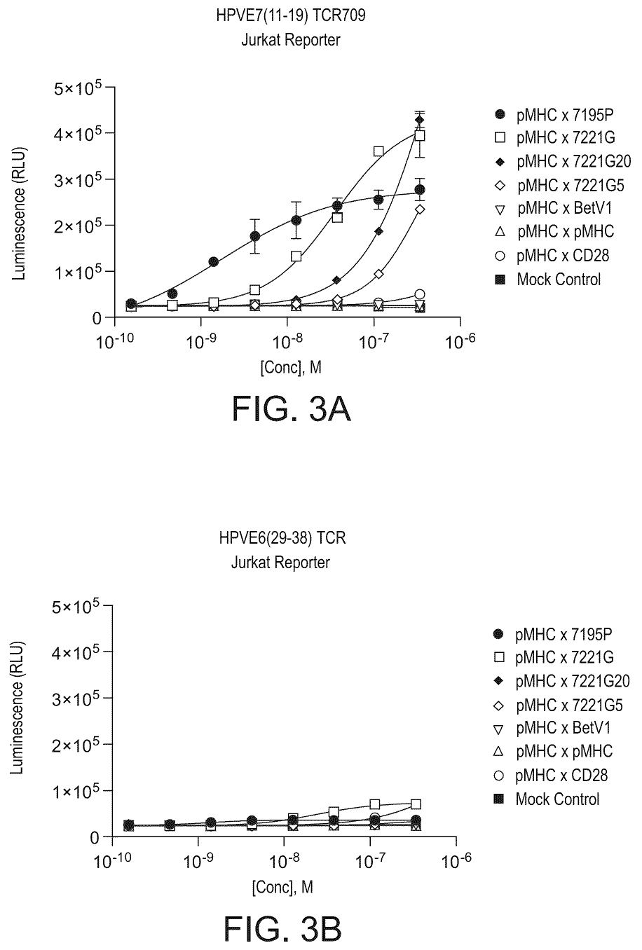

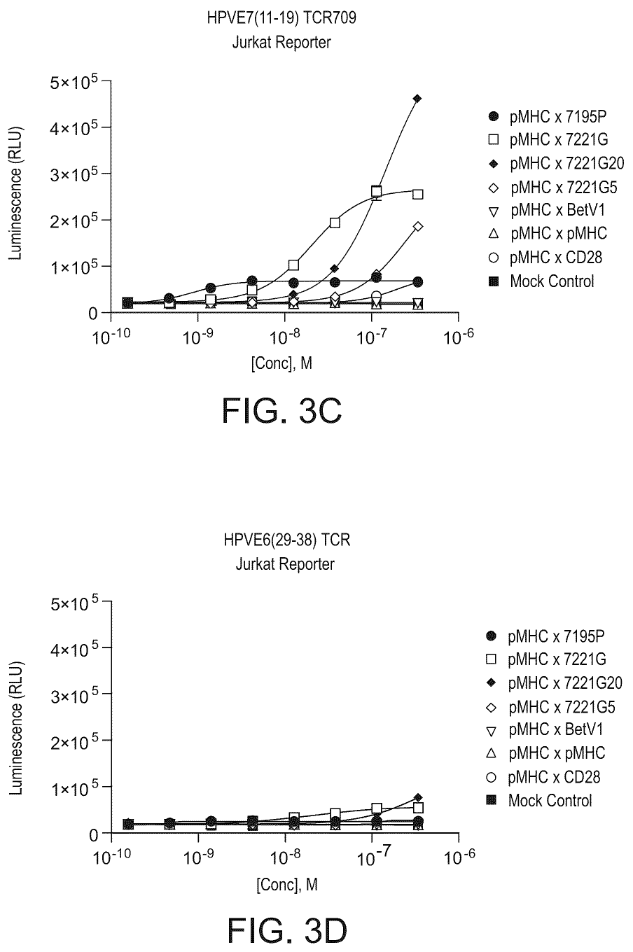

FIGS. 3A-3D show Jurkat reporter cell activation by soluble monovalent T cell activator constructs, each comprising a single pMHC complex. FIG. 3A shows activation of Jurkat/NFAT-Luc/Cl 3C7/HPV16E7(11-19) TCR709 cells by soluble T cell activator constructs, wherein the pMHC complex is connected to the hinge region of the Fc domain via a short linker. FIG. 3B shows activation of Jurkat/NFAT-Luc/Cl 3C7/HPV16E6(29-38) TCR cells by soluble T cell activator constructs, wherein the pMHC complex is connected to the hinge region of the Fc domain via a short linker. FIG. 3C shows activation of Jurkat/NFAT-Luc/Cl 3C7/HPV16E7(11-19) TCR709 cells by soluble T cell activator constructs, wherein the pMHC complex is connected to the hinge region of the Fc domain via a long linker. FIG. 3D shows activation of Jurkat/NFAT-Luc/Cl 3C7/HPV16E6(29-38) TCR cells by soluble T cell activator constructs, wherein the pMHC complex is connected to the hinge region of the Fc domain via a long linker.

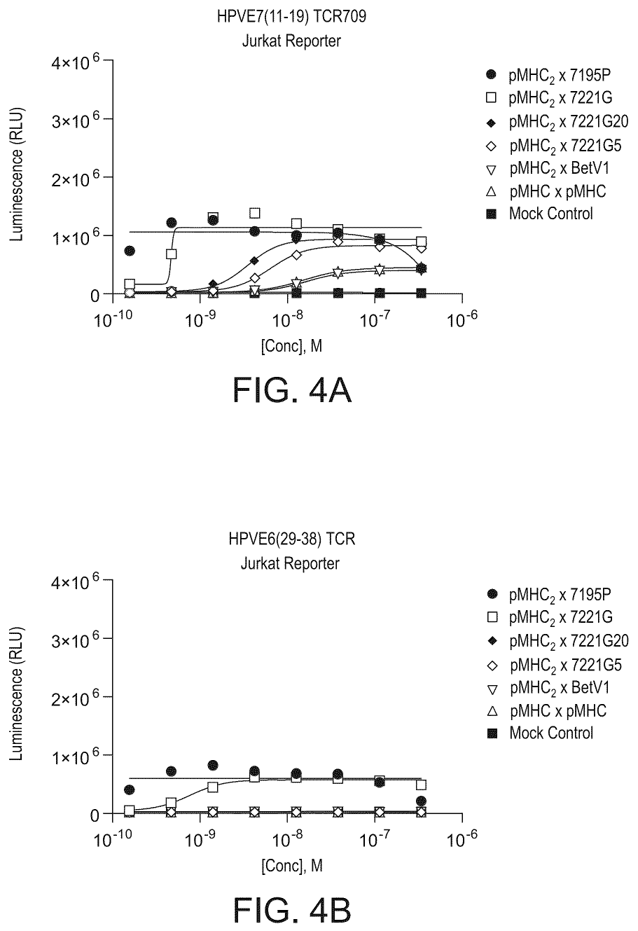

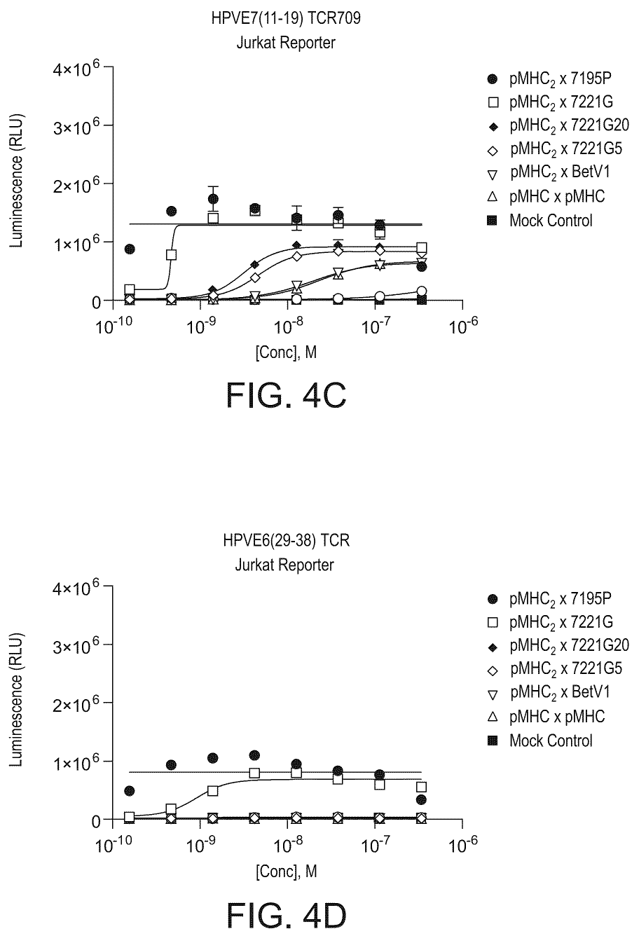

FIGS. 4A-4D show Jurkat reporter cell activation by plate-bound bivalent T cell activator constructs, each comprising two pMHC complexes at the N-terminus of an Fc domain. FIG. 4A shows activation of Jurkat/NFAT-Luc/Cl 3C7/HPV16E7(11-19) TCR709 cells by plate-bound T cell activator constructs, wherein the pMHC complexes are connected to the hinge region of the Fc domain via short linkers. FIG. 4B shows activation of Jurkat/NFAT-Luc/Cl 3C7/HPV16E6(29-38) TCR cells by plate-bound T cell activator constructs, wherein the pMHC complexes are connected to the hinge region of the Fc domain via short linkers. FIG. 4C shows activation of Jurkat/NFAT-Luc/Cl 3C7/HPV16E7(11-19) TCR709 cells by plate-bound T cell activator constructs, wherein the pMHC complexes are connected to the hinge region of the Fc domain via long linkers. FIG. 4D shows activation of Jurkat/NFAT-Luc/Cl 3C7/HPV16E6(29-38) TCR cells by plate-bound T cell activator constructs, wherein the pMHC complexes are connected to the hinge region of the Fc domain via long linkers.

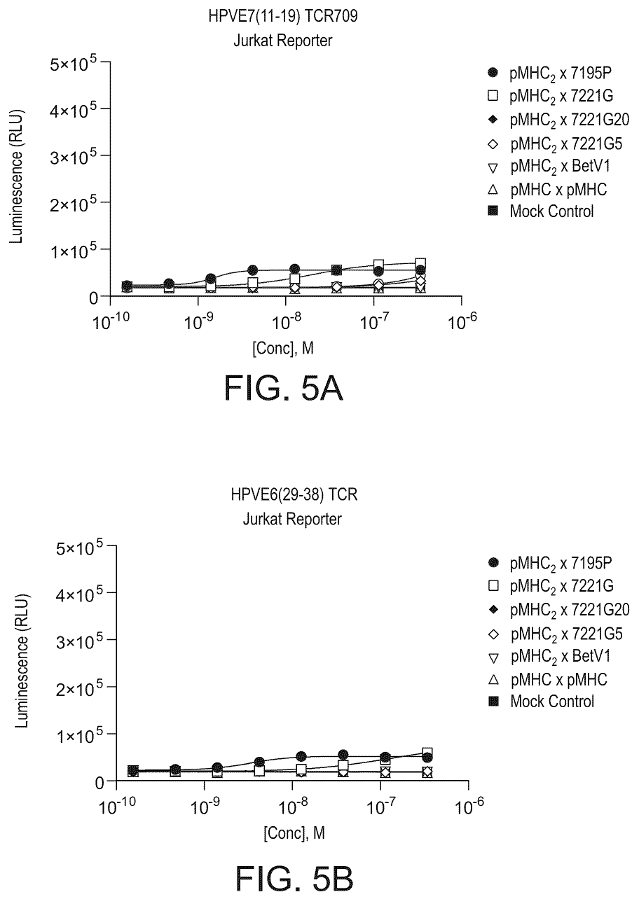

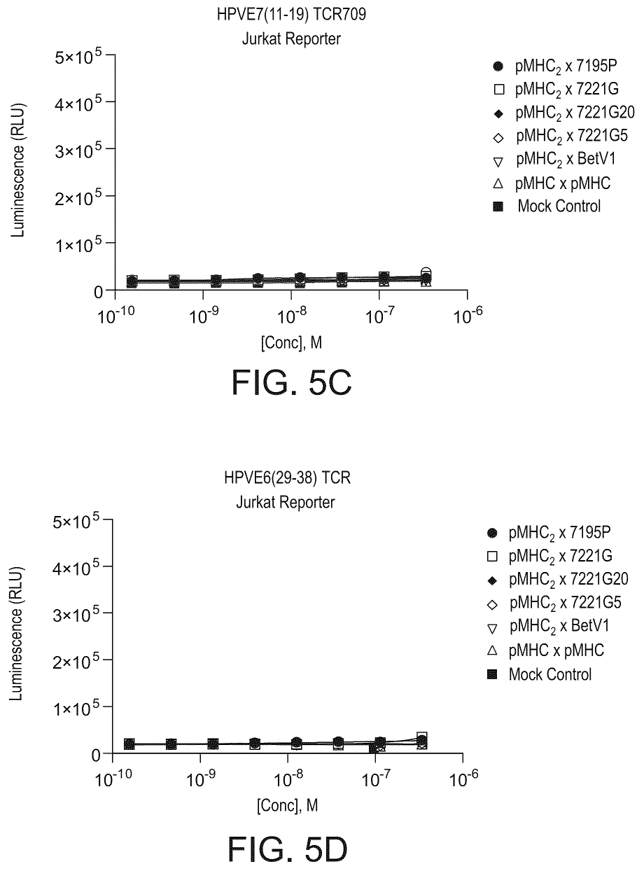

FIGS. 5A-5D show Jurkat reporter cell activation by soluble bivalent T cell activator constructs, each comprising two pMHC complexes at the N-terminus of an Fc domain. FIG. 5A shows activation of Jurkat/NFAT-Luc/Cl 3C7/HPV16E7(11-19) TCR709 cells by soluble T cell activator constructs, wherein the pMHC complexes are connected to the hinge region of the Fc domain via short linkers. FIG. 5B shows activation of Jurkat/NFAT-Luc/Cl 3C7/HPV16E6(29-38) TCR cells by soluble T cell activator constructs, wherein the pMHC complexes are connected to the hinge region of the Fc domain via short linkers. FIG. 5C shows activation of Jurkat/NFAT-Luc/Cl 3C7/HPV16E7(11-19) TCR709 cells by a plate-bound T cell activator construct, wherein the pMHC complexes are connected to the hinge region of the Fc domain via long linkers. FIG. 5D shows activation of Jurkat/NFAT-Luc/Cl 3C7/HPV16E6(29-38) TCR cells by a plate-bound T cell activator construct, wherein the pMHC complexes are connected to the hinge region of the Fc domain via long linkers.

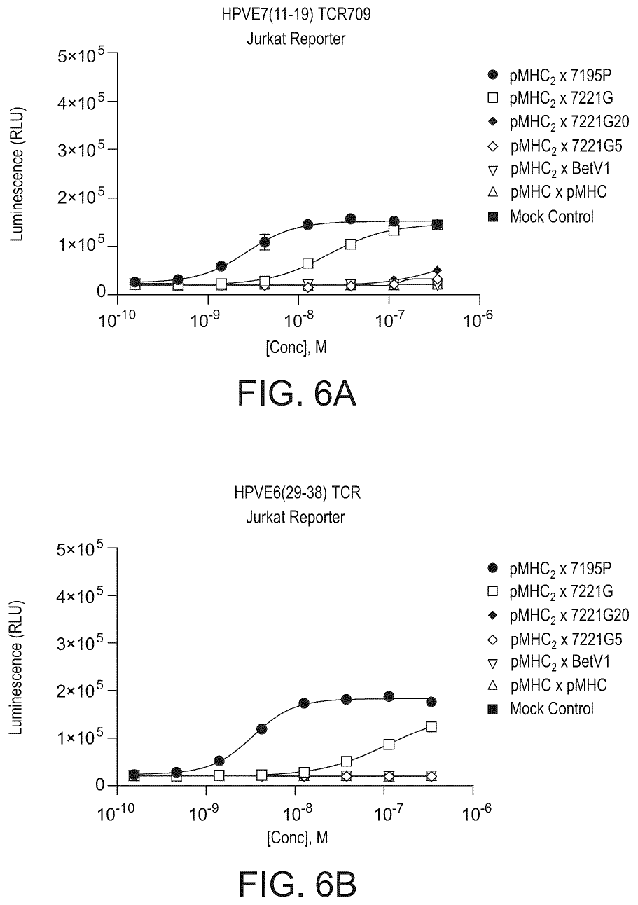

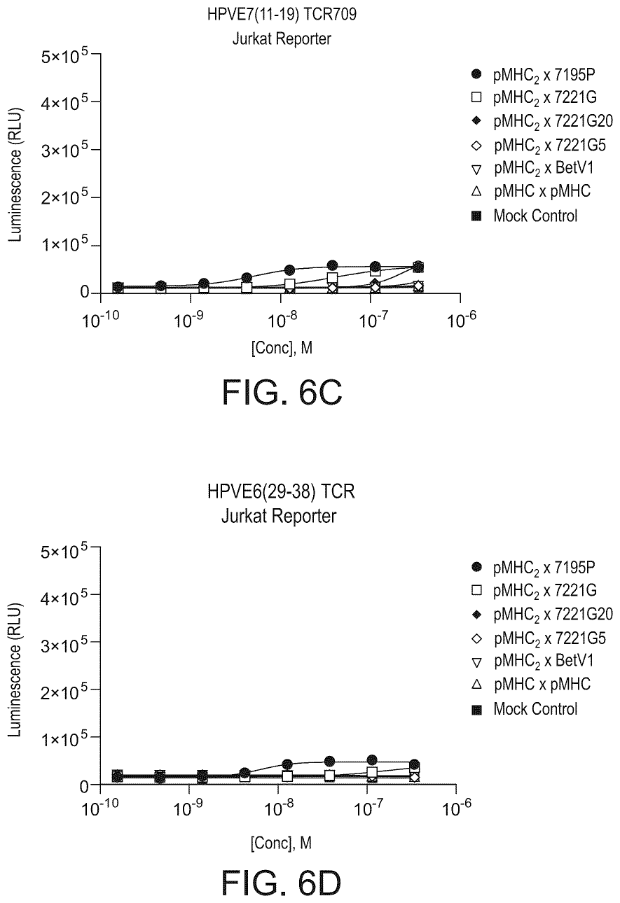

FIGS. 6A-6D show the effect of pre-clustering bivalent T cell activators with anti-Fc on Jurkat reporter cell activation. FIG. 6A shows activation of Jurkat/NFAT-Luc/Cl 3C7/HPV16E7(11-19) TCR709 cells by pre-clustered T cell activator constructs, wherein the pMHC complexes are connected to the hinge region of the Fc domain via short linkers. FIG. 6B shows activation of Jurkat/NFAT-Luc/Cl 3C7/HPV16E6(29-38) TCR cells by pre-clustered T cell activator constructs, wherein the pMHC complexes are connected to the hinge region of the Fc domain via long linkers. FIG. 6C shows activation of Jurkat/NFAT-Luc/Cl 3C7/HPV16E7(11-19) TCR709 cells by pre-clustered T cell activator constructs, wherein the pMHC complexes are connected to the hinge region of the Fc domain via long linkers. FIG. 6D shows activation of Jurkat/NFAT-Luc/Cl 3C7/HPV16E6(29-38) TCR cells by pre-clustered T cell activator constructs, wherein the pMHC complexes are connected to the hinge region of the Fc domain via long linkers.

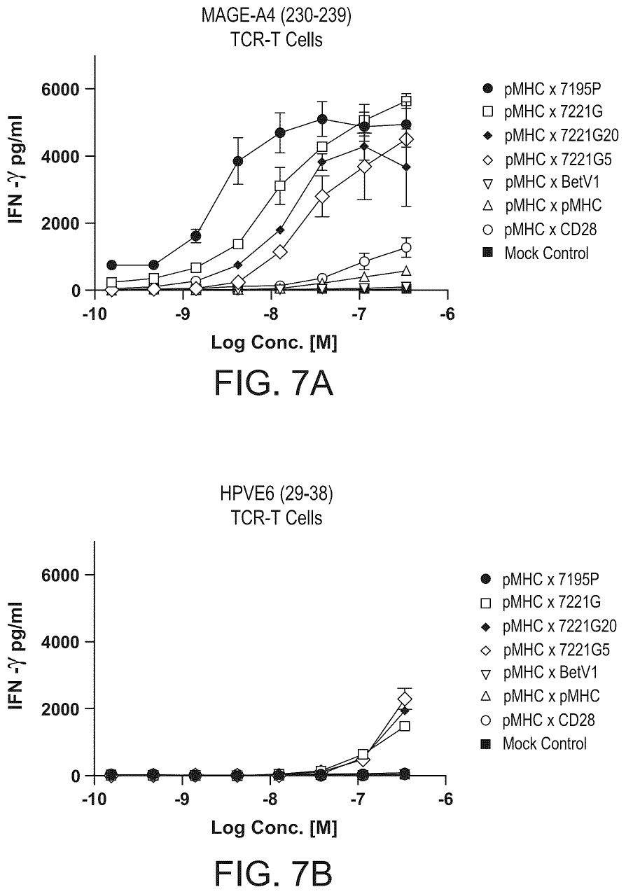

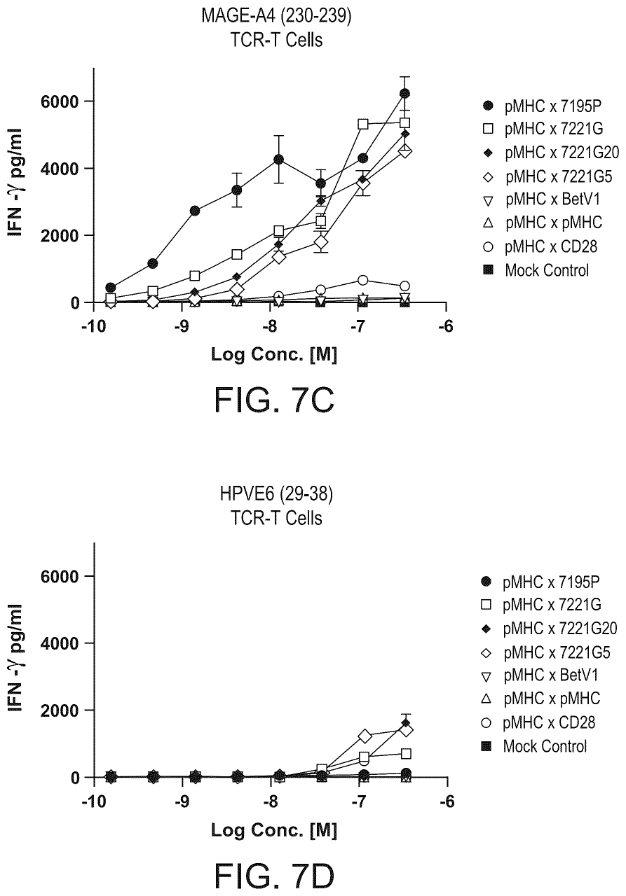

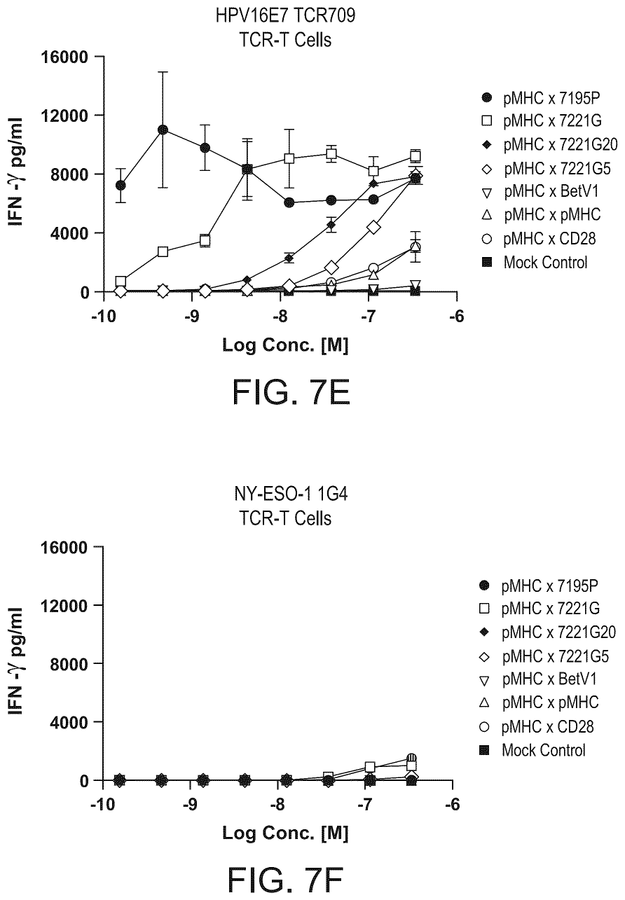

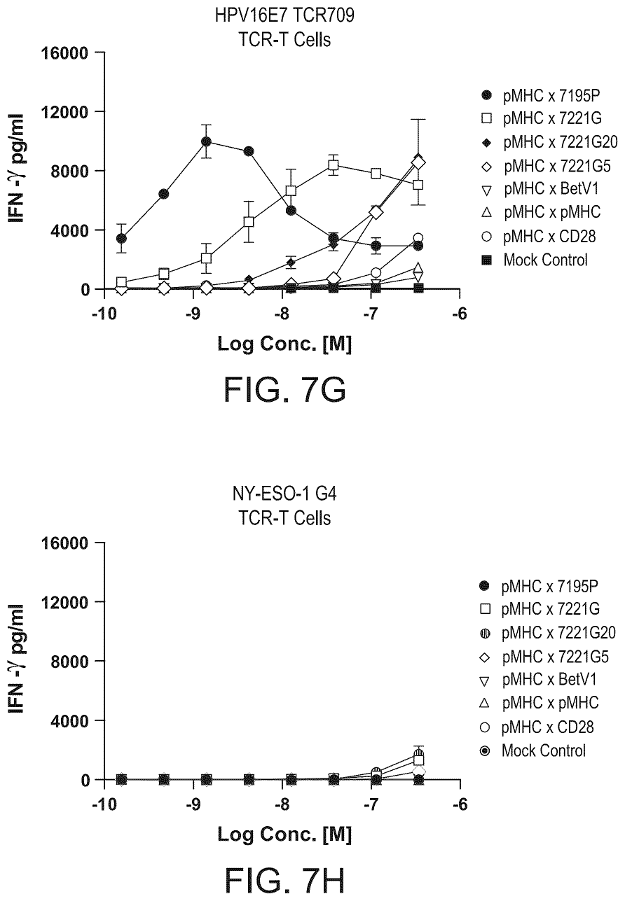

FIGS. 7A-7H show IFN-γ release by primary TCR-T cells upon treatment with monovalent T cell activators of the disclosure. FIG. 7A shows IFN-γ release in MAGE-A4 PN45545 (230-239) TCR-T cells upon treatment with monovalent T cell activators, wherein the pMHC complex of each is connected to the hinge region of the Fc domain via a short linker. FIG. 7B shows IFN-γ release in control HPVE6 TCR-T cells upon treatment with monovalent T cell activators, wherein the pMHC complex of each is connected to the hinge region of the Fc domain via a short linker. FIG. 7C shows IFN-γ release in MAGE-A4 PN45545 (230-239) TCR-T cells upon treatment with monovalent T cell activators, wherein the pMHC complex of each is connected to the hinge region of the Fc domain via a long linker. FIG. 7D shows IFN-γ release in HPVE6 TCR-T negative control cells upon treatment with monovalent T cell activators, wherein the pMHC complex of each is connected to the hinge region of the Fc domain via a long linker. FIG. 7E shows IFN-γ release in HPV16E7 TCR709 TCR-T cells upon treatment with monovalent T cell activators, wherein the pMHC complex of each is connected to the hinge region of the Fc domain via a short linker. FIG. 7F shows IFN-γ release in NY-ESO-1 1G4 TCR-T negative control cells upon treatment with monovalent T cell activators, wherein the pMHC complex of each is connected to the hinge region of the Fc domain via a short linker. FIG. 7G shows IFN-γ release in HPV16E7 TCR709 TCR-T cells upon treatment with monovalent T cell activators, wherein the pMHC complex of each is connected to the hinge region of the Fc domain via a long linker. FIG. 7H shows IFN-γ release in NY-ESO-1 1G4 TCR-T negative control cells upon treatment with monovalent T cell activators, wherein the pMHC complex of each is connected to the hinge region of the Fc domain via a long linker.

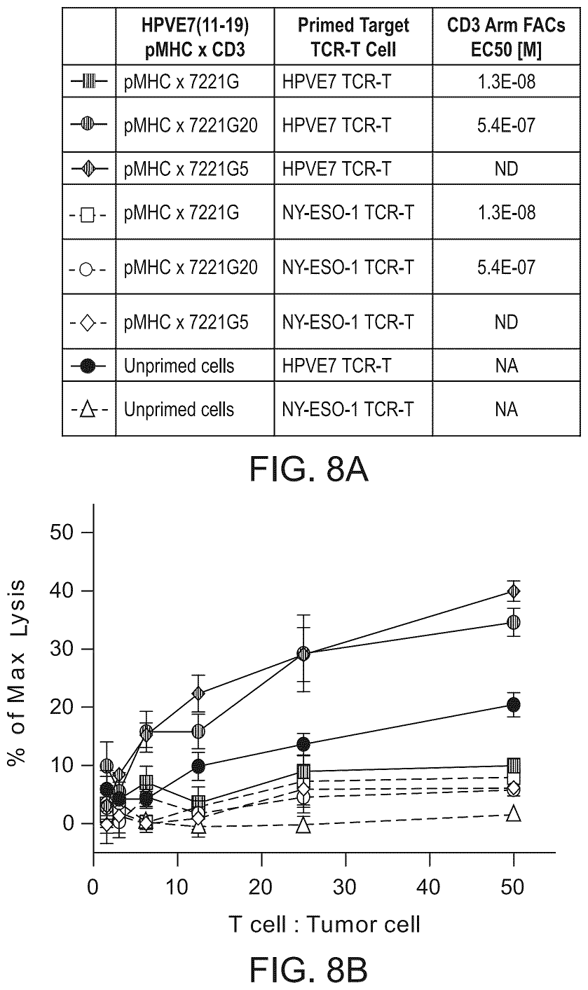

FIGS. 8A-8B show tumor cell killing activity of TCR-T cells primed with T cell activators comprising HPV16E7 pMHC complexes. FIG. 8A is a table showing the primed target TCR-T cell and anti-CD3 moiety FACS EC50 value for each T cell activator. FIG. 8B shows the percent maximum lysis of CaSki tumor cells as a function of T cell: Tumor cell.

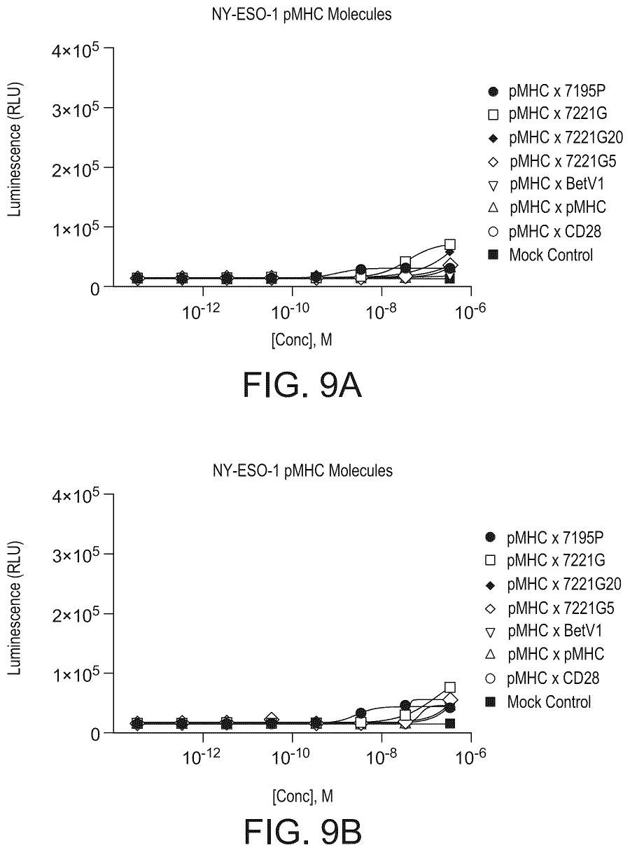

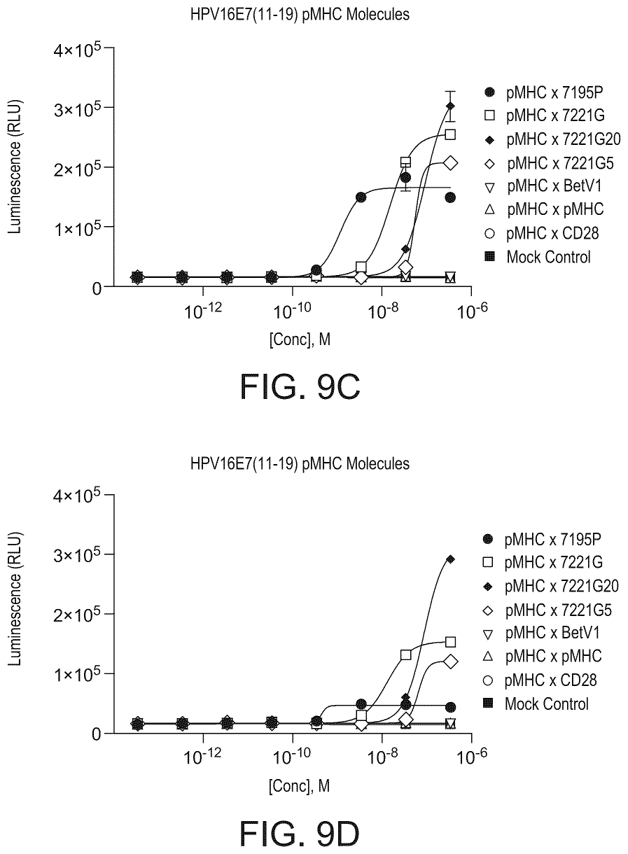

FIGS. 9A-9D demonstrate that reporter cell activation is associated with Cis-binding of T cell activators. FIG. 9A displays the HPV16E7 TCR709 NFAT luciferase reporter cell activity upon application of T cell activators comprising NY-ESO-1(157-165) pMHC and short linkers to mixed Jurkat cells. FIG. 9B displays the HPV16E7 TCR709 NFAT luciferase reporter cell activity upon application of T cell activators comprising NY-ESO-1(157-165) pMHC and long linkers to mixed Jurkat cells. FIGS. 9C and 9D are positive controls displaying the HPV16E7 TCR709 NFAT luciferase reporter cell activity upon application of T cell activators comprising HPVE7(11-19) pMHC and either short or long linkers, respectively.

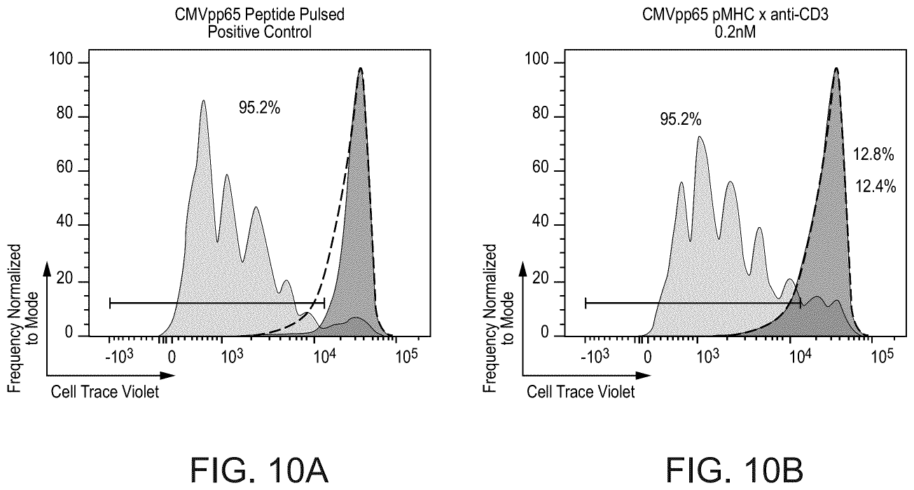

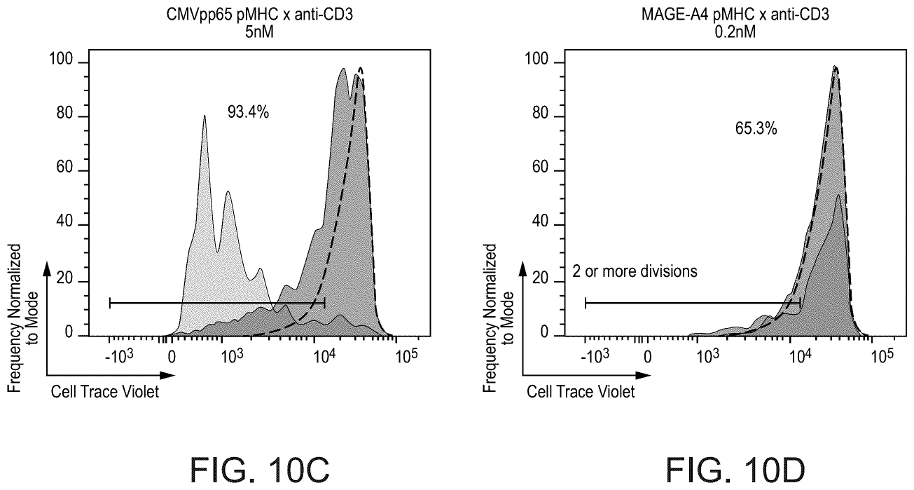

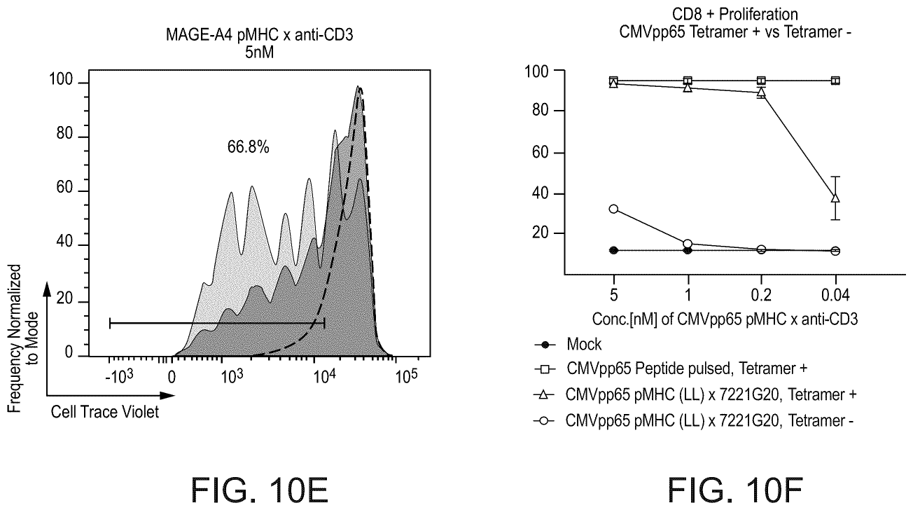

FIGS. 10A-10F are histograms showing antigen-specific expansion of CMVpp65 T cells upon treatment with low concentrations of T cell activators comprising long linkers. CMVpp65+ T cells are represented by light shaded curves, CMVpp65-CD8+ T cells are represented by dark shaded curves, and unexpanded CMV PBMCs (mock control) are represented by the dotted curves. FIG. 10A displays CMVpp65-peptide pulsed cell populations. FIG. 10B displays the populations of cells treated with 0.2 nM CMVpp65 pMHC×CD3 (7221G20) T cell activator comprising a long linker. FIG. 10C displays the populations of cells treated with 5 nM CMVpp65 pMHC×CD3 (7221G20) T cell activator comprising a long linker. FIG. 10D displays the populations of cells treated with 0.2 nM MAGE-A4 pMHC×CD3 (7221G20) comprising a long linker, whereas FIG. 10E displays the populations of cells treated with 5 nM MAGE-A4 pMHC×CD3 comprising a long linker. FIG. 10F shows the percentage of proliferation in tetramer+ and tetramer− cells upon treatment with CMVpp65 pMHC×CD3 (7221G20) T cell activator comprising a long linker.

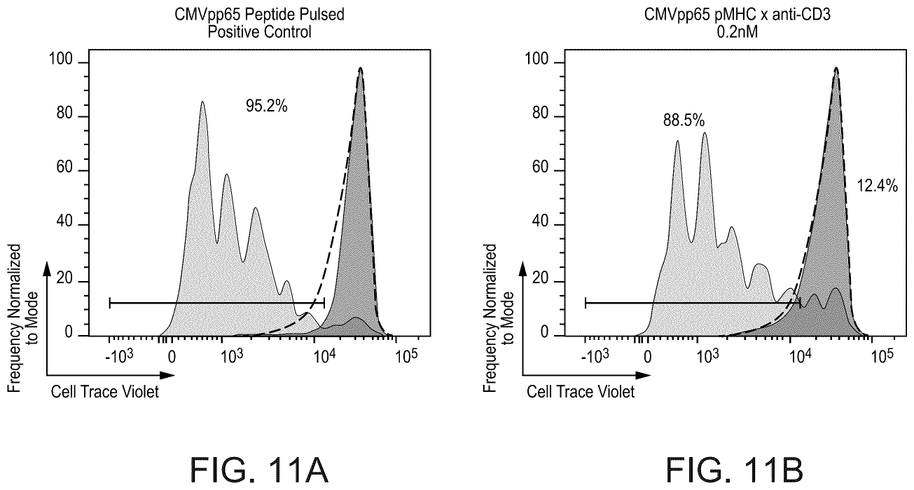

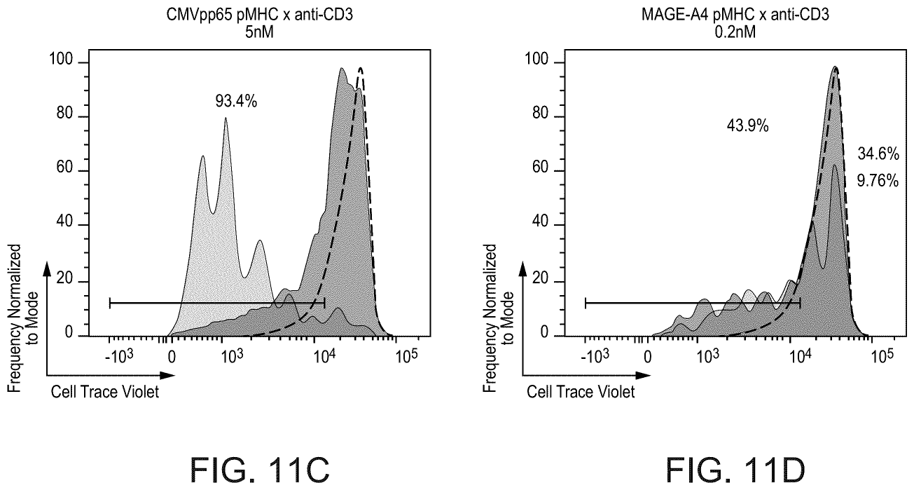

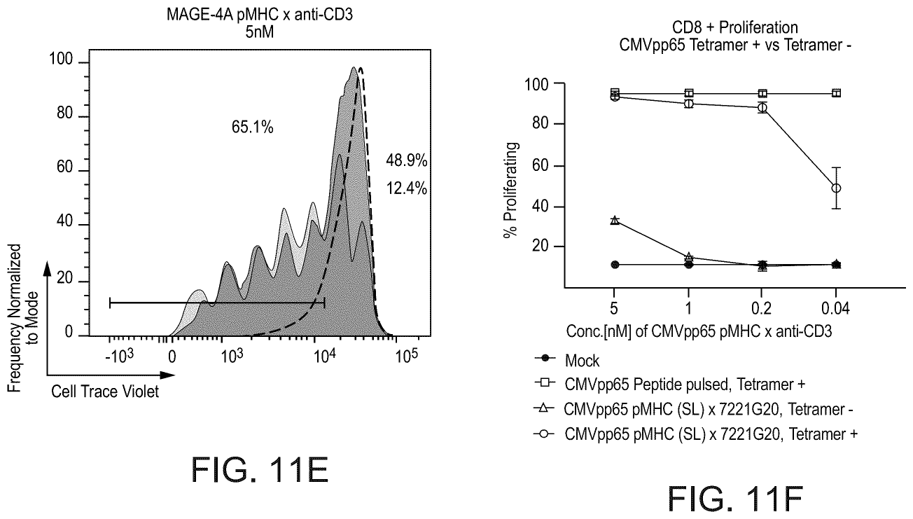

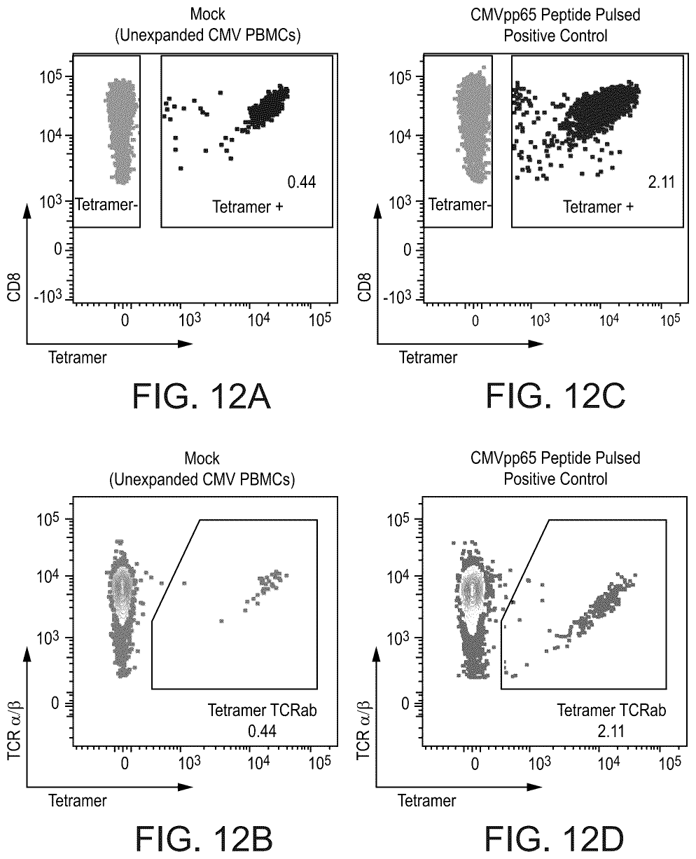

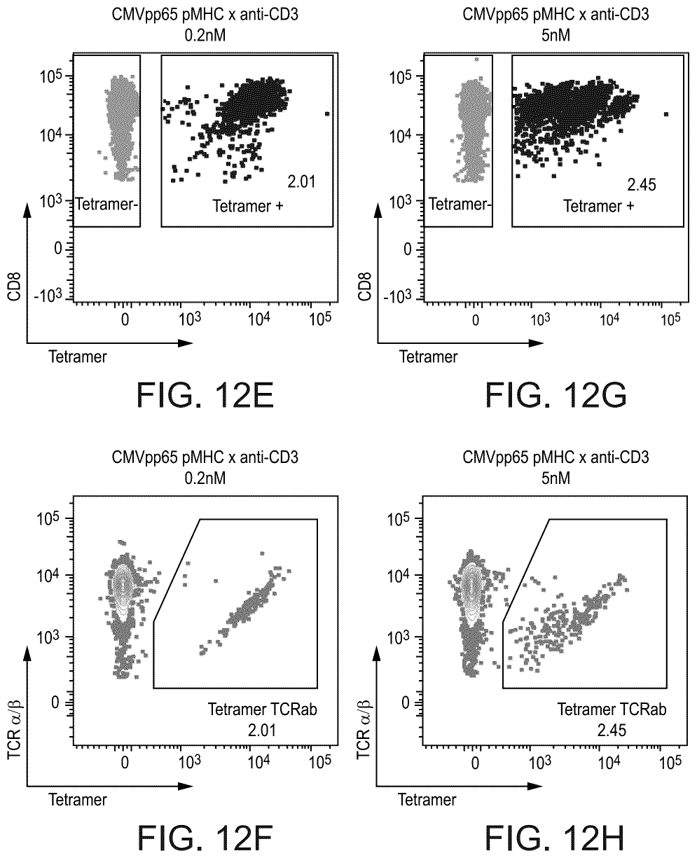

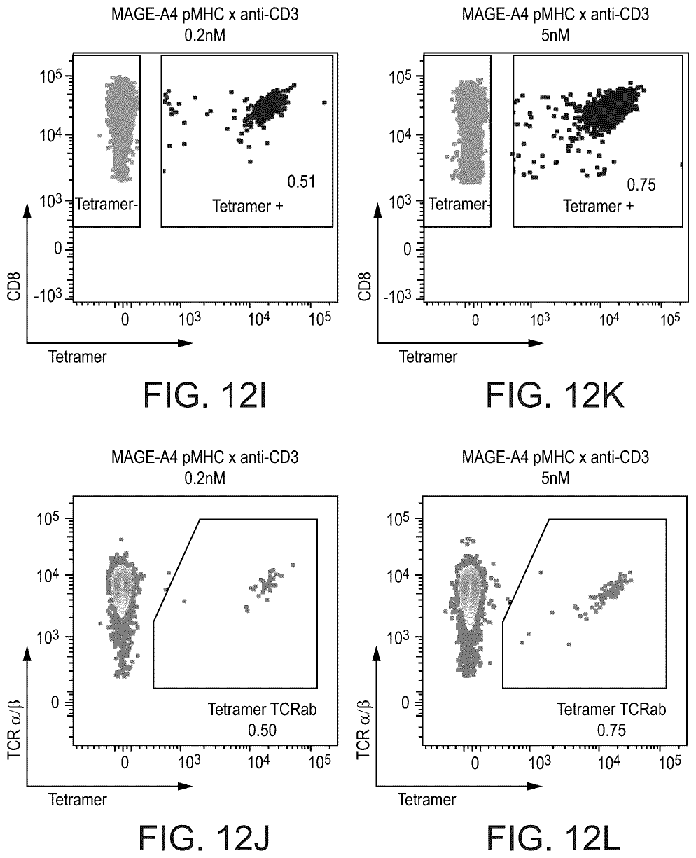

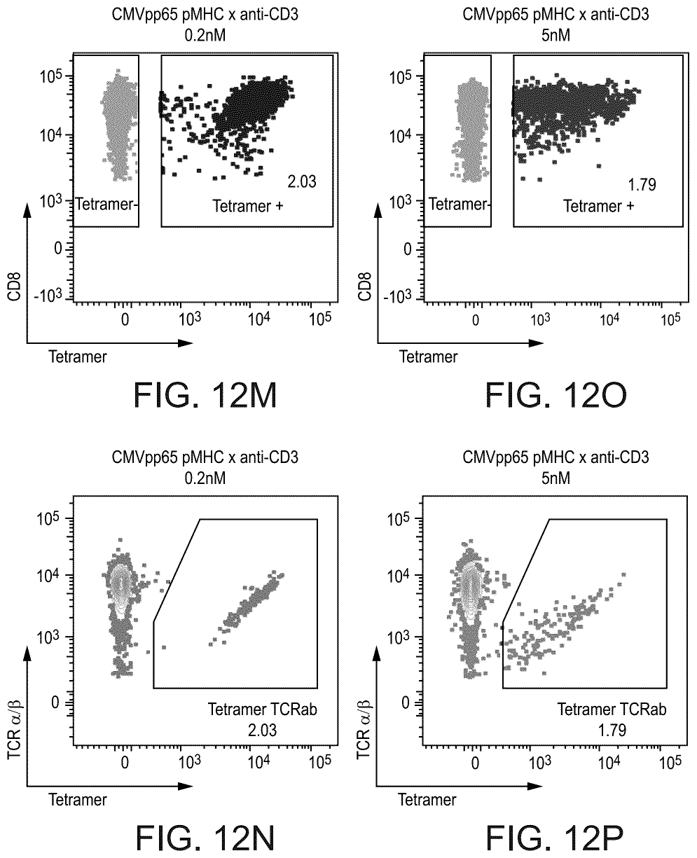

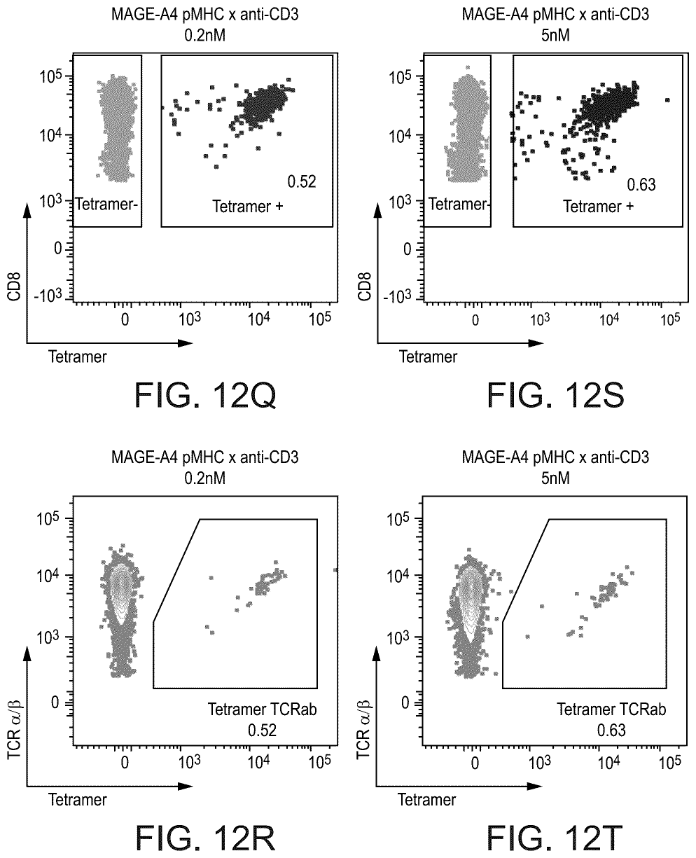

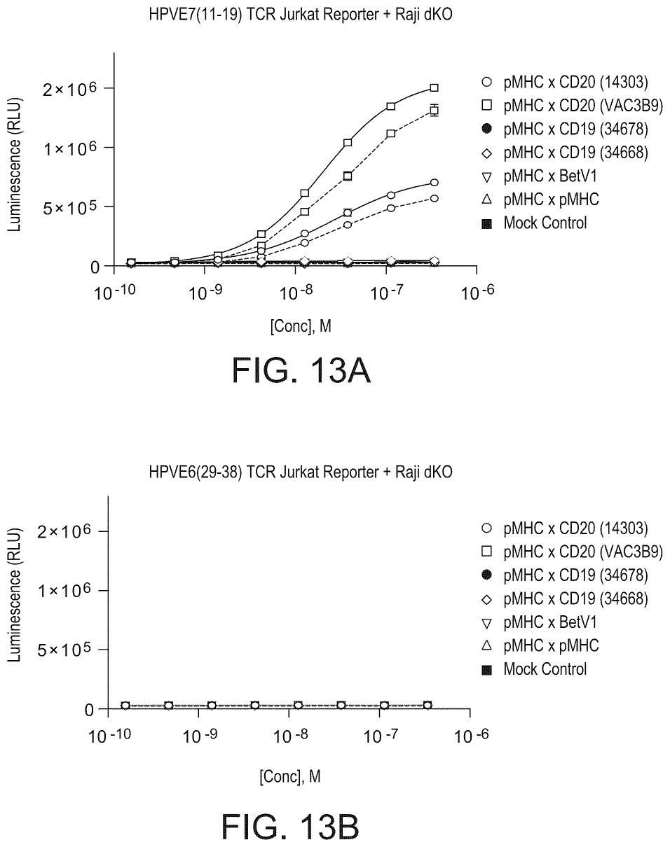

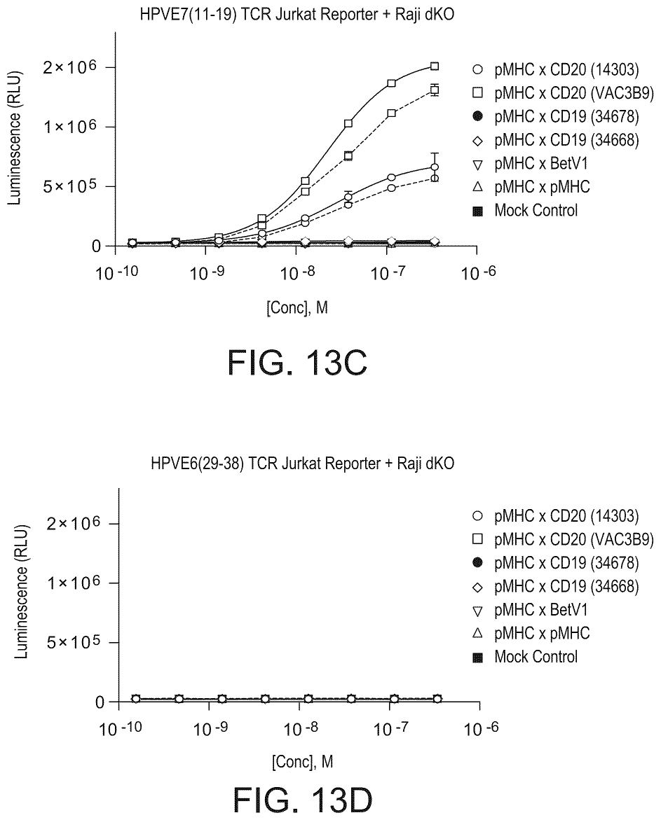

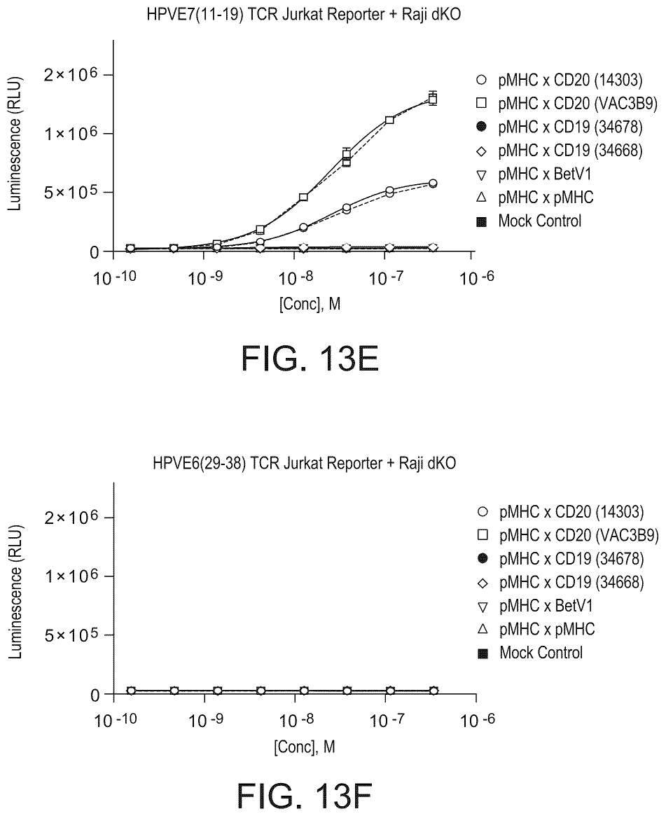

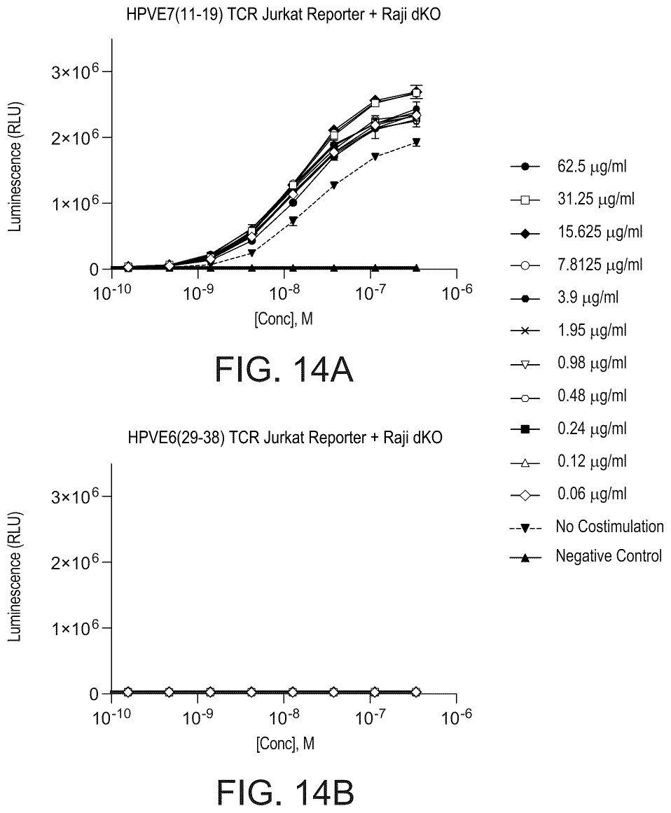

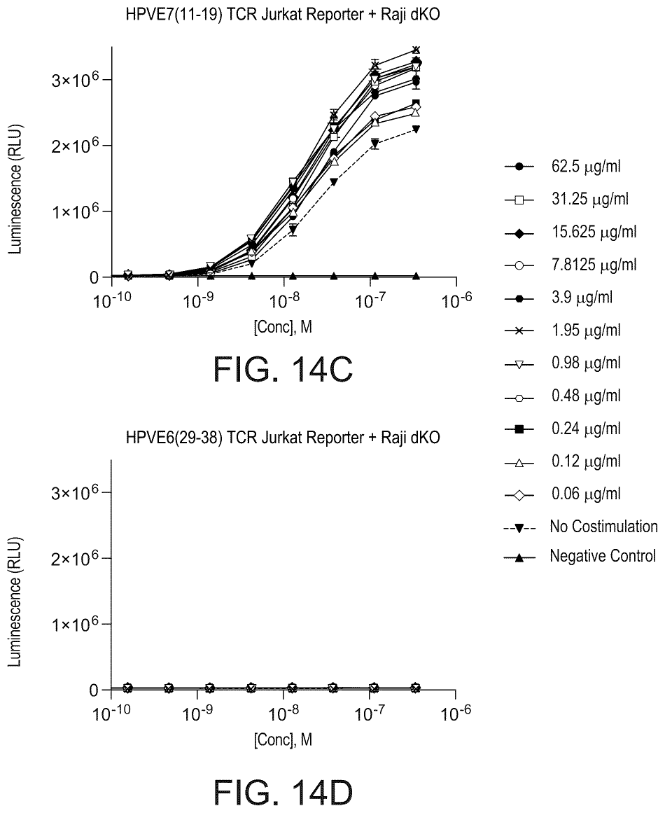

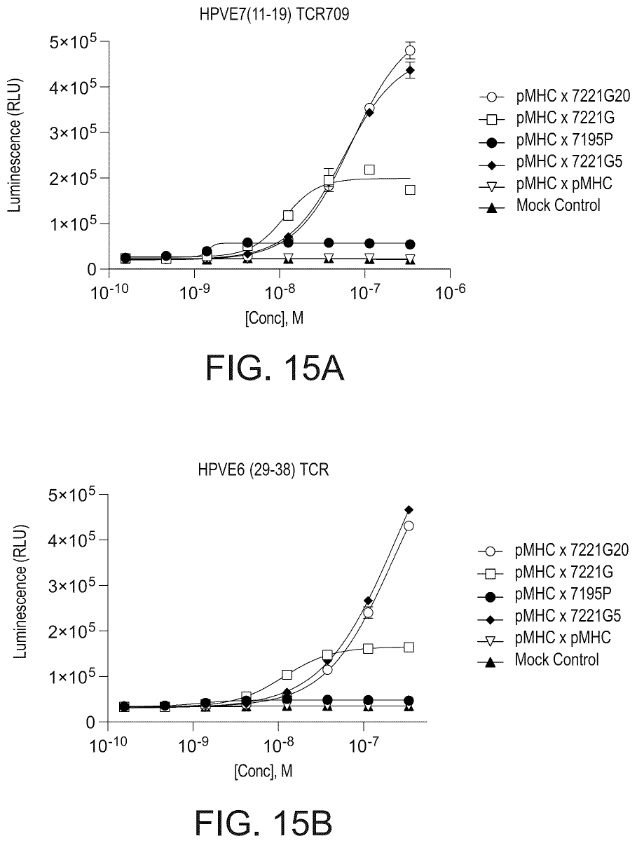

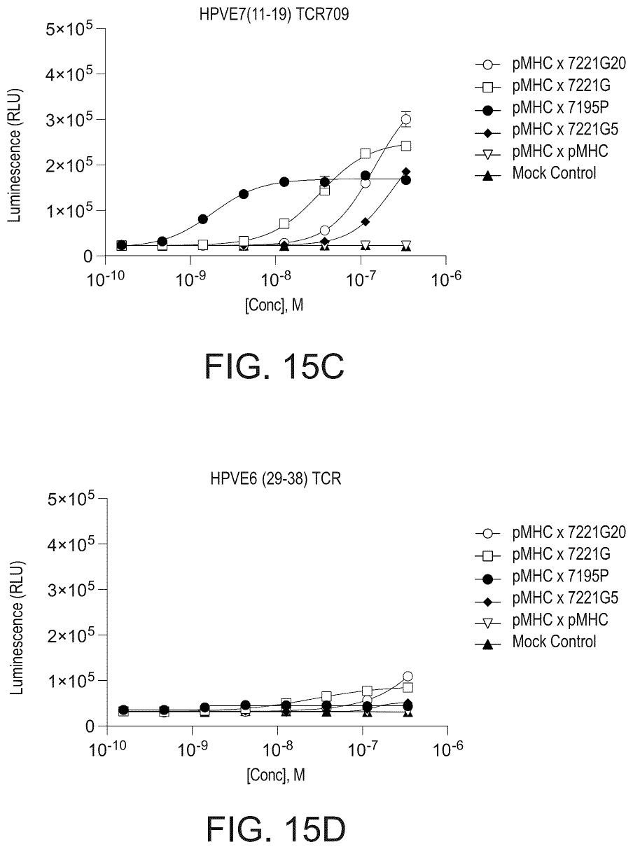

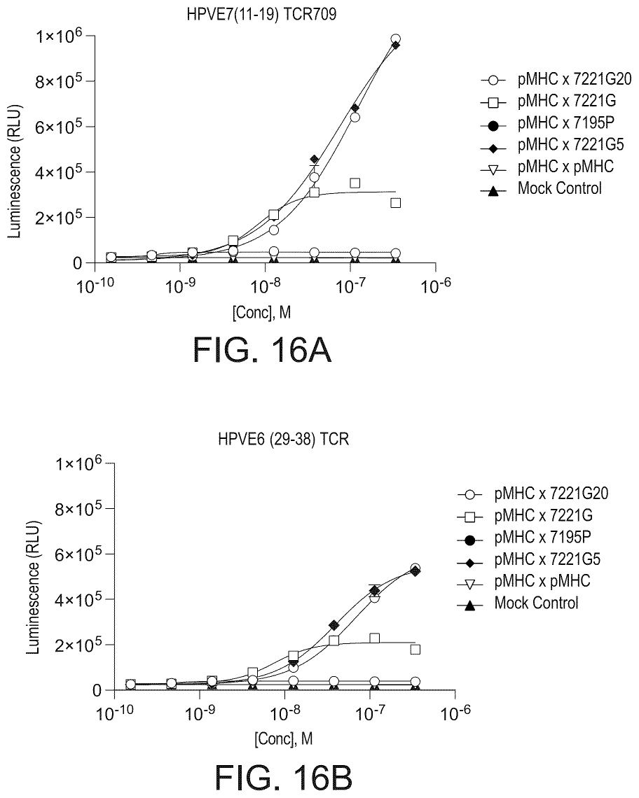

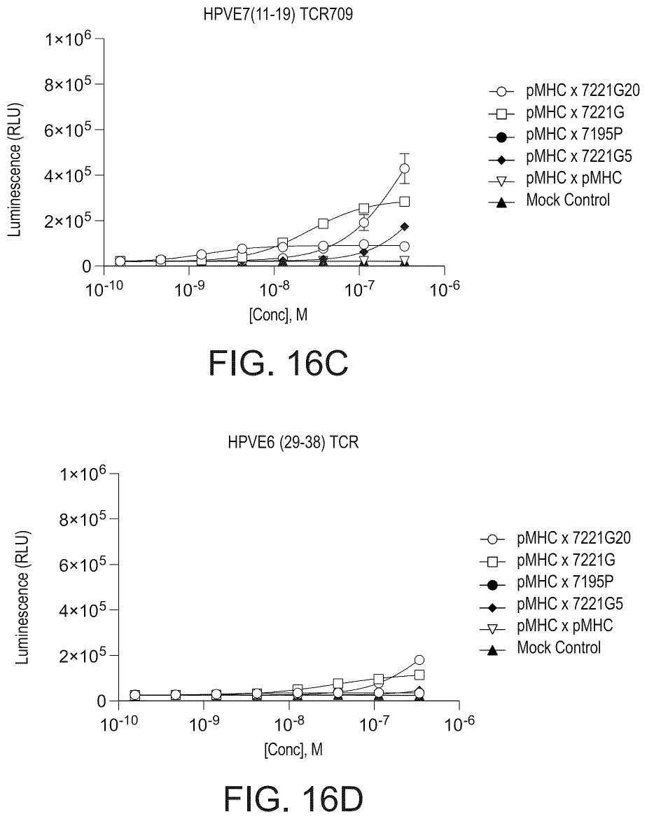

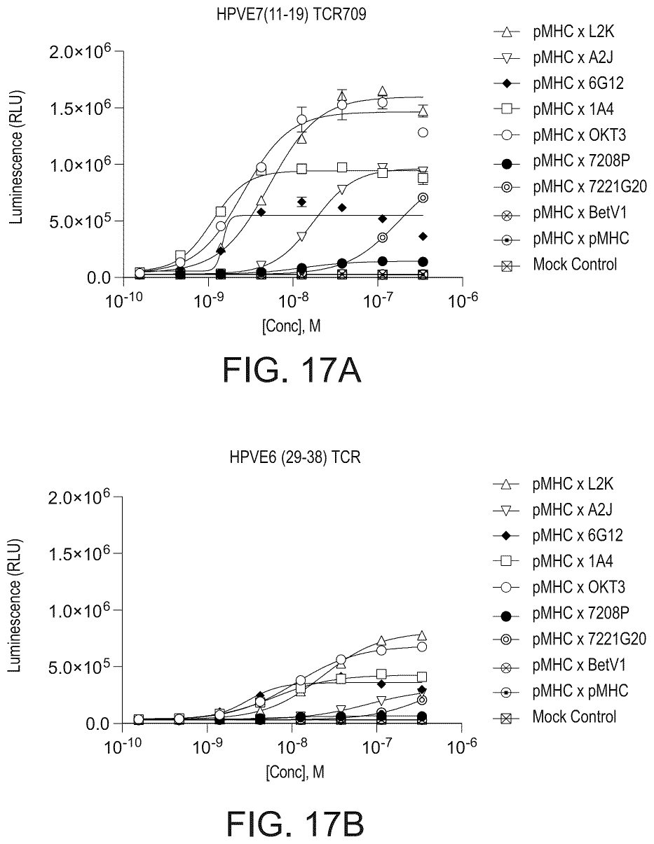

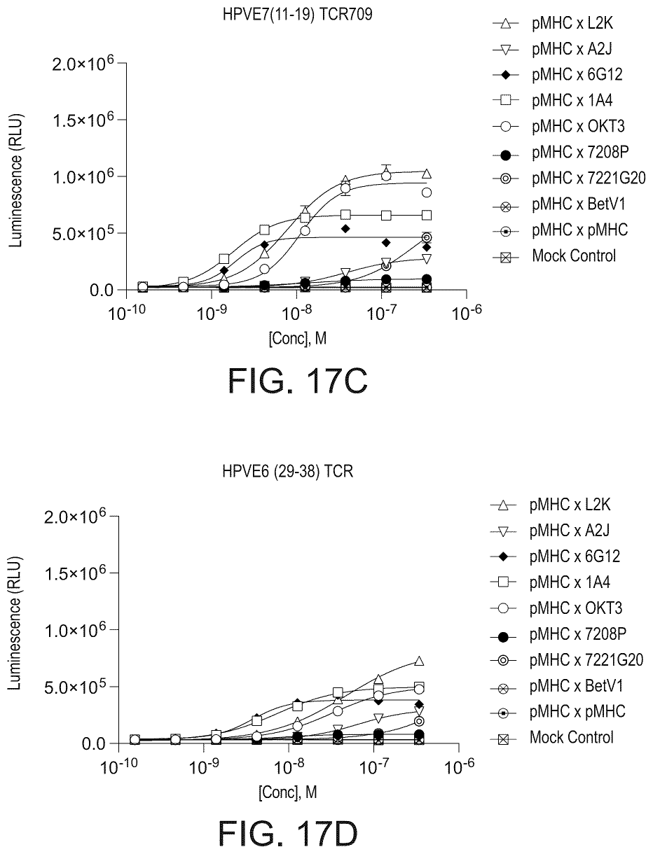

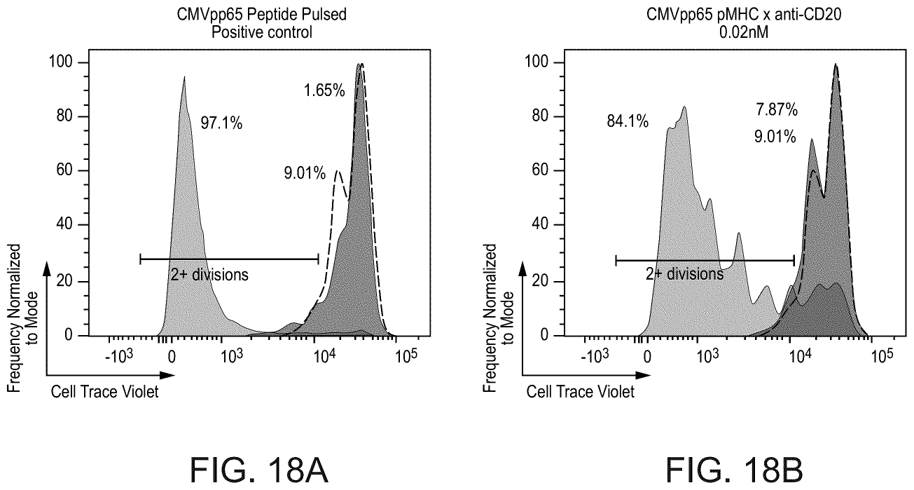

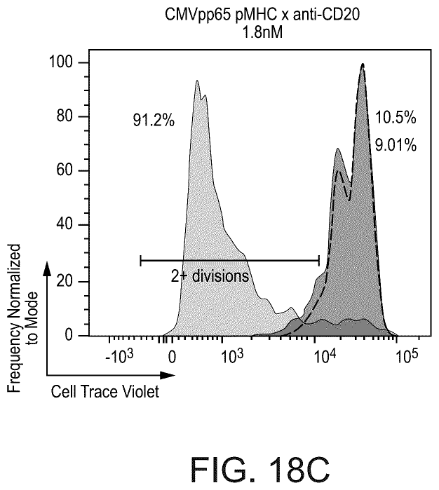

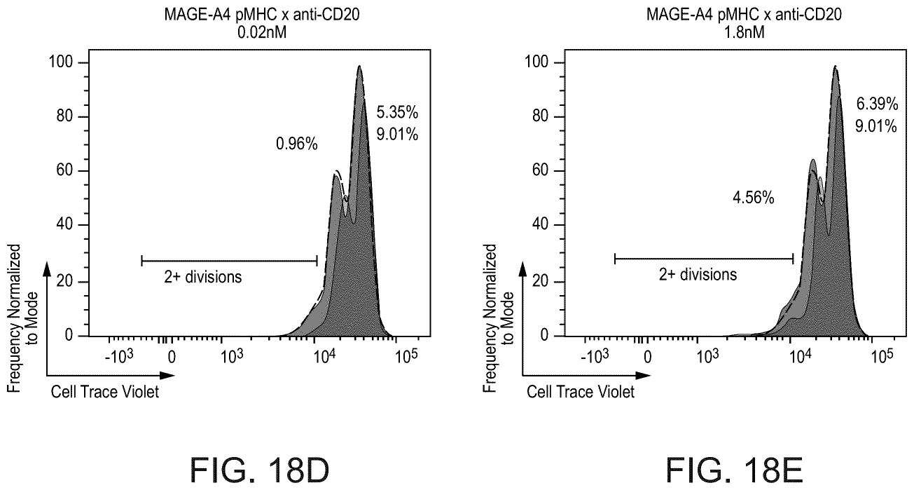

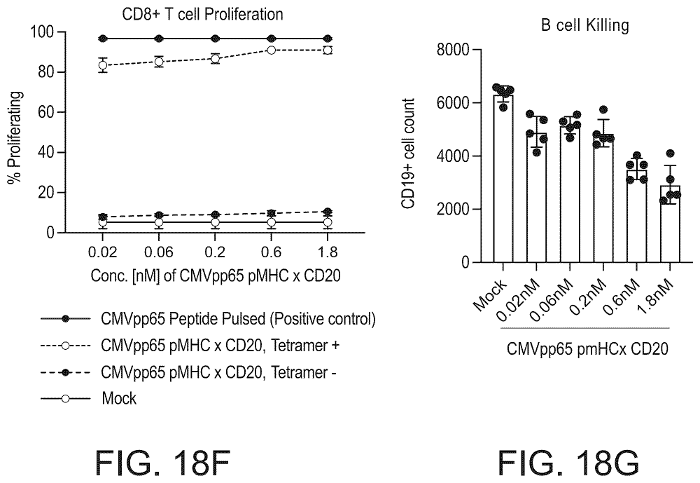

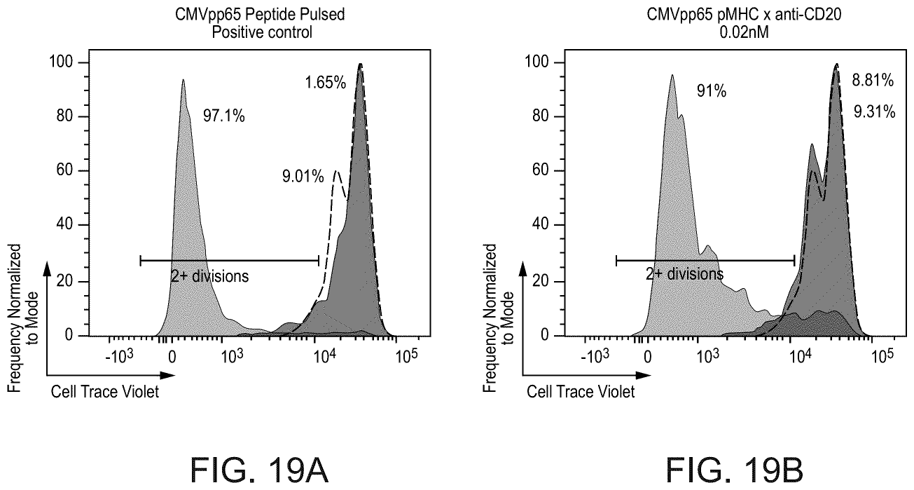

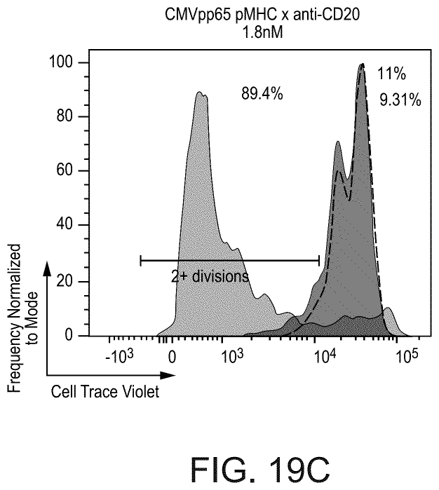

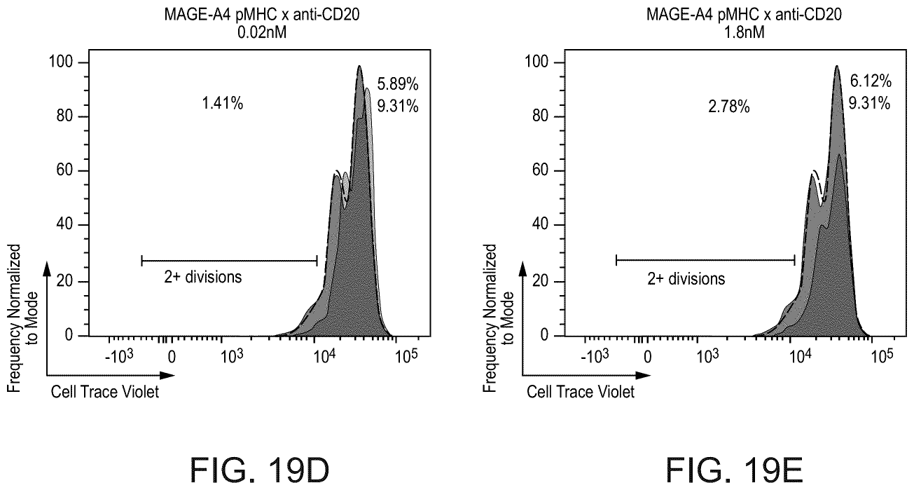

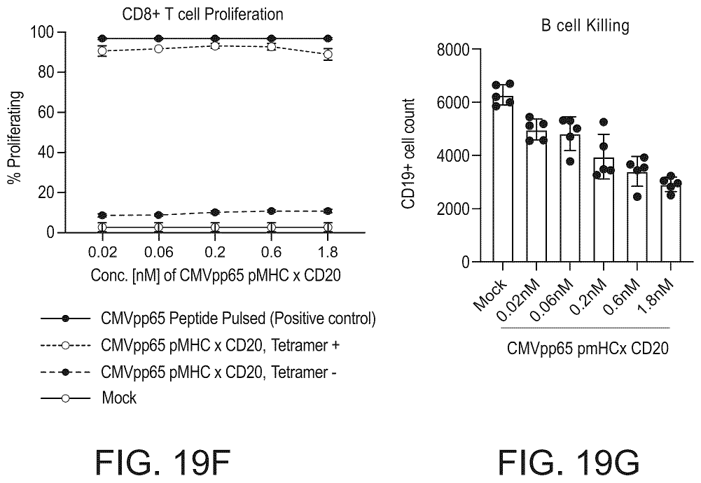

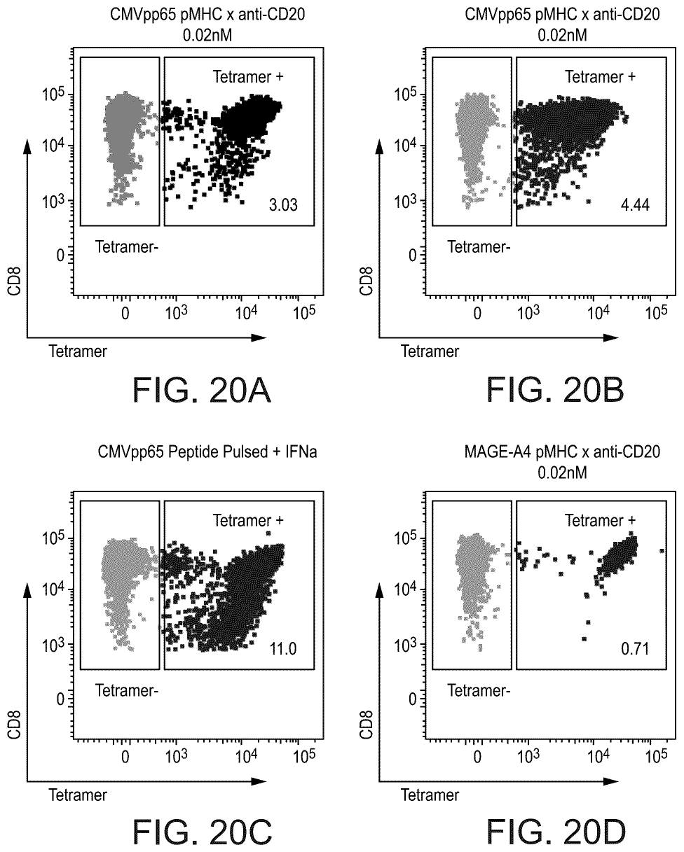

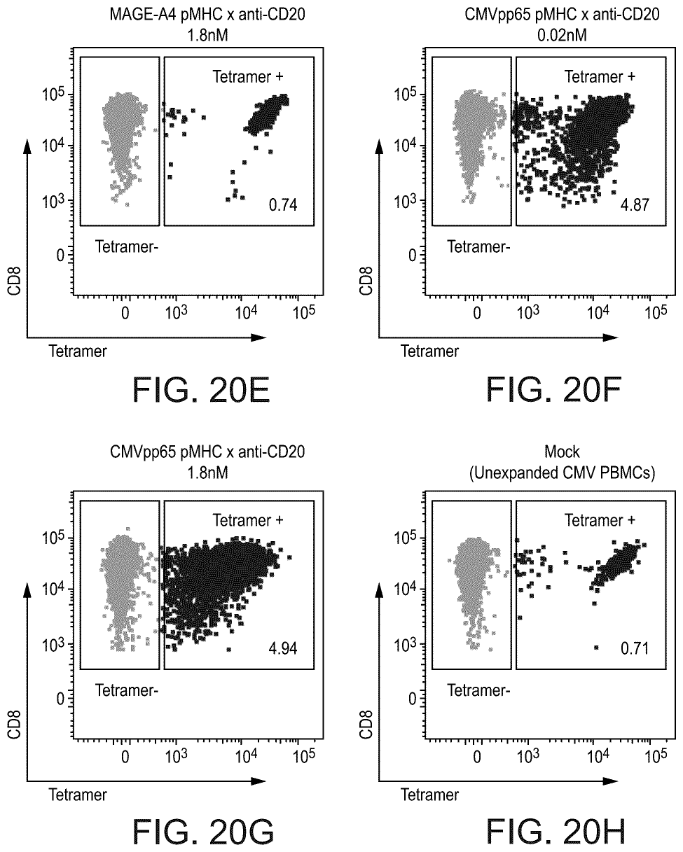

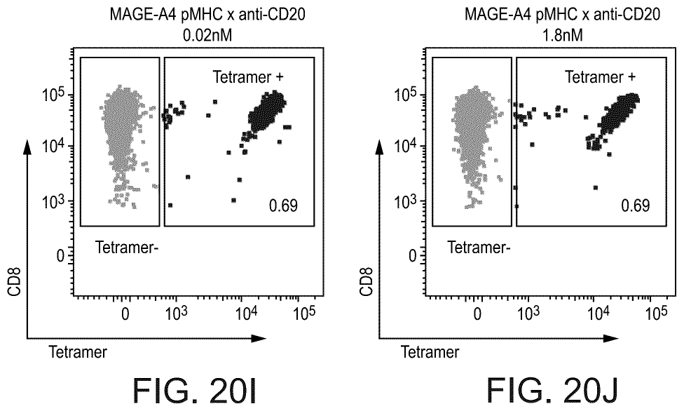

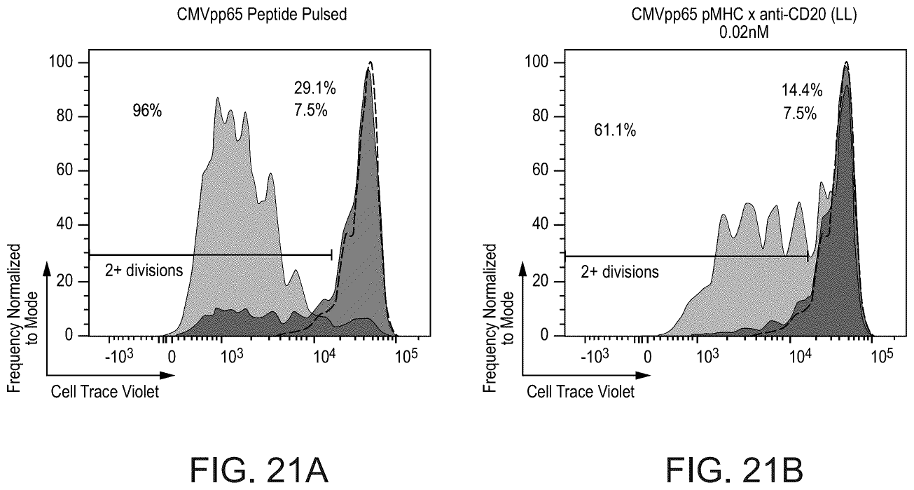

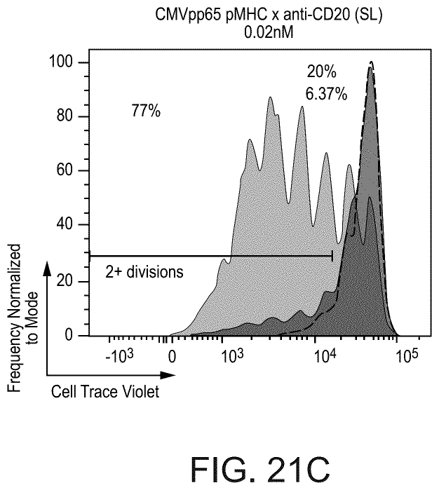

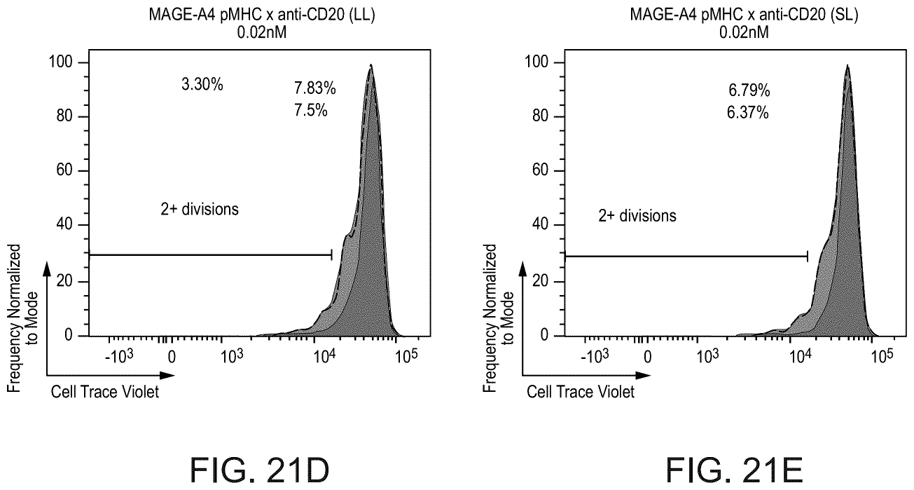

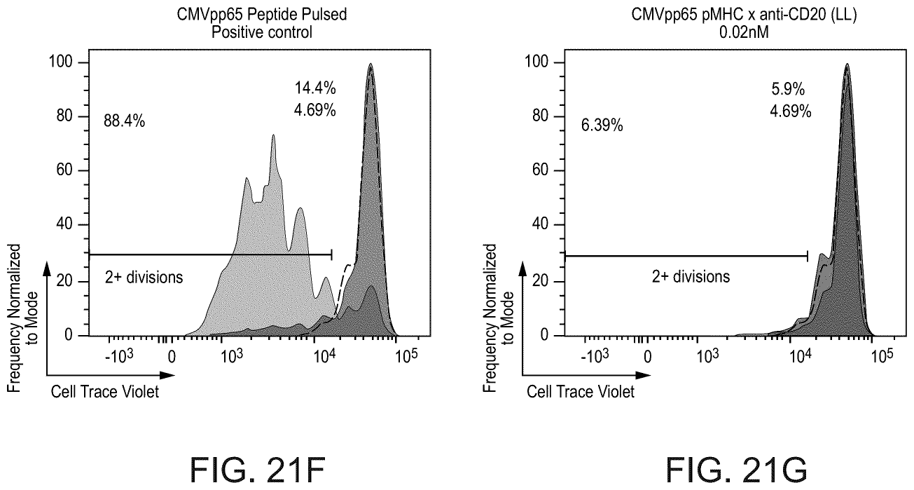

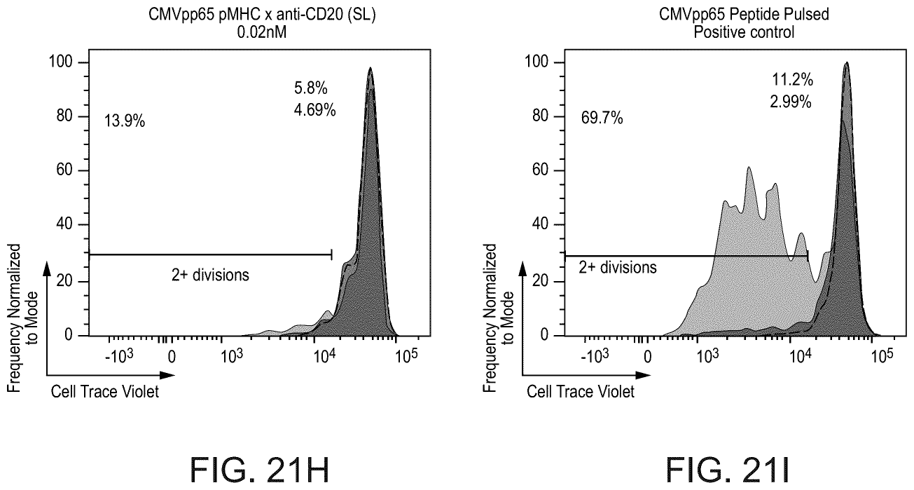

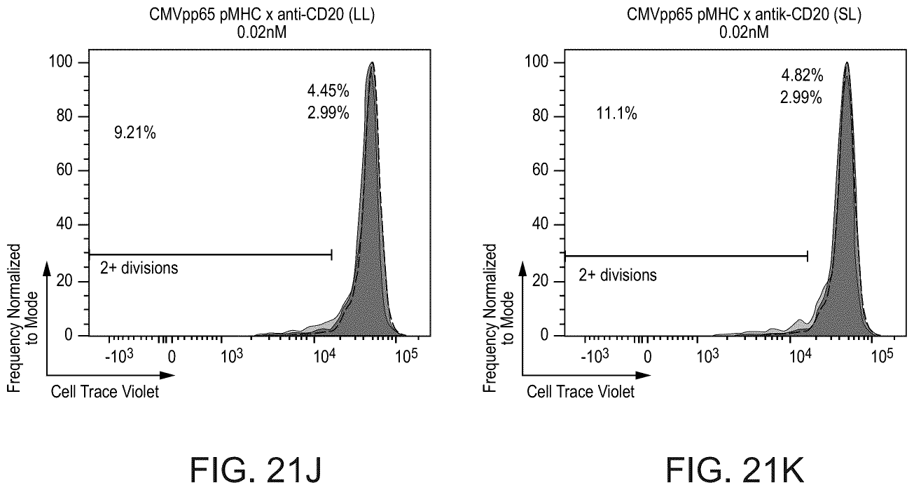

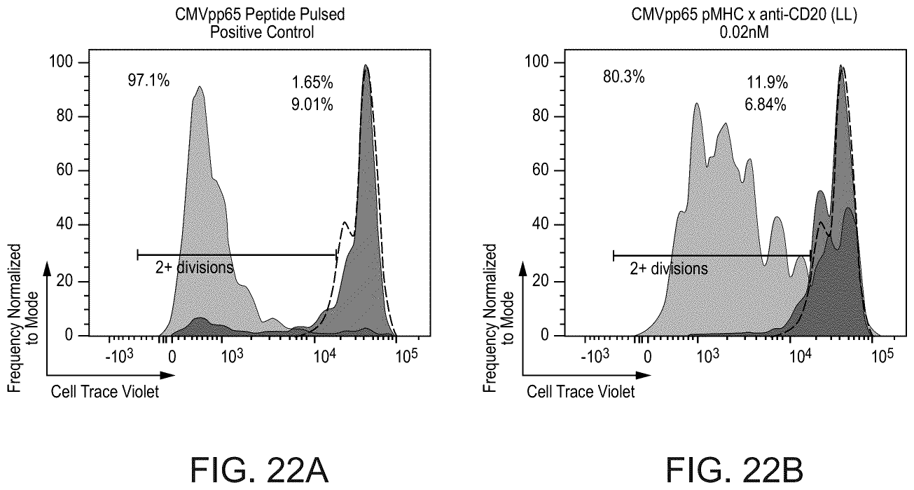

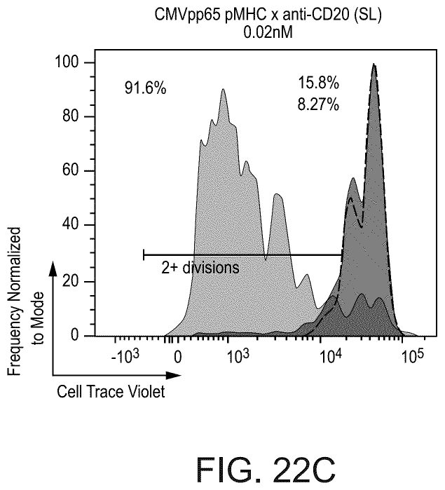

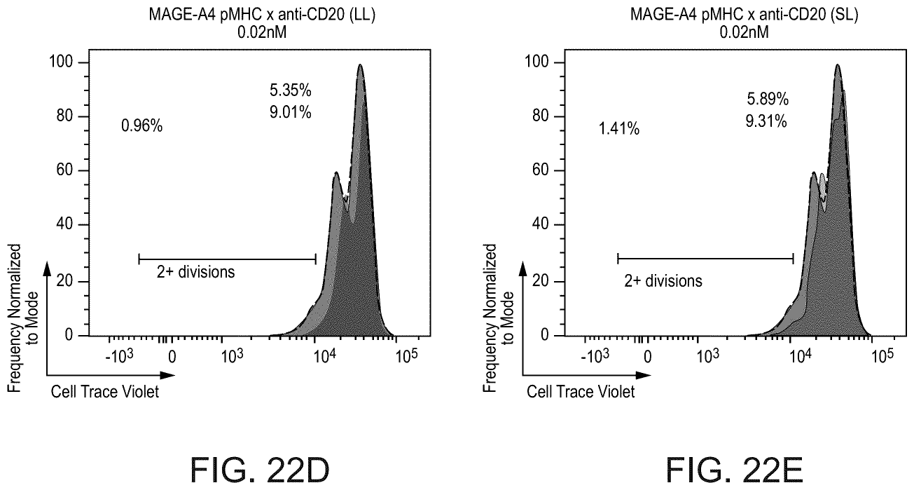

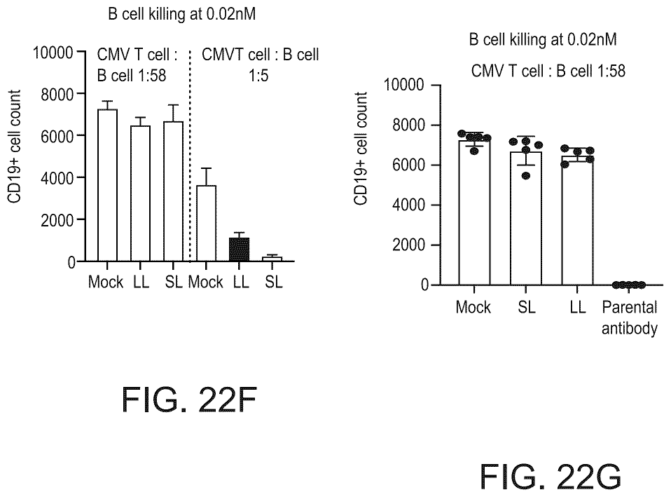

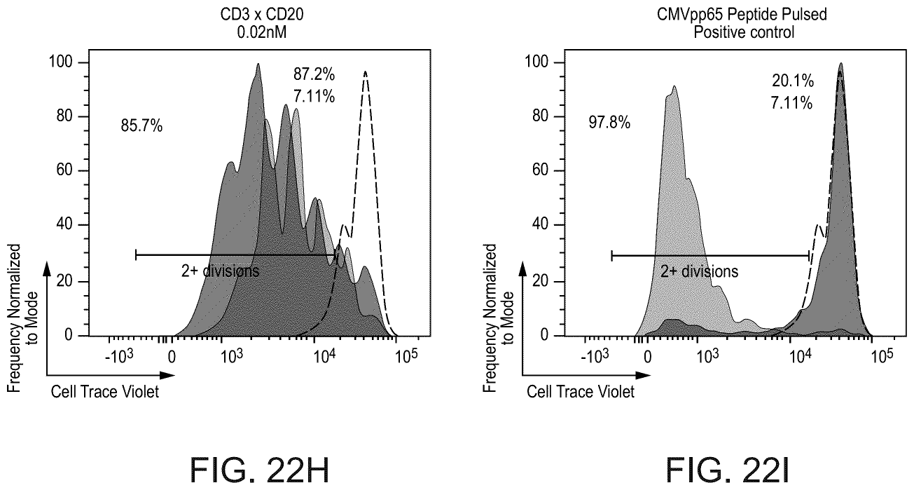

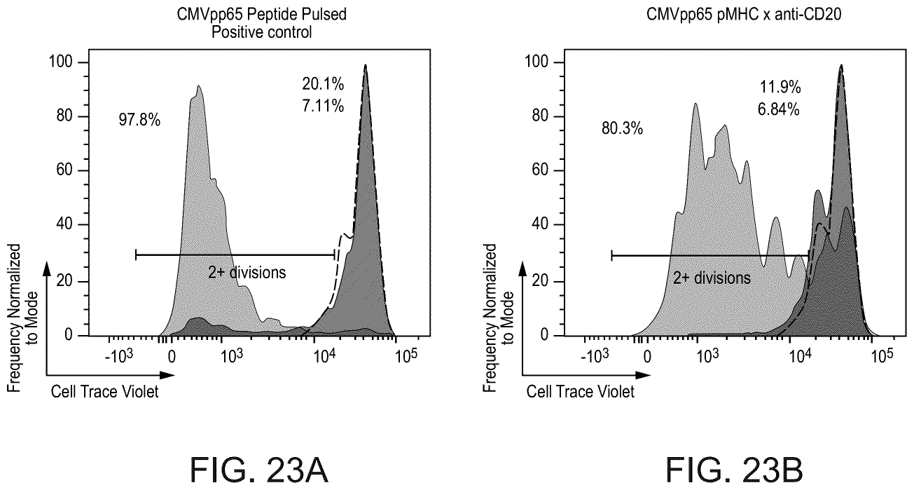

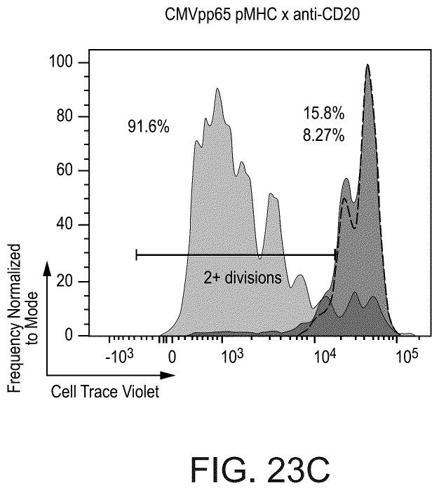

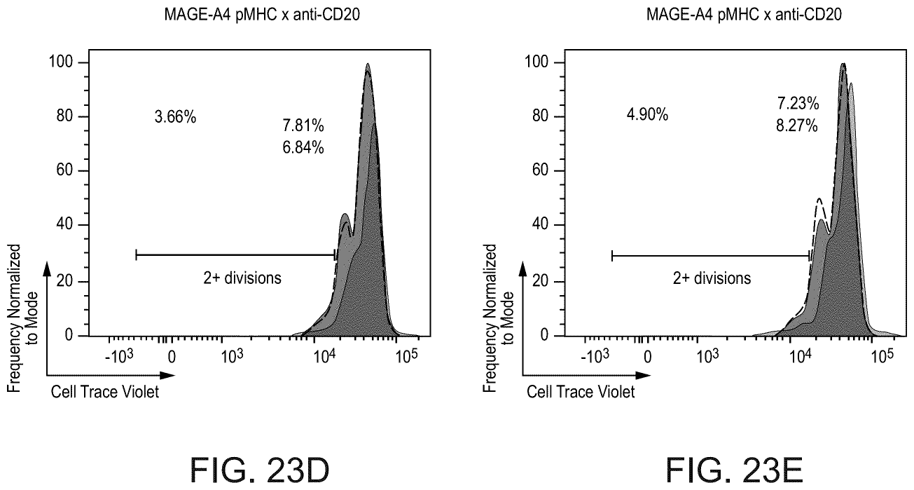

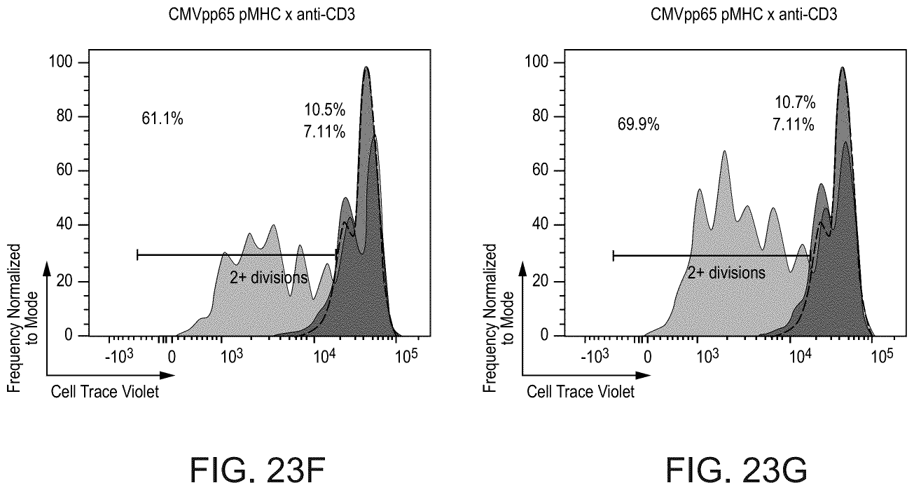

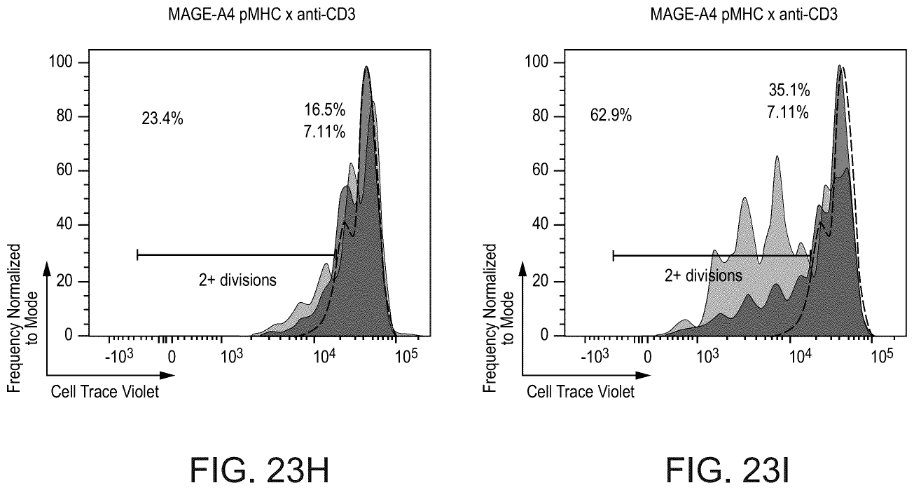

FIGS. 11A-11F are histograms showing antigen-specific expansion of CMVpp65 T cells upon treatment with low concentrations of T cell activators comprising short linkers. CMVpp65+ T cells are represented by light shaded curves, CMVpp65−CD8+ T cells are represented by dark shaded curves, and unexpanded CMV PBMCs (mock control) are represented by the dotted curves. FIG. 11A displays CMVpp65-peptide pulsed cell populations. FIG. 11B displays the populations of cells treated with 0.2 nM CMVpp65 pMHC×CD3 (7221G20) T cell activator comprising a short linker. FIG. 11C displays the populations of cells treated with 5 nM CMVpp65 pMHC×CD3 (7221G20) T cell activator comprising a short linker. FIG. 11D displays the populations of cells treated with 0.2 nM MAGE-A4 pMHC×CD3 (7221G20) comprising a short linker, whereas FIG. 11E displays the populations of cells treated with 5 nM MAGE-A4 pMHC×CD3 (7221G20) comprising a short linker. FIG. 11F shows the percentage of proliferation in tetramer+ and tetramer-cells upon treatment with CMVpp65 pMHC×CD3 (7221G20) T cell activator comprising a short linker.

FIGS. 12A-12T show antigen-specific expansion of CMVpp65 T cells upon treatment with low concentrations of T cell activators comprising CMVpp65 pMHC, displayed as the frequency of T cells as determined by flow cytometry. FIGS. 12A and 12B display the frequency of CD8+ Tetramer+ and TCRα/β+ Tetramer+ cells in unexpanded CMV PBMCs, respectively. FIGS. 12C and 12D display the frequency of CD8+ Tetramer+ and TCRα/β+ Tetramer+ cells in CMVpp65 peptide pulsed CMVpp65+ T cells, respectively. FIGS. 12E and 12F display the frequency of CD8+ Tetramer+ and TCRα/β+ Tetramer+ cells in CMVpp65 cells treated with 0.2 nM of CMVpp65 pMHC×CD3 (7221G20) T cell activator comprising a long linker, respectively. FIGS. 12G and 12H display the frequency of CD8+ Tetramer+ and TCRα/β+ Tetramer+ cells in CMVpp65 cells treated with 5 nM of CMVpp65 pMHC×CD3 (7221G20) T cell activator comprising a long linker, respectively. FIGS. 12I and 12J display the frequency of CD8+ Tetramer+ and TCRα/β+ Tetramer+ cells in CMVpp65 cells treated with 0.2 nM of MAGE-A4 pMHC×CD3 (7221G20) control T cell activator comprising a long linker, respectively. FIGS. 12K and 12L display the frequency of CD8+ Tetramer+ and TCRα/β+ Tetramer+ cells in CMVpp65 cells treated with 5 nM of MAGE-A4 pMHC×CD3 (7221G20) control T cell activator comprising a long linker, respectively. FIGS. 12M and 12N display the frequency of CD8+ Tetramer+ and TCRα/β+ Tetramer+ cells in CMVpp65 cells treated with 0.2 nM of CMVpp65 pMHC×CD3 (7221G20) T cell activator comprising a short linker, respectively. FIGS. 12O and 12P display the frequency of CD8+ Tetramer+ and TCRα/β+ Tetramer+ cells in CMVpp65 cells treated with 5 nM of CMVpp65 pMHC×CD3 (7221G20) T cell activator comprising a short linker, respectively. FIGS. 12Q and 12R display the frequency of CD8+ Tetramer+ and TCRα/β+Tetramer+ cells in CMVpp65 cells treated with 0.2 nM of MAGE-A4 pMHC×CD3 (7221G20) control T cell activator comprising a short linker, respectively. FIGS. 12S and 12T display the frequency of CD8+ Tetramer+ and TCRα/β+ Tetramer+ cells in CMVpp65 cells treated with 5 nM of MAGE-A4 pMHC×CD3 (7221G20) control T cell activator comprising a short linker, respectively.

FIGS. 13A-13F show the effect of CD22×CD28 costimulation on antigen-specific T cell activation by anchored T cell activators. FIG. 13A shows results obtained from a mixture of Jurkat/NFAT-Luc/Cl 3C7/HPV16E7(11-19) TCR709 cells and Raji dKO (CD80 CD86 KO) cells by T cell activator constructs, wherein the pMHC complexes are connected to the hinge region of the Fc domain via short linkers, either costimulated with 31.25 μg/ml CD22×CD28 (solid lines) or without costimulation (dashed lines). FIG. 13B shows results obtained from a mixture of HPV16E6(29-38) TCR Jurkat cells and Raji dKO (CD80 CD86 KO) cells by T cell activator constructs, wherein the pMHC complexes are connected to the hinge region of the Fc domain via short linkers, either costimulated with 31.25 μg/ml CD22×CD28 (solid lines) or without costimulation (dashed lines). FIG. 13C shows results obtained from a mixture of Jurkat/NFAT-Luc/Cl 3C7/HPV16E7(11-19) TCR709 cells and Raji dKO (CD80 CD86 KO) cells by T cell activator constructs, wherein the pMHC complexes are connected to the hinge region of the Fc domain via short linkers, either costimulated with 62.5 μg/ml CD22×CD28 (solid lines) or without costimulation (dashed lines). FIG. 13D shows results obtained from a mixture of HPV16E6(29-38) TCR Jurkat cells and Raji dKO (CD80 CD86 KO) cells by T cell activator constructs, wherein the pMHC complexes are connected to the hinge region of the Fc domain via short linkers, either costimulated with 62.5 μg/ml CD22×CD28 (solid lines) or without costimulation (dashed lines). FIG. 13E shows results obtained from a mixture of Jurkat/NFAT-Luc/Cl 3C7/HPV16E7(11-19) TCR709 cells and Raji dKO (CD80 CD86 KO) cells by T cell activator constructs, wherein the pMHC complexes are connected to the hinge region of the Fc domain via short linkers, either costimulated with 125 μg/ml CD22×CD28 (solid lines) or without costimulation (dashed lines). FIG. 13F shows results obtained from a mixture of HPV16E6(29-38) TCR Jurkat cells and Raji dKO (CD80 CD86 KO) cells by T cell activator constructs, wherein the pMHC complexes are connected to the hinge region of the Fc domain via short linkers, either costimulated with 125 g/ml CD22×CD28 (solid lines) or without costimulation (dashed lines).

FIGS. 14A-14D show the effect of CD22×CD28 costimulation on antigen-specific T cell activation by an anchored pMHC×CD20 T cell activator. FIG. 14A shows results obtained from a mixture of Jurkat/NFAT-Luc/Cl 3C7/HPV16E7(11-19) TCR cells and Raji dKO (CD80 CD86 KO) cells by a pMHC×CD20 T cell activator construct, wherein the pMHC complex is connected to the hinge region of the Fc domain via a short linker, costimulated with a range of concentrations of CD22×CD28. FIG. 14B shows results obtained from a mixture of HPV16E6(29-38) TCR Jurkat cells and Raji dKO (CD80 CD86 KO) cells by a pMHC×CD20 T cell activator construct, wherein the pMHC complex is connected to the hinge region of the Fc domain via a short linker, costimulated with a range of concentrations of CD22×CD28. FIG. 14C shows results obtained from a mixture of Jurkat/NFAT-Luc/Cl 3C7/HPV16E7(11-19) TCR709 cells and Raji dKO (CD80 CD86 KO) cells by a pMHC×CD20 T cell activator construct, wherein the pMHC complex is connected to the hinge region of the Fc domain via a long linker, costimulated with a range of concentrations of CD22×CD28. FIG. 14D shows results obtained from a mixture of HPV16E6(29-38) TCR Jurkat cells and Raji dKO (CD80 CD86 KO) cells by a pMHC×CD20 T cell activator construct, wherein the pMHC complex is connected to the hinge region of the Fc domain via a long linker, costimulated with a range of concentrations of CD22×CD28.

FIGS. 15A-15D show Jurkat reporter cell activation by T cell activator constructs with IgG1 or IgG4 Fc domains, where the pMHC complex of each is connected to the hinge region of the IgG1 or IgG4 Fc domain via a short linker. FIG. 15A shows activation of Jurkat/NFAT-Luc/Cl 3C7/HPV16E7(11-19) TCR709 cells by IgG1-containing T cell activator constructs. FIG. 15B shows activation of Jurkat/NFAT-Luc/Cl 3C7/HPV16E6(29-38) TCR cells by IgG1-containing T cell activator constructs. FIG. 15C shows activation of Jurkat/NFAT-Luc/Cl 3C7/HPV16E7(11-19) TCR709 cells by IgG4-containing T cell activator constructs. FIG. 15D shows activation of Jurkat/NFAT-Luc/Cl 3C7/HPV16E6(29-38) TCR cells by IgG4-containing T cell activator constructs.

FIGS. 16A-16D show Jurkat reporter cell activation by T cell activator constructs with IgG1 or IgG4 Fc domains, where the pMHC complex of each is connected to the hinge region of the IgG1 or IgG4 Fc domain via a long linker. FIG. 16A shows activation of Jurkat/NFAT-Luc/Cl 3C7/HPV16E7(11-19) TCR709 cells by IgG1-containing T cell activator constructs. FIG. 16B shows activation of Jurkat/NFAT-Luc/Cl 3C7/HPV16E6(29-38) TCR cells by IgG1-containing T cell activator constructs. FIG. 16C shows activation of Jurkat/NFAT-Luc/Cl 3C7/HPV16E7(11-19) TCR709 cells by IgG4-containing T cell activator constructs. FIG. 16D shows activation of Jurkat/NFAT-Luc/Cl 3C7/HPV16E6(29-38) TCR cells by IgG4-containing T cell activator constructs.

FIGS. 17A-17D show Jurkat reporter cell activation by T cell activator constructs, each comprising a pMHC complex and an antigen binding domain of an anti-CD3 antibody. FIG. 17A shows activation of Jurkat/NFAT-Luc/Cl 3C7/HPV16E7(11-19) TCR709 cells by T cell activator constructs, where the pMHC complex of each is connected to the hinge region of the Fc domain via a short linker. FIG. 17B shows activation of Jurkat/NFAT-Luc/Cl 3C7/HPV16E6(29-38) TCR cells by T cell activator constructs, where the pMHC complex of each is connected to the hinge region of the Fc domain via a short linker. FIG. 17C shows activation of Jurkat/NFAT-Luc/Cl 3C7/HPV16E7(11-19) TCR709 cells by T cell activator constructs, where the pMHC complex of each is connected to the hinge region of the Fc domain via a long linker. FIG. 17D shows activation of Jurkat/NFAT-Luc/Cl 3C7/HPV16E6(29-38) TCR cells by T cell activator constructs, where the pMHC complex of each is connected to the hinge region of the Fc domain via a long linker.

FIGS. 18A-18G are histograms showing antigen-specific expansion of CMVpp65 T cells upon treatment with low concentrations of T cell activators comprising long linkers. CMVpp65 Tetramer+ T cells are represented by light shaded curves, CMVpp65 Tetramer-CD8+ T cells are represented by dark shaded curves, and unexpanded CD8+ CMV PBMCs (mock control) are represented by the dotted curves. FIG. 18A displays CMVpp65-peptide pulsed cell populations. FIG. 18B displays the populations of cells treated with 0.02 nM CMVpp65 pMHC×CD20 T cell activator comprising a long linker. FIG. 18C displays the populations of cells treated with 1.8 nM CMVpp65 pMHC×CD20 T cell activator comprising a long linker. FIG. 18D displays the populations of cells treated with 0.2 nM MAGE-A4 pMHC×CD20 comprising a long linker. FIG. 18E displays the populations of cells treated with 1.8 nM MAGE-A4 pMHC×CD20 comprising a long linker. FIG. 18F shows the percentage of proliferation in tetramer+ and tetramer-cells upon treatment with CMVpp65 pMHC×CD20 T cell activator comprising a long linker. FIG. 18G shows B cell killing upon treatment with various concentrations of CMVpp65 pMHC×CD20 T cell activator comprising a long linker.

FIGS. 19A-19G are histograms showing antigen-specific expansion of CMVpp65 T cells upon treatment with low concentrations of T cell activators comprising short linkers. CMVpp65 Tetramer+ T cells are represented by light shaded curves, CMVpp65 Tetramer-CD8+ T cells are represented by dark shaded curves, and unexpanded CD8+ CMV PBMCs (mock control) are represented by the dotted curves. FIG. 19A displays CMVpp65-peptide pulsed cell populations. FIG. 19B displays the populations of cells treated with 0.02 nM CMVpp65 pMHC×CD20 T cell activator comprising a short linker. FIG. 19C displays the populations of cells treated with 1.8 nM CMVpp65 pMHC×CD20 T cell activator comprising a short linker. FIG. 19D displays the populations of cells treated with 0.2 nM MAGE-A4 pMHC×CD20 comprising a short linker FIG. 19E displays the populations of cells treated with 1.8 nM MAGE-A4 pMHC×CD20 comprising a short linker. FIG. 19F shows the percentage of proliferation in tetramer+ and tetramer-cells upon treatment with CMVpp65 pMHC×CD20 T cell activator comprising a short linker. FIG. 19G shows B cell killing upon treatment with various concentrations of CMVpp65 pMHC×CD20 T cell activator comprising a short linker.

FIGS. 20A-20J show antigen-specific expansion of CMVpp65 T cells upon treatment with low concentrations of T cell activators comprising CMVpp65 pMHC, displayed as the frequency of T cells as determined by flow cytometry. FIG. 20A displays the frequency of CD8+ Tetramer+ cells in CMVpp65 cells treated with 0.02 nM of CMVpp65 pMHC×CD20 T cell activator comprising a long linker. FIG. 20B displays the frequency of CD8+ Tetramer+ cells in CMVpp65 cells treated with 1.8 nM of CMVpp65 pMHC×CD20 T cell activator comprising a long linker. FIG. 20C displays the frequency of CD8+ Tetramer+ cells in CMVpp65 peptide+IFNα pulsed CMVpp65+ T cells. FIG. 20D shows the frequency of CD8+ Tetramer+ cells in CMVpp65 cells treated with 0.02 nM of MAGE-A4 pMHC×CD20 control T cell activator comprising a long linker. FIG. 20E shows the frequency of CD8+ Tetramer+ cells in CMVpp65 cells treated with 1.8 nM of MAGE-A4 pMHC×CD20 control T cell activator comprising a long linker. FIG. 20F displays the frequency of CD8+ Tetramer+ cells in CMVpp65 cells treated with 0.02 nM of CMVpp65 pMHC×CD20 T cell activator comprising a short linker. FIG. 20G displays the frequency of CD8+ Tetramer+ cells in CMVpp65 cells treated with 1.8 nM of CMVpp65 pMHC×CD20 T cell activator comprising a short linker. FIG. 20H displays the frequency of CD8+ Tetramer+ cells in unexpanded CMV PBMCs. FIG. 20I shows the frequency of CD8+ Tetramer+ cells in CMVpp65 cells treated with 0.02 nM of MAGE-A4 pMHC×CD20 control T cell activator comprising a short linker. FIG. 20J shows the frequency of CD8+ Tetramer+ cells in CMVpp65 cells treated with 1.8 nM of MAGE-A4 pMHC×CD20 control T cell activator comprising a short linker.

FIGS. 21A-21K are histograms showing antigen-specific expansion of CMVpp65 T cells upon treatment with low concentrations of T cell activators in nondepleted PBMCs (FIGS. 21A-21E), PBMCs with B cell depletion (FIGS. 21F-21H), and isolated Pan T cells (FIGS. 21I-21K). CMVpp65 Tetramer+ T cells are represented by light shaded curves, CMVpp65 Tetramer-CD8+ T cells are represented by dark shaded curves, and unexpanded CD8+ CMV PBMCs (mock control) are represented by the dotted curves. FIG. 21A displays CMVpp65-peptide pulsed cell populations. FIG. 21B displays the populations of cells with a CMV T cell:B cell ratio of 1:5 treated with 0.02 nM CMVpp65 pMHC×CD20 T cell activator comprising a long linker. FIG. 21C displays the populations of cells with a CMV T cell:B cell ratio of 1:5 treated with 0.02 nM CMVpp65 pMHC×CD20 T cell activator comprising a short linker. FIG. 21D displays the populations of cells with a CMV T cell:B cell ratio of 1:5, treated with 0.02 nM MAGE-A4 pMHC×CD20 T cell activator comprising a long linker. FIG. 21E displays the populations of cells with a CMV T cell:B cell ratio of 1:5, treated with 0.02 nM MAGE-A4 pMHC×CD20 T cell activator comprising a short linker. FIG. 21F displays CMVpp65-peptide pulsed cell populations. FIG. 21G displays the populations of cells with a CMV T cell:B cell ratio of 1:5 treated with 0.02 nM CMVpp65 pMHC×CD20 T cell activator comprising a long linker. FIG. 21H displays the populations of cells with a CMV T cell:B cell ratio of 1:5 treated with 0.02 nM CMVpp65 pMHC×CD20 T cell activator comprising a short linker. FIG. 21I displays CMVpp65-peptide pulsed cell populations. FIG. 21J displays the populations of cells treated with 0.02 nM CMVpp65 pMHC×CD20 T cell activator comprising a long linker. FIG. 21K displays the populations of cells treated with 0.02 nM CMVpp65 pMHC×CD20 T cell activator comprising a short linker.

FIGS. 22A-22I are histograms showing antigen-specific expansion of CMVpp65 T cells upon treatment of PBMCs with a CMV T cell:B cell ratio of 1:58 with low concentrations of T cell activators. CMVpp65 Tetramer+ T cells are represented by light shaded curves, CMVpp65 Tetramer-CD8+ T cells are represented by dark shaded curves, and unexpanded CD8+ CMV PBMCs (mock control) are represented by the dotted curves. FIG. 22A displays CMVpp65-peptide pulsed cell populations. FIG. 22B displays the populations of cells treated with 0.02 nM CMVpp65 pMHC×CD20 T cell activator comprising a long linker. FIG. 22C displays the populations of cells treated with 0.02 nM CMVpp65 pMHC×CD20 T cell activator comprising a short linker. FIG. 22D displays the populations of cells treated with 0.02 nM MAGE-A4 pMHC×CD20 T cell activator comprising a long linker. FIG. 22E displays the populations of cells treated with 0.02 nM MAGE-A4 pMHC×CD20 T cell activator comprising a short linker. FIG. 22F shows B cell killing upon treatment with various concentrations of CMVpp65 pMHC×CD20 T cell activator. LL: long linker; SL: short linker. FIG. 22G shows B cell killing upon treatment with the parental anti-CD3×anti-CD20 antibody (also referred to as CD3×CD20 or REGN1979 herein) or CMVpp65 pMHC×CD20 T cell activator comprising either short or long linkers. LL: long linker; SL: short linker. FIG. 22H displays the populations of cells treated with the parental antibody, REGN1979. FIG. 22I displays CMVpp65-peptide pulsed cell populations.

FIGS. 23A-23I are histograms showing antigen-specific expansion of CMVpp65 T cells upon treatment with low concentrations of T cell activators with different immune cell antigen targeting moieties. FIG. 23A displays CMVpp65-peptide pulsed cell populations. FIG. 23B displays the populations of cells treated with 0.02 nM CMVpp65 pMHC×CD20 T cell activator comprising a long linker. FIG. 23C displays the populations of cells treated with 0.02 nM CMVpp65 pMHC×CD20 T cell activator comprising a short linker. FIG. 23D displays the populations of cells treated with 0.02 nM MAGE-A4 pMHC×CD20 T cell activator comprising a long linker. FIG. 23E displays the populations of cells treated with 0.02 nM MAGE-A4 pMHC×CD20 T cell activator comprising a short linker. FIG. 23F displays the populations of cells treated with 0.2 nM CMVpp65 pMHC×CD3 T cell activator comprising a long linker. FIG. 23G displays the populations of cells treated with 0.2 nM CMVpp65 pMHC×CD3 T cell activator comprising a short linker. FIG. 23H displays the populations of cells treated with 0.2 nM MAGE-A4 pMHC×CD3 T cell activator comprising a long linker. FIG. 23I displays the populations of cells treated with 0.2 nM MAGE-A4 pMHC×CD3 T cell activator comprising a short linker.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antigen Binding Domain or ABD: The term “antigen binding domain” or “ABD” as used herein refers to the portion of a targeting moiety that is capable of specific, non-covalent, and reversible binding to a target molecule.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding sites and/or antigen-binding sites having non-native configurations, e.g., a molecule that has a Fab or scFv domain C-terminal to the CH3 domain.

Antigenic Peptide: The term “antigenic peptide” (also “antigen peptide” or “antigenic determinant,” used interchangeably) as used herein relates to a portion or fragment of an antigen which is capable of stimulating an immune response, preferably a cellular response against the antigen or against cells characterized by expression of the antigen (e.g., cancer cells). In some embodiments, an antigenic peptide is capable of stimulating a cellular response against a cell characterized by expression or presentation of the antigen and preferably is capable of activating an antigen-responsive cytotoxic T-lymphocyte (CTL). In certain embodiments, an antigenic peptide is a portion or fragment of a tumor antigen including, but not limited to, LCMV derived peptide gp33-41, APF (126-134), BALF (276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAG-A3 (112-120), MAGE-A4 (230-239), MAGE-A4 (286-294), NYESO1 (157-165, C165A), NYESO1 (157-165, C165V), p54 WT (264-272), PAP-3 (136-143), PSMA (4-12), PSMA (135-145), Survivin (96-014), Tyrosinase (369-377, 371D), and WT1 (126-134). In some embodiments, an antigenic peptide is between 7 and 20 amino acids in length, between 7 and 12 amino acids in length, between 8 and 11 amino acids in length, or between 9 and 10 amino acids in length. In some embodiments, an antigenic peptide is 7, 8, 9, 10, 11, 12, 13, 14, 15, 16, 17, 18, 19, or 20 amino acids in length.

Associated: The term “associated” in the context of a T cell activator or a component thereof refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional T cell activator. Examples of associations that might be present in an T cell activator of the disclosure include (but are not limited to) associations between homodimeric or heterodimeric Fc domains in an Fc region (homodimeric or heterodimeric as described in Section 6.7.1), associations between VH and VL regions in a Fab or scFv, associations between CH1 and CL in a Fab, and associations between CH3 and CH3 in a domain substituted Fab.

B Cell Antigen: The term “B cell antigen” as used herein refers to any biological molecule (e.g., protein, carbohydrate, lipid or combination thereof) that is present on and/or expressed by a B cell. In some embodiments, at least a portion of a B cell antigen is extracellular (e.g., is a cell surface protein or transmembrane protein having at least one extracellular domain). Particular B cell antigens contemplated herein include, but are not limited to, CD19, CD20, and CD22.

Bivalent: The term “bivalent” as used herein in reference to a pMHC complex and/or a targeting moiety in a T cell activator means a T cell activator that has two pMHC complexes and/or targeting moieties, respectively. In some embodiments, T cell activators that are bivalent for a pMHC complex and/or a targeting moiety are dimeric (either homodimeric or heterodimeric).

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. For example, in general, there are three CDRs in each heavy chain variable region (CDR-H1, CDR-H2, HCDR-H3) and three CDRs in each light chain variable region (CDR1-L1, CDR-L2, CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGeneTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDRL2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDRH2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

EC50: The term “EC50” refers to the half maximal effective concentration of a molecule (such as an antibody or a T cell activator) which induces a response halfway between the baseline and maximum after a specified exposure time. The EC50 essentially represents the concentration of a molecule (e.g., antibody or T cell activator) where 50% of its maximal effect is observed. In certain embodiments, the EC50 value equals the concentration of a T cell activator that gives half-maximal T cell activation in an assay as described in Section 8.1.2.

Epitope: An epitope, or antigenic determinant, is a portion of an antigen (e.g., target molecule) recognized by an antibody or other antigen-binding moiety as described herein. An epitope can be linear or conformational.

Fab: The term “Fab” in the context of a targeting moiety of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps on that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Half Antibody: The term “half antibody” refers to a molecule that comprises at least one Fc domain and can associate with another molecule comprising an Fc domain through, e.g., a disulfide bridge or molecular interactions. A half antibody can be composed of one polypeptide chain or more than one polypeptide chains (e.g., the two polypeptide chains of a Fab or a pMHC complex whose components are on different polypeptide chains). An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABD. Yet another example of a half antibody is a polypeptide comprising an scFv domain, a CH2 domain and a CH3 domain. Yet another example of a half antibody is a polypeptide comprising a pMHC complex, a CH2 domain and a CH3 domain. When multimerized through Fc domains, the T cell activators of the disclosure typically comprise two half antibodies. As illustrated in FIGS. 1A-11, each half antibody typically includes a pMHC complex and an Fc domain or an immune cell antigen targeting moiety and an Fc domain. Each half antibody may include additional components either interspersed among the components listed above or at the termini of the half antibodies, for example linkers, additional pMHC complexes or immune cell antigen targeting moieties, and TAA targeting moieties.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host Cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

Immune Cell Antigen: The term “immune cell antigen” as used herein refers to any biological molecule (e.g., protein, carbohydrate, lipid or combination thereof) that is present on and/or expressed by an immune cell. In some embodiments, at least a portion of an immune cell antigen is extracellular (e.g., is a cell surface protein or transmembrane protein having at least one extracellular domain). Immune cells include, but are not limited to, T cells, B cells, dendritic cells (DCs), macrophages, natural killer (NK) cells. Particular immune cell antigens contemplated herein include, but are not limited to, T cell antigens (e.g., CD3, CD28), and B cell antigens (e.g., CD20, CD22, CD19).

Immune Response: The term “immune response” refers to an integrated bodily response to an antigen and preferably refers to a cellular immune response or a cellular as well as a humoral immune response. The immune response may be protective (also “preventive” or “prophylactic”) and/or therapeutic. The terms “inducing an immune response,” “eliciting an immune response,” and the like, as used herein, can indicate that there was no immune response against a particular antigen before administration of a particular composition (e.g., T cell activator), but it may also indicate that there was a certain level of immune response against a particular antigen before such administration, and that after induction the immune response is enhanced. Thus, “inducing an immune response” also includes “enhancing an immune response”. Preferably, after inducing an immune response in a subject, said subject is protected from developing a disease (e.g., cancer) or the disease condition is ameliorated (e.g., tumor reduction, reduction in cancer cell number, etc.) by inducing an immune response. For example, an immune response against a tumor antigen may be induced in a patient having cancer or in a subject at risk of developing a cancer. Inducing an immune response in this case may mean that the disease condition of the subject is ameliorated, that the subject does not develop metastases, or that the subject at risk of developing cancer does not develop cancer.

K_{D}: The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

Major Histocompatibility Complex and MHC: These terms refer to naturally occurring MHC molecules, individual chains of MHC molecules (e.g., MHC class I α (heavy) chain, 2 microglobulin, MHC class II α chain, and MHC class II β chain), individual subunits of such chains of MHC molecules (e.g., α1, α2, and/or α3 subunits of MHC class I α chain, α1-α2 subunits of MHC class II α chain, 31-32 subunits of MHC class II β chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a T cell receptor (TCR), e.g., an antigen-specific TCR. An MHC class I molecule comprises a peptide binding groove formed by the α1 and α2 domains of the heavy α chain that can stow a peptide of around 8-10 amino acids. Despite the fact that both classes of MHC bind a core of about 9 amino acids (e.g., 5 to 17 amino acids) within peptides, the open-ended nature of MHC class II peptide binding groove (the α1 domain of a class II MHC a polypeptide in association with the B1 domain of a class II MHC (polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time. Conventional identifications of particular MHC variants are used herein. The terms encompass “human leukocyte antigen” or “HLA”.

Monovalent: The term “monovalent” as used herein in reference to a pMHC complex and/or a targeting moiety in a T cell activator means a T cell activator that has only a single pMHC complex and/or targeting moiety, respectively. In some embodiments, T cell activators that are monovalent for the pMHC complex and/or targeting moiety are heterodimeric.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Peptide-MHC Complex, pMHC Complex, Peptide-in-Groove: The terms “peptide-MHC complex,” “pMHC complex,” and “peptide-in-groove,” used interchangeably herein, refer to a molecule comprising (i) an MHC domain (e.g., a human MHC molecule or portion thereof (e.g., the peptide-binding groove thereof and e.g., the extracellular portion thereof)), (ii) an antigenic peptide, and, optionally, (iii) a β2 microglobulin domain (e.g., a human β2 microglobulin or portion thereof), where the MHC domain, the antigenic peptide and optional β2 microglobulin domain are complexed in such a manner that permits specific binding to a T cell receptor. In some embodiments, a pMHC complex comprises at least the extracellular domains of a human HLA class I/human B2 microglobulin molecule and/or a human HLA class II molecule. A pMHC complex may exist as an isolated molecule or as a component of a molecule comprising one or more additional elements, for example as a component of a T cell activator. A pMHC complex, existing as a component of a T cell activator of the disclosure, is also referred to herein as a “pMHC moiety.”

Prevent: The terms “prevent”, “preventing”, “prevention”, “prophylactic treatment” and the like refer to reducing the probability of developing, or to delaying the development of, a disorder, disease, or condition in a subject who does not have but is at risk of or susceptible to developing the disorder, disease, or condition. Prevention and the like do not exclusively mean preventing a subject from ever getting the specific disorder, disease, or condition. Prevention may require the administration of multiple doses of a treatment disclosed herein (e.g., a pharmaceutical composition comprising a T cell activator). Prevention can include the prevention of a recurrence of a disease in a subject for whom all disease symptoms were eliminated (e.g., a subject in remission). Accordingly, a subject who “does not have” a disorder or condition, as described herein, includes a subject who previously had a disease and for whom all disease symptoms were eliminated. For example, methods for prevention of cancer in a subject include methods for prevention of cancer in a subject who has never had cancer and also includes methods for prevention of recurrence or relapse in a subject who previously had cancer.

Protein, Peptide, and Polypeptide: The terms “polypeptide”, “peptide,” and “protein” are used interchangeably herein to refer to a polymer of amino acid residues.

Single Chain Fv or scFv: The term “single chain Fv” or “scFv” as used herein refers to a polypeptide chain comprising the VH and VL domains of antibody, where these domains are present in a single polypeptide chain.

Specifically (or Selectively) Binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Target Molecule: The term “target molecule” as used herein refers to any biological molecule (e.g., protein, carbohydrate, lipid or combination thereof) that can be specifically bound by a targeting moiety in a T cell activator of the disclosure.

T Cell Activator: The term “T cell activator” as used herein refers to a molecule comprising at least one peptide-MHC complex and at least one immune cell antigen targeting moiety, for example at least one TCA targeting moiety and/or at least one BCA targeting moiety. Generally, a T cell activator is a molecule composed of one or more polypeptide chains (e.g., one, two, three or four polypeptide chains) together comprising at least one peptide-MHC complex and at least one immune cell antigen targeting moiety. It is to be understood that the term “T cell activator” extends also to molecules comprising additional features, e.g., one or more multimerization moieties, one or more linker moieties, one or more tumor-associated antigen (TAA) targeting moieties, and any combination of the foregoing, unless the context dictates otherwise. The use of the term “T cell activator” is intended for convenience and descriptive purposes only and does not connote any particular activity or functional property.

T Cell Antigen: The term “T cell antigen” as used herein refers to any biological molecule (e.g., protein, carbohydrate, lipid or combination thereof) that is present on and/or expressed by a T cell. In some embodiments, at least a portion of a T cell antigen is extracellular (e.g., is a cell surface protein or transmembrane protein having at least one extracellular domain). Particular T cell antigens contemplated herein include, but are not limited to, CD3 and CD28.

Targeting Moiety: The term “targeting moiety” as used herein refers to any molecule or binding portion thereof that can specifically bind to an antigen. In certain aspects, the targeting moiety binds to a region of a protein antigen that is extracellular (e.g., an extracellular domain of a cell surface or transmembrane protein expressed by a cell). Exemplary targeting moieties include, but are not limited to, antibodies and antigen binding portions thereof (e.g., Fab, scFv, etc.). A targeting moiety may be described with reference to the antigen to which it specifically binds. Thus, for example, a “T cell antigen targeting moiety” (or “TCA targeting moiety”) refers to a molecule or binding portion thereof that can specifically bind to a T cell antigen (e.g., CD3, CD28, etc.). The TCA targeting moiety can also have a functional activity in addition to binding a T cell antigen. For example, a TCA targeting moiety that is a CD3 targeting moiety (e.g., an anti-CD3 antibody or an antigen binding portion thereof) may facilitate clustering and activation of CD3 on a surface of a T cell, while a TCA targeting moiety that is a CD28 targeting moiety (e.g., an anti-CD28 antibody or an antigen binding portion thereof) may activate CD28 signaling in a T cell. Similarly a “TCR targeting moiety” refers to a molecule or binding portion thereof that can specifically bind to a T cell receptor, and a “B cell antigen targeting moiety” (or “BCA targeting moiety”) refers to a molecule or binding portion thereof that can specifically bind to a B cell antigen (e.g., CD19, CD20, CD22, etc.)

Treat, Treatment, Treating: As used herein, the terms “treat”, “treatment” and “treating” refer to the reduction or amelioration of the progression, severity and/or duration of a disorder (e.g., proliferative disorder), or the amelioration of one or more symptoms (preferably, one or more discernible symptoms) of the disorder resulting from the administration of one or more T cell activators of the disclosure.

In specific embodiments, in the context of treatment of a proliferative disorder, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” (also “tumor associated antigen”) or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Universal Light Chain: The term “universal light chain” as used herein in the context of a targeting moiety refers to a light chain polypeptide capable of pairing with the heavy chain region of the targeting moiety and also capable of pairing with other heavy chain regions. Universal light chains are also known as “common light chains.”

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 6.2. T Cell Activators

The present disclosure provides multispecific molecules comprising a pMHC complex, one or more immune cell antigen targeting moieties (e.g., a TCA targeting moiety and/or a BCA targeting moiety), and an optional multimerization moiety. Such multispecific molecules are often referred to herein as “T cell activators.” The use of the term “T cell activator” is intended for convenience and descriptive purposes only and does not connote any particular activity or functional property.

In various embodiments, the T cell activators are (a) monovalent or bivalent for the pMHC complex and/or (b) monovalent or bivalent for the immune cell antigen targeting moiety. Thus, in some embodiments, a T cell activator of the disclosure is monovalent for the pMHC complex and monovalent for the immune cell antigen targeting moiety. In other embodiments, a T cell activator of the disclosure is bivalent for the pMHC complex and monovalent for the immune cell antigen targeting moiety. In other embodiments, a T cell activator of the disclosure is monovalent for the pMHC complex and bivalent for the immune cell antigen targeting moiety. In yet further embodiments, a T cell activator of the disclosure is bivalent for the pMHC complex and bivalent for the immune cell antigen targeting moiety.

Exemplary pMHC complexes suitable for use in the T cell activators of the disclosure are described in Section 6.3.

Exemplary immune cell antigen targeting moieties (including TCA targeting moieties and BCA targeting moieties) for use in the T cell activators of the disclosure are described in Section 6.4.

The T cell activator can be a fusion protein comprising the pMHC complex, immune cell antigen targeting moiety, and in some cases a multimerization moiety. Exemplary multimerization moieties are described in Section 6.7 and include Fc domains that confer homodimerization or heterodimerization capability to the T cell activator.

Further, each of the pMHC complex, TCA targeting moiety, and multimerization moiety can itself be a fusion protein. For example, the pMHC complex can be a single fusion polypeptide comprising an antigenic peptide, a linker, an optional β2-microglobulin domain, an optional additional linker, and an MHC domain.

The T cell activator can include one or more linker sequences connecting the various components of the molecule, for example connecting the different portions of a pMHC complex, connecting a TCA targeting moiety to a multimerization moiety, or connecting a pMHC complex to a multimerization moiety. Exemplary linkers are described in Section 6.8.

In certain aspects, the T cell activators of the disclosure, following administration to a subject (e.g., a patient with cancer or at risk of developing cancer), increase activation of T cells specific for the antigenic peptide of the pMHC complex.

Below are some illustrative orientations of T cell activators of the disclosure or individual polypeptide chains thereof, in an N-to-C-terminal orientation:
(1) Orientation 1: pMHC complex—optional linker—multimerization moiety—optional linker—immune cell antigen targeting moiety (or component thereof).
(2) Orientation 2: immune cell antigen targeting moiety (or component thereof)—optional linker—multimerization moiety—optional linker—pMHC complex.
(3) Orientation 3: pMHC complex—optional linker—immune cell antigen targeting moiety (or component thereof)—optional linker—multimerization moiety.
(4) Orientation 4: A heterodimer comprising two polypeptides, A and B:
   Polypeptide A: pMHC complex—optional linker—multimerization moiety; and
   Polypeptide B: immune cell antigen targeting moiety (or component thereof)—optional linker—multimerization moiety.
(5) Orientation 5: A heterodimer comprising two polypeptides, A and B:
   Polypeptide A: pMHC complex—optional linker—multimerization moiety; and
   Polypeptide B: pMHC complex—optional linker—multimerization moiety—optional linker—immune cell antigen targeting moiety (or component thereof).
(6) Orientation 6: A homodimer or heterodimer comprising two polypeptides, A and B:
   Polypeptide A: pMHC complex—optional linker—multimerization moiety—optional linker-immune cell antigen targeting moiety (or component thereof); and
   Polypeptide B: pMHC complex—optional linker—multimerization moiety—optional linker—immune cell antigen targeting moiety (or component thereof).
(7) Orientation 7: A homodimer or heterodimer comprising two polypeptides, A and B:
   Polypeptide A: pMHC complex—optional linker—immune cell antigen targeting moiety (or component thereof)—optional linker—multimerization moiety; and
   Polypeptide B: pMHC complex—optional linker—immune cell antigen targeting moiety (or component thereof)—optional linker—multimerization moiety.
(8) Orientation 8: A heterodimer comprising two polypeptides, A and B:
   Polypeptide A: pMHC complex—optional linker—multimerization moiety;
   Polypeptide B: immune cell antigen targeting moiety (or component thereof)—optional linker—multimerization moiety—optional linker—immune cell antigen targeting moiety (or component thereof).
(9) Orientation 9: A heterodimer comprising two polypeptides, A and B:
   Polypeptide A: pMHC complex—optional linker—multimerization moiety—optional linker—immune cell targeting moiety (or component thereof);
   Polypeptide B: immune cell antigen targeting moiety (or component thereof)—optional linker—multimerization moiety.
(10) Orientation 10: A heterodimer comprising two polypeptides, A and B:
   Polypeptide A: pMHC complex—optional linker—multimerization moiety—optional linker—immune cell targeting moiety (or component thereof);
   Polypeptide B: immune cell antigen targeting moiety (or component thereof)—optional linker—multimerization moiety—optional linker—immune cell antigen targeting moiety (or component thereof).
(11) Orientation 11: A heterodimer comprising two polypeptides, A and B:
   Polypeptide A: pMHC complex—optional linker—multimerization moiety;
   Polypeptide B: immune cell antigen targeting moiety (or component thereof)—optional linker—multimerization moiety—optional linker—tumor antigen targeting moiety (or component thereof).
(12) Orientation 12: A heterodimer comprising two polypeptides, A and B:
   Polypeptide A: pMHC complex—optional linker—multimerization moiety—optional linker—immune cell targeting moiety (or component thereof);
   Polypeptide B: tumor antigen targeting moiety (or component thereof)—optional linker—multimerization moiety—optional linker—tumor antigen targeting moiety (or component thereof).

In the foregoing embodiments, the multimermization moiety can be an Fc domain. Thus, T cell activators that are dimerized through Fc domains may be described as being composed of two half antibodies, and heterodimeric pairings can be achieved through the pairing of Fc heterodimerization variants as described in Section 6.7.1.2.

In certain embodiments, the T cell activators of the disclosure comprise two or more immune cell antigen targeting moieties, for example an Orientation 5, Orientation 6, Orientation 7, Orientation 8, Orientation 9, or Orientation 10 T cell activator. The two or more immune cell antigen targeting moieties may be the same type of immune cell antigen targeting moiety (e.g., two or more TCA targeting moieties, two or more BCA targeting moieties, etc.). A T cell activator comprising two or more TCA targeting moieties may comprise two moieties targeting the same antigen (e.g., two CD3 targeting moieties or two CD28 targeting moieties) or different antigens (e.g., one CD3 targeting moiety and one CD28 targeting moiety). A T cell activator comprising two or more BCA targeting moieties may comprise two moieties targeting the same antigen (e.g., two CD19 targeting moieties, two CD20 targeting moieties, or two CD22 targeting moieties) or different antigens (e.g., one CD19 targeting moiety and one CD22 targeting moiety).

Alternatively, the two or more immune cell antigen targeting moieties may include different types of immune cell antigen targeting moieties (e.g., one TCA targeting moiety and one BCA targeting moiety, two TCA targeting moieties and one BCA targeting moiety, two BCA targeting moieties and one TCA targeting moiety, etc.). In some embodiments, a T cell activator comprises one CD3 targeting moiety and one CD19 targeting moiety. In some embodiments, a T cell activator comprises one CD3 targeting moiety and one CD20 targeting moiety. In some embodiments, a T cell activator comprises one CD3 targeting moiety and one CD22 targeting moiety. In some embodiments, a T cell activator comprises one CD28 targeting moiety and one CD19 targeting moiety. In some embodiments, a T cell activator comprises one CD28 targeting moiety and one CD20 targeting moiety. In some embodiments, a T cell activator comprises one CD28 targeting moiety and one CD22 targeting moiety. In some embodiments, a T cell activator comprises two CD3 targeting moieties and one CD19 targeting moiety. In some embodiments, a T cell activator comprises two CD3 targeting moieties and one CD20 targeting moiety. In some embodiments, a T cell activator comprises two CD3 targeting moieties and one CD22 targeting moiety. In some embodiments, a T cell activator comprises two CD28 targeting moieties and one CD19 targeting moiety. In some embodiments, a T cell activator comprises two CD28 targeting moieties and one CD20 targeting moiety. In some embodiments, a T cell activator comprises two CD28 targeting moieties and one CD22 targeting moiety. In some embodiments, a T cell activator comprises one CD3 targeting moiety and two CD19 targeting moieties. In some embodiments, a T cell activator comprises one CD3 targeting moiety and two CD20 targeting moieties. In some embodiments, a T cell activator comprises one CD3 targeting moiety and two CD22 targeting moieties. In some embodiments, a T cell activator comprises one CD28 targeting moiety and two CD19 targeting moieties. In some embodiments, a T cell activator comprises one CD28 targeting moiety and two CD20 targeting moieties. In some embodiments, a T cell activator comprises one CD28 targeting moiety and two CD22 targeting moieties.

An exemplary Orientation 4 T cell activator is depicted in FIG. 1A. Immune cell antigen targeting moiety (3) of the example T cell activators depicted in FIG. 1A may be a TCA targeting moiety (e.g., a CD3 targeting moiety or a CD28 targeting moiety) or may be a BCA targeting moiety (e.g., a CD19 targeting moiety, a CD20 targeting moiety, or a CD22 targeting moiety).

An exemplary Orientation 5 T cell activator is depicted in FIG. 1B. Immune cell antigen targeting moiety (3) of the example T cell activators depicted in FIG. 1B may be a TCA targeting moiety (e.g., a CD3 targeting moiety or a CD28 targeting moiety) or may be a BCA targeting moiety (e.g., a CD19 targeting moiety, a CD20 targeting moiety, or a CD22 targeting moiety).

An exemplary Orientation 6 T cell activator is depicted in FIG. 1C. Immune cell antigen targeting moieties (3) of the example T cell activator depicted in FIG. 1C may both be a TCA targeting moiety (e.g., a CD3 targeting moiety or a CD28 targeting moiety), may both be a BCA targeting moiety (e.g., a CD19 targeting moiety, a CD20 targeting moiety, or a CD22 targeting moiety), or may be one TCA targeting moiety and one BCA targeting moiety (e.g., one CD28 targeting moiety and one CD20 targeting moiety).

An exemplary Orientation 7 T cell activator is depicted in FIG. 1D. Immune cell antigen targeting moieties (3) of the example T cell activator depicted in FIG. 1D may both be a TCA targeting moiety (e.g., a CD3 targeting moiety or a CD28 targeting moiety), may both be a BCA targeting moiety (e.g., a CD19 targeting moiety, a CD20 targeting moiety, or a CD22 targeting moiety), or may be one TCA targeting moiety and one BCA targeting moiety (e.g., one CD28 targeting moiety and one CD20 targeting moiety).

An exemplary Orientation 8 T cell activator is depicted in FIG. 1E. Immune cell antigen targeting moieties (3) of the example T cell activator depicted in FIG. 1E may both be a TCA targeting moiety (e.g., a CD3 targeting moiety or a CD28 targeting moiety), may both be a BCA targeting moiety (e.g., a CD19 targeting moiety, a CD20 targeting moiety, or a CD22 targeting moiety), or may be one TCA targeting moiety and one BCA targeting moiety (e.g., one CD28 targeting moiety and one CD20 targeting moiety).

An exemplary Orientation 9 T cell activator is depicted in FIG. 1F. Immune cell antigen targeting moieties (3) of the example T cell activator depicted in FIG. 1F may both be a TCA targeting moiety (e.g., a CD3 targeting moiety or a CD28 targeting moiety), may both be a BCA targeting moiety (e.g., a CD19 targeting moiety, a CD20 targeting moiety, or a CD22 targeting moiety), or may be one TCA targeting moiety and one BCA targeting moiety (e.g., one CD28 targeting moiety and one CD20 targeting moiety).

An exemplary Orientation 10 T cell activator is depicted in FIG. 1G. Immune cell antigen targeting moieties (3) of the example T cell activator depicted in FIG. 1G may both be a TCA targeting moiety (e.g., a CD3 targeting moiety or a CD28 targeting moiety), may both be a BCA targeting moiety (e.g., a CD19 targeting moiety, a CD20 targeting moiety, or a CD22 targeting moiety), or may be one TCA targeting moiety and one BCA targeting moiety (e.g., one CD28 targeting moiety and one CD20 targeting moiety).

In the T cell activators of the disclosure, when the immune cell antigen targeting moiety is an antigen binding domain (“ABD”) of an antibody, each immune cell antigen targeting moiety can be composed of two polypeptide chains, one polypeptide chain bearing the heavy chain variable region and the other polypeptide chain bearing the light chain variable region. Thus, the immune cell antigen targeting moiety itself can comprise heavy and light chain variable domains on separate polypeptide chains. For example, a polypeptide comprising the immune cell antigen targeting moiety can be composed of two polypeptide chains, where one chain contains the heavy chain variable domain of a targeting moiety-optional linker-multimerization moiety, and the other chain comprises the light chain variable domain of a targeting moiety.

Alternatively, an scFv can be used in which the heavy and light chain variable regions are fused to one another in a single polypeptide.

When the T cell activators of the disclosure are dimerized through Fc domains, they can be said to be composed of two half antibodies. Below are some illustrative configurations of half antibodies that can be incorporated into a T cell activator of the disclosure:
(1) Half Antibody 1: A first polypeptide chain comprising a pMHC complex—optional linker A—Fc domain—optional linker B—immune cell antigen targeting moiety or component thereof, optionally associated with a second polypeptide chain comprising other components of the immune cell antigen targeting moiety (for example the first polypeptide chain may comprise the VH and CH1 of a immune cell antigen targeting Fab and the second polypeptide chain may comprise the VL and CL of the immune cell antigen targeting Fab, or vice versa).
(2) Half Antibody 2: A first polypeptide chain comprising a immune cell antigen targeting moiety or component thereof—optional linker A—Fc domain—optional linker B—pMHC complex, optionally associated with a second polypeptide chain comprising other components of the immune cell antigen targeting moiety (for example the first polypeptide chain may comprise the VH and CH1 of a immune cell antigen targeting Fab and the second polypeptide chain may comprise the VL and CL of the immune cell antigen targeting Fab, or vice versa).
(3) Half Antibody 3: A first polypeptide chain comprising a pMHC complex—optional linker A—immune cell antigen targeting moiety or component thereof—optional linker B—Fc domain, optionally associated with a second polypeptide chain comprising other components of the immune cell antigen targeting moiety (for example the first polypeptide chain may comprise the VH and CH1 of a immune cell antigen targeting Fab and the second polypeptide chain may comprise the VL and CL of the immune cell antigen targeting Fab, or vice versa).
(4) Half Antibody 4: A polypeptide chain comprising a pMHC complex—optional linker A—Fc domain.
(5) Half Antibody 5: A first polypeptide chain comprising a immune cell antigen targeting moiety or component thereof—optional linker A—Fc domain, optionally associated with a second polypeptide chain comprising other components of the immune cell antigen targeting moiety (for example the first polypeptide chain may comprise the VH and CH1 of a immune cell antigen targeting Fab and the second polypeptide chain may comprise the VL and CL of the immune cell antigen targeting Fab, or vice versa).
(6) Half Antibody 6: A first polypeptide chain comprising a first immune cell antigen targeting moiety or component thereof—optional linker A—Fc domain—optional linker B—a second immune cell antigen targeting moiety or component thereof, optionally associated with a second polypeptide chain comprising other components of the first immune cell antigen targeting moiety (for example the first polypeptide chain may comprise the VH and CH1 of an immune cell antigen targeting Fab and the second polypeptide chain may comprise the VL and CL of the immune cell antigen targeting Fab, or vice versa) and/or with a third polypeptide chain comprising other components of the second immune cell antigen targeting moiety (for example the first polypeptide chain may comprise the VH and CH1 of an immune cell antigen targeting Fab and the third polypeptide chain may comprise the VL and CL of the immune cell antigen targeting Fab, or vice versa).
(7) Half Antibody 7: A first polypeptide chain comprising an immune cell antigen targeting moiety or component thereof—optional linker A—Fc domain—optional linker B—tumor antigen targeting moiety or component thereof, optionally associated with a second polypeptide chain comprising other components of the immune cell antigen targeting moiety (for example the first polypeptide chain may comprise the VH and CH1 of an immune cell antigen targeting Fab and the second polypeptide chain may comprise the VL and CL of the immune cell antigen targeting Fab, or vice versa) and/or with a third polypeptide chain comprising other components of the tumor antigen targeting moiety (for example the first polypeptide chain may comprise the VH and CH1 of a tumor antigen targeting Fab and the third polypeptide chain may comprise the VL and CL of the tumor antigen targeting Fab, or vice versa).
(8) Half Antibody 8: A first polypeptide chain comprising a tumor antigen targeting moiety or component thereof—optional linker A—Fc domain—optional linker B—tumor antigen targeting moiety or component thereof, optionally associated with a second polypeptide chain comprising other components of the tumor antigen targeting moiety (for example the first polypeptide chain may comprise the VH and CH1 of a tumor antigen targeting Fab and the second polypeptide chain may comprise the VL and CL of the tumor antigen targeting Fab, or vice versa) and/or with a third polypeptide chain comprising other components of the tumor antigen targeting moiety (for example the first polypeptide chain may comprise the VH and CH1 of a tumor antigen targeting Fab and the third polypeptide chain may comprise the VL and CL of the tumor antigen targeting Fab, or vice versa).

In some embodiments, a T cell activator of the disclosure comprises or is composed of half antibody 5 and half antibody 4. An embodiment of this T cell activator is depicted in FIG. 1A, with half antibody 5 on the left and half antibody 4 on the right.

In some embodiments, a T cell activator of the disclosure comprises or is composed of half antibody 4 and half antibody 1. An embodiment of this T cell activator is depicted in FIGS. 1B, with half antibody 4 on the left and half antibody 1 on the right.

In some embodiments, a T cell activator of the disclosure comprises or is composed of two of half antibody 1. An embodiment of this T cell activator is depicted in FIG. 1C. Each half antibody 1 may comprise the same type of immune cell antigen targeting moiety (e.g., the T cell activator may comprise two TCA targeting moieties, which may target the same or different TCAs, or two BCA targeting moieties, which may target the same or different BCAs) or may comprise different types of immune cell antigen targeting moieties (e.g., the T cell activator may comprise one TCA targeting moiety and one BCA targeting moiety).

In some embodiments, a T cell activator of the disclosure comprises or is composed of two of half antibody 3. An embodiment of this T cell activator is depicted in FIG. 1D. Each half antibody 3 may comprise the same type of immune cell antigen targeting moiety (e.g., the T cell activator may comprise two TCA targeting moieties, which may target the same or different TCAs, or two BCA targeting moieties, which may target the same or different BCAs) or may comprise different types of immune cell antigen targeting moieties (e.g., the T cell activator may comprise one TCA targeting moiety and one BCA targeting moiety).

In some embodiments, a T cell activator of the disclosure comprises or is composed of half antibody 4 and half antibody 6. An embodiment of this T cell activator is depicted in FIG. 1E, with half antibody 6 on the left and half antibody 4 on the right.

In some embodiments, a T cell activator of the disclosure comprises or is composed of half antibody 1 and half antibody 5. An embodiment of this T cell activator is depicted in FIG. 1F, with half antibody 5 on the left and half antibody 1 on the right.

In some embodiments, a T cell activator of the disclosure comprises or is composed of half antibody 1 and half antibody 6. An embodiment of this T cell activator is depicted in FIG. 1G, with half antibody 6 on the left and half antibody 1 on the right.

In some embodiments, a T cell activator of the disclosure comprises or is composed of half antibody 4 and half antibody 7. An embodiment of this T cell activator is depicted in FIG. 1H, with half antibody 7 on the left and half antibody 4 on the right.

In some embodiments, a T cell activator of the disclosure comprises or is composed of half antibody 1 and half antibody 8. An embodiment of this T cell activator is depicted in FIG. 11, with half antibody 8 on the left and half antibody 1 on the right.

In some embodiments, a T cell activator of the disclosure comprises or is composed of half antibody 2 and half antibody 5. In further embodiments, a T cell activator of the disclosure comprises or is composed of two of half antibody 2.

Heterodimeric T cell activators (e.g., as depicted in FIGS. 1A, 1B, and 1E-1H) can be associated through the pairing of Fc heterodimerization variants as described in Section 6.7.1.2.

Further details of the components of the T cell activators of the disclosure are presented below.

Table A below shows particular embodiments of specific T cell activators, including their corresponding half antibody pairings and their binding domains.

TABLE A

| T Cell | | | |
|---|---|---|---|
| Activator | | | Illustrative |
| No. | Left Half-Antibody | Right Half-Antibody | Figure |
| 1 | Half Antibody 5, with immune cell | Half Antibody 4, e.g., with pMHC | 1A |
| | antigen targeting moiety that | complex as described in Section 6.3 | |
| | binds to a TCA, e.g., as described | | |
| | in Section 6.4.1 | | |
| 2 | Half Antibody 5, with immune cell | Half Antibody 4, e.g., with pMHC | 1A |
| | antigen targeting moiety that | complex as described in Section 6.3 | |
| | binds to CD3 | | |
| 3 | Half Antibody 5, with immune cell | Half Antibody 4, e.g., with pMHC | 1A |
| | antigen targeting moiety that | complex as described in Section 6.3 | |
| | binds to CD28 | | |
| 4 | Half Antibody 5, with immune cell | Half Antibody 4, e.g., with pMHC | 1A |
| | antigen targeting moiety that | complex as described in Section 6.3 | |
| | binds to a BCA, e.g., as described | | |
| | in Section 6.4.2 | | |
| 5 | Half Antibody 5, with immune cell | Half Antibody 4, e.g., with pMHC | 1A |
| | antigen targeting moiety that | complex as described in Section 6.3 | |
| | binds to CD19 | | |
| 6 | Half Antibody 5, with immune cell | Half Antibody 4, e.g., with pMHC | 1A |
| | antigen targeting moiety that | complex as described in Section 6.3 | |
| | binds to CD20 | | |
| 7 | Half Antibody 5, with immune cell | Half Antibody 4, e.g., with pMHC | 1A |
| | antigen targeting moiety that | complex as described in Section 6.3 | |
| | binds to CD22 | | |
| 8 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to a | |
| | | TCA, e.g., as described in Section | |
| | | 6.4.1 | |
| 9 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to CD3 | |
| 10 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to CD28 | |
| 11 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to a | |
| | | BCA, e.g., as described in Section | |
| | | 6.4.2 | |
| 12 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to CD19 | |
| 13 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to CD20 | |
| 14 | Half Antibody 4, e.g., with pMHC | Half Antibody 1, e.g., with pMHC | 1B |
| | complex as described in Section | complex as described in Section 6.3 | |
| | 6.3 | and with immune cell antigen | |
| | | targeting moiety that binds to CD22 | |
| 15 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to a | targeting moiety that binds to a | |
| | TCA, e.g., as described in Section | BCA, e.g., as described in Section | |
| | 6.4.1 | 6.4.2 | |
| 16 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to CD3 | targeting moiety that binds to CD19 | |
| 17 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to CD3 | targeting moiety that binds to CD20 | |
| 18 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to CD3 | targeting moiety that binds to CD22 | |
| 19 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD19 | |
| | CD28 | | |
| 20 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD28 | | |
| 21 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD28 | | |
| 22 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to a | targeting moiety that binds to a | |
| | TCA, e.g., as described in Section | TCA, e.g., as described in Section | |
| | 6.4.1 | 6.4.1 | |
| 23 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to CD3 | targeting moiety that binds to CD3 | |
| 24 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to CD3 | targeting moiety that binds to CD28 | |
| 25 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD28 | |
| | CD28 | | |
| 26 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to a | targeting moiety that binds to a | |
| | BCA, e.g., as described in Section | BCA, e.g., as described in Section | |
| | 6.4.2 | 6.4.2 | |
| 27 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD19 | |
| | CD19 | | |
| 28 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD19 | | |
| 29 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD19 | | |
| 30 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD20 | | |
| 31 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD20 | | |
| 32 | Half Antibody 1, e.g., with pMHC | Half Antibody 1, e.g., with pMHC | 1C |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD22 | | |
| 33 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to a | targeting moiety that binds to a | |
| | TCA, e.g., as described in Section | BCA, e.g., as described in Section | |
| | 6.4.1 | 6.4.2 | |
| 34 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to CD3 | targeting moiety that binds to CD19 | |
| 35 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to CD3 | targeting moiety that binds to CD20 | |
| 36 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to CD3 | targeting moiety that binds to CD22 | |
| 37 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD19 | |
| | CD28 | | |
| 38 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD28 | | |
| 39 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD28 | | |
| 40 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to a | targeting moiety that binds to a | |
| | TCA, e.g., as described in Section | TCA, e.g., as described in Section | |
| | 6.4.1 | 6.4.1 | |
| 41 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to CD3 | targeting moiety that binds to CD3 | |
| 42 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to CD3 | targeting moiety that binds to CD28 | |
| 43 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD28 | |
| | CD28 | | |
| 44 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to a | targeting moiety that binds to a | |
| | BCA, e.g., as described in Section | BCA, e.g., as described in Section | |
| | 6.4.2 | 6.4.2 | |
| 45 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD19 | |
| | CD19 | | |
| 46 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD19 | | |
| 47 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD19 | | |
| 48 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD20 | |
| | CD20 | | |
| 49 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD20 | | |
| 50 | Half Antibody 3, e.g., with pMHC | Half Antibody 3, e.g., with pMHC | 1D |
| | complex as described in Section | complex as described in Section 6.3 | |
| | 6.3 and with immune cell antigen | and with immune cell antigen | |
| | targeting moiety that binds to | targeting moiety that binds to CD22 | |
| | CD22 | | |
| 51 | Half Antibody 4, e.g., with pMHC | Half Antibody 6, with first immune | 1E |
| | complex as described in Section | cell antigen targeting moiety that | |
| | 6.3 | binds to a TCA, e.g., as described | |
| | | in Section 6.4.1, and second | |
| | | immune cell antigen targeting | |
| | | moiety that binds to a BCA, e.g., as | |
| | | described in Section 6.4.2 | |
| 52 | Half Antibody 4, e.g., with pMHC | Half Antibody 6, with first immune | 1E |
| | complex as described in Section | cell antigen targeting moiety that | |
| | 6.3 | binds CD3, and second immune cell | |
| | | antigen targeting moiety that binds | |
| | | to CD19 | |
| 53 | Half Antibody 4, e.g., with pMHC | Half Antibody 6, with first immune | 1E |
| | complex as described in Section | cell antigen targeting moiety that | |
| | 6.3 | binds CD3, and second immune cell | |
| | | antigen targeting moiety that binds | |
| | | to CD20 | |
| 54 | Half Antibody 4, e.g., with pMHC | Half Antibody 6, with first immune | 1E |
| | complex as described in Section | cell antigen targeting moiety that | |
| | 6.3 | binds CD3, and second immune cell | |
| | | antigen targeting moiety that binds | |
| | | to CD22 | |
| 55 | Half Antibody 4, e.g., with pMHC | Half Antibody 6, with first immune | 1E |
| | complex as described in Section | cell antigen targeting moiety that | |
| | 6.3 | binds CD28, and second immune | |
| | | cell antigen targeting moiety that | |
| | | binds to CD19 | |
| 56 | Half Antibody 4, e.g., with pMHC | Half Antibody 6, with first immune | 1E |
| | complex as described in Section | cell antigen targeting moiety that | |
| | 6.3 | binds CD28, and second immune | |
| | | cell antigen targeting moiety that | |
| | | binds to CD20 | |
| 57 | Half Antibody 4, e.g., with pMHC | Half Antibody 6, with first immune | 1E |
| | complex as described in Section | cell antigen targeting moiety that | |
| | 6.3 | binds CD28, and second immune | |
| | | cell antigen targeting moiety that | |
| | | binds to CD22 | |
| 58 | Half Antibody 4, e.g., with pMHC | Half Antibody 6, with first immune | 1E |
| | complex as described in Section | cell antigen targeting moiety that | |
| | 6.3 | binds to a BCA, e.g., as described | |
| | | in Section 6.4.2, and second | |
| | | immune cell antigen targeting | |
| | | moiety that binds to a TCA, e.g., as | |
| | | described in Section 6.4.1 | |
| 59 | Half Antibody 4, e.g., with pMHC | Half Antibody 6, with first immune | 1E |
| | complex as described in Section | cell antigen targeting moiety that | |
| | 6.3 | binds CD19, and second immune | |
| | | cell antigen targeting moiety that | |
| | | binds to CD3 | |
| 60 | Half Antibody 4, e.g., with pMHC | Half Antibody 6, with first immune | 1E |
| | complex as described in Section | cell antigen targeting moiety that | |
| | 6.3 | binds CD20, and second immune | |
| | | cell antigen targeting moiety that | |
| | | binds to CD3 | |
| 61 | Half Antibody 4, e.g., with pMHC | Half Antibody 6, with first immune | 1E |
| | complex as described in Section | cell antigen targeting moiety that | |
| | 6.3 | binds CD22, and second immune | |
| | | cell antigen targeting moiety that | |
| | | binds to CD3 | |
| 62 | Half Antibody 4, e.g., with pMHC | Half Antibody 6, with first immune | 1E |
| | complex as described in Section | cell antigen targeting moiety that | |
| | 6.3 | binds CD19, and second immune | |
| | | cell antigen targeting moiety that | |
| | | binds to CD28 | |
| 63 | Half Antibody 4, e.g., with pMHC | Half Antibody 6, with first immune | 1E |
| | complex as described in Section | cell antigen targeting moiety that | |
| | 6.3 | binds CD20, and second immune | |
| | | cell antigen targeting moiety that | |
| | | binds to CD28 | |
| 64 | Half Antibody 4, e.g., with pMHC | Half Antibody 6, with first immune | 1E |
| | complex as described in Section | cell antigen targeting moiety that | |
| | 6.3 | binds CD22, and second immune | |
| | | cell antigen targeting moiety that | |
| | | binds to CD28 | |
| 65 | Half Antibody 4, e.g., with pMHC | Half Antibody 6, with first immune | 1E |
| | complex as described in Section | cell antigen targeting moiety that | |
| | 6.3 | binds to a TCA, e.g., as described | |
| | | in Section 6.4.1, and second | |
| | | immune cell antigen targeting | |
| | | moiety that binds to a TCA, e.g., as | |
| | | described in Section 6.4.1 | |
| 66 | Half Antibody 4, e.g., with pMHC | Half Antibody 6, with first immune | 1E |
| | complex as described in Section | cell antigen targeting moiety that | |
| | 6.3 | binds to CD3, and second immune | |
| | | cell antigen targeting moiety that | |
| | | binds to CD3 | |
| 67 | Half Antibody 4, e.g., with pMHC | Half Antibody 6, with first immune | 1E |
| | complex as described in Section | cell antigen targeting moiety that | |
| | 6.3 | binds to CD28, and second immune | |
| | | cell antigen targeting moiety that | |
| | | binds to CD28 | |
| 68 | Half Antibody 4, e.g., with pMHC | Half Antibody 6, with first immune | 1E |
| | complex as described in Section | cell antigen targeting moiety that | |
| | 6.3 | binds to CD3, and second immune | |
| | | cell antigen targeting moiety that | |
| | | binds to CD28 | |
| 69 | Half Antibody 4, e.g., with pMHC | Half Antibody 6, with first immune | 1E |
| | complex as described in Section | cell antigen targeting moiety that | |
| | 6.3 | binds to CD28, and second immune | |
| | | cell antigen targeting moiety that | |
| | | binds to CD3 | |
| 70 | Half Antibody 4, e.g., with pMHC | Half Antibody 6, with first immune | 1E |
| | complex as described in Section | cell antigen targeting moiety that | |
| | 6.3 | binds to a BCA, e.g., as described | |
| | | in Section 6.4.2 and second | |
| | | immune cell antigen targeting | |
| | | moiety that binds to a BCA, e.g., as | |
| | | described in Section 6.4.2 | |
| 71 | Half Antibody 4, e.g., with pMHC | Half Antibody 6, with first immune | 1E |
| | complex as described in Section | cell antigen targeting moiety that | |
| | 6.3 | binds to CD19, and second immune | |
| | | cell antigen targeting moiety that | |
| | | binds to CD19 | |
| 72 | Half Antibody 4, e.g., with pMHC | Half Antibody 6, with first immune | 1E |
| | complex as described in Section | cell antigen targeting moiety that | |
| | 6.3 | binds to CD20, and second immune | |
| | | cell antigen targeting moiety that | |
| | | binds to CD20 | |
| 73 | Half Antibody 4, e.g., with pMHC | Half Antibody 6, with first immune | 1E |
| | complex as described in Section | cell antigen targeting moiety that | |
| | 6.3 | binds to CD22, and second immune | |
| | | cell antigen targeting moiety that | |
| | | binds to CD22 | |
| 74 | Half Antibody 4, e.g., with pMHC | Half Antibody 6, with first immune | 1E |
| | complex as described in Section | cell antigen targeting moiety that | |
| | 6.3 | binds to CD19, and second immune | |
| | | cell antigen targeting moiety that | |
| | | binds to CD20 | |
| 75 | Half Antibody 4, e.g., with pMHC | Half Antibody 6, with first immune | 1E |
| | complex as described in Section | cell antigen targeting moiety that | |
| | 6.3 | binds to CD20, and second immune | |
| | | cell antigen targeting moiety that | |
| | | binds to CD19 | |
| 76 | Half Antibody 4, e.g., with pMHC | Half Antibody 6, with first immune | 1E |
| | complex as described in Section | cell antigen targeting moiety that | |
| | 6.3 | binds to CD19, and second immune | |
| | | cell antigen targeting moiety that | |
| | | binds to CD22 | |
| 77 | Half Antibody 4, e.g., with pMHC | Half Antibody 6, with first immune | 1E |
| | complex as described in Section | cell antigen targeting moiety that | |
| | 6.3 | binds to CD22, and second immune | |
| | | cell antigen targeting moiety that | |
| | | binds to CD19 | |
| 78 | Half Antibody 4, e.g., with pMHC | Half Antibody 6, with first immune | 1E |
| | complex as described in Section | cell antigen targeting moiety that | |
| | 6.3 | binds to CD20, and second immune | |
| | | cell antigen targeting moiety that | |
| | | binds to CD22 | |
| 79 | Half Antibody 4, e.g., with pMHC | Half Antibody 6, with first immune | 1E |
| | complex as described in Section | cell antigen targeting moiety that | |
| | 6.3 | binds to CD22, and second immune | |
| | | cell antigen targeting moiety that | |
| | | binds to CD20 | |
| 80 | Half Antibody 1, e.g., with pMHC | Half Antibody 5, with immune cell | 1F |
| | complex as described in Section | antigen targeting moiety that binds | |
| | 6.3 and with immune cell antigen | to a TCA, e.g., as described in | |
| | targeting moiety that binds to a | Section 6.4.1 | |
| | TCA, e.g., as described in Section | | |
| | 6.4.1 | | |
| 81 | Half Antibody 1, e.g., with pMHC | Half Antibody 5, with immune cell | 1F |
| | complex as described in Section | antigen targeting moiety that binds | |
| | 6.3 and with immune cell antigen | to CD3 | |
| | targeting moiety that binds to CD3 | | |
| 82 | Half Antibody 1, e.g., with pMHC | Half Antibody 5, with immune cell | 1F |
| | complex as described in Section | antigen targeting moiety that binds | |
| | 6.3 and with immune cell antigen | to CD28 | |
| | targeting moiety that binds to | | |
| | CD28 | | |
| 83 | Half Antibody 1, e.g., with pMHC | Half Antibody 5, with immune cell | 1F |
| | complex as described in Section | antigen targeting moiety that binds | |
| | 6.3 and with immune cell antigen | to CD28 | |
| | targeting moiety that binds to CD3 | | |
| 84 | Half Antibody 1, e.g., with pMHC | Half Antibody 5, with immune cell | 1F |
| | complex as described in Section | antigen targeting moiety that binds | |
| | 6.3 and with immune cell antigen | to CD3 | |
| | targeting moiety that binds to | | |
| | CD28 | | |
| 85 | Half Antibody 1, e.g., with pMHC | Half Antibody 5, with immune cell | 1F |
| | complex as described in Section | antigen targeting moiety that binds | |
| | 6.3 and with immune cell antigen | to a TCA, e.g., as described in | |
| | targeting moiety that binds to a | Section 6.4.1 | |
| | BCA, e.g., as described in Section | | |
| | 6.4.2 | | |
| 86 | Half Antibody 1, e.g., with pMHC | Half Antibody 5, with immune cell | 1F |
| | complex as described in Section | antigen targeting moiety that binds | |
| | 6.3 and with immune cell antigen | to CD3 | |
| | targeting moiety that binds to | | |
| | CD19 | | |
| 87 | Half Antibody 1, e.g., with pMHC | Half Antibody 5, with immune cell | 1F |
| | complex as described in Section | antigen targeting moiety that binds | |
| | 6.3 and with immune cell antigen | to CD3 | |
| | targeting moiety that binds to | | |
| | CD20 | | |
| 88 | Half Antibody 1, e.g., with pMHC | Half Antibody 5, with immune cell | 1F |
| | complex as described in Section | antigen targeting moiety that binds | |
| | 6.3 and with immune cell antigen | to CD3 | |
| | targeting moiety that binds to | | |
| | CD22 | | |
| 89 | Half Antibody 1, e.g., with pMHC | Half Antibody 5, with immune cell | 1F |
| | complex as described in Section | antigen targeting moiety that binds | |
| | 6.3 and with immune cell antigen | to CD28 | |
| | targeting moiety that binds to | | |
| | CD19 | | |
| 90 | Half Antibody 1, e.g., with pMHC | Half Antibody 5, with immune cell | 1F |
| | complex as described in Section | antigen targeting moiety that binds | |
| | 6.3 and with immune cell antigen | to CD28 | |
| | targeting moiety that binds to | | |
| | CD20 | | |
| 91 | Half Antibody 1, e.g., with pMHC | Half Antibody 5, with immune cell | 1F |
| | complex as described in Section | antigen targeting moiety that binds | |
| | 6.3 and with immune cell antigen | to CD28 | |
| | targeting moiety that binds to | | |
| | CD22 | | |
| 92 | Half Antibody 1, e.g., with pMHC | Half Antibody 5, with immune cell | 1F |
| | complex as described in Section | antigen targeting moiety that binds | |
| | 6.3 and with immune cell antigen | to a BCA, e.g., as described in | |
| | targeting moiety that binds to a | Section 6.4.2 | |
| | TCA, e.g., as described in Section | | |
| | 6.4.1 | | |
| 93 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to CD3 | | |
| 94 | Half Antibody 1, e.g., with pMHC | Half Antibody 5, with immune cell | 1F |
| | complex as described in Section | antigen targeting moiety that binds | |
| | 6.3 and with immune cell antigen | to CD20 | |
| | targeting moiety that binds to CD3 | | |
| 95 | Half Antibody 1, e.g., with pMHC | Half Antibody 5, with immune cell | 1F |
| | complex as described in Section | antigen targeting moiety that binds | |
| | 6.3 and with immune cell antigen | to CD22 | |
| | targeting moiety that binds to CD3 | | |
| 96 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to | | |
| | CD28 | | |
| 97 | Half Antibody 1, e.g., with pMHC | Half Antibody 5, with immune cell | 1F |
| | complex as described in Section | antigen targeting moiety that binds | |
| | 6.3 and with immune cell antigen | to CD20 | |
| | targeting moiety that binds to | | |
| | CD28 | | |
| 98 | Half Antibody 1, e.g., with pMHC | Half Antibody 5, with immune cell | 1F |
| | complex as described in Section | antigen targeting moiety that binds | |
| | 6.3 and with immune cell antigen | to CD22 | |
| | targeting moiety that binds to | | |
| | CD28 | | |
| 99 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to CD3 | | |
| 100 | Half Antibody 1, e.g., with pMHC | Half Antibody 5, with immune cell | 1F |
| | complex as described in Section | antigen targeting moiety that binds | |
| | 6.3 and with immune cell antigen | to a BCA, e.g., as described in | |
| | targeting moiety that binds to a | Section 6.4.2 | |
| | BCA, e.g., as described in Section | | |
| | 6.4.2 | | |
| 101 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to | | |
| | CD19 | | |
| 102 | Half Antibody 1, e.g., with pMHC | Half Antibody 5, with immune cell | 1F |
| | complex as described in Section | antigen targeting moiety that binds | |
| | 6.3 and with immune cell antigen | to CD20 | |
| | targeting moiety that binds to | | |
| | CD20 | | |
| 103 | Half Antibody 1, e.g., with pMHC | Half Antibody 5, with immune cell | 1F |
| | complex as described in Section | antigen targeting moiety that binds | |
| | 6.3 and with immune cell antigen | to CD22 | |
| | targeting moiety that binds to | | |
| | CD22 | | |
| 104 | Half Antibody 1, e.g., with pMHC | Half Antibody 5, with immune cell | 1F |
| | complex as described in Section | antigen targeting moiety that binds | |
| | 6.3 and with immune cell antigen | to CD20 | |
| | targeting moiety that binds to | | |
| | CD19 | | |
| 105 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to | | |
| | CD20 | | |
| 106 | Half Antibody 1, e.g., with pMHC | Half Antibody 5, with immune cell | 1F |
| | complex as described in Section | antigen targeting moiety that binds | |
| | 6.3 and with immune cell antigen | to CD22 | |
| | targeting moiety that binds to | | |
| | CD19 | | |
| 107 | Half Antibody 1, e.g., with pMHC | Half Antibody 5, with immune cell | 1F |
| | complex as described in Section | antigen targeting moiety that binds | |
| | 6.3 and with immune cell antigen | to CD19 | |
| | targeting moiety that binds to | | |
| | CD22 | | |
| 108 | Half Antibody 1, e.g., with pMHC | Half Antibody 5, with immune cell | 1F |
| | complex as described in Section | antigen targeting moiety that binds | |
| | 6.3 and with immune cell antigen | to CD22 | |
| | targeting moiety that binds to | | |
| | CD20 | | |
| 109 | Half Antibody 1, e.g., with pMHC | Half Antibody 5, with immune cell | 1F |
| | complex as described in Section | antigen targeting moiety that binds | |
| | 6.3 and with immune cell antigen | to CD20 | |
| | targeting moiety that binds to | | |
| | CD22 | | |
| 110 | Half Antibody 1, e.g., with pMHC | Half Antibody 6, with first immune | 1G |
| | complex as described in Section | cell antigen targeting moiety that | |
| | 6.3 and with immune cell antigen | binds to a TCA, e.g., as described | |
| | targeting moiety that binds to a | in Section 6.4.1, and second | |
| | BCA, e.g., as described in Section | immune cell antigen targeting | |
| | 6.4.2 | moiety that binds to a BCA, e.g., as | |
| | | described in Section 6.4.2 | |
| 111 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD19 | binds to CD19 | |
| 112 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD19 | binds to CD20 | |
| 113 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD19 | binds to CD22 | |
| 114 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD19 | binds to CD19 | |
| 115 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD19 | binds to CD20 | |
| 116 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD19 | binds to CD22 | |
| 117 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD20 | binds to CD19 | |
| 118 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD20 | binds to CD20 | |
| 119 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD20 | binds to CD22 | |
| 120 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD20 | binds to CD19 | |
| 121 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD20 | binds to CD20 | |
| 122 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD20 | binds to CD22 | |
| 123 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD22 | binds to CD19 | |
| 124 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD22 | binds to CD20 | |
| 125 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD22 | binds to CD22 | |
| 126 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD22 | binds to CD19 | |
| 127 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD22 | binds to CD20 | |
| 128 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD22 | binds to CD22 | |
| 129 | Half Antibody 1, e.g., with pMHC | Half Antibody 6, with first immune | 1G |
| | complex as described in Section | cell antigen targeting moiety that | |
| | 6.3 and with immune cell antigen | binds to a BCA, e.g., as described | |
| | targeting moiety that binds to a | in Section 6.4.2, and second | |
| | BCA, e.g., as described in Section | immune cell antigen targeting | |
| | 6.4.2 | moiety that binds to a TCA, e.g., as | |
| | | described in Section 6.4.1 | |
| 130 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD3 | |
| 131 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD19 | binds to CD3 | |
| 132 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD19 | binds to CD3 | |
| 133 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD28 | |
| 134 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD28 | |
| 135 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD28 | |
| 136 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD3 | |
| 137 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD20 | binds to CD3 | |
| 138 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD20 | binds to CD3 | |
| 139 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD28 | |
| 140 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD28 | |
| 141 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD28 | |
| 142 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD3 | |
| 143 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD22 | binds to CD3 | |
| 144 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD22 | binds to CD3 | |
| 145 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD28 | |
| 146 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD28 | |
| 147 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD28 | |
| 148 | Half Antibody 1, e.g., with pMHC | Half Antibody 6, with first immune | 1G |
| | complex as described in Section | cell antigen targeting moiety that | |
| | 6.3 and with immune cell antigen | binds to a TCA, e.g., as described | |
| | targeting moiety that binds to a | in Section 6.4.1, and second | |
| | BCA, e.g., as described in Section | immune cell antigen targeting | |
| | 6.4.2 | moiety that binds to a TCA, e.g., as | |
| | | described in Section 6.4.1 | |
| 149 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD19 | binds to CD3 | |
| 150 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD19 | binds to CD28 | |
| 151 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD19 | binds to CD3 | |
| 152 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD19 | binds to CD28 | |
| 153 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD20 | binds to CD3 | |
| 154 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD20 | binds to CD28 | |
| 155 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD20 | binds to CD3 | |
| 156 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD20 | binds to CD28 | |
| 157 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD22 | binds to CD3 | |
| 158 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD22 | binds to CD28 | |
| 159 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD22 | binds to CD3 | |
| 160 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD22 | binds to CD28 | |
| 161 | Half Antibody 1, e.g., with pMHC | Half Antibody 6, with first immune | 1G |
| | complex as described in Section | cell antigen targeting moiety that | |
| | 6.3 and with immune cell antigen | binds to a BCA, e.g., as described | |
| | targeting moiety that binds to a | in Section 6.4.2, and second | |
| | BCA, e.g., as described in Section | immune cell antigen targeting | |
| | 6.4.2 | moiety that binds to a BCA, e.g., as | |
| | | described in Section 6.4.2 | |
| 162 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD19 | |
| 163 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD20 | |
| 164 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD19 | binds to CD22 | |
| 165 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD19 | binds to CD19 | |
| 166 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD19 | binds to CD20 | |
| 167 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD19 | binds to CD22 | |
| 168 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD19 | binds to CD19 | |
| 169 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD19 | binds to CD20 | |
| 170 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD19 | binds to CD22 | |
| 17' | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD19 | |
| 172 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD20 | |
| 173 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD20 | binds to CD22 | |
| 174 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD20 | binds to CD19 | |
| 175 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD20 | binds to CD20 | |
| 176 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD20 | binds to CD22 | |
| 177 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD20 | binds to CD19 | |
| 178 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD20 | binds to CD20 | |
| 179 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD20 | binds to CD22 | |
| 180 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD19 | |
| 181 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD20 | |
| 182 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD22 | binds to CD22 | |
| 183 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD22 | binds to CD19 | |
| 184 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD22 | binds to CD20 | |
| 185 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD22 | binds to CD22 | |
| 186 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD22 | binds to CD19 | |
| 187 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD22 | binds to CD20 | |
| 188 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD22 | binds to CD22 | |
| 189 | Half Antibody 1, e.g., with pMHC | Half Antibody 6, with first immune | 1G |
| | complex as described in Section | cell antigen targeting moiety that | |
| | 6.3 and with immune cell antigen | binds to a TCA, e.g., as described | |
| | targeting moiety that binds to a | in Section 6.4.1, and second | |
| | TCA, e.g., as described in Section | immune cell antigen targeting | |
| | 6.4.1 | moiety that binds to a BCA, e.g., as | |
| | | described in Section 6.4.2 | |
| 190 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to CD3 | cell antigen targeting moiety that | |
| | | binds to CD19 | |
| 191 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to CD3 | cell antigen targeting moiety that | |
| | | binds to CD20 | |
| 192 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to CD3 | cell antigen targeting moiety that | |
| | | binds to CD22 | |
| 193 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to CD3 | cell antigen targeting moiety that | |
| | | binds to CD19 | |
| 194 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to CD3 | cell antigen targeting moiety that | |
| | | binds to CD20 | |
| 195 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to CD3 | cell antigen targeting moiety that | |
| | | binds to CD22 | |
| 196 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD28 | binds to CD19 | |
| 197 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD28 | binds to CD20 | |
| 198 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD28 | binds to CD22 | |
| 199 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD28 | binds to CD19 | |
| 200 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD28 | binds to CD20 | |
| 201 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD28 | binds to CD22 | |
| 202 | Half Antibody 1, e.g., with pMHC | Half Antibody 6, with first immune | 1G |
| | complex as described in Section | cell antigen targeting moiety that | |
| | 6.3 and with immune cell antigen | binds to a BCA, e.g., as described | |
| | targeting moiety that binds to a | in Section 6.4.2, and second | |
| | TCA, e.g., as described in Section | immune cell antigen targeting | |
| | 6.4.1 | moiety that binds to a TCA, e.g., as | |
| | | described in Section 6.4.1 | |
| 203 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to CD3 | cell antigen targeting moiety that | |
| | | binds to CD3 | |
| 204 | Half Antibody 1, e.g., with pMHC | Half Antibody 6, with first immune | 1G |
| | complex as described in Section | cell antigen targeting moiety that | |
| | | binds to CD20, and second immune | |
| | 6.3 and with immune cell antigen | cell antigen targeting moiety that | |
| | targeting moiety that binds to CD3 | binds to CD3 | |
| 205 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to CD3 | cell antigen targeting moiety that | |
| | | binds to CD3 | |
| 206 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to CD3 | cell antigen targeting moiety that | |
| | | binds to CD28 | |
| 207 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to CD3 | cell antigen targeting moiety that | |
| | | binds to CD28 | |
| 208 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to CD3 | cell antigen targeting moiety that | |
| | | binds to CD28 | |
| 209 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD28 | binds to CD3 | |
| 210 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD28 | binds to CD3 | |
| 211 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD28 | binds to CD3 | |
| 212 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD28 | binds to CD28 | |
| 213 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD28 | binds to CD28 | |
| 214 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD28 | binds to CD28 | |
| 215 | Half Antibody 1, e.g., with pMHC | Half Antibody 6, with first immune | 1G |
| | complex as described in Section | cell antigen targeting moiety that | |
| | 6.3 and with immune cell antigen | binds to a TCA, e.g., as described | |
| | targeting moiety that binds to a | in Section 6.4.1, and second | |
| | TCA, e.g., as described in Section | immune cell antigen targeting | |
| | 6.4.1 | moiety that binds to a TCA, e.g., as | |
| | | described in Section 6.4.1 | |
| 216 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to CD3 | cell antigen targeting moiety that | |
| | | binds to CD3 | |
| 217 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to CD3 | cell antigen targeting moiety that | |
| | | binds to CD28 | |
| 218 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to CD3 | cell antigen targeting moiety that | |
| | | binds to CD3 | |
| 219 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to CD3 | cell antigen targeting moiety that | |
| | | binds to CD28 | |
| 220 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD28 | binds to CD3 | |
| 221 | Half Antibody 1, e.g., with pMHC | Half Antibody 6, with first immune | 1G |
| | complex as described in Section | cell antigen targeting moiety that | |
| | 6.3 and with immune cell antigen | binds to CD3, and second immune | |
| | targeting moiety that binds to | cell antigen targeting moiety that | |
| | CD28 | binds to CD28 | |
| 222 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD28 | binds to CD3 | |
| 223 | Half Antibody 1, e.g., with pMHC | Half Antibody 6, with first immune | 1G |
| | complex as described in Section | cell antigen targeting moiety that | |
| | 6.3 and with immune cell antigen | binds to CD28, and second immune | |
| | targeting moiety that binds to | cell antigen targeting moiety that | |
| | CD28 | binds to CD28 | |
| 224 | Half Antibody 1, e.g., with pMHC | Half Antibody 6, with first immune | 1G |
| | complex as described in Section | cell antigen targeting moiety that | |
| | 6.3 and with immune cell antigen | binds to a BCA, e.g., as described | |
| | targeting moiety that binds to a | in Section 6.4.2, and second | |
| | TCA, e.g., as described in Section | immune cell antigen targeting | |
| | 6.4.1 | moiety that binds to a BCA, e.g., as | |
| | | described in Section 6.4.2 | |
| 225 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to CD3 | cell antigen targeting moiety that | |
| | | binds to CD19 | |
| 226 | Half Antibody 1, e.g., with pMHC | Half Antibody 6, with first immune | 1G |
| | complex as described in Section | cell antigen targeting moiety that | |
| | | binds to CD19, and second immune | |
| | 6.3 and with immune cell antigen | cell antigen targeting moiety that | |
| | targeting moiety that binds to CD3 | binds to CD20 | |
| 227 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to CD3 | cell antigen targeting moiety that | |
| | | binds to CD22 | |
| 228 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to CD3 | cell antigen targeting moiety that | |
| | | binds to CD19 | |
| 229 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to CD3 | cell antigen targeting moiety that | |
| | | binds to CD20 | |
| 230 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to CD3 | cell antigen targeting moiety that | |
| | | binds to CD22 | |
| 231 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to CD3 | cell antigen targeting moiety that | |
| | | binds to CD19 | |
| 232 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to CD3 | cell antigen targeting moiety that | |
| | | binds to CD20 | |
| 233 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to CD3 | cell antigen targeting moiety that | |
| | | binds to CD22 | |
| 234 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD28 | binds to CD19 | |
| 235 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD28 | binds to CD20 | |
| 236 | Half Antibody 1, e.g., with pMHC | Half Antibody 6, with first immune | 1G |
| | complex as described in Section | cell antigen targeting moiety that | |
| | 6.3 and with immune cell antigen | binds to CD19, and second immune | |
| | targeting moiety that binds to | cell antigen targeting moiety that | |
| | CD28 | binds to CD22 | |
| 237 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD28 | binds to CD19 | |
| 238 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD28 | binds to CD20 | |
| 239 | Half Antibody 1, e.g., with pMHC | Half Antibody 6, with first immune | 1G |
| | complex as described in Section | cell antigen targeting moiety that | |
| | 6.3 and with immune cell antigen | binds to CD20, and second immune | |
| | targeting moiety that binds to | cell antigen targeting moiety that | |
| | CD28 | binds to CD22 | |
| 240 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD28 | binds to CD19 | |
| 241 | Half Antibody 1, e.g., with pMHC | Half Antibody 6, with first immune | 1G |
| | complex as described in Section | cell antigen targeting moiety that | |
| | 6.3 and with immune cell an tigen | binds to CD22, and second immune | |
| | targeting moiety that binds to | cell antigen targeting moiety that | |
| | CD28 | binds to CD20 | |
| 242 | Half Antibody 1, e.g., with pMHC | Half Antibody 6, with first immune | 1G |
| | complex as described in Section | cell antigen targeting moiety that | |
| | 6.3 and with immune cell antigen | binds to CD22, and second immune | |
| | targeting moiety that binds to | cell antigen targeting moiety that | |
| | CD28 | binds to CD22 | |
| 243 | Half Antibody 4, e.g., with pMHC | Half Antibody 7, with first immune | 1H |
| | complex as described in Section | cell antigen targeting moiety that | |
| | 6.3 | binds to a TCA, e.g., as described | |
| | | in Section 6.4.1.1, and tumor | |
| | | antigen targeting moiety, e.g., as | |
| | | described in Section 6.5 | |
| 244 | Half Antibody 4, e.g., with pMHC | Half Antibody 7, with first immune | 1H |
| | complex as described in Section | cell antigen targeting moiety that | |
| | 6.3 | binds to CD3, and tumor antigen | |
| | | targeting moiety, e.g., as described | |
| | | in Section 6.5 | |
| 245 | Half Antibody 4, e.g., with pMHC | Half Antibody 7, with first immune | 1H |
| | complex as described in Section | cell antigen targeting moiety that | |
| | 6.3 | binds to CD28, and tumor antigen | |
| | | targeting moiety, e.g., as described | |
| | | in Section 6.5 | |
| 246 | Half Antibody 4, e.g., with pMHC | Half Antibody 7, with first immune | 1H |
| | complex as described in Section | cell antigen targeting moiety that | |
| | 6.3 | binds to a BCA, e.g., as described | |
| | | in Section 6.4.2, and tumor antigen | |
| | | targeting moiety, e.g., as described | |
| | | in Section 6.5 | |
| 247 | Half Antibody 4, e.g., with pMHC | Half Antibody 7, with first immune | 1H |
| | complex as described in Section | cell antigen targeting moiety that | |
| | 6.3 | binds to CD19, and tumor antigen | |
| | | targeting moiety, e.g., as described | |
| | | in Section 6.5 | |
| 248 | Half Antibody 4, e.g., with pMHC | Half Antibody 7, with first immune | 1H |
| | complex as described in Section | cell antigen targeting moiety that | |
| | 6.3 | binds to CD20, and tumor antigen | |
| | | targeting moiety, e.g., as described | |
| | | in Section 6.5 | |
| 249 | Half Antibody 4, e.g., with pMHC | Half Antibody 7, with first immune | 1H |
| | complex as described in Section | cell antigen targeting moiety that | |
| | 6.3 | binds to CD22, and tumor antigen | |
| | | targeting moiety, e.g., as described | |
| | | in Section 6.5 | |
| 250 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to a | second tumor antigen targeting | |
| | TCA, e.g., as described in Section | moiety, e.g., as described in Section | |
| | 6.4.1 | 6.5 | |
| 251 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to CD3 | second tumor antigen targeting | |
| | | moiety, e.g., as described in Section | |
| | | 6.5 | |
| 252 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to | second tumor antigen targeting | |
| | CD28 | moiety, e.g., as described in Section | |
| | | 6.5 | |
| 253 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to a | second tumor antigen targeting | |
| | BCA, e.g., as described in Section | moiety, e.g., as described in Section | |
| | 6.4.2 | 6.5 | |
| 254 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to | second tumor antigen targeting | |
| | CD19 | moiety, e.g., as described in Section | |
| | | 6.5 | |
| 255 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | targeting moiety that binds to | second tumor antigen targeting | |
| | CD20 | moiety, e.g., as described in Section | |
| | | 6.5 | |
| 256 | Half Antibody 1, e.g., with pMHC | Half Antibody 8, with first tumor | 1I |
| | complex as described in Section | antigen targeting moiety, e.g., as | |
| | 6.3 and with immune cell antigen | described in Section 6.5, and | |
| | | second tumor antigen targeting | |
| | targeting moiety that binds to | moiety, e.g., as described in Section | |
| | CD22 | 6.5 | |
| 257 | Half Antibody 2, with immune cell | Half Antibody 5, with immune cell | N/A |
| | antigen targeting moiety that | antigen targeting moiety that binds | |
| | binds to a TCA, e.g., as described | to a TCA, e.g., as described in | |
| | in Section 6.4.1 and e.g., with | Section 6.4.1 | |
| | pMHC complex as described in | | |
| | Section 6.3 | | |
| 258 | Half Antibody 2, with immune cell | Half Antibody 5, with immune cell | N/A |
| | antigen targeting moiety that | antigen targeting moiety that binds | |
| | binds to CD3 and e.g., with pMHC | to CD3 | |
| | complex as described in Section | | |
| | 6.3 | | |
| 259 | Half Antibody 2, with immune cell | Half Antibody 5, with immune cell | N/A |
| | antigen targeting moiety that | antigen targeting moiety that binds | |
| | binds to CD3 and e.g., with pMHC | to CD28 | |
| | complex as described in Section | | |
| | 6.3 | | |
| 260 | Half Antibody 2, with immune cell | Half Antibody 5, with immune cell | N/A |
| | antigen targeting moiety that | antigen targeting moiety that binds | |
| | binds to CD28 and e.g., with | to CD3 | |
| | pMHC complex as described in | | |
| | Section 6.3 | | |
| 261 | Half Antibody 2, with immune cell | Half Antibody 5, with immune cell | N/A |
| | antigen targeting moiety that | antigen targeting moiety that binds | |
| | binds to CD28 and e.g., with | to CD3 | |
| | pMHC complex as described in | | |
| | Section 6.3 | | |
| 262 | Half Antibody 2, with immune cell | Half Antibody 5, with immune cell | N/A |
| | antigen targeting moiety that | antigen targeting moiety that binds | |
| | binds to a TCA, e.g., as described | to a BCA, e.g., as described in | |
| | in Section 6.4.1 and e.g., with | Section 6.4.2 | |
| | pMHC complex as described in | | |
| | Section 6.3 | | |
| 263 | Half Antibody 2, with immune cell | Half Antibody 5, with immune cell | N/A |
| | antigen targeting moiety that | antigen targeting moiety that binds | |
| | binds to CD3 and e.g., with pMHC | to CD19 | |
| | complex as described in Section | | |
| | 6.3 | | |
| 264 | Half Antibody 2, with immune cell | Half Antibody 5, with immune cell | N/A |
| | antigen targeting moiety that | antigen targeting moiety that binds | |
| | binds to CD3 and e.g., with pMHC | to CD20 | |
| | complex as described in Section | | |
| | 6.3 | | |
| 265 | Half Antibody 2, with immune cell | Half Antibody 5, with immune cell | N/A |
| | antigen targeting moiety that | antigen targeting moiety that binds | |
| | binds to CD3 and e.g., with pMHC | to CD22 | |
| | complex as described in Section | | |
| | 6.3 | | |
| 266 | Half Antibody 2, with immune cell | Half Antibody 5, with immune cell | N/A |
| | antigen targeting moiety that | antigen targeting moiety that binds | |
| | binds to CD28 and e.g., with | to CD19 | |
| | pMHC complex as described in | | |
| | Section 6.3 | | |
| 267 | Half Antibody 2, with immune cell | Half Antibody 5, with immune cell | N/A |
| | antigen targeting moiety that | antigen targeting moiety that binds | |
| | binds to CD28 and e.g., with | to CD20 | |
| | pMHC complex as described in | | |
| | Section 6.3 | | |
| 268 | Half Antibody 2, with immune cell | Half Antibody 5, with immune cell | N/A |
| | antigen targeting moiety that | antigen targeting moiety that binds | |
| | binds to CD28 and e.g., with | to CD22 | |
| | pMHC complex as described in | | |
| | Section 6.3 | | |
| 269 | Half Antibody 2, with immune cell | Half Antibody 5, with immune cell | N/A |
| | antigen targeting moiety that | antigen targeting moiety that binds | |
| | binds to a BCA, e.g., as described | to a TCA, e.g., as described in | |
| | in Section 6.4.2 and e.g., with | Section 6.4.1 | |
| | pMHC complex as described in | | |
| | Section 6.3 | | |
| 270 | Half Antibody 2, with immune cell | Half Antibody 5, with immune cell | N/A |
| | antigen targeting moiety that | antigen targeting moiety that binds | |
| | binds to CD19 and e.g., with | to CD3 | |
| | pMHC complex as described in | | |
| | Section 6.3 | | |
| 271 | Half Antibody 2, with immune cell | Half Antibody 5, with immune cell | N/A |
| | antigen targeting moiety that | antigen targeting moiety that binds | |
| | binds to CD20 and e.g., with | to CD3 | |
| | pMHC complex as described in | | |
| | Section 6.3 | | |
| 272 | Half Antibody 2, with immune cell | Half Antibody 5, with immune cell | N/A |
| | antigen targeting moiety that | antigen targeting moiety that binds | |
| | binds to CD22 and e.g., with | to CD3 | |
| | pMHC complex as described in | | |
| | Section 6.3 | | |
| 273 | Half Antibody 2, with immune cell | Half Antibody 5, with immune cell | N/A |
| | antigen targeting moiety that | antigen targeting moiety that binds | |
| | binds to CD19 and e.g., with | to CD28 | |
| | pMHC complex as described in | | |
| | Section 6.3 | | |
| 274 | Half Antibody 2, with immune cell | Half Antibody 5, with immune cell | N/A |
| | antigen targeting moiety that | antigen targeting moiety that binds | |
| | binds to CD20 and e.g., with | to CD28 | |
| | pMHC complex as described in | | |
| | Section 6.3 | | |
| 275 | Half Antibody 2, with immune cell | Half Antibody 5, with immune cell | N/A |
| | antigen targeting moiety that | antigen targeting moiety that binds | |
| | binds to CD22 and e.g., with | to CD28 | |
| | pMHC complex as described in | | |
| | Section 6.3 | | |
| 276 | Half Antibody 2, with immune cell | Half Antibody 5, with immune cell | N/A |
| | antigen targeting moiety that | antigen targeting moiety that binds | |
| | binds to a BCA, e.g., as described | to a BCA, e.g., as described in | |
| | in Section 6.4.2 and e.g., with | Section 6.4.2 | |
| | pMHC complex as described in | | |
| | Section 6.3 | | |
| 277 | Half Antibody 2, with immune cell | Half Antibody 5, with immune cell | N/A |
| | antigen targeting moiety that | antigen targeting moiety that binds | |
| | binds to CD19 and e.g., with | to CD19 | |
| | pMHC complex as described in | | |
| | Section 6.3 | | |
| 278 | Half Antibody 2, with immune cell | Half Antibody 5, with immune cell | N/A |
| | antigen targeting moiety that | antigen targeting moiety that binds | |
| | binds to CD19 and e.g., with | to CD20 | |
| | pMHC complex as described in | | |
| | Section 6.3 | | |
| 279 | Half Antibody 2, with immune cell | Half Antibody 5, with immune cell | N/A |
| | antigen targeting moiety that | antigen targeting moiety that binds | |
| | binds to CD19 and e.g., with | to CD22 | |
| | pMHC complex as described in | | |
| | Section 6.3 | | |
| 280 | Half Antibody 2, with immune cell | Half Antibody 5, with immune cell | N/A |
| | antigen targeting moiety that | antigen targeting moiety that binds | |
| | binds to CD20 and e.g., with | to CD19 | |
| | pMHC complex as described in | | |
| | Section 6.3 | | |
| 281 | Half Antibody 2, with immune cell | Half Antibody 5, with immune cell | N/A |
| | antigen targeting moiety that | antigen targeting moiety that binds | |
| | binds to CD20 and e.g., with | to CD20 | |
| | pMHC complex as described in | | |
| | Section 6.3 | | |
| 282 | Half Antibody 2, with immune cell | Half Antibody 5, with immune cell | N/A |
| | antigen targeting moiety that | antigen targeting moiety that binds | |
| | binds to CD20 and e.g., with | to CD22 | |
| | pMHC complex as described in | | |
| | Section 6.3 | | |
| 283 | Half Antibody 2, with immune cell | Half Antibody 5, with immune cell | N/A |
| | antigen targeting moiety that | antigen targeting moiety that binds | |
| | binds to CD22 and e.g., with | to CD19 | |
| | pMHC complex as described in | | |
| | Section 6.3 | | |
| 284 | Half Antibody 2, with immune cell | Half Antibody 5, with immune cell | N/A |
| | antigen targeting moiety that | antigen targeting moiety that binds | |
| | binds to CD22 and e.g., with | to CD20 | |
| | pMHC complex as described in | | |
| | Section 6.3 | | |
| 285 | Half Antibody 2, with immune cell | Half Antibody 5, with immune cell | N/A |
| | antigen targeting moiety that | antigen targeting moiety that binds | |
| | binds to CD22 and e.g., with | to CD22 | |
| | pMHC complex as described in | | |
| | Section 6.3 | | |
| 286 | Half Antibody 2, with immune cell | Half Antibody 2, with immune cell | N/A |
| | antigen targeting moiety that | antigen targeting moiety that binds | |
| | binds to a TCA, e.g., as described | to a TCA, e.g., as described in | |
| | in Section 6.4.1 and e.g., with | Section 6.4.1 and e.g., with pMHC | |
| | pMHC complex as described in | complex as described in Section 6.3 | |
| | Section 6.3 | | |
| 287 | Half Antibody 2, with immune cell | Half Antibody 2, with immune cell | N/A |
| | antigen targeting moiety that | antigen targeting moiety that binds | |
| | binds to CD3 and e.g., with pMHC | to CD3 and e.g., with pMHC | |
| | complex as described in Section | complex as described in Section 6.3 | |
| | 6.3 | | |
| 288 | Half Antibody 2, with immune cell | Half Antibody 2, with immune cell | N/A |
| | antigen targeting moiety that | antigen targeting moiety that binds | |
| | binds to CD3 and e.g., with pMHC | to CD28 and e.g., with pMHC | |
| | complex as described in Section | complex as described in Section 6.3 | |
| | 6.3 | | |
| 289 | Half Antibody 2, with immune cell | Half Antibody 2, with immune cell | N/A |
| | antigen targeting moiety that | antigen targeting moiety that binds | |
| | binds to CD28 and e.g., with | to CD3 and e.g., with pMHC | |
| | pMHC complex as described in | complex as described in Section 6.3 | |
| | Section 6.3 | | |
| 290 | Half Antibody 2, with immune cell | Half Antibody 2, with immune cell | N/A |
| | antigen targeting moiety that | antigen targeting moiety that binds | |
| | binds to CD28 and e.g., with | | |
| | pMHC complex as described in | to CD28 and e.g., with pMHC | |
| | Section 6.3 | complex as described in Section 6.3 | |
| 291 | Half Antibody 2, with immune cell | Half Antibody 2, with immune cell | N/A |
| | antigen targeting moiety that | antigen targeting moiety that binds | |
| | binds to a TCA, e.g., as described | to a BCA, e.g., as described in | |
| | in Section 6.4.1 and e.g., with | Section 6.4.2 and e.g., with pMHC | |
| | pMHC complex as described in | complex as described in Section 6.3 | |
| | Section 6.3 | | |
| 292 | Half Antibody 2, with immune cell | Half Antibody 2, with immune cell | N/A |
| | antigen targeting moiety that | antigen targeting moiety that binds | |
| | binds to CD3 and e.g., with pMHC | to CD19 and e.g., with pMHC | |
| | complex as described in Section | complex as described in Section 6.3 | |
| | 6.3 | | |
| 293 | Half Antibody 2, with immune cell | Half Antibody 2, with immune cell | N/A |
| | antigen targeting moiety that | antigen targeting moiety that binds | |
| | binds to CD3 and e.g., with pMHC | to CD20 and e.g., with pMHC | |
| | complex as described in Section | complex as described in Section 6.3 | |
| | 6.3 | | |
| 294 | Half Antibody 2, with immune cell | Half Antibody 2, with immune cell | N/A |
| | antigen targeting moiety that | antigen targeting moiety that binds | |
| | binds to CD3 and e.g., with pMHC | to CD22 and e.g., with pMHC | |
| | complex as described in Section | complex as described in Section 6.3 | |
| | 6.3 | | |
| 295 | Half Antibody 2, with immune cell | Half Antibody 2, with immune cell | N/A |
| | antigen targeting moiety that | antigen targeting moiety that binds | |
| | binds to CD28 and e.g., with | to CD19 and e.g., with pMHC | |
| | pMHC complex as described in | complex as described in Section 6.3 | |
| | Section 6.3 | | |
| 296 | Half Antibody 2, with immune cell | Half Antibody 2, with immune cell | N/A |
| | antigen targeting moiety that | antigen targeting moiety that binds | |
| | binds to CD28 and e.g., with | to CD20 and e.g., with pMHC | |
| | pMHC complex as described in | complex as described in Section 6.3 | |
| | Section 6.3 | | |
| 297 | Half Antibody 2, with immune cell | Half Antibody 2, with immune cell | N/A |
| | antigen targeting moiety that | antigen targeting moiety that | |
| | binds to CD28 and e.g., with | binds to CD22 and e.g., with pMHC | |
| | pMHC complex as described in | complex as described in Section 6.3 | |
| | Section 6.3 | | |
| 298 | Half Antibody 2, with immune cell | Half Antibody 2, with immune cell | N/A |
| | antigen targeting moiety that | antigen targeting moiety that binds | |
| | binds to a BCA, e.g., as described | to a TCA, e.g., as described in | |
| | in Section 6.4.2 and e.g., with | Section 6.4.1 and e.g., with pMHC | |
| | pMHC complex as described in | complex as described in Section 6.3 | |
| | Section 6.3 | | |
| 299 | Half Antibody 2, with immune cell | Half Antibody 2, with immune cell | N/A |
| | antigen targeting moiety that | antigen targeting moiety that binds | |
| | binds to CD19 and e.g., with | to CD3 and e.g., with pMHC | |
| | pMHC complex as described in | complex as described in Section 6.3 | |
| | Section 6.3 | | |
| 300 | Half Antibody 2, with immune cell | Half Antibody 2, with immune cell | N/A |
| | antigen targeting moiety that | antigen targeting moiety that binds | |
| | binds to CD19 and e.g., with | to CD28 and e.g., with pMHC | |
| | pMHC complex as described in | complex as described in Section 6.3 | |
| | Section 6.3 | | |
| 301 | Half Antibody 2, with immune cell | Half Antibody 2, with immune cell | N/A |
| | antigen targeting moiety that | antigen targeting moiety that binds | |
| | binds to CD20 and e.g., with | to CD3 and e.g., with pMHC | |
| | pMHC complex as described in | complex as described in Section 6.3 | |
| | Section 6.3 | | |
| 302 | Half Antibody 2, with immune cell | Half Antibody 2, with immune cell | N/A |
| | antigen targeting moiety that | antigen targeting moiety that binds | |
| | binds to CD20 and e.g., with | to CD28 and e.g., with pMHC | |
| | pMHC complex as described in | complex as described in Section 6.3 | |
| | Section 6.3 | | |
| 303 | Half Antibody 2, with immune cell | Half Antibody 2, with immune cell | N/A |
| | antigen targeting moiety that | antigen targeting moiety that binds | |
| | binds to CD22 and e.g., with | to CD3 and e.g., with pMHC | |
| | pMHC complex as described in | complex as described in Section 6.3 | |
| | Section 6.3 | | |
| 304 | Half Antibody 2, with immune cell | Half Antibody 2, with immune cell | N/A |
| | antigen targeting moiety that | antigen targeting moiety that binds | |
| | binds to CD22 and e.g., with | to CD28 and e.g., with pMHC | |
| | pMHC complex as described in | complex as described in Section 6.3 | |
| | Section 6.3 | | |
| 305 | Half Antibody 2, with immune cell | Half Antibody 2, with immune cell | N/A |
| | antigen targeting moiety that | antigen targeting moiety that binds | |
| | binds to a BCA, e.g., as described | to a BCA, e.g., as described in | |
| | in Section 6.4.2 and e.g., with | Section 6.4.2 and e.g., with pMHC | |
| | pMHC complex as described in | complex as described in Section 6.3 | |
| | Section 6.3 | | |
| 306 | Half Antibody 2, with immune cell | Half Antibody 2, with immune cell | N/A |
| | antigen targeting moiety that | antigen targeting moiety that binds | |
| | binds to CD19 and e.g., with | to CD19 and e.g., with pMHC | |
| | pMHC complex as described in | complex as described in Section 6.3 | |
| | Section 6.3 | | |
| 307 | Half Antibody 2, with immune cell | Half Antibody 2, with immune cell | N/A |
| | antigen targeting moiety that | antigen targeting moiety that binds | |
| | binds to CD19 and e.g., with | | |
| | pMHC complex as described in | to CD20 and e.g., with pMHC | |
| | Section 6.3 | complex as described in Section 6.3 | |
| 308 | Half Antibody 2, with immune cell | Half Antibody 2, with immune cell | N/A |
| | antigen targeting moiety that | antigen targeting moiety that binds | |
| | binds to CD19 and e.g., with | to CD22 and e.g., with pMHC | |
| | pMHC complex as described in | complex as described in Section 6.3 | |
| | Section 6.3 | | |
| 309 | Half Antibody 2, with immune cell | Half Antibody 2, with immune cell | N/A |
| | antigen targeting moiety that | antigen targeting moiety that binds | |
| | binds to CD20 and e.g., with | to CD19 and e.g., with pMHC | |
| | pMHC complex as described in | complex as described in Section 6.3 | |
| | Section 6.3 | | |
| 310 | Half Antibody 2, with immune cell | Half Antibody 2, with immune cell | N/A |
| | antigen targeting moiety that | antigen targeting moiety that binds | |
| | binds to CD20 and e.g., with | to CD20 and e.g., with pMHC | |
| | pMHC complex as described in | complex as described in Section 6.3 | |
| | Section 6.3 | | |
| 311 | Half Antibody 2, with immune cell | Half Antibody 2, with immune cell | N/A |
| | antigen targeting moiety that | antigen targeting moiety that binds | |
| | binds to CD20 and e.g., with | to CD22 and e.g., with pMHC | |
| | pMHC complex as described in | complex as described in Section 6.3 | |
| | Section 6.3 | | |
| 312 | Half Antibody 2, with immune cell | Half Antibody 2, with immune cell | N/A |
| | antigen targeting moiety that | antigen targeting moiety that binds | |
| | binds to CD22 and e.g., with | to CD19 and e.g., with pMHC | |
| | pMHC complex as described in | complex as described in Section 6.3 | |
| | Section 6.3 | | |
| 313 | Half Antibody 2, with immune cell | Half Antibody 2, with immune cell | N/A |
| | antigen targeting moiety that | antigen targeting moiety that binds | |
| | binds to CD22 and e.g., with | to CD20 and e.g., with pMHC | |
| | pMHC complex as described in | complex as described in Section 6.3 | |
| | Section 6.3 | | |
| 314 | Half Antibody 2, with immune cell | Half Antibody 2, with immune cell | N/A |
| | antigen targeting moiety that | antigen targeting moiety that binds | |
| | binds to CD22 and e.g., with | to CD22 and e.g., with pMHC | |
| | pMHC complex as described in | complex as described in Section 6.3 | |
| | Section 6.3 | | |

### 6.3. Peptide-MHC Complexes

Aspects of the disclosure relate to molecules (e.g., T cell activators) comprising a peptide-MHC complex (a “pMHC complex”), comprising a peptide complexed with an MHC class I domain or a peptide complexed with an MHC class II domain, optionally with a β2 microglobulin (β2M) domain.

In some embodiments, a pMHC complex comprises (i) an antigenic peptide; (ii) a class I MHC polypeptide or a fragment, mutant or derivative thereof (e.g., the extracellular domain), and optionally, (iii) a β2 microglobulin polypeptide or a fragment, mutant or derivative thereof. In the context of a T cell activator of the disclosure, the components of a pMHC complex can be present in a single polypeptide chain. For example, the pMHC complex can comprise, from the N- to C-terminus, (i) an antigenic peptide, (ii) a β2M sequence, and (iii) a class I α (heavy) chain sequence. Alternatively, the pMHC complex can comprise, from the N- to C-terminus, (i) an antigenic peptide, (ii) a class I α (heavy) chain sequence, and (iii) a β2M sequence.

In certain embodiments, the antigenic peptide and the MHC sequence and/or the MHC sequence and the β2M domain are linked to one another via a peptide linker, e.g., as described in Section 6.8. In some embodiments, a single-chain pMHC complex can comprise a first flexible linker between the peptide segment and the β2 microglobulin segment. For example, linkers can extend from and connect the carboxy terminal of the peptide to the amino terminal of the β2 microglobulin segment. In some embodiments, the linkers are structured to allow the peptide to fold into the binding groove resulting in a functional pMHC complex. In some embodiments, this linker can comprise at least 3 amino acids, up to about 15 amino acids (e.g., 20 amino acids). The pMHC linker can comprise a second flexible linker inserted between the β2 microglobulin and MHC I heavy chain segment. For example, linkers can extend from and connect the carboxy terminal of the β2 microglobulin segment to the amino terminal of the MHC I heavy chain segment. In certain embodiments, the β2 microglobulin and the MHC I heavy chain can fold into the binding groove resulting in a molecule which can function in promoting T cell expansion.

In further embodiments, the single-chain pMHC complex can comprise a peptide covalently attached to an MHC class I α (heavy) chain via a disulfide bridge (i.e., a disulfide bond between two cysteines). See, e.g., U.S. Pat. Nos. 8,992,937 and 8,895,020, each of which is incorporated in its entirety by reference. In certain embodiments, the disulfide bond comprises a first cysteine, that is positioned within a linker extending from the carboxy terminal of the peptide, and a second cysteine that is positioned within an MHC class I heavy (e.g., an MHC class I α (heavy) chain which has a non-covalent binding site for the antigen peptide). In certain embodiments, the second cysteine can be a mutation (addition or substitution) in the MHC class I α (heavy) chain. Preferably, the pMHC complex can comprise one contiguous polypeptide chain as well as a disulfide bridge. Alternatively, the pMHC complex can comprise two contiguous polypeptide chains which are attached via the disulfide bridge as the only covalent linkage. In some embodiments, the linking sequences can comprise at least one amino acid in addition to the cysteine, including one or more glycines, one or more, alanines, and/or one or more serines. In some embodiments, the single-chain molecule comprises from N-terminus to C-terminus an MHC class I peptide (e.g., an antigenic peptide), a first linker that comprises a first cysteine, a 32-microglobulin sequence, a second linker, and a MHC class I heavy chain sequence comprising a second cysteine, wherein the first cysteine and the second cysteine comprise a disulfide bridge. In some embodiments, the second cysteine is a substitution of an amino acid of the MHC class I heavy chain selected from the group consisting of T80C, Y84C and N86C (Y84C refers to a mutation at position 108 in a mature protein, where the mature protein lacks a signal sequence. Alternatively, when the protein still includes a 24 mer signal sequence, the position is instead referred to as Y108C).

In certain embodiments, the disulfide bridge can link a peptide in the class I groove of the pMHC complex if the pMHC complex comprises a first cysteine in a Gly-Ser linker extending between the C-terminus of the peptide and the β2 microglobulin, and a second cysteine in a proximal heavy chain position.

### 6.3.1. MHCs

Naturally-occurring MHCs are encoded by a cluster of genes on human chromosome 6. MHCs include, but are not limited to, HLA specificities such as A (e.g., A1-A74), B (e.g., B1-B77), C (e.g., C1-C11), D (e.g., D1-D26), DR (e.g., DR1-DR8), DQ (e.g., DQ1-DQ9) and DP (e.g., DP1-DP6). HLA specificities include A1, A2, A3, All, A23, A24, A28, A30, A33, B7, B8, B35, B44, B53, B60, B62, DR1, DR2, DR3, DR4, DR7, DR8, and DR11.

Naturally occurring MHC class I molecules bind peptides derived from proteolytically degraded proteins. Small peptides obtained accordingly are transported into the endoplasmic reticulum where they associate with nascent MHC class I molecules before being routed through the Golgi apparatus and displayed on the cell surface for recognition by cytotoxic T lymphocytes.

Naturally occurring MHC class I molecules consist of an a (heavy) chain associated with β2 microglobulin. The heavy chain consists of subunits α1-α3. The β2 microglobulin protein and α3 subunit of the heavy chain are associated. In certain embodiments, β2 microglobulin and α3 subunit are associated by covalent binding. In certain embodiments, β2 microglobulin and α3 subunit are associated non-covalently. The α1 and α2 subunits of the heavy chain fold to form a groove for a peptide, e.g., antigenic determinant, to be displayed and recognized by TCR.

Class I molecules generally associate with, e.g., bind, peptides of about 8-9 amino acids (e.g., 7-11 amino acids) in length. All humans have between three and six different class I molecules, which can each bind many different types of peptides. In one specific embodiment, the class I MHC polypeptide is a human class I MHC polypeptide selected from the group consisting of HLA-A, HLA-B, HLA-C, HLA-E, HLA-F, and HLA-G.

In some embodiments, the pMHC complex comprises an MHC class I α heavy chain extracellular domain (human α1, α2, and/or α3 domains) without a transmembrane domain. In some embodiments, the class I α heavy chain polypeptide is HLA-A, HLA-B, HLA-C, HLA-E, HLA-F, HLA-G, HLA-K, or HLA-L.

Non-limiting examples of HLA-A alleles include, without limitation, A*0101, A*0201, A*0202, A*0301, A*1101, A*2301, A*2402, A*2501, A*2601, A*2901, A*2902, A*3101, A*3201, A*3301, A*3401, A*3601, A*4301, A*6601, A*6801, A*6901, A*7401, and A*8001. Non-limiting examples of HLA-B alleles include, without limitation, B*0702. B*0801, B*1301, B*1401, B*1402, B*1501, B*1801, B*1802, B*2701, B*2702, B*3501, B*3502, B*3701, B*3801, B*3901, B*4001, B*4101, B*4201, B*4402, B*4501, B*4601, B*4701, B*4801, B*4901, B*5001, B*5101, B*5201, B*5301, B*5401, B*5501, B*5502, B*5601, B*5701, B*5801, B*5901, B*6701, B*7301, B*1517, B*8101, B*8201, and B*8301. Non-limiting examples of HLA-C alleles include, without limitation, Cw*0101, Cw*0202, Cw*0303, Cw*0401, Cw*0501, Cw*0602, Cw*0701, Cw*0702, Cw*0802, Cw*1203, Cw*1401, Cw*1502, Cw*1601, Cw*1701, and. Cw*1801. Non-limiting examples of HLA-DR alleles include, without limitation, DRB1*0101, DRB1*0103, DRB1*1501, DRB1*1502, DRB1*1601, DRB1*1602, DRB1*0301, DRB1*0401, DRB1*0404, DRB1*1101, DRB1*1201, DRB1*1301, DRB1*1302, DRB1*1401, DRB1*1402, DRB1*0701, DRB1*0801, DRB1*0802, DRB1*0803, DRB1*0901, and DRB1*1001. In some embodiments, the MHC class I molecule may be selected from HLA-A*02, HLA-A*01, HLA-A*03, HLA-A*11, HLA-A*23, HLA-A*24, HLA-B*07, HLA-B*08, HLA-B*40, HLA-B*44, HLA-B*15, HLA-C*04, HLA*C*03 HLA-C*07. Numerous allelic variants of the above HLA types are recognized in the art, which variants are encompassed by the present disclosure. In some embodiments, the MHC molecule is HLA-A*02 or HLA-A*11.

In some embodiments, the HLA-A sequence can be an HLA-A*0201 sequence, as disclosed below:

HLA-A*0201-
(SEQ ID NO: 251)
GSHSMRYFFTSVSRPGRGEPRFIAVGYVDDTQFVRFDSDAASQRMEPRA
PWIEQEGPEYWDGETRKVKAHSQTHRVDLGTLRGCYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPAGDGTFQKWA
AVVVPSGQEQRYTCHVQHEGLPKPLTLRWEP

In some aspects, the MHC sequence has at least about 90% sequence identity to the amino acid sequence of SEQ ID NO:251. In some aspects, the MHC sequence has at least about 91% sequence identity to the amino acid sequence of SEQ ID NO:251. In some aspects, the MHC sequence has at least about 92% sequence identity to the amino acid sequence of SEQ ID NO:251. In some aspects, the MHC sequence has at least about 93% sequence identity to the amino acid sequence of SEQ ID NO:251. In some aspects, the MHC sequence has at least about 94% sequence identity to the amino acid sequence of SEQ ID NO:251. In some aspects, the MHC sequence has at least about 95% sequence identity to the amino acid sequence of SEQ ID NO:251. In some aspects, the MHC sequence has at least about 96% sequence identity to the amino acid sequence of SEQ ID NO:251. In some aspects, the MHC sequence has at least about 97% sequence identity to the amino acid sequence of SEQ ID NO:251. In some aspects, the MHC sequence has at least about 98% sequence identity to the amino acid sequence of SEQ ID NO:251. In some aspects, the MHC sequence has at least about 99% sequence identity to the amino acid sequence of SEQ ID NO:251. In some aspects, the MHC sequence has 100% sequence identity to the amino acid sequence of SEQ ID NO:251.

As an alternative to type I MHC-based pMHC complexes, the T cell activators of the disclosure can include a class II MHC-based pMHC complex. A class II MHC-based pMHC complex generally includes a class I MHC polypeptide or a fragment, mutant or derivative thereof. In one specific embodiment, the MHC comprises a and B polypeptides of a class II MHC molecule or a fragment, mutant or derivative thereof. In one specific embodiment, the a and B polypeptides are linked by a peptide linker. In one specific embodiment, the MHC comprises a and B polypeptides of a human class II MHC molecule selected from the group consisting of HLA-DP, HLA-DR, HLA-DQ, HLA-DM and HLA-DO.

MHC class II molecules generally consist of two polypeptide chains, a and B. The chains may come from the DP, DQ, or DR gene groups. There are about 40 known different human MHC class II molecules. All have the same basic structure but vary subtly in their molecular structure. MHC class II molecules bind peptides of 13-18 amino acids in length.

In some embodiments, the pMHC complex comprises one or more MHC class II α chains or an extracellular portion thereof. In some embodiments, the class II α chain is HLA-DMA, HLA-DOA, HLA-DPA, HLA-DQA or HLA-DRA.

In other embodiments, the pMHC complex comprises one or more MHC class II β chains or an extracellular portion thereof. In some embodiments, the class II β chain is HLA-DMB, HLA-DOB, HLA-DPB, HLA-DQB or HLA-DRB.

Class II MHC-based pMHC complexes include those described in PCT Pub. No. WO 2021/113297 A1, incorporated herein by reference.

### 6.3.2. Antigenic Peptides

The peptide in the pMHC complex can have the amino acid sequence of a peptide which can be associated with, e.g., presented by, an MHC molecule.

In some embodiments, the peptide in the pMHC complex comprises the amino acid sequence of a peptide associated with a class I MHC molecule. In certain embodiments, the sequence can comprise from 6 to 20 contiguous amino acids. In certain embodiments, a peptide sequence can be that of a protein fragment, wherein the protein is a derived from, e.g., a portion of, a cellular protein, such as, for example, a protein associated with cancer or cancer neoantigen, and wherein the peptide can be bound to the MHC class I heavy chain.

In other embodiments, the peptide in the pMHC complex comprises the amino acid sequence of a peptide associated with, e.g., presented by, a class II MHC molecule, for example as described in WO 2021/113297 A1.

The peptide in a pMHC complex can be any peptide that is capable of binding to an MHC protein in a manner such that the pMHC complex can bind to a TCR, e.g., in a specific manner.

Examples include peptides produced by hydrolysis and most typically, synthetically produced peptides, including randomly generated peptides, specifically designed peptides, and peptides where at least some of the amino acid positions are conserved among several peptides and the remaining positions are random.

In nature, peptides that are produced by hydrolysis undergo hydrolysis prior to binding of the antigen to an MHC protein. Class I MHC typically present peptides derived from proteins actively synthesized in the cytoplasm of the cell. In contrast, class II MHC typically present peptides derived either from exogenous proteins that enter a cell's endocytic pathway or from proteins synthesized in the ER. Intracellular trafficking permits a peptide to become associated with an MHC protein.

The binding of a peptide to an MHC peptide binding groove can control the spatial arrangement of MHC and/or peptide amino acid residues recognized by a TCR, or pMHC-binding protein produced by an animal genetically modified as disclosed herein. Such spatial control is due in part to hydrogen bonds formed between a peptide and an MHC protein. Based on the knowledge on how peptides bind to various MHCs, the major MHC anchor amino acids and the surface exposed amino acids that are varied among different peptides can be determined. In some embodiments, the length of an MHC-binding peptide is from 5 to 40 amino acid residues, e.g., from 6 to 30 amino acid residues, e.g., from 8 to 20 amino acid residues, e.g., between 9 and 11 amino acid residues, including any size peptide between 5 and 40 amino acids in length, in whole integer increments (i.e., 5, 6, 7, 8, 9 . . . 40). While naturally MHC Class II-bound peptides vary from about 9-40 amino acids, in nearly all cases the peptide can be truncated to a 9-11 amino acid core without loss of MHC binding activity or T cell recognition.

The peptides in the pMHC complexes for incorporation into a T cell activator of the disclosure typically at least a portion, e.g., an antigenic determinant, of proteins of infectious agents (e.g., bacterial, viral or parasitic organisms), allergens, and tumor associated proteins. In some embodiments, the pMHC complexes comprise an antigenic determinant of cancer cells. Exemplary antigenic determinants of cancer cells are listed in Table 2-A and include LCMV derived peptide gp33-41, APF (126-134), BALF (276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAGE-A3 (112-120), MAGE-A4 (230-239), MAGE-A4 (286-294), NYESO1 (157-165, C165A), NYESO1 (157-165, C165V), p54 WT (264-272), PAP-3 (136-143), PSMA (4-12), PSMA (135-145), Survivin (96-104), Tyrosinase (369-377, 371D), and WT1 (126-134). An exemplary HPV E7 (11-19) peptide sequence is YMLDLQPET (SEQ ID NO:7), SEQ ID NO:537 of International Patent Publication WO 2019/005897. An exemplary HPV E7 (82-90) peptide sequence is LLMGTLGIV (SEQ ID NO:8), SEQ ID NO:538 of International Patent Publication WO 2019/005897. The contents of International Patent Publication WO 2019/005897 are incorporated by reference in their entirety herein.

TABLE 1-A

| Peptide | HLA allele | Peptide sequence |
|---|---|---|
| BALF-4 (276-284) | A 02: 01 | FLDKGTYTL (SEQ ID NO: 1) |
| CEA (571-579) | A 02: 01 | YLSGANLNL (SEQ ID NO: 2) |
| CMV pp65 | A 02: 01 | NLVPMVATV (SEQ ID NO: 3) |
| (495-503) | | |
| FLU-M1 (58-66) | A 02: 01 | GILGFVFTL (SEQ ID NO: 4) |
| gp100 (154-162) | A 02: 01 | KTWGQYWQV (SEQ ID NO: 5) |
| HBV Core (18-27) | A 02: 01 | FLPSDFFPSV (SEQ ID NO: 6) |
| Her2/neu | A 02: 01 | KIFGSLAFL (SEQ ID NO: 9) |
| (369-377; V2v9) | | |
| HPV E7 (11-20) | A 02: 01 | YMLDLQPETT (SEQ ID NO: 29) |
| HPV E7 (11-19) | A 02: 01 | YMLDLQPET (SEQ ID NO: 7) |
| HPV E7 (82-90) | A 02: 01 | LLMGTLGIV (SEQ ID NO: 8) |
| KLK4 (11-19) | A 02: 01 | FLGYLILGV (SEQ ID NO: 30) |
| LMP1 (125-133) | A 02: 01 | YLLEMLWRL (SEQ ID NO: 31) |
| MAGE-A3 | A 02: 01 | KVAELVHFL (SEQ ID NO: 32) |
| (112-120) | | |
| MAGE-A4 | A 02: 01 | GVYDGREHTV (SEQ ID NO: 33) |
| (230-239) | | |
| MAGE-A4 | A 02: 01 | KVLEHVVRV (SEQ ID NO: 34) |
| (286-294) | | |
| NY-ESO-1 | A 02: 01 | SLLMWITQV (SEQ ID NO: 35) |
| (157-165, C165V) | | |
| NY-ESO-1 | A 02: 01 | SLLMWITQA (SEQ ID NO: 36) |
| (157-165, C165A) | | |
| p53 (264-272) | A 02: 01 | LLGRNSFEV (SEQ ID NO: 37) |
| PSMA (4-12) | A 02: 01 | LLHETDSAV (SEQ ID NO: 38) |
| Survivin (96-104) | A 02: 01 | LTLGEFLKL (SEQ ID NO: 39) |
| Tyrosinase | A 02: 01 | YMDGTMSQV (SEQ ID NO: 40) |
| (369-377, 371D) | | |
| WT1 (126-134) | A 02: 01 | RMFPNAPYL (SEQ ID NO: 41) |

In some embodiments, a pMHC complex incorporated into a T cell activator of the present disclosure comprises a peptide selected from those recited in Table 1-A.

Other antigenic determinants of cancer cells suitable use in pMHC complexes incorporated into a T cell activator of the disclosure are cancer neoantigens listed in Table 1-B below and their corresponding HLA allele. The neoantigens contain either mutations relative to a wild type allele or result from the expression of a new open reading frame in cancer cells, as shown in Table 1 of Fritsch et al., 2014, Cancer Immunol Res 2:522-529 and incorporated by reference herein in its entirety.

TABLE 1-B

| | HLA | |
|---|---|---|
| Gene | allele | Neoantigen |
| ECGF-1 | B 07:02 | RPHAIRRPLAL (SEQ ID NO: 42) |
| ME-1 | A 02:01 | FLDEFMEGV (SEQ ID NO: 43) |
| PLEKHM2 | A 01:01 | LTDDRLFTCY (SEQ ID NO: 44) |
| FNDC3B | A 02:01 | VVMSWAPPV (SEQ ID NO: 45) |
| PRDX5 | A 02:01 | LLLDDLLVSI (SEQ ID NO: 46) |
| MATN2 | A 11:01 | KTLTSVFQK (SEQ ID NO: 47) |
| DDX21 | A 68:01 | EAFIQPITR (SEQ ID NO: 48) |
| RBAF | B 07:02 | RPHVPESAF (SEQ ID NO: 49) |
| GAS7 | A 02:01 | SLADEAEVYL (SEQ ID NO: 50) |
| ATR | A 03:01 | KLYEEPLLK (SEQ ID NO: 51) |
| SIRT2 | A 03:01 | KIFSEVTLK (SEQ ID NO: 52) |
| EF2 | A 68:02 | ETVSEQSNV (SEQ ID NO: 53) |
| KIAA0223 | A 02:01 | VLHDDLLEA (SEQ ID NO: 54) |
| GAPDH | A 02:01 | GIVEGLITTV (SEQ ID NO: 55) |
| BCL2A1 | A 24:02 | DYLQYVLQI (SEQ ID NO: 56) |
| HSP70 | A 02:01 | SLFEGIDIYT (SEQ ID NO: 57) |
| ACTININ | A 02:01 | FIASNGVKLV (SEQ ID NO: 58) |
| CDK12 | A 11:01 | CILGKLFTK (SEQ ID NO: 59) |
| KIAA1440 | A 01:01 | QTACEVLDY (SEQ ID NO: 60) |
| HAUS3 | A 02:01 | ILNAMIAKI (SEQ ID NO: 61) |
| hERV 4700 | A 02:01 | MVGPWPRPV (SEQ ID NO: 62) |
| BCL2A1 | B 44:03 | KEFEDDIINW (SEQ ID NO: 63) |
| PPP1R3B | A 01:01 | YTDFHCQYV (SEQ ID NO: 64) |
| HB-1 | B 44:03 | EEKRGSLHVW (SEQ ID NO: 65) |
| MUM-2 | B 44:02 | SELFRSGLDSY (SEQ ID NO: 66) |
| KIAA0205 | B 44:03 | AEPIDIQTW (SEQ ID NO: 67) |
| GPNMB | A 03:01 | TLDWLLQTPK (SEQ ID NO: 68) |
| CSNK1A1 | A 02:01 | GLFGDIYLAI (SEQ ID NO: 69) |
| CLPP | A 02:01 | ILDKVLVHL (SEQ ID NO: 70) |
| CTNNB1 | A 24:02 | SYLDSGIHF (SEQ ID NO: 71) |
| SNRP116 | A 03:01 | KILDAVVAQK (SEQ ID NO: 72) |
| OS9 | B 44:03 | KELEGILLL (SEQ ID NO: 73) |
| MYH2 | A 03:01 | KINKNPKYK (SEQ ID NO: 74) |
| MART-2 | A 01:01 | FLEGNEVGKTY (SEQ ID NO: 75) |
| NFYC | B 52:01 | AQQITKTEV (SEQ ID NO: 76) |
| CDK4 | A 02:01 | ACDPHSGHFV (SEQ ID NO: 77) |
| PARF14- | A 11:01 | AVCPWTWLR (SEQ ID NO: 78) |
| ORF3 | | |
| HMSD-n | B 44:03 | MEIFIEVFSHF (SEQ ID NO: 79) |
| PANE-1 | A 03:01 | RVWDLPGVLK (SEQ ID NO: 80) |
| MUM1 | B 44:02 | EEKLIVVLF (SEQ ID NO: 81) |
| P2x5 | B 07:02 | TPNQRQNVC (SEQ ID NO: 82) |

In some embodiments, a pMHC complex incorporated into a T cell activator of the present disclosure comprises a peptide selected from those recited in Table 2-B.

Additional antigenic determinants of cancer cells suitable for use in a pMHC complex incorporated into a T cell activator of the disclosure include those described in the Cancer Epitope Database and Analysis Resource (CEDAR), accessible on the web at cedar.iedb.org, a subset of the Immune Epitope Database (IEDB) which is described in Vita et al., 2018, Nucleic Acids Res. 8; 47(D1):D339-D343, incorporated herein by reference in its entirety.

Other antigenic determinants suitable for incorporation into a pMHC complex of the disclosure include those listed in Table 1-C below.

TABLE 1-C

| Peptide | HLA allele | Peptide sequence |
|---|---|---|
| KRAS (6-14) G12V | A 02:01 | LVVVGAVGV (SEQ ID NO: 83) |
| KRAS (5-14) G12D | A 02:01 | KLVVVGADGV (SEQ ID NO: 84) |
| KRAS (5-14) G12V | A 02:01 | KLVVVGAVGV (SEQ ID NO: 85) |
| KRAS (10-18) G12D | C 08:02 | GADGVGKSA (SEQ ID NO: 86) |
| KRAS (10-19) G12D | C 08:02 | GADGVGKSAL (SEQ ID NO: 87) |
| KRAS (8-16) G12D | A 11:01 | VVGADGVGK (SEQ ID NO: 88) |
| KRAS (8-16) G12V | A 11:01 | VVGAVGVGK (SEQ ID NO: 89) |
| KRAS (7-16) G12D | A 11:01 | VVVGADGVGK (SEQ ID NO: 90) |
| KRAS (7-16) G12V | A 11:01 | VVVGAVGVGK (SEQ ID NO: 91) |
| MAGE-A3 (168-176) | A 01:01 | EVDPIGHLY (SEQ ID NO: 92) |
| MAGE-A3 (271-279) | A 02:01 | FLWGPRALV (SEQ ID NO: 93) |
| MAGE-A3 (108-116) | A 02:01 | ALSRKVAEL (SEQ ID NO: 94) |
| MAGE-A10 (137-145) | A 02:01 | KVTDLVQFL (SEQ ID NO: 95) |
| MAGE-A10 (254-262) | A 02:01 | GLYDGMEHL (SEQ ID NO: 96) |
| MAGE-A10 (310-318) | A 02:01 | SLLKFLAKV (SEQ ID NO: 97) |
| WT-1 (10-18) | A 02:01 | ALLPAVPSL (SEQ ID NO: 98) |
| PRAME (312-320) | A 02:01 | RLDQLLRHV (SEQ ID NO: 99) |
| PRAME (425-433) | A 02:01 | SLLQHLIGL (SEQ ID NO: 100) |
| PSA (79-89) | A 02:01 | SLFHPEDTGQV (SEQ ID NO: 101) |
| PSA (159-169) | A 11:01 | SIEPEEFLTPK (SEQ ID NO: 102) |
| PSA (241-249) | A 11:01 | SLYTKVVHY (SEQ ID NO: 103) |
| EBNA-1 (562-570) | A 02:01 | FMVFLQTHI (SEQ ID NO: 104) |
| LMP2 (426-434) | A 02:01 | CLGGLLTMV (SEQ ID NO: 105) |
| DNTT (475-483) | A 02:01 | ALYDKTKRI (SEQ ID NO: 106) |
| DNTT (250-258) | A 02:01 | KLFTSVFGV (SEQ ID NO: 107) |
| MPO (132-140) | A 02:01 | SLWRRPFNV (SEQ ID NO: 108) |
| MPO (549-557) | A 02:01 | GLMATPAKL (SEQ ID NO: 109) |
| MPO (571-579) | A 02:01 | RLFEQVMRI (SEQ ID NO: 110) |
| AFP (58-68) | A 11:01 | ATYKEVSKMVK (SEQ ID NO: 111) |
| AFP (173-181) | A 24:02 | LYAPTILLW (SEQ ID NO: 112) |
| IGF2BP3 (552-560) | A 02:01 | KIQEILTQV (SEQ ID NO: 113) |
| KLK4 (215-223) | A 24:02 | GYLQGLVSF (SEQ ID NO: 114) |
| KLK4 (105-113) | B 07:02 | HPEYNRPLL (SEQ ID NO: 115) |
| ACP3 (PAP) (112-120) | A 02:01 | TLMSAMTNL (SEQ ID NO: 116) |
| KLK3 (PSA) (82-90) | HLA-B | HPEDTGQVF (SEQ ID NO: 117) |
| HIV P17 (77-85) | A 02:01 | SLYNTVATL (SEQ ID NO: 118) |
| HIV RT (896-904) | A 02:01 | ILKEPVHGV (SEQ ID NO: 119) |
| HIV GAG (41-49) | HLA-E | AISPRTLNA (SEQ ID NO: 120) |
| ENV (183-191) | A 02:01 | FLLTRILTI (SEQ ID NO: 121) |
| HBV Pol (616-626 | A 11:01 | GTLPQDHIVQK (SEQ ID NO: 122) |
| variant 5) | | |
| IGHE Immunoglobulin | A 02:01 | YATISLLTV (SEQ ID NO: 123) |
| heavy constant epsilon | | |
| (66-74) | | |
| alpha1 (α1) gliadin | DQ2.5 | PFPQPELPY (SEQ ID NO: 124) |
| (61-69); Q66E | | |
| alpha 2 (α2) gliadin | DQ2.5 | PQPELPYPQ (SEQ ID NO: 125) |
| (63-71); Q66E | | |
| PPI (15-24) | A 02:01 | ALWGPDPAAA (SEQ ID NO: 126) |

In some embodiments, a pMHC complex incorporated into a T cell activator of the present disclosure comprises a peptide selected from those recited in Table 1-C.

Additional antigenic determinants suitable for use in a pMHC complex incorporated into a T cell activator of the disclosure are those disclosed in PCT Pub. No. WO 2021/003357 A1 as SEQ ID NOs: 269, 270, and 291, incorporated herein by reference. Yet additional antigenic determinants suitable for use in a pMHC complex incorporated into a T cell activator of the disclosure are those disclosed in US Pub. No. 2022/0409732 A1 as SEQ ID NOs:44, 45, 46, 69, 70, 71, 72, and 73, incorporated herein by reference. Further antigenic determinants suitable for use in a pMHC complex incorporated into a T cell activator of the disclosure include those disclosed in PCT Pub. No. WO 2023/240085 A1, US Pub. No. 2007/0020327 A1, and US Pub. No. 2006/0079453 A1, the contents of which are incorporated by reference herein in their entirety. Additional antigenic determinants contemplated herein include, for example, those described in the IEDB, accessible via the web at iedb.org and described in Vita et al., 2018, Nucleic Acids Res. 8; 47(D1): D339-D343.

### 6.3.3. β2 Microglobulin

As discussed above, a pMHC complex may, optionally, comprise a 32 microglobulin (β2M). When β2M is present, the pMHC complex can include mutations in β2M and in the MHC class I α heavy chain domain such that a disulfide bond may form between them. Exemplary amino acid pairs that can be substituted with cysteines to allow for disulfide bonding between the two domains are identified in Table 2 below or as described in PCT Pub. WO 2015/195531, incorporated herein by reference in its entirety:

TABLE 2

| β2M Domain | MHC Domain |
|---|---|
| 12 | 236 |
| 12 | 237 |
| 8 | 234 |
| 10 | 235 |
| 24 | 236 |
| 28 | 232 |
| 98 | 192 |
| 99 | 234 |
| 3 | 120 |
| 31 | 96 |
| 53 | 35 |
| 60 | 96 |
| 60 | 122 |
| 63 | 27 |
| R3 | G120 |
| H31 | Q96 |
| D53 | R35 |
| W60 | Q96 |
| W60 | D122 |
| Y63 | Y27 |
| K6 | E232 |
| Q8 | R234 |
| Y10 | P235 |
| S11 | Q242 |
| N24 | A236 |
| S28 | E232 |
| D98 | H192 |
| M99 | R234 |
| R12 | G237 |

When present, the β2 microglobulin sequence can comprise a full-length (human or non-human) β2 microglobulin sequence. An exemplary human β2 microglobulin sequence is Genbank accession no. AF072097.1, the amino acid sequence of which is presented below:

Human ß2 microglobulin (Full length)-
(SEQ ID NO: 249)
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM

In some aspects, the β2 microglobulin sequence has at least about 90% sequence identity to the amino acid sequence of full length human β2 microglobulin (SEQ ID NO:249). In some aspects, the β2 microglobulin sequence has at least about 91% sequence identity to the amino acid sequence of full length human β2 microglobulin (SEQ ID NO:249). In some aspects, the β2 microglobulin sequence has at least about 92% sequence identity to the amino acid sequence of full length human β2 microglobulin (SEQ ID NO:249). In some aspects, the β2 microglobulin sequence has at least about 93% sequence identity to the amino acid sequence of full length human β2 microglobulin (SEQ ID NO:249). In some aspects, the β2 microglobulin sequence has at least about 94% sequence identity to the amino acid sequence of full length human β2 microglobulin (SEQ ID NO:249). In some aspects, the β2 microglobulin sequence has at least about 95% sequence identity to the amino acid sequence of full length human 2 microglobulin (SEQ ID NO:249). In some aspects, the β2 microglobulin sequence has at least about 96% sequence identity to the amino acid sequence of full length human β2 microglobulin (SEQ ID NO:249). In some aspects, the β2 microglobulin sequence has at least about 97% sequence identity to the amino acid sequence of full length human β2 microglobulin (SEQ ID NO:249). In some aspects, the β2 microglobulin sequence has at least about 98% sequence identity to the amino acid sequence of full length human β2 microglobulin (SEQ ID NO:249). In some aspects, the β2 microglobulin sequence has at least about 99% sequence identity to the amino acid sequence of full length human β2 microglobulin (SEQ ID NO:249). In some aspects, the β2 microglobulin sequence has 100% sequence identity to the amino acid sequence of full length human β2 microglobulin (SEQ ID NO:249).

In certain embodiments, the β2 microglobulin sequence lacks the leader peptide sequence. As such, the β2 microglobulin sequence can comprise about 99 amino acids. An exemplary human β2 microglobulin sequence lacking the leader peptide sequence is presented below:

Human ß2 microglobulin (no leader peptide
sequence)-
(SEQ ID NO: 250)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M

In some aspects, the 2 microglobulin sequence has at least about 90%, at least about 91%, at least about 92%, at least about 93%, at least about 94%, at least about 95%, at least about 96%, at least about 97%, at least about 98%, at least about 99%, or 100% sequence identity to the amino acid sequence of human β2 microglobulin lacking the leader peptide sequence (SEQ ID NO:250).

In some aspects, the β2 microglobulin sequence has at least about 90% sequence identity to the amino acid sequence of human β2 microglobulin lacking the leader peptide sequence (SEQ ID NO:250). In some aspects, the β2 microglobulin sequence has at least about 91% sequence identity to the amino acid sequence of human β2 microglobulin lacking the leader peptide sequence (SEQ ID NO:250). In some aspects, the β2 microglobulin sequence has at least about 92% sequence identity to the amino acid sequence of human β2 microglobulin lacking the leader peptide sequence (SEQ ID NO:250). In some aspects, the β2 microglobulin sequence has at least about 93% sequence identity to the amino acid sequence of human β2 microglobulin lacking the leader peptide sequence (SEQ ID NO:250). In some aspects, the β2 microglobulin sequence has at least about 94% sequence identity to the amino acid sequence of human β2 microglobulin lacking the leader peptide sequence (SEQ ID NO:250). In some aspects, the β2 microglobulin sequence has at least about 95% sequence identity to the amino acid sequence of human β2 microglobulin lacking the leader peptide sequence (SEQ ID NO:250). In some aspects, the β2 microglobulin sequence has at least about 96% sequence identity to the amino acid sequence of human β2 microglobulin lacking the leader peptide sequence (SEQ ID NO:250). In some aspects, the β2 microglobulin sequence has at least about 97% sequence identity to the amino acid sequence of human β2 microglobulin lacking the leader peptide sequence (SEQ ID NO:250). In some aspects, the β2 microglobulin sequence has at least about 98% sequence identity to the amino acid sequence of human β2 microglobulin lacking the leader peptide sequence (SEQ ID NO:250). In some aspects, the β2 microglobulin sequence has at least about 99% sequence identity to the amino acid sequence of human β2 microglobulin lacking the leader peptide sequence (SEQ ID NO:250). In some aspects, the β2 microglobulin sequence has 100% sequence identity to the amino acid sequence of human β2 microglobulin lacking the leader peptide sequence (SEQ ID NO:250).

### 6.3.4. Exemplary Peptide-MHC Complexes

In particular embodiments, a pMHC complex suitable for use in a T cell activator of the disclosure comprises, in N- to C-terminal orientation, an antigenic peptide (e.g., as described in Section 6.3.2), a first linker (optionally comprising a cysteine, which can be advantageous for disulfide linkages as described above), a human β2 microglobulin sequence (e.g., as described in Section 6.3.3), a second linker, and an MHC sequence (e.g., as described in Section 6.3.1). An exemplary pMHC sequence, minus the peptide sequence, is as follows, where the bolded regions are the first and second linker, the italicized region is the human β2 microglobulin sequence, and the underlined region is the human HLA-A*0201 sequence:

(SEQ ID NO: 252)
GCGGSGGGGSGGGGSIQRTPKIQVYSRHPAENGKSNFLNCYVSGFHPSD
IEVDLLKNGERIEKVEHSDLSFSKDWSFYLLYYTEFTPTEKDEYACRVN
HVTLSQPKIVKWDRDMGGGGSGGGGSGGGGSGGGGSGSHSMRYFFTSVS
RPGRGEPRFIAVGYVDDTQFVRFDSDAASQRMEPRAPWIEQEGPEYWDG
ETRKVKAHSQTHRVDLGTLRGCYNQSEAGSHTVQRMYGCDVGSDWRFLR
GYHQYAYDGKDYIALKEDLRSWTAADMAAQTTKHKWEAAHVAEQLRAYL
EGTCVEWLRRYLENGKETLQRTDAPKTHMTHHAVSDHEATLRCWALSFY
PAEITLTWQRDGEDQTQDTELVETRPAGDGTFQKWAAVVVPSGQEQRYT
CHVQHEGLPKPLTLRWEP

In some aspects, the pMHC complex comprises an amino acid sequence having at least about 90% sequence identity to the amino acid sequence of SEQ ID NO:252. In some aspects, the pMHC complex comprises an amino acid sequence having at least about 91% sequence identity to the amino acid sequence of SEQ ID NO:252. In some aspects, the pMHC complex comprises an amino acid sequence having at least about 92% sequence identity to the amino acid sequence of SEQ ID NO:252. In some aspects, the pMHC complex comprises an amino acid sequence having at least about 93% sequence identity to the amino acid sequence of SEQ ID NO:252. In some aspects, the pMHC complex comprises an amino acid sequence having at least about 94% sequence identity to the amino acid sequence of SEQ ID NO:252. In some aspects, the pMHC complex comprises an amino acid sequence having at least about 95% sequence identity to the amino acid sequence of SEQ ID NO:252. In some aspects, the pMHC complex comprises an amino acid sequence having at least about 96% sequence identity to the amino acid sequence of SEQ ID NO:252. In some aspects, the pMHC complex comprises an amino acid sequence having at least about 97% sequence identity to the amino acid sequence of SEQ ID NO:252. In some aspects, the pMHC complex comprises an amino acid sequence having at least about 98% sequence identity to the amino acid sequence of SEQ ID NO:252. In some aspects, the pMHC complex comprises an amino acid sequence having at least about 99% sequence identity to the amino acid sequence of SEQ ID NO:252. In some aspects, the pMHC complex comprises an amino acid sequence having 100% sequence identity to the amino acid sequence of SEQ ID NO:252.

Various antigenic peptides, including those described in Section 6.3.2 and recognized in the art, may be included directly N-terminal to a sequence having at least 90%, 91%, 92%, 93%, 94%, 95%, 96%, 97%, 98%, 99%, or 100% sequence identity to SEQ ID NO:252. Certain exemplary pMHC complexes (which include an antigenic peptide) suitable for incorporation into a T cell activator of the disclosure are presented in Table P, below, along with their amino acid sequence.

TABLE P
Exemplary pMHC Complexes

| Identifier | Sequence | SEQ ID NO |
|---|---|---|
| HPV 16E7 | YMLDLQPETGCGGSGGGGSGGGGSIQRTPKIQVYSRH | 241 |
| (11-19) pMHC | PAENGKSNFLNCYVSGFHPSDIEVDLLKNGERIEKVEHS | |
| (HPV 16E7 | DLSFSKDWSFYLLYYTEFTPTEKDEYACRVNHVTLSQPK | |
| (11-19) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |
| NYESO-1 | SLLMWITQCGCGGSGGGGSGGGGSIQRTPKIQVYSRH | 243 |
| (157-165) pMHC | PAENGKSNFLNCYVSGFHPSDIEVDLLKNGERIEKVEHS | |
| (NYESO-1 | DLSFSKDWSFYLLYYTEFTPTEKDEYACRVNHVTLSQPK | |
| (157-165) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |
| MAGEA4 | GVYDGREHTVGCGGSGGGGSGGGGSIQRTPKIQVYSR | 245 |
| (230-239) pMHC | HPAENGKSNFLNCYVSGFHPSDIEVDLLKNGERIEKVEH | |
| (MAGEA4 | SDLSFSKDWSFYLLYYTEFTPTEKDEYACRVNHVTLSQP | |
| (230-239) peptide- | KIVKWDRDMGGGGSGGGGSGGGGSGGGGSGSHSMR | |
| linker-hB2M- | YFFTSVSRPGRGEPRFIAVGYVDDTQFVRFDSDAASQR | |
| linker-HLA-A2) | MEPRAPWIEQEGPEYWDGETRKVKAHSQTHRVDLGTL | |
| | RGCYNQSEAGSHTVQRMYGCDVGSDWRFLRGYHQYA | |
| | YDGKDYIALKEDLRSWTAADMAAQTTKHKWEAAHVAEQ | |
| | LRAYLEGTCVEWLRRYLENGKETLQRTDAPKTHMTHHA | |
| | VSDHEATLRCWALSFYPAEITLTWQRDGEDQTQDTELV | |
| | ETRPAGDGTFQKWAAVVVPSGQEQRYTCHVQHEGLPK | |
| | PLTLRWEP | |
| CMVpp65 | NLVPMVATVGCGGSGGGGSGGGGSIQRTPKIQVYSRH | 247 |
| (465-503) pMHC | PAENGKSNFLNCYVSGFHPSDIEVDLLKNGERIEKVEHS | |
| (CMVpp65 | DLSFSKDWSFYLLYYTEFTPTEKDEYACRVNHVTLSQPK | |
| (465-503) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |

In some aspects, the pMHC complex comprises an amino acid sequence having at least about 90% sequence identity to the amino acid sequence of SEQ ID NO:241. In some aspects, the pMHC complex comprises an amino acid sequence having at least about 91% sequence identity to the amino acid sequence of SEQ ID NO:241. In some aspects, the pMHC complex comprises an amino acid sequence having at least about 92% sequence identity to the amino acid sequence of SEQ ID NO:241. In some aspects, the pMHC complex comprises an amino acid sequence having at least about 93% sequence identity to the amino acid sequence of SEQ ID NO:241. In some aspects, the pMHC complex comprises an amino acid sequence having at least about 94% sequence identity to the amino acid sequence of SEQ ID NO:241. In some aspects, the pMHC complex comprises an amino acid sequence having at least about 95% sequence identity to the amino acid sequence of SEQ ID NO:241. In some aspects, the pMHC complex comprises an amino acid sequence having at least about 96% sequence identity to the amino acid sequence of SEQ ID NO:241. In some aspects, the pMHC complex comprises an amino acid sequence having at least about 97% sequence identity to the amino acid sequence of SEQ ID NO:241. In some aspects, the pMHC complex comprises an amino acid sequence having at least about 98% sequence identity to the amino acid sequence of SEQ ID NO:241. In some aspects, the pMHC complex comprises an amino acid sequence having at least about 99% sequence identity to the amino acid sequence of SEQ ID NO:241. In some aspects, the pMHC complex comprises an amino acid sequence having 100% sequence identity to the amino acid sequence of SEQ ID NO:241. In certain aspects of each of the foregoing embodiments, the peptide in the pMHC complex is identical to or has at most one or two amino acid substitutions to the peptide component of SEQ ID NO:241.

In some aspects, the pMHC complex comprises an amino acid sequence having at least about 90% sequence identity to the amino acid sequence of SEQ ID NO:243. In some aspects, the pMHC complex comprises an amino acid sequence having at least about 91% sequence identity to the amino acid sequence of SEQ ID NO:243. In some aspects, the pMHC complex comprises an amino acid sequence having at least about 92% sequence identity to the amino acid sequence of SEQ ID NO:243. In some aspects, the pMHC complex comprises an amino acid sequence having at least about 93% sequence identity to the amino acid sequence of SEQ ID NO:243. In some aspects, the pMHC complex comprises an amino acid sequence having at least about 94% sequence identity to the amino acid sequence of SEQ ID NO:243. In some aspects, the pMHC complex comprises an amino acid sequence having at least about 95% sequence identity to the amino acid sequence of SEQ ID NO:243. In some aspects, the pMHC complex comprises an amino acid sequence having at least about 96% sequence identity to the amino acid sequence of SEQ ID NO:243. In some aspects, the pMHC complex comprises an amino acid sequence having at least about 97% sequence identity to the amino acid sequence of SEQ ID NO:243. In some aspects, the pMHC complex comprises an amino acid sequence having at least about 98% sequence identity to the amino acid sequence of SEQ ID NO:243. In some aspects, the pMHC complex comprises an amino acid sequence having at least about 99% sequence identity to the amino acid sequence of SEQ ID NO:243. In some aspects, the pMHC complex comprises an amino acid sequence having 100% sequence identity to the amino acid sequence of SEQ ID NO:243. In certain aspects of each of the foregoing embodiments, the peptide in the pMHC complex is identical to or has at most one or two amino acid substitutions to the peptide component of SEQ ID NO:243.

In some aspects, the pMHC complex comprises an amino acid sequence having at least about 90% sequence identity to the amino acid sequence of SEQ ID NO:245. In some aspects, the pMHC complex comprises an amino acid sequence having at least about 91% sequence identity to the amino acid sequence of SEQ ID NO:245. In some aspects, the pMHC complex comprises an amino acid sequence having at least about 92% sequence identity to the amino acid sequence of SEQ ID NO:245. In some aspects, the pMHC complex comprises an amino acid sequence having at least about 93% sequence identity to the amino acid sequence of SEQ ID NO:245. In some aspects, the pMHC complex comprises an amino acid sequence having at least about 94% sequence identity to the amino acid sequence of SEQ ID NO:245. In some aspects, the pMHC complex comprises an amino acid sequence having at least about 95% sequence identity to the amino acid sequence of SEQ ID NO:245. In some aspects, the pMHC complex comprises an amino acid sequence having at least about 96% sequence identity to the amino acid sequence of SEQ ID NO:245. In some aspects, the pMHC complex comprises an amino acid sequence having at least about 97% sequence identity to the amino acid sequence of SEQ ID NO:245. In some aspects, the pMHC complex comprises an amino acid sequence having at least about 98% sequence identity to the amino acid sequence of SEQ ID NO:245. In some aspects, the pMHC complex comprises an amino acid sequence having at least about 99% sequence identity to the amino acid sequence of SEQ ID NO:245. In some aspects, the pMHC complex comprises an amino acid sequence having 100% sequence identity to the amino acid sequence of SEQ ID NO:245. In certain aspects of each of the foregoing embodiments, the peptide in the pMHC complex is identical to or has at most one or two amino acid substitutions to the peptide component of SEQ ID NO:245.

In some aspects, the pMHC complex comprises an amino acid sequence having at least about 90% sequence identity to the amino acid sequence of SEQ ID NO:247. In some aspects, the pMHC complex comprises an amino acid sequence having at least about 91% sequence identity to the amino acid sequence of SEQ ID NO:247. In some aspects, the pMHC complex comprises an amino acid sequence having at least about 92% sequence identity to the amino acid sequence of SEQ ID NO:247. In some aspects, the pMHC complex comprises an amino acid sequence having at least about 93% sequence identity to the amino acid sequence of SEQ ID NO:247. In some aspects, the pMHC complex comprises an amino acid sequence having at least about 94% sequence identity to the amino acid sequence of SEQ ID NO:247. In some aspects, the pMHC complex comprises an amino acid sequence having at least about 95% sequence identity to the amino acid sequence of SEQ ID NO:247. In some aspects, the pMHC complex comprises an amino acid sequence having at least about 96% sequence identity to the amino acid sequence of SEQ ID NO:247. In some aspects, the pMHC complex comprises an amino acid sequence having at least about 97% sequence identity to the amino acid sequence of SEQ ID NO:247. In some aspects, the pMHC complex comprises an amino acid sequence having at least about 98% sequence identity to the amino acid sequence of SEQ ID NO:247. In some aspects, the pMHC complex comprises an amino acid sequence having at least about 99% sequence identity to the amino acid sequence of SEQ ID NO:247. In some aspects, the pMHC complex comprises an amino acid sequence having 100% sequence identity to the amino acid sequence of SEQ ID NO:247. In certain aspects of each of the foregoing embodiments, the peptide in the pMHC complex is identical to or has at most one or two amino acid substitutions to the peptide component of SEQ ID NO:247.

### 6.4. The Immune Cell Antigen Targeting Moiety

Aspects of the disclosure relate to molecules (e.g., T cell activators) comprising an immune cell antigen targeting moiety, i.e., a targeting moiety that specifically binds to an antigen present on or expressed by an immune cell. The immune cell antigen targeting moiety generally binds to a specific immune cell antigen and in particular cases to an epitope of the immune cell antigen that is present on the surface of a cell (e.g., extracellular domain of a cell surface or transmembrane protein). An immune cell antigen targeting moiety of the disclosure may be of a particular “type,” that is, may specifically bind to an antigen expressed by a particular type of immune cell (e.g., a T cell, a B cell, a dendritic cell, etc.). A T cell activator of the disclosure may comprise one, two, three, or more immune cell antigen targeting moieties. Where a T cell activator comprises multiple immune cell antigen targeting moieties, each may be of the same type or of different types. Types of immune cell antigen targeting moieties contemplated herein include, but are not limited to, T cell antigen targeting moieties and B cell antigen targeting moieties.

### 6.4.1. The T Cell Antigen Targeting Moiety

Certain T cell activators of the present disclosure comprise one or more T cell antigen (TCA) targeting moieties. Without being bound by theory, it is understood that the incorporation of T cell antigen (TCA) targeting moieties in the multispecific molecules of the disclosure permits, in some embodiments, activation of a T cell even in the absence of an antigen-presenting cell. For example, a T cell activator of the disclosure may bind to a T cell receptor on a T cell via the pMHC complex and also bind to a T cell antigen (e.g., CD3, TCR, CD28) on the T cell via the TCA targeting moiety, thereby activating the T cell in a manner that is specific for the particular antigen included in the pMHC complex.

The TCA targeting moieties of the disclosure include targeting moieties (e.g., antibodies and antigen-binding fragments thereof) that bind the TCA (e.g., CD3, CD28) with high affinity. The TCA targeting moieties of the disclosure also include targeting moieties (e.g., antibodies and antigen-binding fragments thereof) that bind the TCA (e.g., CD3, CD28) with moderate or low affinity, depending on the therapeutic context and particular targeting properties that are desired. In some embodiments, “low affinity” (also “weak affinity”) describes TCA targeting moieties that bind the TCA with a K_{D} or EC50 (e.g., as measured in a surface plasmon resonance assay) of greater than 10⁻⁶ M, greater than 10⁻⁷M, greater than 10⁻⁸ M, or greater than 10⁻⁹ M.

The present disclosure also includes TCA targeting moieties that bind the TCA with no measurable affinity. For example, in the context of a T cell activator comprising a pMHC complex and a CD3 targeting moiety, it may be desirable for the CD3 targeting moiety to bind to CD3 with only moderate or low affinity or no measurable affinity. In this manner, preferential binding of the pMHC to the TCR on a T cell may be achieved while avoiding general/untargeted CD3 binding. Accordingly, in some embodiments, a TCA targeting moiety of the present disclosure has a K_{D} as measured in a surface plasmon resonance assay of greater than 10⁻⁶ M, greater than 10⁻⁷M, greater than 10⁻⁸ M, or greater than 10⁻⁹ M, including any range or value derivable therein (e.g., between 10⁻⁶ M and 10⁻⁷M, between 10⁻⁶ M and 10⁻⁸ M, between 10⁻⁶ M and 10⁻⁹ M, between 10⁻⁷ M and 10⁻⁸ M, or between 10⁻⁷ M and 10⁻⁹ M, between 10⁻⁸ M and 10⁻⁸ M). Such TCA targeting moieties include those having no detectable binding as measured in a surface plasmon resonance assay; a TCA targeting moiety described as having “greater than” a particular K_{D} includes a TCA targeting moiety having no detectable binding.

The TCA targeting moiety generally binds to a specific T cell antigen. Exemplary target molecules recognized by the TCA targeting moieties of the disclosure are described in Section 6.4.1.

Suitable targeting moiety formats are described in Section 6.4.3. The targeting moiety is preferably an antigen binding moiety, for example an antibody or an antigen-binding portion of an antibody, e.g., an scFv, as described in Section 6.4.3.1, or a Fab, as described in Section 6.4.3.2.

### 6.4.1.1. T Cell Antigens

In general, a target for a TCA targeting moiety is any molecule present on or expressed by a T cell. In certain embodiments, a TCA targeting moiety of the disclosure targets a cell surface molecule of a T cell. Example targets for a TCA targeting moiety of the disclosure include, but are not limited to, CD3, the T cell receptor (e.g., TCRαβ or TCRγδ), CD27, CD28, 4-1BB (CD137), OX40, CD30, CD40, ICOS, lymphocyte function-associated antigen-1 (LFA-1), CD2, CD7, LIGHT, NKG2C, and B7-H3. In some embodiments, the target for the TCA targeting moiety is CD3. In some embodiments, the target for the TCA targeting moiety is the T cell receptor (e.g., TCRαβ or TCRγδ). In some embodiments, the target for the TCA targeting moiety is CD28. The epitope of the TCA targeting moiety can be an individual polypeptide (e.g., CD3 epsilon) or a multimeric component of a protein complex (e.g., the TCRαβ dimer or the TCRγδ dimer of the T cell receptor complex).

### 6.4.1.1.1. CD3 and TCR Targeting Moieties

In particular embodiments, a TCA targeting moiety of the present disclosure is a CD3 targeting moiety and/or a TCR targeting moiety. A CD3 targeting moiety may be or comprise an antigen-binding domain from an anti-CD3 antibody. A TCR targeting moiety may be or comprise an antigen-binding domain from an anti-TCR antibody.

Exemplary anti-CD3 and anti-TCR antibodies or antibody sequences are set forth in Table G-1 below, upon which the TCA targeting moiety can be based.

TABLE G-1
Exemplary Anti-CD3 and Anti-TCR Antibodies

| Target | Antibody Name and/or Binding Sequences |
|---|---|
| CD3 | The CD3-binding portion of Catumaxomab |
| CD3 | The CD3-binding portion of ertumaxomab |
| CD3 | The CD3-binding portion of anti-PSMA/anti-CD3 antibodies described in |
| | WO2011121110A1 |
| CD3 | Anti-CD3 antibody sequences in US10266593B2 |
| CD3 | Anti-CD3 antibody sequences in U.S. Pat. No. 8846042B2 |
| CD3 | Anti-CD3 antibody sequences U.S. 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in U.S. Pat. No. 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in U.S. Pat. No. 10,066,016 |
| CD3 | Anti-CD3 antibody sequences in U.S. 2010/0150918 |
| CD3 | The CD3-binding portion of MT110 |
| CD3 | The CD3-binding portion of Acapatamab (AMG160) |
| CD3 | The CD3-binding portion of AMG199 |
| CD3 | The CD3-binding portion of AMG330 |
| CD3 | The CD3-binding portion of AMG427 (Emirodatamab) |
| CD3 | The CD3-binding portion of AMG562 |
| CD3 | The CD3-binding portion of AMG596 |
| CD3 | The CD3-binding portion of AMG673 |
| CD3 | The CD3-binding portion of AMG701 (Pavurutamab) |
| CD3 | The CD3-binding portion of Tarlatamab (AMG757) |
| CD3 | The CD3-binding portion of AMG910 (Gresonitamab) |
| CD3 | The CD3-binding portion of BAY2010112 (Pasotuxizumab) |
| CD3 | The CD3-binding portion of AMG420 |
| CD3 | The CD3-binding portion of AMG424 |
| CD3 | The CD3-binding portion of AMG509 |
| CD3 | The CD3-binding portion of AMV564 |
| CD3 | The CD3-binding portion of APVO436 |
| CD3 | The CD3-binding portion of Alnuctamab (CC-93269; BMS-986349) |
| CD3 | The CD3-binding portion of ERY974 |
| CD3 | The CD3-binding portion of A-319 |
| CD3 | The CD3-binding portion of GEM333 |
| CD3 | The CD3-binding portion of GEM3PSCA |
| CD3 | The CD3-binding portion of Cevostamab |
| CD3 | The CD3-binding portion of Runimotamab |
| CD3 | The CD3-binding portion of GEN1044 |
| CD3 | Epcoritamab (GEN3013) |
| CD3 | The CD3-binding portion of HPN424 |
| CD3 | The CD3-binding portion of ISB1302 |
| CD3 | The CD3-binding portion of ISB1342 |
| CD3 | The CD3-binding portion of IGM-2323 |
| CD3 | The CD3-binding portion of IMC-F106C |
| CD3 | The CD3-binding portion of IMC-C103C |
| CD3 | The CD3-binding portion of IMCnyeso |
| CD3 | The CD3-binding portion of JNJ-63709178 |
| CD3 | The CD3-binding portion of JNJ-63898081 (JNJ-081) |
| CD3 | The CD3-binding portion of Teclistamab |
| CD3 | The CD3-binding portion of Talquetamab (JNJ-64407564) |
| CD3 | The CD3-binding portion of JNJ-67571244 |
| CD3 | The CD3-binding portion of MGD007 |
| CD3 | The CD3-binding portion of Orlotamab (MGD009) |
| CD3 | The CD3-binding portion of Duvortuxizumab |
| CD3 | The CD3-binding portion of Flotetuzumab (MGD006) |
| CD3 | The CD3-binding portion of MCLA-117 |
| CD3 | The CD3-binding portion of PF-06671008 |
| CD3 | The CD3-binding portion of Elranatamab |
| CD3 | The CD3-binding portion of Odronextamab |
| CD3 | The CD3-binding portion of REGN5458 |
| CD3 | The CD3-binding portion of REGN5459 |
| CD3 | The CD3-binding portion of REGN4018 |
| CD3 | The CD3-binding portion of Glofitamab (RO7082859) |
| CD3 | The CD3-binding portion of RO6958688 (RG7802) |
| CD3 | The CD3-binding portion of SAR440234 |
| CD3 | The CD3-binding portion of TNB-383B |
| CD3 | The CD3-binding portion of M802 |
| CD3 | The CD3-binding portion of Xmab13676 |
| CD3 | The CD3-binding portion of Xmab 18087 |
| CD3 | The CD3-binding portion of Vibecotamab (XmAb14045) |
| CD3 | The CD3-binding portion of Nivatrotamab (Hu3F8-BsAb) |
| CD3 | Anti-CD3 antibody sequences in US20190211100 |
| CD3 | Anti-CD3 antibody sequences in EP1629011B |
| CD3 | VH of SEQ ID NOS. 90 and 98 disclosed in U.S. 2021/0206865 A1 |
| | CDR-H1 of SEQ ID NO: 92 and 100 disclosed in U.S. 2021/0206865 A1 |
| | CDR-H2 of SEQ ID NO: 94 and 102 disclosed in U.S. 2021/0206865 A1 |
| | CDR-H3 of SEQ ID NO: 96 and 104 disclosed in U.S. 2021/0206865 A1 |
| | HC of SEQ ID NO: 127 or SEQ ID NO: 128 disclosed in U.S. 2021/0206865 A1 |
| | LC of SEQ ID NO: 129 or SEQ ID NO: 132 disclosed in U.S. 2021/0206865 A1 |
| CD3 | Anti-CD3 Heavy chain of SEQ ID NO: 2 disclosed in U.S. 2021/0206870 A1 |
| | Anti-CD3 VH SEQ ID NO: 5 disclosed in U.S. 2021/0206870 A1 |
| | Anti-CD3 VL of SEQ ID NO: 6 disclosed in U.S. 2021/0206870 A1 |
| | Anti-CD3 CDR-H1 of SEQ ID NO: 10 disclosed in U.S. 2021/0206870 A1 |
| | Anti-CD3 CDR-H2 of SEQ ID NO: 11 disclosed in U.S. 2021/0206870 A1 |
| | Anti-CD3 CDR-H3 of SEQ ID NO: 12 disclosed in U.S. 2021/0206870 A1 |
| CD3 | Anti-CD3 VH of SEQ ID NO: 92, 102, 112, 122, 132, 142, 156, 166, 176, 186, 196 |
| | or 206 disclosed in U.S. 2022/0119525 A1 |
| | Anti-CD3 CDR-H1 of SEQ ID NO: 93, 103, 113, 123, 133, 143, 157, 167, 177, |
| | 187, 197 or 207 disclosed in U.S. 2022/0119525 A1 |
| | Anti-CD3 CDR-H2 of SEQ ID NO: 94, 104, 114, 124, 134, 144, 158, 168, 178, |
| | 188, 198 or 208 disclosed in U.S. 2022/0119525 A1 |
| | Anti-CD3 CDR-H3 of SEQ ID NO: 95, 105, 115, 125, 135, 145, 159, 169, 179, |
| | 189, 199 or 209 disclosed in U.S. 2022/0119525 A1 |
| | Anti-CD3 VL of SEQ ID NO: 96, 106, 116, 126, 136, 146, 152, 162, 172, 182, 192 |
| | or 202 disclosed in U.S. 2022/0119525 A1 |
| | Anti-CD3 CDR-L1 of SEQ ID NO: 97, 107, 117, 127, 137, 147, 153, 163, 173, 183, |
| | 193 or 203 disclosed in U.S. 2022/0119525 A1 |
| | Anti-CD3 CDR-L2 of SEQ ID NO. 98, 108, 118, 128, 138, 148, 154, 164, 174, 184, |
| | 194 or 204 disclosed in U.S. 2022/0119525 A1 |
| | Anti-CD3 CDR-L3 of SEQ ID NO. 99, 109, 119, 129, 139, 149, 155, 165, 175, 185, |
| | 195 or 205 disclosed in U.S. 2022/0119525 A1 |
| CD3 | L2K |
| CD3 | A2J |
| CD3 | 6G12 |
| CD3 | 1A4 |
| CD3 | OKT3 (Ortho Kung T3; Muromonab-CD3) |
| CD3 | Teplizumab (PRV-031; MGA03) |
| CD3 | Otelixizumab (TRX4) |
| CD3 | Anti-CD3 VH of SEQ ID NO: 2, 18, 34, 50, 66, 82, 98, 114, 130, 146, 162, 178, |
| | 194, 210, 226, 242, 258, 274, 290, 306, 322, 338, 354, 370, 386, 402, 418, 434, |
| | 450, 466, 482, 498, 514, 530, 546, 562, 578, 594, 610, 626, 642, 658, 674, 690, |
| | 706, 722, 738, 754, 770, 786, 802, 818, 834, 850, 866, 882, 898, 914, 930, 946, |
| | 962, 978, 994, 1010, 1026, 1042, 1050, 1058, 1066, 1074, 1082, 1090, 1098, |
| | 1106, 1114, 1122, 1130, 1138, 1146, 1154, 1162, 1170, 1178, 1186, 1194, 1202, |
| | 1210, 1218, or 1226 disclosed in U.S. Pat. No. 9657102 B2 |
| | Anti-CD3 CDR-H1 of SEQ ID NO: 4, 20, 36, 52, 68, 84, 100, 116, 132, 148, 164, |
| | 180, 196, 212, 228, 244, 260, 276, 292, 308, 324, 340, 356, 372, 388, 404, 420, |
| | 436, 452, 468, 484, 500, 516, 532, 548, 564, 580, 596, 612, 628, 644, 660, 676, |
| | 692, 708, 724, 740, 756, 772, 788, 804, 820, 836, 852, 868, 884, 900, 916, 932, |
| | 948, 964, 980, 996, 1012, 1028, 1044, 1052, 1060, 1068, 1076, 1084, 1092, 1100, |
| | 1108, 1116, 1124, 1132, 1140, 1148, 1156, 1164, 1172, 1180, 1188, 1196, 1204, |
| | 1212, or 1220, 1228 disclosed in U.S. Pat. No. 9657102 B2 |
| | Anti-CD3 CDR-H2 of SEQ ID NO: 6, 22, 38, 54, 70, 86, 102, 118, 134, 150, 166, |
| | 182, 198, 214, 230, 246, 262, 278, 294, 310, 326, 342, 358, 374, 390, 406, 422, |
| | 438, 454, 470, 486, 502, 518, 534, 550, 566, 582, 598, 614, 630, 646, 662, 678, |
| | 694, 710, 726, 742, 758, 774, 790, 806, 822, 838, 854, 870, 886, 902, 918, 934, |
| | 950, 966, 982, 998, 1014, 1030, 1046, 1054, 1062, 1070, 1078, 1086, 1094, 1102, |
| | 1110, 1118, 1126, 1134, 1142, 1150, 1158, 1166, 1174, 1182, 1190, 1198, 1206, |
| | 1214, or 1222, 1230 disclosed in U.S. Pat. No. 9657102 B2 |
| | Anti-CD3 CDR-H3 of SEQ ID NO: 8, 24, 40, 56, 72, 88, 104, 120, 136, 152, 168, |
| | 184, 200, 216, 232, 248, 264, 280, 296, 312, 328, 344, 360, 376, 392, 408, 424, |
| | 440, 456, 472, 488, 504, 520, 536, 552, 568, 584, 600, 616, 632, 648, 664, 680, |
| | 696, 712, 728, 744, 460, 776, 792, 808, 824, 840, 856, 872, 888, 904, 920, 936, |
| | 952, 968, 984, 1000, 1016, 1032, 1048, 1056, 1064, 1072, 1080, 1088, 1096, |
| | 1104, 1112, 1120, 1128, 1136, 1144, 1152, 1160, 1168, 1176, 1184, 1192, 1200, |
| | 1208, 1216, or 1224, 1232 disclosed inU.S. Pat. No. 9657102 B2 |
| | Anti-CD3 VL of SEQ ID NO: 10, 26, 42, 58, 74, 90, 106, 122, 138, 154, 170, 186, |
| | 202, 218, 234, 250, 266, 282, 298, 314, 330, 346, 362, 378, 394, 410, 426, 442, |
| | 458, 474, 490, 506, 522, 538, 554, 570, 586, 602, 618, 634, 650, 666, 682, 698, |
| | 714, 730, 746, 762, 778, 794, 810, 826, 842, 858, 874, 890, 906, 922, 938, 954, |
| | 970, 986, 1002, 1018, 1034, 1234, 1234, 1234, 1234, 1234, 1234, 1234, 1234, |
| | 1234, 1234, 1234, 1234, 1234, 1234, 1234, 1234, 1234, 1234, 1234, 1234, 1234, |
| | 1234, or 1234, 1234 disclosed in U.S. Pat. No. 9657102 B2 |
| | Anti-CD3 CDR-L1 of SEQ ID NO: 12, 28, 44, 60, 76, 92, 108, 124, 140, 156, 172, |
| | 188, 204, 220, 236, 252, 268, 284, 300, 316, 332, 348, 364, 380, 396, 412, 428, |
| | 444, 460, 476, 492, 508, 524, 540, 556, 572, 588, 604, 620, 636, 652, 668, 684, |
| | 700, 716, 732, 748, 764, 780, 796, 812, 828, 844, 860, 876, 892, 908, 924, 940, |
| | 956, 972, 988, 1004, 1020, 1036, 1236, 1236, 1236, 1236, 1236, 1236, 1236, |
| | 1236, 1236, 1236, 1236, 1236, 1236, 1236, 1236, 1236, 1236, 1236, 1236, 1236, |
| | 1236, 1236, or 1236, 1236 disclosed in U.S. Pat. No. 9657102 B2 |
| | Anti-CD3 CDR-L2 of SEQ ID NO. 14, 30, 46, 62, 78, 94, 110, 126, 142, 158, 174, |
| | 190, 206, 222, 238, 254, 270, 286, 302, 318, 334, 350, 366, 382, 398, 414, 430, |
| | 446, 462, 478, 494, 510, 526, 542, 558, 574, 590, 606, 622, 638, 654, 670, 686, |
| | 702, 718, 734, 750, 766, 782, 798, 814, 830, 846, 862, 878, 894, 910, 926, 942, |
| | 958, 974, 990, 1006, 1022, 1038, 1238, 1238, 1238, 1238, 1238, 1238, 1238, |
| | 1238, 1238, 1238, 1238, 1238, 1238, 1238, 1238, 1238, 1238, 1238, 1238, 1238, |
| | 1238, 1238, or 1238, 1238 disclosed in U.S. Pat. No. 9657102 B2 |
| | Anti-CD3 CDR-L3 of SEQ ID NO. 16, 32, 48, 64, 80, 96, 112, 128, 144, 160, 176, |
| | 192, 208, 224, 240, 256, 272, 288, 304, 320, 336, 352, 368, 384, 400, 416, 432, |
| | 448, 464, 480, 496, 512, 528, 544, 560, 576, 592, 608, 624, 640, 656, 672, 688, |
| | 704, 720, 736, 752, 768, 784, 800, 816, 832, 848, 864, 880, 896, 912, 928, 944, |
| | 960, 976, 992, 1008, 1024, 1040, 1240, 1240, 1240, 1240, 1240, 1240, 1240, |
| | 1240, 1240, 1240, 1240, 1240, 1240, 1240, 1240, 1240, 1240, 1240, 1240, 1240, |
| | 1240, 1240, or 1240, 1240 disclosed in U.S. Pat. No. 9657102 B2 |
| | (see also US 9657102 B2 at Table 1, incorporated herein by reference) |
| CD3 | VH: |
| | EVQLVESGGGLVQPGRSLRLSCAASGFTFADYTMHWVRQAPGKGLEWVSDIS |
| | WNSGSIAYADSVKGRFTISRDNAKNSLYLQMNSLRTEDTAFYYCAKDSRGYGHY |
| | KYLGLDVWGQGTTVTVSS (SEQ ID NO: 231) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPPITFGQGTRLEIK |
| | (SEQ ID NO: 236) |
| CD3 | VH: |
| | EVQLVESGGGLVQPGRSLRLSCAASGFTFDDYSMHWVRQAPGKGLEWVSGIS |
| | WNSGSKGYADSVKGRFTISRDNAKNSLYLQMNSLRAEDTALYYCAKYGSGYGK |
| | FYHYGLDVWGQGTTVTVSS (SEQ ID NO: 232) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPPITFGQGTRLEIK |
| | (SEQ ID NO: 236) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKDGSGYGKF |
| | YYYGMDVWGQGTTVTVSS (SEQ ID NO: 233) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPPITFGQGTRLEIK |
| | (SEQ ID NO: 236) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKYGSGYGKF |
| | YYYGMDVWGQGTTVTVSS (SEQ ID NO: 234) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPPITFGQGTRLEIK |
| | (SEQ ID NO: 236) |
| TCRαβ | BMA031 sequences disclosed in U.S. 2012/0034221 |
| TCRγδ | 6TCS1 antibody disclosed in U.S. Pat. No. 5,980,892 |

In some aspects, the TCA targeting moiety competes with an antibody set forth in Table G-1 for binding to the target (e.g., CD3 or a T cell receptor). In further aspects, the TCA targeting moiety comprises CDRs having CDR sequences of an antibody set forth in Table G-1. In some embodiments, the TCA targeting moiety comprises all 6 CDR sequences of an antibody set forth in Table G-1. In other embodiments, the TCA targeting moiety comprises at least the heavy chain CDR sequences (CDR-H1, CDR-H2, CDR-H3) or an antibody set forth in Table G-1 and the light chain CDR sequences of a universal light chain. In further aspects, a TCA targeting moiety comprises a VH comprising the amino acid sequence of the VH of an antibody set forth in Table G-1. In some embodiments, the TCA targeting moiety further comprises a VL comprising the amino acid sequence of the VL of an antibody set forth in Table G-1. In other embodiments, the TCA targeting moiety further comprises a universal light chain VL sequence.

The CD3 targeting moieties of the disclosure include targeting moieties (e.g., antibodies and antigen-binding fragments thereof) that bind CD3 (e.g., human CD3) with high affinity. The CD3 targeting moieties of the disclosure also include targeting moieties (e.g., antibodies and antigen-binding fragments thereof) that bind the CD3 (e.g., human CD3) with moderate or low affinity, depending on the therapeutic context and particular targeting properties that are desired. The present disclosure also includes CD3 targeting moieties that bind CD3 (e.g., human CD3) with no measurable affinity. Accordingly, in some embodiments, a CD3 targeting moiety of the present disclosure has a K_{D} as measured in a surface plasmon resonance assay (e.g., at 25° C.) of greater than 10⁻⁶ M, greater than 10⁻⁷ M, greater than 10⁻⁸ M, or greater than 10⁻⁹ M, including any range or value derivable therein (e.g., between 10⁻⁶ M and 10⁻⁷M, between 10⁻⁶ M and 10⁻⁸ M, between 10⁻⁶ M and 10⁻⁹ M, between 10⁻⁷ M and 10⁻⁸ M, or between 10⁻⁷M and 10⁻⁹ M, or between 10⁻⁸ M and 10⁻⁸ M). Such CD3 targeting moieties include those having no detectable binding as measured in a surface plasmon resonance assay; a CD3 targeting moiety described as having “greater than” a particular K_{D} includes a CD3 targeting moiety having no detectable binding. For example, in the context of a T cell activator comprising a CD3 targeting moiety and a pMHC complex, it may be desirable for the CD3 targeting moiety to bind CD3 with moderate or low affinity thereby since, as disclosed herein, a T cell activator having a CD3 binding moiety with moderate to weak affinity provides superior antigen-specific T cell activation compared with those having a CD3 binding moiety with strong affinity (see, e.g., Examples 1-4 herein). In other embodiments, a CD3 targeting moiety of the present disclosure has a K_{D} as measured in a surface plasmon resonance assay (e.g., at 25° C.) of less than 10⁻⁹ M, less than 10⁻¹⁰ M, less than 10⁻¹¹ M, or less than 10⁻¹² M, including any range or value derivable therein (e.g., between 10⁻⁹ M and 10⁻¹² M, between 10⁻¹⁰ M and 10⁻¹² M, between 10⁻¹¹ M and 10⁻¹² M, between 10⁻⁹ M and 10⁻¹⁰ M, between 10⁻⁹ M and 10⁻¹¹ M, or between 10⁻¹⁰ M and 10⁻¹¹ M.

### 6.4.1.1.2. CD28 Targeting Moieties

In particular embodiments, a TCA targeting moiety of the present disclosure is a CD28 targeting moiety. A CD28 targeting moiety may be or comprise an antigen-binding domain from an anti-CD28 antibody.

Exemplary anti-CD28 antibodies or antibody sequences are set forth in Table G-2 below, upon which the TCA targeting moiety can be based.

TABLE G-2
Exemplary Anti-CD28 Antibodies

| Target | Antibody Name and/or Binding Sequences |
|---|---|
| CD28 | Theralizumab (TGN1412) |
| CD28 | FK734 |
| CD28 | CD28A-49 |
| CD28 | Anti-CD28 antibody sequences in WO 2020/127618 A1 |
| CD28 | Anti-CD28 antibodies and CD28 portion of anti-PSMA/anti-CD28 antibodies |
| | described in WO 2019/246514 A1 |
| CD28 | The CD28 portion of anti-CD3/anti-CD28 antibodies described in WO 2021/260064 |
| | A1 |
| CD28 | The CD28 portion of anti-CD3/anti-CD28 antibodies described in WO 2009/062001 |
| | A1 |
| CD28 | The CD28 portion of anti-EGFR/anti-CD28 antibody described in WO 2020/198009 |
| | A1 |
| CD28 | The CD28 portion of anti-CD3/anti-CD28/anti-CD137 antibodies described in WO |
| | 2021/173307 A1 |
| CD28 | The CD28 portion of anti-CD38/anti-CD28/anti-CD3 antibodies described in WO |
| | 2019/074973 A2 |
| CD28 | The CD28 portion of anti-CEA/anti-CD3/anti-CD28 antibodies described in WO |
| | 2005/095456 A1 |
| CD28 | The CD28 portion of anti-MUC16/anti-CD28 antibodies described in WO |
| | 2020/132024 A1 |
| CD28 | The CD28 portion of anti-CD28/anti-CD22 antibodies described in WO |
| | 2020/132066 A1 |
| CD28 | Anti-CD28 antibody sequences in WO 2020/127628 A1 |
| CD28 | Anti-CD28 antibody sequences in WO 2022/061098 A1 |
| CD28 | Anti-CD28 antibody sequences in WO 2003/042231 A2 |
| CD28 | Anti-CD28 antibody sequences in WO 2014/209868 A1 |
| CD28 | Anti-CD28 antibody sequences in WO 2022/096536 A1 |
| CD28 | VH: |
| | EVQLVESGGGLVQPGGSLRLSCAASGFTFSRNNMHWVRQAPGKGLEYVSGISS |
| | NGGRTYYADSVKGRFTISRDNSKNTLYLQMGGLRAADMAVYFCTRDDELLSFDY |
| | WGQGTLVTVSS (SEQ ID NO: 237) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPPITFGQGTRLEIK |
| | (SEQ ID NO: 236) |

In some aspects, the TCA targeting moiety competes with an antibody set forth in Table G-2 for binding to the target (e.g., CD28). In further aspects, the TCA targeting moiety comprises CDRs having CDR sequences of an antibody set forth in Table G-2. In some embodiments, the TCA targeting moiety comprises all 6 CDR sequences of an antibody set forth in Table G-2. In other embodiments, the TCA targeting moiety comprises at least the heavy chain CDR sequences (CDR-H1, CDR-H2, CDR-H3) or an antibody set forth in Table G-2 and the light chain CDR sequences of a universal light chain. In further aspects, a TCA targeting moiety comprises a VH comprising the amino acid sequence of the VH of an antibody set forth in Table G-2. In some embodiments, the TCA targeting moiety further comprises a VL comprising the amino acid sequence of the VL of an antibody set forth in Table G-2. In other embodiments, the TCA targeting moiety further comprises a universal light chain VL sequence.

The CD28 targeting moieties of the disclosure include targeting moieties (e.g., antibodies and antigen-binding fragments thereof) that bind CD28 (e.g., human CD28) with high affinity. The CD28 targeting moieties of the disclosure also include targeting moieties (e.g., antibodies and antigen-binding fragments thereof) that bind the CD28 (e.g., human CD28) with moderate or low affinity, depending on the therapeutic context and particular targeting properties that are desired. The present disclosure also includes CD28 targeting moieties that bind CD28 (e.g., human CD28) with no measurable affinity. Accordingly, in some embodiments, a CD28 targeting moiety of the present disclosure has a K_{D} as measured in a surface plasmon resonance assay of greater than 10⁻⁶ M, greater than 10⁻⁷ M, greater than 10⁻⁸ M, or greater than 10⁻⁹ M, including any range or value derivable therein (e.g., between 10⁻⁶ M and 10⁻⁷M, between 10⁻⁶ M and 10⁻⁸ M, between 10⁻⁶ M and 10⁻⁹ M, between 10⁻⁷ M and 10⁻⁸ M, or between 10⁻⁷ M and 10⁻⁹ M, between 10⁻⁸ M and 10⁻⁸ M). Such CD28 targeting moieties include those having no detectable binding as measured in a surface plasmon resonance assay; a CD28 targeting moiety described as having “greater than” a particular K_{D} includes a CD28 targeting moiety having no detectable binding. For example, in the context of a T cell activator comprising a CD28 targeting moiety and a pMHC complex, it may be desirable for the CD28 targeting moiety to bind to CD28 with only moderate or low affinity. In this manner, preferential targeting of the T cell activator to T cells expressing a TCR specific for the pMHC complex may be achieved while avoiding general/untargeted CD28 binding and the consequent adverse side effects associated therewith.

In other embodiments, a CD28 targeting moiety of the present disclosure has a K_{D} as measured in a surface plasmon resonance assay (e.g., at 25° C.) of less than 10⁻⁹ M, less than 10⁻¹⁰ M, less than 10⁻¹¹ M, or less than 10⁻¹² M, including any range or value derivable therein (e.g., between 10⁻⁹ M and 10⁻¹² M, between 10⁻¹⁰ M and 10⁻¹² M, between 10⁻¹¹ M and 10⁻¹² M, between 10⁻⁹ M and 10⁻¹⁰ M, between 10⁻⁹ M and 10⁻¹¹ M, or between 10⁻¹⁰ M and 10⁻¹¹ M.

### 6.4.2. The B Cell Antigen Targeting Moiety

Certain T cell activators of the present disclosure comprise one or more B cell antigen (BCA) targeting moieties. The BCA targeting moiety generally binds to a specific B cell antigen. Exemplary target molecules recognized by the targeting moieties of the disclosure are described in Section 6.4.2.1.

Suitable targeting moiety formats are described in Section 6.4.3. The targeting moiety is preferably an antigen binding moiety, for example an antibody or an antigen-binding portion of an antibody, e.g., an scFv, as described in Section 6.4.3.1, or a Fab, as described in Section 6.4.3.2.

In some embodiments, a T cell activator of the disclosure that comprises a BCA targeting moiety lacks a TCA (e.g., CD3) targeting moiety. In other embodiments, a T cell activator of the disclosure that comprises a BCA targeting moiety also comprises a TCA (e.g., CD3) targeting moiety.

### 6.4.2.1. B Cell Antigens

In general, a target for a BCA targeting moiety is any molecule present on or expressed by a B cell. In certain embodiments, a BCA targeting moiety of the disclosure targets a cell surface molecule of a B cell. Example targets for a BCA targeting moiety of the disclosure include, but are not limited to, CD19, CD20, and CD22. In some embodiments, the target for the BCA targeting moiety is CD19. In some embodiments, the target for the BCA targeting moiety is CD20. In some embodiments, the target for the BCA targeting moiety is CD22. The epitope of the BCA targeting moiety can be an individual polypeptide or a multimeric component of a protein complex.

### 6.4.2.1.1. CD19 Targeting Moieties

In particular embodiments, a BCA targeting moiety of the present disclosure is a CD19 targeting moiety. A CD19 targeting moiety may be or comprise an antigen-binding domain from an anti-CD19 antibody.

Exemplary anti-CD19 antibodies or antibody sequences are set forth in Table G-3 below, upon which the BCA targeting moiety can be based.

TABLE G-3
Exemplary Anti-CD19 Antibodies

| Target | Antibody Name and/or Binding Sequences |
|---|---|
| CD19 | Anti-CD19 antibody sequences in WO 2017/055328 A1 |
| CD19 | Anti-CD19 antibody sequences in WO 2006/121852 A2 |
| CD19 | Anti-CD19 antibody sequences in WO 2010/053716 A1 |
| CD19 | Anti-CD19 antibody sequences in U.S. Pat. No.: 8,097,703 |
| CD19 | Anti-CD19 antibody sequences in U.S. Pat. No.: 7,462,352 |
| CD19 | Anti-CD19 antibody sequences in WO 2010/102276 A2 |
| CD19 | Anti-CD19 antibody sequences in WO 2007/076950 A1 |
| CD19 | Anti-CD19 antibody sequences in WO 2006/133450 A2 |
| CD19 | The CD19 portion of anti-CD3/anti-CD19 antibodies |
| | described in WO 2004/106381 A1 |
| CD19 | Anti-CD19 antibody sequences in WO 2017/055541 A1 |
| CD19 | Anti-CD19 antibody sequences in WO 2019/057100 A1 |
| CD19 | Blincyto ™ (blinatumomab) |
| CD19 | SGN-CD19A |

In some aspects, the BCA targeting moiety competes with an antibody set forth in Table G-3 for binding to the target. In further aspects, the BCA targeting moiety comprises CDRs having CDR sequences of an antibody set forth in Table G-3. In some embodiments, the BCA targeting moiety comprises all 6 CDR sequences of an antibody set forth in Table G-3. In other embodiments, the BCA targeting moiety comprises at least the heavy chain CDR sequences (CDR-H1, CDR-H2, CDR-H3) or an antibody set forth in Table G-3 and the light chain CDR sequences of a universal light chain. In further aspects, a BCA targeting moiety comprises a VH comprising the amino acid sequence of the VH of an antibody set forth in Table G-3. In some embodiments, the BCA targeting moiety further comprises a VL comprising the amino acid sequence of the VL of an antibody set forth in Table G-3. In other embodiments, the BCA targeting moiety further comprises a universal light chain VL sequence.

### 6.4.2.1.2. CD20 Targeting Moieties

In particular embodiments, a BCA targeting moiety of the present disclosure is a CD20 targeting moiety. A CD20 targeting moiety may be or comprise an antigen-binding domain from an anti-CD20 antibody.

Exemplary anti-CD20 antibodies or antibody sequences are set forth in Table G-4 below, upon which the BCA targeting moiety can be based.

TABLE G-4
Exemplary Anti-CD20 Antibodies

| Target | Antibody Name and/or Binding Sequences |
|---|---|
| CD20 | Anti-CD20 antibody sequences in WO 2011/100403 A1 |
| CD20 | Anti-CD20 antibody sequences in WO 2005/044859 A2 |
| CD20 | Anti-CD20 antibody sequences in U.S. Pat. No. 7, 151, 164 |
| CD20 | Anti-CD20 antibody sequences in WO 2004/035607 A2 |
| CD20 | Anti-CD20 antibody sequences in U.S. Pat. No. 9,045,543 |
| CD20 | The CD20 portion of anti-CD3/anti-CD20 antibodies described in WO |
| | 2016/110576A1 |
| CD20 | Anti-CD20 antibody sequences in WO 2006/084264 A2 |
| CD20 | Anti-CD20 antibody sequences in U.S. Pat. No. 9, 175,086 |
| CD20 | Anti-CD20 antibody sequences in WO 2018/041067 A1 |
| CD20 | Anti-CD20 antibody sequences in WO 2005/000901 A2 |
| CD20 | The CD20 portion of anti-CD3/anti-CD20 antibodies described in U.S. Pat. No. |
| | 11,155,621 |
| CD20 | The CD20 portion of anti-CD3/anti-CD20 antibodies described in WO 2018/114748 |
| | A1 |
| CD20 | Bexxar ™ (tositumomab) |
| | VH: the VH sequence of the heavy chain of SEQ ID NO: 124 of U.S. Pat. No. U.S. |
| | 2017/0002060 A1 |
| | VL: the VL sequence of the light chain of SEQ ID NO: 125 of U.S. Pat. No. U.S. |
| | 2017/0002060 A1 |
| CD20 | Zevalin ™ (ibritumomab tiuxetan) |
| | VH: SEQ ID NO: 9 of U.S. Pat. No. 5,736, 137 |
| | VL: SEQ ID NO: 6 of U.S. Pat. No. 5,736, 137 |
| CD20 | Rituxan ™ (rituximab) |
| | VH: SEQ ID NO: 9 of U.S. Pat. No. 5,736, 137 |
| | VL: SEQ ID NO: 6 of U.S. Pat. No. 5,736, 137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva™ (obinutuzumab) |
| CD20 | VH: SEQ ID NO: 4 of U.S. 2021/0206870 A1 |
| | VL of SEQ ID NO: 6 of U.S. 2021/0206870 A1 |
| CD20 | epcoritamab |
| CD20 | VH: SEQ ID NO: 1242 of U.S. Pat. No. 9,657, 102 |
| | VL: SEQ ID NO: 1250 or 1266 of U.S. Pat. No. 9,657, 102 |
| CD20 | VH: SEQ ID NO: 1338 of U.S. Pat. No. 9,657, 102 |
| | VL: SEQ ID NO: 1250 or 1266 of U.S. Pat. No. 9,657, 102 |
| CD20 | VH: |
| | EVQLVESGGGLVQPGRSLRLSCVASGFTFNDYAMHWVRQAPGKGLEWVSVISWN |
| | SDSIGYADSVKGRFTISRDNAKNSLYLQMHSLRAEDTALYYCAKDNHYGSGSYYYY |
| | QYGMDVWGQGTTVTVSS (SEQ ID NO: 238) |
| | VL: |
| | EIVMTQSPATLSVSPGERATLSCRASQSVSSNLAWYQQKPGQAPRLLIYGASTRAT |
| | GIPARFSGSGSGTEFTLTISSLQSEDFAVYYCQHYINWPLTFGGGTKVEIK (SEQ ID |
| | NO: 239) |
| CD20 | VH: |
| | QVHLVESGGGVVQPGRSLRLSCSVSGLTFNSYGMHWVRQAPGKGLEWVTMISYD |
| | GSDKYYLDSVKGRFTISRDNSKNILYLQINSLRPEDTAVYYCVNWDNWRGNFDYW |
| | GQGTLVTVSS (SEQ ID NO: 238) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPPITFGQGTRLEIK (SEQ |
| | ID NO: 236) |

In some aspects, the BCA targeting moiety competes with an antibody set forth in Table G-4 for binding to the target. In further aspects, the BCA targeting moiety comprises CDRs having CDR sequences of an antibody set forth in Table G-4. In some embodiments, the BCA targeting moiety comprises all 6 CDR sequences of an antibody set forth in Table G-4. In other embodiments, the BCA targeting moiety comprises at least the heavy chain CDR sequences (CDR-H1, CDR-H2, CDR-H3) or an antibody set forth in Table G-4 and the light chain CDR sequences of a universal light chain. In further aspects, a BCA targeting moiety comprises a VH comprising the amino acid sequence of the VH of an antibody set forth in Table G-4. In some embodiments, the BCA targeting moiety further comprises a VL comprising the amino acid sequence of the VL of an antibody set forth in Table G-4. In other embodiments, the BCA targeting moiety further comprises a universal light chain VL sequence.

### 6.4.2.1.3. CD22 Targeting Moieties

In particular embodiments, a BCA targeting moiety of the present disclosure is a CD22 targeting moiety. A CD22 targeting moiety may be or comprise an antigen-binding domain from an anti-CD22 antibody.

Exemplary anti-CD22 antibodies or antibody sequences are set forth in Table G-5 below, upon which the BCA targeting moiety can be based.

TABLE G-5
Exemplary Anti-CD22 Antibodies

| Target | Antibody Name and/or Binding Sequences |
|---|---|
| CD22 | Anti-CD22 antibody sequences in U.S. Pat. No.: 9,139,649 |
| CD22 | The CD22 portion of anti-CD28/anti-CD22 antibodies |
| | described in WO 2020/132066 A1 |
| CD22 | Anti-CD2 antibody sequences in WO 2020/185763 A1 |
| Target | Antibody Name and/or Binding Sequences |
| CD22 | Anti-CD2 antibody sequences in WO 2007/103470 A2 |
| CD22 | Anti-CD2 antibody sequences in U.S. Pat. No.: 8,968,741 |
| CD22 | Anti-CD2 antibody sequences in WO 2014/011518 A1 |
| CD22 | Anti-CD2 antibody sequences in WO 2008/070569 A2 |
| CD22 | Anti-CD2 antibody sequences in U.S. Pat. No.: 10,259,871 |
| CD22 | Anti-CD2 antibody sequences in WO 2007/103469 A2 |
| CD22 | Anti-CD2 antibody sequences in WO 2013/163519 A1 |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |

In some aspects, the BCA targeting moiety competes with an antibody set forth in Table G-5 for binding to the target. In further aspects, the BCA targeting moiety comprises CDRs having CDR sequences of an antibody set forth in Table G-5. In some embodiments, the BCA targeting moiety comprises all 6 CDR sequences of an antibody set forth in Table G-5. In other embodiments, the BCA targeting moiety comprises at least the heavy chain CDR sequences (CDR-H1, CDR-H2, CDR-H3) or an antibody set forth in Table G-5 and the light chain CDR sequences of a universal light chain. In further aspects, a BCA targeting moiety comprises a VH comprising the amino acid sequence of the VH of an antibody set forth in Table G-5. In some embodiments, the BCA targeting moiety further comprises a VL comprising the amino acid sequence of the VL of an antibody set forth in Table G-5. In other embodiments, the BCA targeting moiety further comprises a universal light chain VL sequence.

### 6.4.3. Targeting Moiety Format

In certain aspects, the targeting moiety can be any type of antibody or fragment thereof that retains specific binding to an antigenic determinant. In one embodiment the antigen binding moiety is a full-length antibody. In one embodiment the antigen binding moiety is an immunoglobulin molecule, particularly an IgG class immunoglobulin molecule, more particularly an IgG1 or IgG4 immunoglobulin molecule. Antibody fragments include, but are not limited to, VH (or V_{H}) fragments, VL (or V_{L}) fragments, Fab fragments, F(ab′)₂ fragments, scFv fragments, Fv fragments, VHH fragments, minibodies, intrabodies, diabodies, triabodies, and tetrabodies.

6.4.3.1. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.8.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.8 (typically a repeat of a sequence containing the amino acids glycine and serine, such as the amino acid sequence (Gly4˜Ser)₃ (SEQ ID NO: 127), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.4.3.2. Fab

Fab domains were traditionally produced by proteolytic cleavage of immunoglobulin molecules using enzymes such as papain. In the T cell activators of the disclosure, the Fab domains are typically recombinantly expressed as part of the T cell activator.

The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the molecules of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table 3 below can be used:

TABLE 3
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + hole or | Wozniak-Knopp et al., |
| exchanged | | hole mutation | | knob mutation | 2018, |
| | | | | | PLOSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of a T cell activator of the disclosure. In various embodiments, employing a common light chain as described herein reduces the number of inappropriate species of T cell activators as compared to employing original cognate VLs. In various embodiments, the VL domains of the T cell activators are identified from monospecific antibodies comprising a common light chain. In various embodiments, the VH regions of the T cell activators comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

### 6.5. Tumor Antigen Targeting Moieties

The T cell activators of the disclosure may optionally comprise at least one targeting moiety that binds specifically to a target molecule expressed by a tumor cell or in the tumor cell environment, e.g., an extracellular matrix (“ECM”) antigen, a tumor reactive lymphocyte antigen, a cell surface molecule of tumor or viral lymphocytes, a checkpoint inhibitor, or a tumor-associated antigen (TAA), collectively referred to herein as a “tumor antigen targeting moieties.” The skilled artisan would recognize that the foregoing categories of target molecules are not mutually exclusive and thus a given target molecule may fall into more than one of the foregoing categories of target molecules. For example, some molecules may be considered both TAAs and ECM proteins. Preferably, the ECM antigen, tumor reactive lymphocyte antigen, cell surface molecule of tumor or viral lymphocytes, checkpoint inhibitor, or TAA is a human antigen. The antigen may or may not be present on normal cells. Particular aspects are directed to T cell activators comprising at least one targeting moiety that binds specifically to a TAA.

It is anticipated that any type of tumor and any type of ECM antigen, tumor reactive lymphocyte antigen, cell surface molecule of tumor or viral lymphocytes, checkpoint inhibitor, or TAA may be targeted by the T cell activators of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3. In some embodiments, where the target molecule is a checkpoint inhibitor, the tumor antigen targeting moiety is non-blocking or poorly-blocking of ligand-receptor binding. Examples of non-blocking or poorly-blocking anti-PD1 antibodies includes antibodies having VH/VL amino acid sequences of SEQ ID Nos: 2/10 of PCT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

In certain embodiments, the target molecule of a tumor antigen targeting moiety is a TAA. Exemplary TAAs are set forth in Table H below, together with references to exemplary antibodies or antibody sequences upon which the tumor antigen targeting moiety can be based.

TABLE H
Exemplary Anti-TAAs

| Target | Antibody Name and/or Binding Sequences |
|---|---|
| 1-92-LFA-3 | Amevive ™ (alefacept) |
| 5T4 | GEN1044 |
| Activin Receptor | Bimagrumab |
| Type II | VH: SEQ ID Nos: 107, 109 of U.S. Pat. No. 8,388,968 B2 |
| | VL: SEQ ID Nos: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID |
| | NO: 21, 26 or 31 of US 2021/0171641 A1. |
| | VL: the VL sequence of the light chain of SEQ ID NO: 20, |
| | 22 or 30 of US 2021/0171641 A1. |
| B7-H3 (CD276) | VH: the VH sequence of the heavy chain of SEQ ID |
| | NO: 21, 29 or 37 of US 2019/0002563 A1. |
| | VL: the VL sequence of the light chain of SEQ ID NO: 17, |
| | 25 or 33 of US 2019/0002563 A1. |
| B7-H3 (CD276) | VH: the VH sequence of the heavy chain of SEQ ID |
| | NO: 146, 147 or 148 of U.S. Pat. No. 10,640,563. |
| | VL: the VL sequence of the light chain of SEQ ID NO: 143, |
| | 144 or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of U.S. Pat. |
| Stimulator | No. 7,138,501 |
| | VL: amino acids 139-249 of SEQ ID NO: 327 of U.S. Pat. |
| | No. 7,138,501. |
| BAFF/B Lymphocyte | VH: amino acids 1-126 of SEQ ID NO: 1321 of U.S. Pat. |
| Stimulator | No. 7,605,236; |
| | VL: amino acids 143-251 of SEQ ID NO: 1049 of U.S. |
| | Pat. No. 7,605,236. |
| BAFF/B Lymphocyte | Belimumab |
| Stimulator | |
| BCMA | VH: the VH sequence of the heavy chain of SEQ ID NO. |
| | 126 of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| Cadherin | The antibodies described in US Pub. No. US |
| | 2006/0039915. |
| N-cadherin | An antibody that binds to the amino acid sequence of |
| | SEQ ID NO: 10, 17 or 18 of US Pub. No. US |
| | 2010/0278821. |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of SEQ ID NO: 125 |
| | of US Patent Pub. US 2017/0002060 A1 |
| CD20 | Zevalin ™ (ibritumomab tiuxetan) |
| | VH: SEQ ID NO: 9 of U.S. Pat. No. 5,736, 137 |
| | VL: SEQ ID NO: 6 of U.S. Pat. No. 5,736, 137 |
| CD20 | Rituxan ™ (rituximab) |
| | VH: SEQ ID NO: 9 of U.S. Pat. No. 5,736, 137 |
| | VL: SEQ ID NO: 6 of U.S. Pat. No. 5,736, 137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD38 | IB4, HB7 CS/2, clone 90 and NIM-R5 as disclosed in PCT |
| | Pub. WO2015/009726A2 and references cited therein. |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | Flotetuzumab |
| CD206 | Anti-CD206 antibodies having a VH a of SEQ ID NO. 2 |
| | and a VL of SEQ ID NO: 4 of WO2003/040169A2 |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | Nos: 4-6 of U.S. Pat. No. 8,771,690 B2 |
| | CDR-L1, CDR-L2 and CDR-L3 sequences of SEQ ID |
| | Nos: 1-3 of U.S. Pat. No. 8,771,690 B2 |
| CEA | CEA binding portion of RO6958688/RG7802 from clinical |
| | trial NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Clec9a | Anti-Clec9a antibodies having VH and VL amino acids of |
| | SEQ ID Nos. 43 and 48 of PCT Pub. No. |
| | WO2009/026660A1 |
| Clec9a | Anti-Clec9a antibodies having a VH a of SEQ ID NO. 38 |
| | and a VL of SEQ ID NO: 37 or SEQ ID NO: 43 of PCT Pub. |
| | No. WO2022/073062A1 |
| | Anti-Clec9a antibodies having a VH a of SEQ ID NO. 8 |
| | and a VL of SEQ ID NO: 7 of PCT Pub. No. |
| | WO2022/073062A1 |
| Collagen alpha-4 chain | TRC093 (MT293) |
| Collagen | The collagen binding antibody fragment described in |
| | Liang et al., 2016, Sci. Rep. 5, 18205; doi: |
| | 10.1038/srep18205 (2016). |
| Collagen Type I | Cetuximab (Erbitux) |
| Collagen type X | The amino acid sequences of SEQ ID NO: 1 or 2 of PCT |
| | Pub No. WO 2019/020797. |
| Collagen type X | The amino acid sequences of SEQ ID NO: 1 of PCT Pub |
| | No. WO 2014/180992. |
| Collagen type X | Antibody X34 as described in I. Girkontaite et al., |
| | “Immunolocalization of type X collagen in normal fetal and |
| | adult osteoarthritic cartilage with monoclonal |
| | antibodies,” Matrix Biol 15, 231-238 (1996). |
| Collagen type X | Antibodies X53 or 1H8 or ARC0659 or JF0961 collagen X |
| | polyclonal antibody sold under catalog number PA5- |
| | 115039 or PA5-116871 or PA5-97603 or PA5-49198 from |
| | ThermoFisher Scientific. |
| Collagen type X | Antibody sold under catalog number RDI-COLL10abr from |
| | RDI. |
| Complement C5 | Soliris ™ (eculizumab) |
| | VH: amino acids 1-122 of SEQ ID NO: 10 of U.S. Pat. |
| | No. 6,355,245 |
| | VL: amino acids 3-110 of SEQ ID NO: 9 of U.S. Pat. No. |
| | 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DEC-205 | Anti-DEC-205 antibodies having a VH/VL pair of SEQ ID |
| | NOS. 4/10, 16/22, 28/34, 40/46, 52/58, and 76/82 of PCT |
| | Pub. WO2009/061996A2 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID Nos: 80, 84, 88, 92 or 96 |
| | VL: SEQ ID Nos: 82, 86, 90, 94 or 98 |
| EpCAM | G8.8 |
| | HC: SEQ ID NO: 4 of US Patent Pub. No. US |
| | 2020/0317806 A1 |
| | HL: SEQ ID NO: 3 of US Patent Pub. No. US |
| | 2020/0317806 A1 |
| EpCAM | VH: SEQ ID Nos: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| F protein of RSV | Synagic ™ (palivizumab) |
| GD2 | 3F8 |
| Glycoprotein | ReoPro ™ (abiciximab) |
| receptor IIb/IIIa | |
| gpA33 | MGD007 |
| GPC3 | ERY974 |
| GUCY2C | PF-07062119 |
| Heparanase | An antibody selected from HP130, HP 239, HP 108.264, |
| | HP 115.140, HP 152. 197, HP 110.662, HP 144.141, HP |
| | 108.371, HP 135.108, HP 151.316, HP 117.372, HP |
| | 37/33, HP3/17, HP 201 or HP 102 or an amino acid |
| | sequence of SEQ ID NO: 1-11 described in US Patent |
| | Pub. US 2004/0170631. |
| Her2 | Herceptin ™ (trastuzumab) |
| Her2 | Aldesleukin (proleukine) |
| Her2 | Sargramustim (Leucine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446, 175. |
| | VL: SEQ ID NO: 2 of U.S. Pat. No. 7,446, 175 |
| IL12/IFN3 | Stelara ™ (ustekinumab) |
| IL1Ra | Antril ™, Kineret ™ (ankinra) |
| IFNR | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IFN3 p40 | Stelara ™ (ustekinumab) |
| subunit | VH: SEQ ID NO: 7 of U.S. Pat. No. 6,902,734 |
| | VL: SEQ ID NO: 8 of U.S. Pat. No. 6,902,734 |
| Integrin α4 | Tysabri ™ (natalizumab) |
| | VH: SEQ ID Nos: 11-13 of U.S. Pat. No. 5,840,299 |
| | VL: SEQ ID Nos: 7-8 of U.S. Pat. No. 5,840,299 |
| Integrin α4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrin α5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US |
| | Patent Pub. US 2022/0089744. |
| | VL: SEQ ID NO: 4, 16, 18, 20, 22, 44-57 or 107-116 of US |
| | Patent Pub. US 2022/0089744. |
| KIR | Anti-KIR antibodies having a VH of SEQ ID NO: 5 and a |
| | VL of SEQ ID NO: 3 of PCT Pub. WO2014/066532A1 |
| KIR | Anti-KIR antibodies having a VH of SEQ ID NO: 1 and a |
| | VL of SEQ ID NO: 2 of PCT Pub. WO2012/160448A2 |
| | Anti-KIR antibodies having a VH of SEQ ID NO: 3 and a |
| | VL of SEQ ID NO: 4 of PCT Pub. WO2012/160448A2 |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. 9,902,772. |
| | VL: SEQ ID NO: 2, 3, 4, 5, 6, 7, 9, 11, 13 or 14 of U.S. Pat. |
| | No. 9,902,772. |
| LAG3 | VH: SEQ ID NO: 182 of US Patent Pub. US 2021/0095026. |
| | VL: SEQ ID NO: 88 of US Patent Pub. US 2021/0095026. |
| LAG3 | Antibodies having VH/VL amino acid sequences of SEQ ID |
| | Nos 23/24, 3/4 and 11/12 of US Pub. US2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences |
| | having the amino acid sequences of any one of the |
| | following SEQ ID NO: pairs from US 2018/0118848A1: |
| | 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274, 160. |
| | VL: SEQ ID NO: 2 of U.S. Pat. No. 11,274,160. |
| NGF | (tanezumab) |
| NKH1A | The monoclonal antibody deposited with ATCC and |
| | assigned accession no. HB8564, as described in U.S. |
| | Pat. No. 4,772,552A |
| NKP46 | Anti-NKP46 antibodies having CDR-H1, CDR-H2 and |
| | CDR-H3 sequences of SEQ ID Nos: 4, 6 and 8 and CDR- |
| | L1, CDR-L2 and CDR-L3 sequences of SEQ ID Nos: 12, |
| | 14 and 16 of PCT Pub. WO2018/047154A1 |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106 / BMS-936558 (nivolumab), a human IgG4 mAb |
| | with the structure described in WHO Drug Information, Vol. |
| | 27, No. 1, pages 68-69 (2013) and whose heavy and light |
| | chain sequences are disclosed in FIG. 7 of US Pub. No. |
| | US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with the |
| | structure described in WHO Drug Information, Vol. 27, No. 2, |
| | pages 161-162 (2013) and whose heavy and light chain |
| | sequences are disclosed in FIG. 6 of US Pub. No. |
| | US20190270812A1 |
| | HC: SEQ ID NO: 21 of US Pub. No. US20190270812A1 |
| | LC: SEQ ID NO: 22 of US Pub. No. US20190270812A1 |
| PD1 | REGN2810 (disclosed as H4H7798N in US Pub No. |
| | 20150203579) |
| | HC: SEQ ID NO: 330 of US Pub. No. 20150203579 |
| | LC: SEQ ID NO: 331 of US Pub. No. 20150203579 |
| PD1 | H2M7791N disclosed in US Pub No. 20150203579 as |
| | having the following heavy and light chain variable |
| | domains: |
| | VH: SEQ ID NO: 98 of US Pub. No. 20150203579 |
| | VL: SEQ ID NO: 106 of US Pub. No. 20150203579 |
| PD1 | Anti-PD1 antibodies having CDR H1-H3 and CDR L1-L3 |
| | sequences corresponding to the following SEQ ID Nos. of |
| | U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID Nos: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 and |
| | 314/186 of PCT Pub. WO2015112800A1. |
| PD1 | Anti-PD1 antibodies disclosed in U.S. Pat. No. 10,294,299 |
| | B2 as having the following SEQ ID NO. pairs for heavy and |
| | light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb whose |
| | heavy and light chain sequences are disclosed as BAP049- |
| | Clone-E in U.S. Pat. No.: 9683048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and light |
| | chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 |
| | as having the following SEQ ID NO. pairs for heavy and light |
| | chain full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 |
| | as having the following SEQ ID NO. pairs for heavy and light |
| | chain variable domains: |
| | SEQ ID Nos. 4/5 |
| | SEQ ID Nos. 12/13 |
| | SEQ ID Nos. 18/19 |
| | SEQ ID Nos. 40/41 |
| | SEQ ID Nos. 47/48 |
| | SEQ ID Nos. 26/27 |
| | SEQ ID Nos. 34/35 |
| | SEQ ID Nos. 55/56 |
| | SEQ ID Nos. 67/68 |
| PD1 | Anti-PD1 antibodies described in PCT Pub. No. |
| | WO2004/004771 |
| PD1 | Anti-PD1 antibodies described in PCT Pub. No. |
| | WO2004/056875 as having the following SEQ ID NO. pairs |
| | for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. |
| | WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT Publication |
| | No: WO 2015/085847 as having the following heavy and |
| | light chain variable domains: |
| | HC: SEQ ID NO: 9 |
| | LC: SEQ ID NO: 10 |
| PD1 | 4A11 described in U.S. Pat. No. 8,008,449 as having |
| | the following heavy and light chain variable domains: |
| | VH: SEQ ID NO: 5 |
| | VL: SEQ ID NO: 12 |
| PD1 | h1H3 Var 6 described in U.S. Pat. No. 9,205, 148 as |
| | having the following heavy and light chain variable |
| | domains: |
| | VH: SEQ ID NO: 106 |
| | VL: SEQ ID NO: 98 |
| PD1 | PD1AB-6 described in U.S. Pat. No. 10,428,145 as |
| | having the following heavy and light chain variable |
| | domains: |
| | VH: SEQ ID NO: 13 |
| | VL: SEQ ID NO: 8 |
| PD1 | A1.5 described in U.S. Pat. No. 10,513,558 as having |
| | the following heavy and light chain variable domains: |
| | VH: SEQ ID NO: 21 |
| | VL: SEQ ID NO: 47 |
| PD1 | Anti-PD1 antibodies 244C8-2 and 388D4-3, described in |
| | U.S. Pat. No. 10,544,217 as having the following SEQ |
| | ID NO. pairs for heavy and light chain variable domains, |
| | respectively: |
| | SEQ ID NOs: 85/93 |
| | SEQ ID NOs: 88/96 |
| PD1 | Anti-PD1 antibodies 9B2C6C9 and 76G5B3, described in |
| | PCT Publication No. WO 2017/016497 as having the |
| | following SEQ ID NO. pairs for heavy and light chain |
| | variable domains, respectively: |
| | SEQ ID NOs: 1/5 |
| | SEQ ID NOs: 9/13 |
| PD1 | Anti-PD1 antibodies 1.153.7 hAb and 1.103.11 hAbv2 |
| | described in PCT Publication No. WO 2017/025051 as |
| | having the following SEQ ID NO. pairs for heavy and light |
| | chain variable domains, respectively: |
| | SEQ ID NOs: 61/63 |
| | SEQ ID NOs: 53/55 |
| PD1 | Clone 19 described in U.S. Pat. No. 8,927,697 as |
| | having the following heavy and light chain variable |
| | domains: |
| | VH: SEQ ID NO: 14 |
| | VL: SEQ ID NO: 12 |
| PD1 | PD1-0103-312 described in PCT Publication No. WO |
| | 2017/055443 as having the following heavy and light |
| | chain variable domains: |
| | VH: SEQ ID NO: 57 |
| | VL: SEQ ID NO: 58 |
| PD1 | Anti-PD1 antibodies h4B10-3 and 1353-G08 described in |
| | PCT Publication No. WO 2016/077397 as having the |
| | following SEQ ID NO. pairs for heavy and light chain |
| | variable domains, respectively: |
| | SEQ ID NOs: 264/288 |
| | SEQ ID NOs: 251/275 |
| PD1 | PD-1-27 described in PCT Publication No. WO |
| | 2016/210129 as having the following heavy and light |
| | chain variable domains: |
| | VH: SEQ ID NO: 51 |
| | VL: SEQ ID NO: 49 |
| PD1 | Anti-PD1 antibodies RG1H10 and RG1H10-16C |
| | described in PCT Publication No. WO 2014/194302 as |
| | having the following SEQ ID NO. pairs for heavy and light |
| | chain variable domains, respectively: |
| | SEQ ID NOs: 23/24 |
| | SEQ ID NOs: 40/24 |
| PD1 | HuEH12-VH4-VK3 described in U.S. Pat. No. |
| | 9,102,727 as having the following heavy and light chain |
| | variable domains: |
| | VH: SEQ ID NO: 28 |
| | VL: SEQ ID NO: 32 |
| PD1 | Clone 39 described in U.S. Pat. No. 10,066,013 as |
| | having the following heavy and light chain variable |
| | domains: |
| | VH: SEQ ID NO: 35 |
| | VL: SEQ ID NO: 34 |
| PD1 | tislelizumab |
| PD1 | retifanlimab |
| PD1 | balstilimab |
| PD1 | dostarlimab |
| PD1 | sasanlimab |
| PD1 | sintilimab |
| PD1 | cemiplimab |
| PDL1 | Durvalumab (MEDI4736) |
| | HC: SEQ ID NO: 26 of PCT application No. |
| | WO2020225552 |
| | LC: SEQ ID NO: 27 of PCT application No. |
| | WO2020225552 |
| PDL1 | Atezolizumab (Tecentriq, MPDL3280A, RG7446) |
| | HC: SEQ ID NO: 20 of U.S. Pat. No. 8,217, 149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. |
| | 7,943,743 as having the following SEQ ID NO. pairs for |
| | heavy and light chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No.: 9,624,298 as |
| | having the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID |
| | Nos: 52, 56, and 77 of PCT Pub. No. 2018/080812 |
| | CDR-L1, CDR-L2 and CDR-L3 sequences of SEQ ID Nos: |
| | 65, 42, and 71 of PCT Pub. No. 2018/080812 |
| PDL1 | KN035 (Envafolimab) is a nanobody described as Hu56V2 |
| | in U.S. Pat. No. 11,225,522 as having the VHH SEQ ID |
| | NO: 34 |
| PDL1 | LY3434172 |
| PDL1 | LY3300054 (Iodapolimab) described in PCT Pub No: WO |
| | 2017/034916 as having the following heavy and light chain |
| | variable domains: |
| | HC: SEQ ID NO: 10 |
| | L: SEQ ID NO: 11 |
| PDL1 | LDP (Iesabelimab, ADG104) described in CN Patent No: |
| | 114225023 as having the following heavy and light chain |
| | variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No.: |
| | 10,118,963 as having the following heavy and light chain |
| | variable domains: |
| | HC: SEQ ID NO: 1 |
| | LC: SEQ ID NO: 2 |
| PDL1 | BGB-A333 (garivulimab) described in U.S. Pat. No.: |
| | 11,512,132 as having the following heavy and light chain |
| | variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. |
| | 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or 145 |
| | of U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, Abgenix |
| | 4.248.2, Abgenix 4.360.3, Abgenix 4.7.1, Abgenix 4.4.1, |
| | Abgenix 4.177.3, Abgenix 4.16.1, Abgenix 4.22.3, |
| | Abgenix 4.28.3, Abgenix 4.40.2, Abgenix 4.48.3, Abgenix |
| | 4.49.1, Abgenix 4.209.3, Abgemx 4.219.3, Abgenix |
| | 4.288.1, Abgenix 4.333.1, Abgemx 4.54.1, Abgenix |
| | 4.153.1, Abgenix 4.232.3, Abgenix 4.292.3, Abgenix |
| | 4.304.1, Abgenix 4.78.1 and Abgenix 4.152.1 described in |
| | WO2003034903A2 |
| | A hybridoma cell line such as: PSMA 3.7 (PTA-3257), |
| | PSMA 3.8, PSMA 3.9 (PTA- 3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247), PSMA |
| | 1.8.3 (PTA-3906), PSMA A3.1.3 (PTA- 3904), PSMA |
| | A3.3.1 (PTA-3905), Abgenix 4.248.2 (PTA-4427), Abgenix |
| | 4.360.3 (PTA- 4428), Abgenix 4.7.1 (PTA-4429), Abgenix |
| | 4.4.1 (PTA-4556), Abgenix 4.177.3 (PTA-4557), Abgenix |
| | 4.16.1 (PTA-4357), Abgenix 4.22.3 (PTA-4358), Abgenix |
| | 4.28.3 (PTA-4359), Abgenix 4.40.2 (PTA-4360), Abgenix |
| | 4.48.3 (PTA-4361), Abgenix 4.49.1 (PTA-4362), Abgenix |
| | 4.209.3 (PTA-4365), Abgenix 4.219.3 (PTA-4366), |
| | Abgenix 4.288.1 (PTA-4367), Abgenix 4.333.1 (PTA- |
| | 4368), Abgenix 4.54.1 (PTA-4363), Abgenix 4.153.1 |
| | (PTA-4388), Abgenix 4.232.3 (PTA-4389), Abgenix |
| | 4.292.3 (PTA-4390), Abgenix 4.304.1 (PTA-4391), |
| | Abgenix 4.78.1 (PTA-4652), and Abgemx 4.152.1(PTA- |
| | 4653) described in WO 2003/034903A2. |
| | VH of SEQ ID Nos: 2-7 described in WO 2003/034903A2 |
| | VL of SEQ ID Nos: 8-13 described in WO 2003/034903A2 |
| PMSA | VH: SEQ ID Nos: 225, 239, 253, 267, 281, 295, 309, 323, |
| | 337, 351, 365, 379, 393, 407, 421, 435, 449, 463, 477, |
| | 491, 505, 519, 533, 547, 561, 575, 589, 603 or 617 |
| | described in WO 2011/121110A1. |
| | VL SEQ ID Nos: 230, 244, 258, 272, 286, 300, 314, 328, |
| | 342, 356, 370, 384, 398, 412, 426, 440, 454, 468, 482, |
| | 496, 510, 524, 538, 552, 566, 580, 594, 608 or 622 |
| | described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 or |
| | 627 described in WO 2011/121110A1. |
| PMSA | An anti-PMSA antibody having a VL amino acid sequence |
| | of any one of SEQ ID Nos: 229-312 of US 2022/0119525 |
| | A1 and a VH of SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of US Patent Pub. 2017/0002060 |
| | VL: SEQ ID NO: 52 of US Patent Pub. 2017/0002060 |
| SLAMF7 | Empliciti ™ (elotuzumab) |
| SSTR2 | XmAb ®18087 |
| STEAP1 | VHCDR1 SEQ ID Nos: 14, 33, 182, 184 or 185 described |
| | in US20210179731A1. |
| | VHCDR2 SEQ ID Nos: 15, 21, 34, 182, 184 or 185 |
| | described in US20210179731A1. |
| | VHCDR3 SEQ ID Nos: 16 and 35 described in |
| | US20210179731A1. |
| | VH SEQ ID Nos: 182 or 184 described in |
| | US20210179731A1. |
| | VLCDR1 SEQ ID Nos: 11 or 30 described in |
| | US20210179731A1. |
| | VLCDR2 SEQ ID Nos: 12 or 31 described in |
| | US20210179731A1. |
| | VLCDR3 SEQ ID Nos: 13 or 32 described in |
| | US20210179731A1. |
| | VL SEQ ID Nos: 183 or 186 described in |
| | US20210179731A1. |
| STEAP1 | AMG509 |
| STEAP2 | Anti-STEAP 2 antibodies having CDR-H1, CDR-H2, CDR- |
| | H3, CDR-L1, CDR-L2 and CDR-L3 sequences selected |
| | from SEQ ID NOS: (1) 4-6-8-12-14-16; (2) 20-22-24-28- |
| | 30-32; (3) 36-38-40-44-46-48; (4) 52-54-56-60-62-64; (5) |
| | 68-70-72-60-62-64; (6) 76-78-80-60-62-64; (7) 84-86-88- |
| | 60-62-64; (8) 92-94-96-60-62-64; (9) 100-102-104-60-62- |
| | 64; (10) 108-110-112-116-118-120; (11) 124-126-128- |
| | 132-134-136; (12) 140-142-144-148-150-152; (13) 156- |
| | 158-160-164-166-168; (14) 172-174-176-180-182-184; |
| | (15) 188-190-192-196-198-200; (16) 204-206-208-212- |
| | 214-216; (17) 220-222-224-228-230-232; (18) 236-238- |
| | 240-244-246-248; (19) 252-254-256-260-262-264; (20) |
| | 268-270-272-276-278-280; (21) 284-286-288-292-294- |
| | 296; (22) 300-302-304-308-310-312; (23) 316-318-320- |
| | 324-326-328; (24) 332-334-336-340-342-344; (25) 348- |
| | 350-352-356-358-360; (26) 364-366-368-372-374-376; |
| | and (27) 380-382-384-388-390-392 of U.S. Pat. No. |
| | 10,772,972 B2. |
| | Anti-STEAP 2 antibodies having (a) a VH comprising the |
| | amino acid of any one of SEQ ID Nos: 2, 18, 34, 50, 66, |
| | 74, 82, 90, 98, 106, 122, 138, 154, 170, 186, 202, 218, |
| | 234, 250, 266, 282, 298, 314, 330, 346, 362, and 378 of |
| | U.S. Pat. No. 10,772,972 B2; and (b) a VL comprising |
| | the amino acid sequence of any one of SEQ ID Nos: 10; |
| | 26; 42; 58; 114; 130; 146; 162; 178; 194; 210; 226, 242; |
| | 258; 274; 290; 306; 322; 338; 354; 370; and 386 of U.S. |
| | Pat. No. 10,772,972 B2. |
| | Anti-STEAP 2 antibodies having a VH/VL pair comprising |
| | the amino acid sequences of any of the following pairs of |
| | SEQ ID Nos of U.S. Pat. No. 10,772,972 B2: 2/10; 18/26; |
| | 34/42; 50/58; 66/58; 74/58; 82/58; 90/58; 98/58; 106/114; |
| | 122/130; 138/146; 154/162; 170/178; 186/194; 202/210; |
| | 218/226; 234/242; 250/258; 266/274; 282/290; 298/306; |
| | 314/322; 330/338; 346/354; 362/370; and 378/386. |
| Syndecan-1 (CD 138) | The B-B4 antibody described in Wijdenes et al. (1996) Br. |
| | J. Haematol., 94: 318-323 |
| Syndecan-4 | The amino acid sequence of amino acids 93 and 121 of |
| | SEQ ID NO: 1 or the amino acid sequence of amino acids |
| | 92 and 122 of SEQ ID NO: 2 described in European Patent |
| | Pub. EP 2 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication |
| | WO2013/A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |
| XCR1 | Anti-XCR1 antibodies disclosed in U.S. Pat. |
| | No. 9,371,389 B2, including: |
| | The antibodies designated 2H6, 5G7, 11H2, |
| | HK1L2 and HK5L5 |
| | Antibodies having CDR-H1, CDR-H2 and CDR-H3 |
| | sequences of SEQ ID Nos: 53-55 and CDR-L1, |
| | CDR-L2 and CDR-L3 sequences of SEQ ID |
| | Nos: 56-58. |
| | Antibodies having CDR-H1, CDR-H2 and CDR-H3 |
| | sequences of SEQ ID Nos: 41-43 and CDR-L1, |
| | CDR-L2 and CDR-L3 sequences of SEQ ID |
| | Nos: 44-46. |

In some aspects, the tumor antigen targeting moiety competes with an antibody set forth in Table H for binding to the TAA. In further aspects, the tumor antigen targeting moiety comprises CDRs having CDR sequences of an anti-TAA antibody set forth in Table H. In some embodiments, the tumor antigen targeting moiety comprises all 6 CDR sequences of the anti-TAA antibody set forth in Table H. In other embodiments, the tumor antigen targeting moiety comprises at least the heavy chain CDR sequences (CDR-H1, CDR-H2, CDR-H3 and the light chain CDR sequences of a universal light chain. In further aspects, a tumor antigen targeting moiety comprises a VH comprising the amino acid sequence of the VH of an anti-TAA antibody set forth in Table H. In some embodiments, the tumor antigen targeting moiety further comprises a VL comprising the amino acid sequence of the VL of the anti-TAA antibody set forth in Table H. In other embodiments, the tumor antigen targeting moiety further comprises a universal light chain VL sequence. Additional TAAs that can be targeted by a tumor antigen targeting moiety are disclosed in, e.g., Hafeez et al., 2020, Molecules 25:4764, doi: 10.3390/molecules25204764, particularly in Table 1. Table 1 of Hafeez et al. is incorporated by reference in its entirety here.

Yet additional exemplary TAAs include Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-β2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFβR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostate specific antigen (PSA), PAP, LAGA-1a, p53, prostein, PSMA, surviving and telomerase, prostate-carcinoma tumor antigen-1 (PCTA-1), ELF2M, neutrophil elastase, ephrin β2, insulin growth factor (IGF1)-I, IGF-II, IGFI receptor, 5T4, ROR1, Nkp30, NKG2D, tumor stromal antigens, the extra domain A (EDA) and extra domain B (EDB) of fibronectin and the A1 domain of tenascin-C (TnC A1).

### 6.6. Multispecific Antigen Binding Molecules

Aspects of the present disclosure are directed to compositions comprising a T cell activator and a multispecific antigen binding molecule, as well as to methods comprising administration (together or separately) of a T cell activator and a multispecific antigen binding molecule. As used herein, a multispecific antigen binding molecule describes a molecule comprising: (a) at least one tumor antigen targeting moiety or at least one BCA targeting moiety; and (b) at least one TCA targeting moiety. A multispecific antigen binding molecule may further comprise one or more additional tumor antigen targeting moieties, one or more additional BCA targeting moieties, and/or one or more additional TCA targeting moieties. In general, a “multispecific antigen binding molecule,” as used herein, describes a molecule that does not comprise a pMHC complex. Also disclosed are pharmaceutical compositions comprising such multispecific antigen binding molecules, in some cases together also comprising a T cell activator disclosed herein. Further disclosed are methods for use of such multispecific antigen binding molecules in treatment of cancer, in some embodiments in combination with a T cell activator disclosed herein.

Certain example tumor-associated antigens and associated indications are provided in Table K-2. In some embodiments, a multispecific antigen binding molecule comprises a tumor antigen targeting moiety that specifically binds to a TAA of Table K-1. In some embodiments, the TAA is BCMA. In some embodiments, the TAA is CD19. In some embodiments, the TAA is CD20. In some embodiments, the TAA is CD22. In some embodiments, the TAA is EGFR.

TABLE K-1
Examples of Tumor-Associated Antigens and Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| hERV 4700 | clear-cell renal cell carcinoma |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

In some embodiments, a multispecific antigen binding molecule comprises a BCA targeting moiety that specifically binds to CD19, CD20, or CD22. In some embodiments, the BCA targeting moiety specifically binds to CD19. In some embodiments, the BCA targeting moiety specifically binds to CD20. In some embodiments, the BCA targeting moiety specifically binds to CD22.

Certain example multispecific antigen binding molecules are provided in Table K-2. In some embodiments, a multispecific antigen binding molecule of the disclosure is a bispecific antigen binding molecule of Table K-2. In some embodiments, a multispecific antigen binding molecule comprises one or more CDR, VH, and/or VL sequences from a bispecific antigen binding molecule of Table K-2.

TABLE K-2
Exemplary Bispecific Antibodies

| Targets | Antibody Name and/or Binding Sequences |
|---|---|
| CD3 × BCMA | Bispecific antibodies bsAb25441D and bsAb25442D described in US |
| | 2022/0306758 A1 and US 2021/0206865 A1 |
| CD28 × CD38 | Bispecific antibodies bsAb6031 and bsAb7945 described in US |
| | 2022/0089766 A1 |
| CD3 × CD20 | Bispecific antibodies BS3/20-001, BS3/20-002, BS3/20-003, BS3/20-004, |
| | BS3/20-005, BS3/20-007, and BS3/20-009 described in US 2018/0215823 A1 |
| CD3 × CD20 | Bispecific antibodies Antibody 1 and Antibody 2 described in US |
| | 20180194841 A1 |
| CD28 × EGFR | Bispecific antibodies bsAb7075, bsAb6321, bsAb6322, and bsAb6323 |
| | described in US 2020/0299388 A1 |
| CD3 × MUC16 | Bispecific antibody BSMUC16/CD3-001 described in US 2020/0399371 A1 |
| CD3 × PSMA | Bispecific antibody PSMA/CD3-005 described in US 2020/0399372 A1 |
| CD3 × CD33 | Bispecific antibodies mAb2 G1 C-LC DANAPA IgG1, mAb2 D5 N-LC |
| | DANAPA IgG1 and mAb2 D5 C-LC DANAPA IgG1 described in US |
| | 2019/0153096A1 |
| CD3 × CLEC12A | Bispecific antibody 5196 × 4327 DM-Fc bsAb described in WO 2017/010874 A1 |
| CD3 × PSMA | Bispecific antibodies BSPSMA/CD3-001, BSPSMA/CD3-002, BSPSMA/CD3- |
| | 003, BSPSMA/CD3-200, BSPSMA/CD3-300, BSPSMA/CD3-400, |
| | BSPSMA/CD3-004, BSPSMA/CD3-800, BSPSMA/CD3-900, BSPSMA/CD3- |
| | 1000, BSPSMA/CD3-1100, BSPSMA/CD3-1200, BSPSMA/CD3-1300, |
| | BSPSMA/CD3-1400, BSPSMA/CD3-1500, BSPSMA/CD3-1600, |
| | BSPSMA/CD3-1700, BSPSMA/CD3-1800, BSPSMA/CD3-1900, |
| | BSPSMA/CD3-005, BSPSMA/CD3-2100 described in US 2021/0403595 A1 |
| CD3 × BCMA | Bispecific antibodies BCMB72, BC3B7, BC3B8, BC3B9, BC3B10, BC3B11, |
| | BC3B12 described in WO 2017/031104 A1 |
| CD28 × MUC16 | Bispecific antibodies bs24963D and bs32897D described in US 11453721 B2 |
| CD28 × PSMA | Bispecific antibodies bs16429D, bs16430D, and bs 16431D described in US |
| | 2019/0389951 A1 |
| CD28 × CD22 | Bispecific antibodies REGN5837 and REGN5838 described in WO |
| | 2020/132066 A1 |
| CD3 × EpCAM | Catumaxomab, MT110 |
| CD3 × EpCAM | MT110 |
| CD3 × HER2/neu | ertumaxomab |
| CD3 × HER2 | ISB1302 |
| CD3 × HER2 | Runimotamab |
| CD3 × HER2 | M802 |
| CD3 × PSMA | Acapatamab |
| CD3 × PSMA | BAY2010112 (Pasotuxizumab) |
| CD3 × PSMA | JNJ-63898081 (JNJ-081) |
| CD3 × MUC17 | AMG199 |
| CD3 × CD33 | AMG330 |
| CD3 × CD33 | AMG673 |
| CD3 × CD33 | AMV564 |
| CD3 × CD33 | GEM333 |
| CD3 × CD33 | JNJ-67571244 |
| CD3 × FLT3 | AMG427 (Emirodatamab) |
| CD3 × CD19 | AMG562 |
| CD3 × CD19 | A-319 |
| CD3 × CD19 | Duvortuxizumab |
| CD3 × EGFRvIII | AMG596 |
| CD3 × BCMA | Alnuctamab (CC-93269, BMS-986349) |
| CD3 × BCMA | AMG701 (Pavurutamab) |
| CD3 × BCMA | AMG420 |
| CD3 × BCMA | Teclistamab |
| CD3 × BCMA | Elranatamab |
| CD3 × BCMA | REGN5458 |
| CD3 × BCMA | REGN5459 |
| CD3 × BCMA | TNB-383B |
| CD3 × NY-ESO-1 | IMCnyeso |
| CD3 × MAGE-A4 | IMC-C103C |
| CD3 × PRAME | IMC-F106C |
| CD3 × 5T4 | GEN1044 |
| CD3 × DLL3 | AMG757 |
| CD3 × CLDN18.2 | AMG910 (Gresonitamab) |
| CD3 × GPC3 | ERY974 |
| CD3 × gpA33 | MGD007 |
| CD3 × B7-H3 | Orlotamab (MGD007) |
| CD3 × SSTR2 | XmAb-18087 |
| CD3 × PSCA | GEM3PSCA |
| CD3 × CD38 | AMG424 |
| CD3 × CD38 | ISB1342 |
| CD3 × STEAP1 | AMG509 |
| CD3 × FCRL5 | Cevostamab |
| CD3 × CD123 | APVO436 |
| CD3 × CD123 | JNJ-63709178 |
| CD3 × CD123 | Flotetuzumab (MGD006) |
| CD3 × CD123 | SAR440234 |
| CD3 × CD123 | Vibecotamab (XmAb14045) |
| CD3 × CD20 | Epcoritamab (GEN3013) |
| CD3 × CD20 | IGM-2323 |
| CD3 × CD20 | Odronextamab |
| CD3 × CD20 | Glofitamab (RO7082859) |
| CD3 × CD20 | XmAb13676 |
| CD3 × GPRC5D | Talquetamab (JNJ-64407564) |
| CD3 × CLEC12A | MCLA-117 |
| CD3 × MUC16 | REGN4018 |
| CD3 × CEA | RO6958688 (RG7802) |
| CD3 × GD2 | Nivatrotamab (Hu3F8-BsAb) |

### 6.7. The Multimerization Moiety

Aspects of the present disclosure are directed to multispecific molecules (e.g., T cell activators) comprising a multimerization moiety. A “multimerization moiety” describes any polypeptide or other molecule or portion thereof capable of multimerization (e.g., dimerization). Such multimerization includes, for example, non-covalent association of two or more multimerization moieties. Various multimerization moieties are recognized in the art and contemplated herein. Exemplary multimerization moieties of the present disclosure are described further below.

### 6.7.1. Fc Domains

The multispecific molecules (e.g., T cell activators) of the disclosure typically include a pair of Fc domains that associate to form an Fc region. In native antibodies, Fc regions comprise hinge regions at their N-termini to form a constant domain. Throughout this disclosure, the reference to an Fc domain encompasses an Fc domain with or without a hinge domain. In some embodiments, the Fc domain comprises a hinge domain at its N-terminus.

The Fc domains can be derived from any suitable species operably linked to a pMHC complex, a targeting moiety, or a component thereof. In one embodiment the Fc domain is derived from a human Fc domain. In preferred embodiments, the pMHC complex, targeting moiety, or component thereof is fused to an IgG Fc molecule. A pMHC complex, targeting moiety, or component thereof may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific molecules, the Fc domains might advantageously be different to allow for heterodimerization, as described in Section 6.7.1.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In multispecific molecules of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the multispecific molecules of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild-type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the multispecific molecules of the present disclosure do not comprise a tailpiece.

The Fc domains that are incorporated into the multispecific molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.7.1.1.

The Fc domains can also be altered to include modifications that improve manufacturability of asymmetric multispecific molecules, for example by allowing heterodimerization, which is the preferential pairing of non-identical Fc domains over identical Fc domains. Heterodimerization permits the production of multispecific molecules in which different polypeptide components are connected to one another by an Fc region containing Fc domains that differ in sequence. Examples of heterodimerization strategies are exemplified in Section 6.7.1.2.

It will be appreciated that any of the modifications mentioned above can be combined in any suitable manner to achieve the desired functional properties and/or combined with other modifications to alter the properties of the multispecific molecules.

Example Fc domain sequences are provided in Table F-1, below.

TABLE F-1
Fc Sequences

| | | SEQ |
|---|---|---|
| Fo | Sequence | ID NO |
| hIgG1 Fc | EPKSCDKTHTCPPCPAPELLGGPSVFLFPPKPKDTLMISRTPEVTCV | 10 |
| (amino acids 99- | VVDVSHEDPEVKFNWYVDGVEVHNAKTKPREEQYNSTYRVVSVLT | |
| 330 of UniprotKB | VLHQDWLNGKEYKCKVSNKALPAPIEKTISKAKGQPREPQVYTLPPS | |
| P01857-1) | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hIgG2 Fc | ERKCCVECPPCPAPPVAGPSVFLFPPKPKDTLMISRTPEVTCVVVDV | 11 |
| (amino acids 99- | SHEDPEVQFNWYVDGVEVHNAKTKPREEQFNSTFRVVSVLTVVHQ | |
| 326 of UniprotKB | DWLNGKEYKCKVSNKGLPAPIEKTISKTKGQPREPQVYTLPPSREE | |
| P01859-1) | MTKNQVSLTCLVKGFYPSDISVEWESNGQPENNYKTTPPMLDSDGS | |
| | FFLYSKLTVDKSRWQQGNVFSCSVMHEALHNHYTQKSLSLSPGK | |
| hIgG3 Fc | ELKTPLGDTTHTCPRCPEPKSCDTPPPCPRCPEPKSCDTPPPCPRC | 12 |
| (amino acids 99- | PEPKSCDTPPPCPRCPAPELLGGPSVFLFPPKPKDTLMISRTPEVTC | |
| 377 of UniprotKB | VVVDVSHEDPEVQFKWYVDGVEVHNAKTKPREEQYNSTFRVVSVL | |
| P01860-1) | TVLHQDWLNGKEYKCKVSNKALPAPIEKTISKTKGQPREPQVYTLPP | |
| | SREEMTKNQVSLTCLVKGFYPSDIAVEWESSGQPENNYNTTPPMLD | |
| | SDGSFFLYSKLTVDKSRWQQGNIFSCSVMHEALHNRFTQKSLSLSP | |
| | GK | |
| hIgG4 Fc | ESKYGPPCPSCPAPEFLGGPSVFLFPPKPKDTLMISRTPEVTCVVVD | 13 |
| (amino acids 99- | VSQEDPEVQFNWYVDGVEVHNAKTKPREEQFNSTYRVVSVLTVLH | |
| 327 of UniprotKB | QDWLNGKEYKCKVSNKGLPSSIEKTISKAKGQPREPQVYTLPPSQE | |
| P01861-1) | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| hIgG4s Fc | ESKYGPPCPPCPAPPVAGPSVFLFPPKPKDTLMISRTPEVTCVVVDV | 14 |
| | SQEDPEVQFNWYVDGVEVHNAKTKPREEQFNSTYRVVSVLTVLHQ | |
| | DWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| hIgG1 PVA Fc | EPKSCDKTHTCPPCPAPPVAGPSVFLFPPKPKDTLMISRTPEVTCVV | 15 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| hIgG1 PVA star | EPKSCDKTHTCPPCPAPPVAGPSVFLFPPKPKDTLMISRTPEVTCVV | 16 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| hIgG1s | DKKVEPKSCDKTHTCPPCPAPPVAGPSVFLFPPKPKDTLMISRTPEV | 17 |
| | TCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTL | |
| | PPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSL | |
| | SPGK | |
| hIgG1 N180G, | EPKSCDKTHTCPPCPAPELLGGPSVFLFPPKPKDTLMISRTPEVTCV | 18 |
| also referred to | VVDVSHEDPEVKFNWYVDGVEVHNAKTKPREEQYGSTYRVVSVLT | |
| as N297G | VLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hIgG2 variant | ERKCCVECPPCPAPPVAGPSVFLFPPKPKDTLMISRTPEVTCVVVDV | 19 |
| | SHEDPEVQFNWYVDGVEVHNAKTKPREEQFNSTFRVVSVLTVVHQ | |
| | DWLNGKEYKCKVSNKGLPAPIEKTISKTKGQPREPQVYTLPPSREE | |
| | MTKNQVSLTCLVKGFYPSDIAVEWESNGQPENNYKTTPPMLDSDGS | |
| | FFLYSKLTVDKSRWQQGNVFSCSVMHEALHNHYTQKSLSLSPGK | |
| hIgG4 S108P | ESKYGPPCPPCPAPEFLGGPSVFLFPPKPKDTLMISRTPEVTCVVVD | 20 |
| | VSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| IgG1PVA_hinge- | EPKSCDKTHTCPPCPAPPVAGPSVFLFPPKPKDTLMISRTPEVTCVV | 21 |
| Fc knob | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 22 |
| Fc knob_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 23 |
| Fc knob_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 24 |
| Fc knob_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 25 |
| Fc Hole | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 26 |
| Fc Hole_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 27 |
| Fc Hole_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 28 |
| Fc Hole_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to any one of the sequences disclosed in Table F-1.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 10. In cases where an Fc domain comprises at least 90% sequence identity and less than 100% sequence identity to SEQ ID NO: 10 (e.g., between 90% and 99% sequence identity to SEQ ID NO: 10), an Fc domain may also comprise one or more amino acid substitutions described herein, for example one or more substitutions that reduce effector function (e.g., as described in Section 6.7.1.1) and/or one or more substitutions that promote Fc heterodimerization (e.g., as described in Section 6.7.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 11. In cases where an Fc domain comprises at least 90% sequence identity and less than 100% sequence identity to SEQ ID NO: 11 (e.g., between 90% and 99% sequence identity to SEQ ID NO: 11), an Fc domain may also comprise one or more amino acid substitutions described herein, for example one or more substitutions that reduce effector function (e.g., as described in Section 6.7.1.1) and/or one or more substitutions that promote Fc heterodimerization (e.g., as described in Section 6.7.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:11. In cases where an Fc domain comprises at least 90% sequence identity and less than 100% sequence identity to SEQ ID NO: 11 (e.g., between 90% and 99% sequence identity to SEQ ID NO: 11), an Fc domain may also comprise one or more amino acid substitutions described herein, for example one or more substitutions that reduce effector function (e.g., as described in Section 6.7.1.1) and/or one or more substitutions that promote Fc heterodimerization (e.g., as described in Section 6.7.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 12. In cases where an Fc domain comprises at least 90% sequence identity and less than 100% sequence identity to SEQ ID NO: 12 (e.g., between 90% and 99% sequence identity to SEQ ID NO: 12), an Fc domain may also comprise one or more amino acid substitutions described herein, for example one or more substitutions that reduce effector function (e.g., as described in Section 6.7.1.1) and/or one or more substitutions that promote Fc heterodimerization (e.g., as described in Section 6.7.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 13.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 14.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 15.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 16.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 17.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 18.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 19.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:20.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:21.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:22.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:23.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:24.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:25.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:26.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:27.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:28.

6.7.1.1. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain or the Fc region (e.g., one or both Fc domains of a multispecific molecules that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table I below. In some embodiments, the Fc domain includes only the bolded portion of the sequences shown below:

TABLE I

| Fc Domain | Sequence |
|---|---|
| SEQ ID NO: 1 of | Asp Lys Arg Val Glu Ser Lys Tyr Gly Pro Cys Pro Pro Cys |
| WO2014/121087 | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gin Asp Trp Leu |
| | Asn Gly Lys Glu Tyr Lys Cys Lys Val Ser Asn Lys Gly Leu |
| | Pro Ser Ser Ile Glu Lys Thr Ile Ser Lys Ala Lys Gly Gln Pro |
| | Arg Glu Pro Gln Val Tyr Thr Leu Pro Pro Ser Gln Glu Glu |
| | Met Thr Lys Asn Gln Val Ser Leu Thr Cys Leu Val Lys Gly |
| | Phe Tyr Pro Ser Asp Ile Ala Val Glu Trp Glu Ser Asn Gly |
| | Gln Pro Glu Asn Asn Tyr Lys Thr Thr Pro Pro Val Leu Asp |
| | Ser Asp Gly Ser Phe Phe Leu Tyr Ser Arg Leu Thr Val Asp |
| | Lys Ser Arg Trp Gln Glu Gly Asn Val Phe Ser Cys Ser Val |
| | Met His Glu Ala Leu His Asn His Tyr Thr Gln Lys Ser Leu |
| | Ser Leu Ser Leu Gly Lys (SEQ ID NO: 128) |
| SEQ ID NO: 2 of | Asp Lys Lys Val Glu Pro Lys Ser Cys Asp Lys Thr His Thr |
| WO2014/121087 | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Arg Asp Glu Leu Thr Lys Asn Gln Val Ser Leu Thr Cys |
| | Leu Val Lys Gly Phe Tyr Pro Ser Asp Ile Ala Val Glu Trp Glu |
| | Ser Asn Gly Gln Pro Glu Asn Asn Tyr Lys Thr Thr Pro Pro |
| | Val Leu Asp Ser Asp Gly Ser Phe Phe Leu Tyr Ser Lys Leu |
| | Thr Val Asp Lys Ser Arg Trp Gln Gln Gly Asn Val Phe Ser |
| | Cys Ser Val Met His Glu Ala Leu His Asn His Tyr Thr Gln |
| | Lys Ser Leu Ser Leu Ser Pro Gly Lys (SEQ ID NO: 17) |
| SEQ ID NO: 30 of | Ala Ser Thr Lys Gly Pro Ser Val Phe Pro Leu Ala Pro Ser |
| WO2014/121087 | Ser Lys |
| | Ser Thr Ser Gly Gly Thr Ala Ala Leu Gly Cys Leu Val Lys |
| | Asp Tyr |
| | Phe Pro Glu Pro Val Thr Val Ser Trp Asn Ser Gly Ala Leu |
| | Thr Ser Gly Val His Thr Phe Pro Ala Val Leu Gln Ser Ser |
| | Gly Leu Tyr Ser Leu Ser Ser Val Val Thr Val Pro Ser Ser |
| | Ser Leu Gly Thr Gln Thr Tyr Ile Cys Asn Val Asn His Lys |
| | Pro Ser Asn Thr Lys Val Asp Lys Lys Val Glu Pro Lys Ser |
| | Cys Asp Lys Thr His Thr Cys Pro Pro Cys Pro Ala Pro |
| | Pro Val Ala Gly Pro Ser Val Phe Leu Phe Pro Pro Lys |
| | Pro Lys Asp Thr Leu Met Ile Ser Arg Thr Pro Glu Val Thr |
| | Cys Val Val Val Asp Val Ser Gln Glu Asp Pro Glu Val Gln |
| | Phe Asn Trp Tyr Val Asp Gly Val Glu Val His Asn Ala |
| | Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly |
| | Leu Pro Ser Ser Ile Glu Lys Thr Ile Ser Lys Ala Lys Gly |
| | Gln Pro Arg Glu Pro Gln Val Tyr Thr Leu Pro Pro Ser Arg |
| | Asp Glu Leu Thr Lys Asn Gln Val Ser Leu Thr Cys Leu |
| | Val Lys Gly Phe Tyr Pro Ser Asp Ile Ala Val Glu Trp Glu |
| | Ser Asn Gly Gln Pro Glu Asn Asn |
| | Tyr Lys Thr Thr Pro Pro Val Leu Asp Ser Asp Gly Ser |
| | Phe Phe Leu Tyr Ser Lys Leu Thr Val Asp Lys Ser Arg |
| | Trp Gln Gln Gly Asn Val Phe Ser Cys Ser Val Met His |
| | Glu Ala Leu His Asn His Tyr Thr Gln Lys Ser Leu Ser |
| | Leu Ser Pro Gly Lys (SEQ ID NO: 129) |
| SEQ ID NO: 31 of | Ala Ser Thr Lys Gly Pro Ser Val Phe Pro Leu Ala Pro Cys |
| WO2014/121087 | Ser Arg Ser Thr Ser Glu Ser Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met Ile Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Gln Glu Glu Met Thr Lys Asn |
| | Gln Val Ser Leu Thr Cys Leu Val Lys Gly Phe Tyr Pro |
| | Ser Asp Ile Ala Val Glu Trp Glu Ser Asn Gly Gln Pro Glu |
| | Asn Asn Tyr Lys Thr Thr Pro Pro Val Leu Asp Ser Asp |
| | Gly Ser Phe Phe Leu Tyr Ser Arg Leu Thr Val Asp Lys |
| | Ser Arg Trp Gln Glu Gly Asn Val Phe Ser Cys Ser Val |
| | Met His Glu Ala Leu His Asn His Tyr Thr Gln Lys Ser |
| | Leu Ser Leu Ser Leu Gly Lys (SEQ ID NO: 130) |
| SEQ ID NO: 37 of | Ala Ser Thr Lys Gly Pro Ser Val Phe Pro Leu Ala Pro Ser |
| WO2014/121087 | Ser Lys |
| | Ser Thr Ser Gly Gly Thr Ala Ala Leu Gly Cys Leu Val Lys |
| | Asp Tyr |
| | Phe Pro Glu Pro Val Thr Val Ser Trp Asn Ser Gly Ala Leu |
| | Thr Ser Gly Val His Thr Phe Pro Ala Val Leu Gln Ser Ser |
| | Gly Leu Tyr Ser Leu Ser Ser Val Val Thr Val Pro Ser Ser |
| | Ser Leu Gly Thr Gln Thr Tyr Ile Cys Asn Val Asn His Lys |
| | Pro Ser Asn Thr Lys Val Asp Lys Lys Val Glu Pro Lys Ser |
| | Cys Asp Lys Thr His Thr Cys Pro Pro Cys Pro Ala Pro |
| | Pro Val Ala Gly Pro Ser Val Phe Leu Phe Pro Pro Lys |
| | Pro Lys Asp Thr Leu Met Ile Ser Arg Thr Pro Glu Val Thr |
| | Cys Val Val Val Asp Val Ser Gln Glu Asp Pro Glu Val Gln |
| | Phe Asn Trp Tyr Val Asp Gly Val Glu Val His Asn Ala |
| | Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly |
| | Leu Pro Ser Ser Ile Glu Lys Thr Ile Ser Lys Ala Lys Gly |
| | Gln Pro Arg Glu Pro Gln Val Tyr Thr Leu Pro Pro Ser Arg |
| | Asp Glu Leu Thr Lys Asn Gln Val Ser Leu Thr Cys Leu |
| | Val Lys Gly Phe Tyr Pro Ser Asp Ile Ala Val Glu Trp Glu |
| | Ser Asn Gly Gln Pro Glu Asn Asn |
| | Tyr Lys Thr Thr Pro Pro Val Leu Asp Ser Asp Gly Ser |
| | Phe Phe Leu Tyr Ser Lys Leu Thr Val Asp Lys Ser Arg |
| | Trp Gln Gln Gly Asn Val Phe Ser Cys Ser Val Met His |
| | Glu Ala Leu His Asn Arg Phe Thr Gln Lys Ser Leu Ser |
| | Leu Ser Pro Gly Lys (SEQ ID NO: 131) |
| SEQ ID NO: 38 of | Ala Ser Thr Lys Gly Pro Ser Val Phe Pro Leu Ala Pro Cys |
| WO2014/121087 | Ser Arg Ser Thr Ser Glu Ser Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met Ile Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Gln Glu Glu Met Thr Lys Asn |
| | Gln Val Ser Leu Thr Cys Leu Val Lys Gly Phe Tyr Pro |
| | Ser Asp Ile Ala Val Glu Trp Glu Ser Asn Gly Gln Pro Glu |
| | Asn Asn Tyr Lys Thr Thr Pro Pro Val Leu Asp Ser Asp |
| | Gly Ser Phe Phe Leu Tyr Ser Arg Leu Thr Val Asp Lys |
| | Ser Arg Trp Gln Glu Gly Asn Val Phe Ser Cys Ser Val |
| | Met His Glu Ala Leu His Asn Arg Phe Thr Gln Lys Ser |
| | Leu Ser Leu Ser Leu Gly Lys (SEQ ID NO: 132) |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.7.1.2. Fc Heterodimerization Variants

Certain multispecific molecules entail dimerization between two Fc domains that, unlike a native immunoglobulin, are operably linked to non-identical N-terminal regions, e.g., one Fc domain connected to a targeting moiety (e.g., a Fab) and the other Fc domain connected to a pMHC complex. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the multispecific molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides, in some aspects, multispecific molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the multispecific molecules comprise a first CH3 domain and a second Ig CH3 domain, wherein the first and second Ig CH3 domains differ from one another by at least one amino acid, and wherein at least one amino acid difference reduces binding of the multispecific molecule to Protein A as compared to a corresponding multispecific molecule lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.7.1.3. Hinge Domains

The multispecific molecules of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric multispecific molecule formed by the association of two Fc domains) can comprise two associated hinge sequences on separate polypeptide chains. Sometimes, the two associated hinge sequences are referred to as a “hinge region”.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, a multispecific molecule of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the multispecific molecules of the disclosure comprise a modified hinge region that reduces binding affinity for an Fcγ receptor relative to a wild-type hinge region of the same isotype (e.g., human IgG1 or human IgG4).

In one embodiment, the multispecific molecules of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO: 133). The core hinge region of human IgG4 contains the sequence CPSC (SEQ ID NO: 134) compared to IgG1 that contains the sequence CPPC (SEQ ID NO: 133). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.7.1.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:135, previously disclosed as SEQ ID NO:8 of WO2014/121087, which is incorporated by reference in its entirety herein) or ESKYGPPCPPCPAPPVA (SEQ ID NO:136, previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.7.1.1).

6.7.1.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 137, previously disclosed as SEQ ID NO:1 of WO2016161010A2), CPPCPAPGG--GPSVF (SEQ ID NO: 138, previously disclosed as SEQ ID NO:2 of WO2016161010A2), CPPCPAPG---GPSVF (SEQ ID NO:139, previously disclosed as SEQ ID NO:3 of WO2016161010A2), or CPPCPAP----GPSVF (SEQ ID NO: 140, previously disclosed as SEQ ID NO:4 of WO2016161010A2).

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.7.1.1).

### 6.8. Linkers

In certain aspects, the present disclosure provides multispecific molecules (e.g., T cell activators) in which two or more components of a multispecific molecule are connected to one another by a peptide linker. By way of example and not limitation, linkers can be used to connect (a) a pMHC complex and a multimerization moiety; (b) a pMHC complex and an immune cell antigen targeting moiety; (c) an immune cell antigen targeting moiety and a multimerization moiety (e.g., a Fab domain and an Fc domain); (d) a tumor antigen targeting moiety and a multimerization moiety (e.g., a Fab domain and an Fc domain), (e) different components of a pMHC complex (e.g., an antigenic peptide and an MHC molecule); or (f) different domains within an immune cell antigen targeting moiety (e.g., the VH and VL domains in a scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the multispecific molecules of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO:141) or SGₙ (SEQ ID NO: 142), where n is an integer from 1 to 10, e.g., 12, 3, 4, 5, 6, 7, 8, 9 or 10. In one embodiment, the linker is or comprises a monomer or multimer of repeat of G4S (SEQ ID NO:143) e.g., (GGGGS)n (SEQ ID NO:144), where n is an integer from 1 to 10.

Polyglycine linkers can suitably be used in the T cell activators of the disclosure. In some embodiments, a peptide linker comprises two consecutive glycines (2Gly), three consecutive glycines (3Gly), four consecutive glycines (4Gly) (SEQ ID NO:145), five consecutive glycines (5Gly) (SEQ ID NO:146), six consecutive glycines (6Gly) (SEQ ID NO:147), seven consecutive glycines (7Gly) (SEQ ID NO: 148), eight consecutive glycines (8Gly) (SEQ ID NO: 149) or nine consecutive glycines (9Gly) (SEQ ID NO: 150).

A linker of the disclosure (e.g., a linker between a pMHC complex and an Fc domain, a linker between an immune cell antigen targeting moiety and an Fc domain, etc.) may in some embodiments be a “short” linker (i.e., a linker less than or equal to 7 amino acids in length). A short linker may be at most or exactly 7, 6, 5, 4, 3, or 2 amino acids in length. In some embodiments, a T cell activator of the present disclosure comprises a pMHC complex and an Fc domain operably linked by a short linker. Alternatively, a linker of the disclosure may in some embodiments be a “long” linker (i.e., a linker greater than 7 amino acids in length). A long linker may be at least or exactly 8, 9, 10, 11, 12, 13, 14, 15, 16, 17, 18, 19, or 20 amino acids in length, or more. In some embodiments, a T cell activator of the present disclosure comprises a pMHC complex and an Fc domain operably linked by a long linker.

Exemplary linker sequences are set forth in Table L below. A T cell activator of the disclosure may comprise one or more linkers of Table L.

TABLE L
Linker Sequences

| Linker Sequence | Designation | Linker Type | SEQ ID NO: |
|---|---|---|---|
| (GGGGS)ₙ | L1 | short (n = 1); | 143; |
| | | long (n > 1) | 151 |
| (GGGS)ₙ | L2 | short (n = 1); | 152; |
| | | long (n > 1) | 153 |
| (GGS)ₙ | L3 | short (n < 3); | 154; |
| | | long (n > 2) | 155 |
| (GS)ₙ | L4 | short (n < 3); | 156; |
| | | long (n > 2) | 157 |
| (GSGGS)ₙ | L5 | long | 158 |
| AAAGG | L6 | short | 159 |
| ADAAP | L7 | short | 160 |
| ADAAPTVSIFP | L8 | long | 161 |
| ADAAPTVSIFPP | L9 | long | 162 |
| AKTTAP | L10 | short | 163 |
| AKTTAPSVYPLAP | L11 | long | 164 |
| AKTTPKLEEGEFSEARV | L12 | long | 165 |
| AKTTPKLGG | L13 | long | 166 |
| AKTTPP | L14 | short | 167 |
| AKTTPPSVTPLAP | L15 | long | 168 |
| ASTKGP | L16 | short | 169 |
| ASTKGPSVFPLAPASTKGPSVFPLAP | L17 | long | 170 |
| EGKSSGSGSESKST | L18 | long | 171 |
| GEGESGEGESGEGES | L19 | long | 172 |
| GEGESGEGESGEGESGEGES | L20 | long | 173 |
| GEGGSGEGGSGEGGS | L21 | long | 174 |
| GENKVEYAPALMALS | L22 | long | 175 |
| GGEGSGGEGSGGEGS | L23 | long | 176 |
| GGGESGGEGSGEGGS | L24 | long | 177 |
| GGGESGGGESGGGES | L25 | long | 178 |
| GGGGSGGGGS | L26 | long | 151 |
| GGGGSGGGGSGGGGS | L27 | long | 127 |
| GGGGSGGGGSGGGGSGGGGS | L28 | long | 179 |
| GGGKSGGGKSGGGKS | L29 | long | 180 |
| GGGKSGGKGSGKGGS | L30 | long | 181 |
| GGGS | L31 | short | 152 |
| GGGSG | L32 | short | 182 |
| GGKGSGGKGSGGKGS | L33 | long | 183 |
| GGS | L34 | short | N/A |
| GGSG | L35 | short | 184 |
| GGSGG | L36 | short | 185 |
| GGSGG | L37 | short | 185 |
| GGSGGGGSG | L38 | long | 186 |
| GGSGGGGSGGGGS | L39 | long | 187 |
| GHEAAAVMQVQYPAS | L40 | long | 188 |
| GKGGSGKGGSGKGGS | L41 | long | 189 |
| GKGKSGKGKSGKGKS | L42 | long | 190 |
| GKGKSGKGKSGKGKSGKGKS | L43 | long | 191 |
| GKPGSGKPGSGKPGS | L44 | long | 192 |
| GKPGSGKPGSGKPGSGKPSGS | L45 | long | 193 |
| GPAKELTPLKEAKVS | L46 | long | 194 |
| GSAGSAAGSGEF | L47 | long | 195 |
| GSGGG | L48 | short | 196 |
| GSGSG | L49 | short | 197 |
| GSS | L50 | short | N/A |
| GSSG | L51 | short | 198 |
| GSSGGSGGSG | L52 | long | 199 |
| GSSGGSGGSGG | L53 | long | 200 |
| GSSGGSGGSGGS | L54 | long | 201 |
| GSSGGSGGSGGSG | L55 | long | 202 |
| GSSGGSGGSGGSGGGS | L56 | long | 203 |
| GSSGGSGGSGS | L57 | long | 204 |
| GSSGT | L58 | short | 205 |
| GSSSG | L59 | short | 206 |
| GSTSGSGKPGSGEGSTKG | L60 | long | 207 |
| GTAAAGAGAAGGAAAGAAG | L61 | long | 208 |
| GTSGSSGSGSGGSGSGGGG | L62 | long | 209 |
| IRPRAIGGSKPRVA | L63 | long | 210 |
| KESGSVSSEQLAQFRSLD | L64 | long | 211 |
| KTTPKLEEGEFSEAR | L65 | long | 212 |
| PRGASKSGSASQTGSAPGS | L66 | long | 213 |
| QPKAAP | L67 | short | 214 |
| QPKAAPSVTLFPP | L68 | long | 215 |
| RADAAAA(G4S)4 | L69 | long | 216 |
| RADAAAAGGPGS | L70 | long | 217 |
| RADAAP | L71 | short | 218 |
| RADAAPTVS | L72 | long | 219 |
| SAKTTP | L73 | short | 220 |
| SAKTTPKLEEGEFSEARV | L74 | long | 221 |
| SAKTTPKLGG | L75 | long | 222 |
| STAGDTHLGGEDFD | L76 | long | 223 |
| TVAAP | L77 | short | 224 |
| TVAAPSVFIFPP | L78 | long | 225 |
| TVAAPSVFIFPPTVAAPSVFIFPP | L79 | long | 226 |

In some embodiments, the T cell activator comprises linker L1. In some embodiments, the T cell activator comprises linker L2. In some embodiments, the T cell activator comprises linker L3. In some embodiments, the T cell activator comprises linker L4. In some embodiments, the T cell activator comprises linker L5. In some embodiments, the T cell activator comprises linker L6. In some embodiments, the T cell activator comprises linker L7. In some embodiments, the T cell activator comprises linker L8. In some embodiments, the T cell activator comprises linker L9. In some embodiments, the T cell activator comprises linker L10. In some embodiments, the T cell activator comprises linker L11. In some embodiments, the T cell activator comprises linker L12. In some embodiments, the T cell activator comprises linker L13. In some embodiments, the T cell activator comprises linker L14. In some embodiments, the T cell activator comprises linker L15. In some embodiments, the T cell activator comprises linker L16. In some embodiments, the T cell activator comprises linker L17. In some embodiments, the T cell activator comprises linker L18. In some embodiments, the T cell activator comprises linker L19. In some embodiments, the T cell activator comprises linker L20. In some embodiments, the T cell activator comprises linker L21. In some embodiments, the T cell activator comprises linker L22. In some embodiments, the T cell activator comprises linker L23. In some embodiments, the T cell activator comprises linker L24. In some embodiments, the T cell activator comprises linker L25. In some embodiments, the T cell activator comprises linker L26. In some embodiments, the T cell activator comprises linker L27. In some embodiments, the T cell activator comprises linker L28. In some embodiments, the T cell activator comprises linker L29. In some embodiments, the T cell activator comprises linker L30. In some embodiments, the T cell activator comprises linker L31. In some embodiments, the T cell activator comprises linker L32. In some embodiments, the T cell activator comprises linker L33. In some embodiments, the T cell activator comprises linker L34. In some embodiments, the T cell activator comprises linker L35. In some embodiments, the T cell activator comprises linker L36. In some embodiments, the T cell activator comprises linker L37. In some embodiments, the T cell activator comprises linker L38. In some embodiments, the T cell activator comprises linker L39. In some embodiments, the T cell activator comprises linker L40. In some embodiments, the T cell activator comprises linker L41. In some embodiments, the T cell activator comprises linker L42. In some embodiments, the T cell activator comprises linker L43. In some embodiments, the T cell activator comprises linker L44. In some embodiments, the T cell activator comprises linker L45. In some embodiments, the T cell activator comprises linker L46. In some embodiments, the T cell activator comprises linker L47. In some embodiments, the T cell activator comprises linker L48. In some embodiments, the T cell activator comprises linker L49. In some embodiments, the T cell activator comprises linker L50. In some embodiments, the T cell activator comprises linker L51. In some embodiments, the T cell activator comprises linker L52. In some embodiments, the T cell activator comprises linker L53. In some embodiments, the T cell activator comprises linker L54. In some embodiments, the T cell activator comprises linker L55. In some embodiments, the T cell activator comprises linker L56. In some embodiments, the T cell activator comprises linker L57. In some embodiments, the T cell activator comprises linker L58. In some embodiments, the T cell activator comprises linker L59. In some embodiments, the T cell activator comprises linker L60. In some embodiments, the T cell activator comprises linker L61. In some embodiments, the T cell activator comprises linker L62. In some embodiments, the T cell activator comprises linker L63. In some embodiments, the T cell activator comprises linker L64. In some embodiments, the T cell activator comprises linker L65. In some embodiments, the T cell activator comprises linker L66. In some embodiments, the T cell activator comprises linker L67. In some embodiments, the T cell activator comprises linker L68. In some embodiments, the T cell activator comprises linker L69. In some embodiments, the T cell activator comprises linker L70. In some embodiments, the T cell activator comprises linker L71. In some embodiments, the T cell activator comprises linker L72. In some embodiments, the T cell activator comprises linker L73. In some embodiments, the T cell activator comprises linker L74. In some embodiments, the T cell activator comprises linker L75. In some embodiments, the T cell activator comprises linker L76. In some embodiments, the T cell activator comprises linker L77. In some embodiments, the T cell activator comprises linker L78. In some embodiments, the T cell activator comprises linker L79.

6.8.1. pMHC Linkers

For pMHC complexes, suitable linkers can range from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. In addition to the linkers above, pMHC linkers include glycine polymers (G)n, glycine-serine polymers (including, for example, (GS)n, (GSGGS)n (SEQ ID NO:158) and (GGGS)n (SEQ ID NO: 152), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, 1992, Rev. Computational Chem. 1 1173-142, incorporated herein in its entirety by reference). Exemplary linkers can comprise amino acid sequences including, but not limited to, GGSG (SEQ ID NO:184), GGSGG (SEQ ID NO:185), GSGSG (SEQ ID NO:197), GSGGG (SEQ ID NO:196), GGGSG (SEQ ID NO:182), GSSSG (SEQ ID NO:206), GCGASGGGGSGGGGS (SEQ ID NO:227), GGGGSGGGGS (SEQ ID NO:151), GGGASGGGGSGGGGS (SEQ ID NO:228), GGGGSGGGGSGGGGS (SEQ ID NO:127), GGGASGGGGS (SEQ ID NO:229), GGGGSGGGGSGGGGSGGGGS (SEQ ID NO:179), GCGGS (SEQ ID NO:230) and the like. In some embodiments, a linker polypeptide includes a cysteine residue that can form a disulfide bond with a cysteine residue present in another portion of the pMHC complex. In certain embodiments, the linker comprises the amino acid sequence GCGGS (SEQ ID NO:230). The substitution of a glycine in the G4S (SEQ ID NO: 143) linker with cysteine can result in the formation of a disulfide bond, for example an MHC targeting moiety with a corresponding cysteine substitution in HLA.A2 that stabilizes the MHC peptide within the MHC complex.

### 6.9. Nucleic Acids and Host Cells

In another aspect, the disclosure provides nucleic acids encoding the multispecific molecules (e.g., T cell activators) of the disclosure. In some embodiments, the multispecific molecules are encoded by a single nucleic acid. In other embodiments, for example in the case of a heterodimeric molecule or a molecule comprising a component (e.g., pMHC complex, immune cell antigen targeting moiety) composed of more than one polypeptide chain, the multispecific molecules can be encoded by a plurality (e.g., two, three, four or more) nucleic acids.

A single nucleic acid can encode a multispecific molecule that comprises a single polypeptide chain, a multispecific molecule that comprises two or more polypeptide chains, or a portion of a multispecific molecule that comprises more than two polypeptide chains (for example, a single nucleic acid can encode two polypeptide chains of a multispecific molecule comprising three, four or more polypeptide chains, or three polypeptide chains of a multispecific molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a multispecific molecule comprising two or more polypeptide chains is encoded by two or more nucleic acids. The number of nucleic acids encoding a multispecific molecule can be equal to or less than the number of polypeptide chains in the multispecific molecule (for example, when more than one polypeptide chains are encoded by a single nucleic acid).

The nucleic acids of the disclosure can be DNA (e.g., plasmid) or RNA (e.g., mRNA).

In some aspects, the disclosure provides methods for delivery of T cell activators to an individual in need thereof by administering nucleic acids of the disclosure. Nucleic acids may be delivered to an individual via various pharmaceutical compositions, including prophylactic compositions, examples of which are described in Section 6.10.

In another aspect, the disclosure provides host cells and vectors containing the nucleic acids of the disclosure. The nucleic acids may be present in a single vector or separate vectors present in the same host cell or separate host cell, as described in more detail herein below.

### 6.9.1. Vectors

The disclosure provides vectors comprising nucleotide sequences encoding a multispecific molecule or a multispecific molecule component described herein, for example one or two of the polypeptide chains of a T cell activator. The vectors include, but are not limited to, a virus, plasmid, cosmid, lambda phage or a yeast artificial chromosome (YAC). Vectors encoding a T cell activator of the disclosure (or component thereof) may be useful in expression and/or delivery of T cell activators.

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.9.2. Host Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The host cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 6.10. Pharmaceutical Compositions

### 6.10.1. Pharmaceutical Compositions Comprising T Cell Activator Polypeptide

The T cell activators of the disclosure may be in the form of compositions comprising the T cell activator and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the T cell activator and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an T cell activator of the disclosure per dose. The quantity of T cell activator included in a unit dose will depend on the disease being treated, as well as other factors as are well known in the art. Such unit dosages may be in the form of a lyophilized dry powder containing an amount of T cell activator suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of T cell activator suitable for a single administration.

The pharmaceutical compositions may also be supplied in bulk from containing quantities of T cell activator suitable for multiple administrations.

Pharmaceutical compositions may be prepared for storage as lyophilized formulations or aqueous solutions by mixing a T cell activator having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of T cell activator.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.10.2. Pharmaceutical Compositions for Delivery of T Cell Activator Encoding Nucleic Acids

A T cell activator of the disclosure can be delivered by means of a nucleic acid encoding the T cell activator, for example as a plasmid, DNA, mRNA or through viral vectors encoding the T cell activator under the control of a suitable promoter.

Exemplary vectors include adenovirus- or AAV-based therapeutics. Non-limiting examples of adenovirus-based or AAV-based therapeutics for use in the methods, uses or compositions herein include, but are not limited to: rAd-p53, which is a recombinant adenoviral vector encoding the wild-type human tumor suppressor protein p53, for example, for the use in treating a cancer (also known as Gendicine®, Genkaxin®, Qi et al., 2006, Modern Oncology, 14:1295-1297); Ad5_d11520, which is an adenovirus lacking the E1B gene for inactivating host p53 (also called H101 or ONYX-015; see, e.g., Russell et al., 2012, Nature Biotechnology 30:658-670); AD5-D24-GM-CSF, an adenovirus containing the cytokine GM-CSF, for example, for the use in treating a cancer (Cerullo et al., 2010, Cancer Res. 70:4297); rAd-HSVtk, a replication deficient adenovirus with HSV thymidine kinase gene, for example, for the treatment of cancer (developed as Cerepro®, Ark Therapeutics, see e.g. U.S. Pat. No. 6,579,855; developed as ProstAtak™ by Advantagene; International PCT Appl. No. WO2005/049094); rAd-TNFα, a replication-deficient adenoviral vector expressing human tumor necrosis factor alpha (TNFα) under the control of the chemoradiation-inducible EGR-1 promoter, for example, for the treatment of cancer (TNFerade™, GenVec; Rasmussen et al., 2002, Cancer Gene Ther. 9:951-7; Ad-IFNβ, an adenovirus serotype 5 vector from which the E1 and E3 genes have been deleted expressing the human interferon-beta gene under the direction of the cytomegalovirus (CMV) immediate-early promoter, for example for treating cancers (BG00001 and H5.110CMVhIFN-β, Biogen; Sterman et al., 2010, Mol. Ther. 18:852-860). Additional vectors are recognized in the art and include, for example, lentiviral vectors (e.g., VSV), retroviral vectors, and others.

Any now-known or future-developed delivery vector, natural or engineered, can be used in the delivery of a T cell activator of the disclosure. In some embodiments, the delivery vector is a viral vector, e.g., comprises a virus, viral capsid, viral genome etc. In some embodiments, the delivery vector is a naked nucleic acid, e.g., an episome. In some embodiments, the delivery vector comprises a nucleic acid complex. Exemplary non-limiting nucleic acid complexes for use as a delivery vector include lipoplexes, polymersomes, polypexes, dendrimers, inorganic nanoparticles (e.g., polynucleotide coated gold, silica, iron oxide, calcium phosphate, etc.). In some embodiments, a delivery vector as described herein comprises a combination of a viral vector, naked nucleic acids, and nucleic acid complexes.

In one embodiment, the delivery vector is a virus, including a retrovirus, adenovirus, herpes simplex virus, pox virus, vaccinia virus, lentivirus, or an adeno-associated virus. In one embodiment, the delivery vector is an adeno-associated virus (AAV), including serotypes AAV1, AAV2, AAV3, AAV4, AAV5, AAV6, AAV7, AAV8, AAV9, AAV10, and AAV11, or engineered or naturally selected variants thereof.

In one embodiment, a nucleic acid encoding a T cell activator (or component thereof) also contains adeno-associated virus (AAV) nucleic acid sequence. In one embodiment, the vector is a chimeric adeno-associated virus containing genetic elements from two or more serotypes. For example, an AAV vector with rep genes from AAV1 and cap genes from AAV2 (designated as AAV1/2 or AAV RC1/2) may be used as a delivery vector to deliver a T cell activator expressing nucleic acid to a cell or a cell of a patient in need. In one embodiment, the delivery vector is an AAV1/2, AAV1/3, AAV1/4, AAV1/5, AAV1/6, AAV1/7, AAV1/8, AAV1/9, AAV1/10, AAV1/11, AAV2/1, AAV2/3, AAV2/4, AAV2/5, AAV2/6, AAV2/7, AAV2/8, AAV2/9, AAV2/10, AAV2/11, AAV3/1, AAV3/2, AAV3/4, AAV3/5, AAV3/6, AAV3/7, AAV3/8, AAV3/9, AAV3/10, AAV3/10, AAV4/1, AAV4/2, AAV4/3, AAV4/5, AAV4/6, AAV4/7, AAV4/8, AAV4/9, AAV4/10, AAV4/11, AAV5/1, AAV5/2, AAV5/3, AAV5/4, AAV5/6, AAV5/7, AAV5/8, AAV5/9, AAV5/10, AAV5/11, AAV6/1, AAV6/2, AAV6/3, AAV6/4, AAV6/5, AAV6/7, AAV6/8, AAV6/9, AAV6/10, AAV6/10, AAV7/1, AAV7/2, AAV7/3, AAV7/4, AAV7/5, AAV7/6, AAV7/8, AAV7/9, AAV7/10, AAV7/11, AAV8/1, AAV8/2, AAV8/3, AAV8/4, AAV8/5, AAV8/6, AAV8/7, AAV8/9, AAV8/10, AAV8/11, AAV9/1, AAV9/2, AAV9/3, AAV9/4, AAV9/5, AAV9/6, AAV9/7, AAV9/8, AAV9/10, AAV9/11, AAV10/1, AAV10/2, AAV10/3, AAV10/4, AAV10/5, AAV10/6, AAV10/7, AAV10/8, AAV10/9, AAV10/11, AAV11/1, AAV11/2, AAV11/3, AAV11/4, AAV11/5, AAV11/6, AAV11/7, AAV11/8, AAV11/9, AAV11/10, chimeric viral vector or, derivative thereof. Gao et al., “Novel adeno-associated viruses from rhesus monkeys as vectors for human gene therapy,” PNAS 99(18): 11854-11859, Sep. 3, 2002, is incorporated herein by reference for AAV vectors and chimeric viral vectors useful as delivery vectors, and their construction and use.

### 6.10.2.1. Nucleic Acid Formulations

Nucleic acids of the present disclosure (e.g., encoding a T cell activator or component thereof) may be formulated or administered in combination with one or more pharmaceutically-acceptable excipients. In addition to traditional excipients such as any and all solvents, dispersion media, diluents, or other liquid vehicles, dispersion or suspension aids, surface active agents, isotonic agents, thickening or emulsifying agents, and preservatives, excipients can include, without limitation, lipidoids, liposomes, lipid nanoparticles, polymers, lipoplexes, core-shell nanoparticles, peptides, proteins, cells transfected with nucleic acids (e.g., for transplantation into a subject), hyaluronidase, nanoparticle mimics, and combinations thereof.

In some embodiments, the nucleic acids disclosed herein (i.e. nucleic acids encoding a T cell activator or component thereof) may be formulated as nanoparticles. Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less. Accordingly, the present disclosure also provides nanoparticle compositions comprising (i) a lipid composition comprising a delivery agent, and (ii) one or more nucleic acids encoding a T cell activator of the disclosure or component thereof. In such a nanoparticle composition, the lipid composition disclosed herein can encapsulate the nucleic acid(s). In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels. In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid.

In some embodiments, a nucleic acid as described herein comprises complexes such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof.

In some embodiments, the nucleic acids disclosed herein may be formulated as self-assembled nanoparticles. As a non-limiting example, nucleic acids may be used to make nanoparticles which may be used in a delivery system for the nucleic acids (See e.g., International Pub. No. WO2012125987; herein incorporated by reference in its entirety). In some embodiments, the nucleic acid self-assembled nanoparticles may comprise a core of the nucleic acids disclosed herein and a polymer shell. The polymer shell may be any of the polymers described herein and are known in the art. In an additional embodiment, the polymer shell may be used to protect the nucleic acids in the core.

In some embodiment, these self-assembled nanoparticles may be microsponges formed of long polymers of nucleic acid hairpins which form into crystalline ‘pleated’ sheets before self-assembling into microsponges. These microsponges are densely-packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269; herein incorporated by reference in its entirety). Additionally, these microsponges may be able to exhibit an extraordinary degree of protection from degradation by ribonucleases. In another embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, nucleic acids.

In some embodiments, nucleic acids may be formulated in inorganic nanoparticles (U.S. Pat. No. 8,257,745, herein incorporated by reference in its entirety). The inorganic nanoparticles may include, but are not limited to, clay substances that are water swellable. As a non-limiting example, the inorganic nanoparticle may include synthetic smectite clays which are made from simple silicates (See e.g., U.S. Pat. Nos. 5,585,108 and 8,257,745 each of which are herein incorporated by reference in their entirety).

In some embodiments, a nucleic acid may be formulated in water-dispersible nanoparticle comprising a semiconductive or metallic material (U.S. Pub. No. 20120228565; herein incorporated by reference in its entirety) or formed in a magnetic nanoparticle (U.S. Pub. No. 20120265001 and 20120283503; each of which is herein incorporated by reference in its entirety). The water-dispersible nanoparticles may be hydrophobic nanoparticles or hydrophilic nanoparticles.

In some embodiments, the nucleic acids disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous. As a non-limiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410-12413; herein incorporated by reference in its entirety). In some embodiment, the polynucleotide may be encapsulated in a lipid nanoparticle and then the lipid nanoparticle may be encapsulated into a hydrogel.

In some embodiments, the nucleic acids disclosed herein may be encapsulated into a fibrin gel, fibrin hydrogel or fibrin glue. In another embodiment, the nucleic acids may be formulated in a lipid nanoparticle or a rapidly eliminated lipid nanoparticle prior to being encapsulated into a fibrin gel, fibrin hydrogel or a fibrin glue. In yet another embodiment, the nucleic acids may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85; each of which is herein incorporated by reference in its entirety). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128; each of which is herein incorporated by reference in its entirety).

In some embodiments, a nucleic acid disclosed herein may include cations or anions. In one embodiment, the formulations include metal cations such as, but not limited to, Zn2+, Ca2+, Cu2+, Mg+ and combinations thereof. As a non-limiting example, formulations may include polymers and a polynucleotide complexed with a metal cation (See e.g., U.S. Pat. Nos. 6,265,389 and 6,555,525, each of which is herein incorporated by reference in its entirety).

The nucleic acid molecule (e.g., plasmid, mRNA, DNA) or virus can be formulated as the sole pharmaceutically active ingredient in a pharmaceutical composition or can be combined with other active agents for the particular disorder treated. Optionally, other medicinal agents, pharmaceutical agents, carriers, adjuvants, diluents can be included in the compositions provided herein. For example, any one or more of a wetting agents, emulsifiers and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 6.11. Methods of Use

T cell activators of the disclosure are useful in eliciting immune responses and treating or preventing disease states where stimulation of the immune system of the host is beneficial, in particular conditions where an enhanced cellular immune response is desirable. These may include disease states where the host immune response is insufficient or deficient. Disease states for which the T cell activators of the disclosure can be administered for purposes of treatment or prevention comprise, for example, a tumor or infection where a cellular immune response would be a critical mechanism for specific immunity. Specific disease states for which T cell activators of the present disclosure can be employed include cancer. The T cell activators of the disclosure may be administered per se or in any suitable pharmaceutical composition, including a composition as described in Section 6.10.1 (e.g., when administered as a T cell activator polypeptide) or in Sections 6.10.2 and 6.10.2.1 (e.g., when administered as one or more nucleic acids encoding a T cell activator).

In one aspect, T cell activators of the disclosure for use as a medicament are provided. In further aspects, T cell activators of the disclosure for use in treating a disease are provided. In certain embodiments, T cell activators of the disclosure for use in a method of treatment are provided. In one embodiment, the disclosure provides a T cell activator as described herein for use in the treatment of a disease in a subject in need thereof. In certain embodiments, the disclosure provides a T cell activator for use in a method of treating a subject having a disease comprising administering to the individual a therapeutically effective amount of the T cell activator. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer, such as a multispecific antigen binding molecule described in Section 6.6.

In further embodiments, the disclosure provides a T cell activator for use in stimulating the immune system, particularly in an antigen-specific manner. In certain embodiments, the disclosure provides a T cell activator for use in a method of stimulating the immune system in a subject comprising administering to the individual an effective amount of the T cell activator to stimulate the immune system. An “individual” according to any of the above embodiments is a mammal, preferably a human. “Stimulation of the immune system” according to any of the above embodiments may include any one or more of a general increase in immune function, an increase in T cell function, an increase in T cell proliferation (particularly antigen-specific T cell proliferation), an increase in B cell function, a restoration of lymphocyte function, an increase in T cell responsiveness, an increase in natural killer cell activity or lymphokine-activated killer (LAK) cell activity, and the like. In particular embodiments, the disclosure provides a T cell activator for use in increasing antigen-specific T cell proliferation, that is for use in increasing proliferation of T cells expressing a TCR specific for the antigen (or portion thereof) present in the pMHC complex of the T cell activator.

In further embodiments, the disclosure provides methods for preventing a disorder or condition associated with a particular antigen comprising administration of a T cell activator of the present disclosure to an individual. As disclosed herein, a T cell activator may be used to activate T cells in an antigen-specific manner, thereby inducing a protective immune response in an individual. Such methods are also referred to herein as “prophylactic methods” and comprise administration of a T cell activator of the disclosure to a subject at risk for developing a disease, disorder, or condition associated with an antigen, where the T cell activator comprises a pMHC complex comprising the antigen or a portion thereof. Antigens include viral, bacterial, and tumor antigens, and prophylactic methods of the disclosure may include, for example, prevention of a malignancy. In some embodiments, the disclosure provides a T cell activator for use in a prophylactic method. Exemplary nucleic acids and formulations for delivery of a T cell activator are described in Sections 6.10.2.1 and 6.10.2, respectively.

In certain embodiments, a prophylactic method of the disclosure comprises administration of a T cell activator comprising a tumor antigen (or portion thereof), thereby preventing a malignancy in an individual. Such tumor antigens (or portions thereof; also referred to herein as “antigenic determinants of cancer cells” or “antigenic peptides”) include, but are not limited to, LCMV derived peptide gp33-41, APF (126-134), BALF(276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAG-A3 (112-120), MAGE-A4 (230-239), MAGE-A4 (286-294), NYESO1 (157-165, C165A), NYESO1 (157-165, C165V), p54 WT (264-272), PAP-3 (136-143), PSMA (4-12), PSMA (135-145), Survivin (96-014), Tyrosinase (369-377, 371D), and WT1 (126-134). Additional tumor antigens contemplated herein include those listed in Table 2. An “individual” according to any of the above embodiments is a mammal, preferably a human.

In a further aspect, the disclosure provides for the use of a T cell activator of the disclosure in the manufacture or preparation of a medicament for the treatment or prevention of a disease in a subject in need thereof. In one embodiment, the medicament is for use in a method of treating a disease comprising administering to a subject having the disease a therapeutically effective amount of the medicament. In certain embodiments, the disease to be treated is a proliferative disorder. In a preferred embodiment the disease is cancer. In one such embodiment, the method further comprises administering to the individual a therapeutically effective amount of at least one additional therapeutic agent, e.g., an anti-cancer agent if the disease to be treated is cancer such as a multispecific antigen binding molecule described in Section 6.6. In an additional embodiment, the medicament is for use in a method of preventing a disease comprising administering to a subject not having the disease, in some cases at risk for developing the disease, a prophylactically effective amount of the medicament. In certain embodiments, the disease to be prevented is a proliferative disorder.

In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments is a mammal, preferably a human. “Stimulation of the immune system” according to any of the above embodiments may include any one or more of a general increase in immune function, an increase in T cell function, an increase in T cell proliferation (particularly antigen-specific T cell proliferation), an increase in B cell function, a restoration of lymphocyte function, an increase in T cell responsiveness, an increase in natural killer cell activity or lymphokine-activated killer (LAK) cell activity, and the like.

In a further aspect, the disclosure provides a method for treating a disease in a subject, comprising administering to said individual a therapeutically effective amount of a T cell activator of the disclosure. In one embodiment a composition is administered to said individual, comprising the T cell activator of the disclosure in a pharmaceutically acceptable form. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer such as a multispecific antigen binding molecule described in Section 6.6. In a further aspect, the disclosure provides a method for stimulating the immune system in a subject, comprising administering to the individual an effective amount of a T cell activator to stimulate the immune system. An “individual” according to any of the above embodiments is a mammal, preferably a human.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a T cell activator of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. Similarly, other cell proliferation disorders can also be treated by the T cell activators of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above A skilled artisan readily recognizes that in many cases the T cell activators, when used in the context of therapeutic treatment, may not provide a cure but may only provide partial benefit. In some embodiments, a physiological change having some benefit is also considered therapeutically beneficial. Thus, in some embodiments, an amount of T cell activator that provides a physiological change is considered an “effective amount” or a “therapeutically effective amount”. A skilled artisan further recognizes that in many cases the T cell activators, when used in the context of a prophylactic treatment may not provide a cure but may completely prevent a subject from ever getting a specific disease or disorder, but may reduce the probability of developing the disease or disorder. The subject, patient, or individual in need of treatment is typically a mammal, more specifically a human.

For the prevention or treatment of disease, the appropriate dosage of a T cell activator of the disclosure (when used alone or in combination with one or more other additional therapeutic agents) will depend on the type of disease to be treated, the route of administration, the body weight of the patient, the particular T cell activator, the severity and course of the disease, whether the T cell activator is administered for preventive or therapeutic purposes, previous or concurrent therapeutic interventions, the patient's clinical history and response to the T cell activator, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The T cell activators of the disclosure will generally be used in an amount effective to achieve the intended purpose. For use to treat or prevent a disease condition, the T cell activators of the disclosure, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the T cell activators which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the T cell activators may not be related to plasma concentration. One having skill in the art will be able to optimize therapeutically effective local dosages without undue experimentation.

A therapeutically effective dose of the T cell activators described herein will generally provide therapeutic benefit without causing substantial toxicity. Toxicity and therapeutic efficacy of a T cell activator can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. T cell activators that exhibit large therapeutic indices are preferred. In one embodiment, the T cell activator according to the present disclosure exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with T cell activators of the disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### 6.12. Combination Therapy

The T cell activators according to the disclosure may be administered in combination with one or more other agents in therapy. For instance, a T cell activator of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In certain embodiments, the additional therapeutic is a multispecific antigen binding molecule as described in Section 6.6, including but not limited to a multispecific antigen binding molecule of Table K-2.

Such other agents are suitably present in combination in amounts that are effective for the purpose intended. The effective amount of such other agents depends on the amount of T cell activator used, the type of disorder or treatment, and other factors discussed above. The T cell activators are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the T cell activator of the disclosure can occur prior to, simultaneously, and/or following, administration of the additional therapeutic agent and/or adjuvant. T cell activators of the disclosure can also be used in combination with radiation therapy.

### 7. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

In particular aspects of the numbered embodiments below and the claims which follow, the Fc domains, MHC domains, β2M, and the variants thereof preferably comprise the amino acid sequences of human Fc domains, human MHC domains, human β2M, and variants thereof, for example variants with at least about 90%, at least about 95%, at least about 96%, at least about 97%, at least about 98%, at least about 99% or 100% sequence identity to such human sequence.

1. A multispecific molecule comprising:
(a) a peptide-MHC (pMHC) complex comprising an MHC domain and an antigenic peptide;
(b) an immune cell antigen (ICA) targeting moiety; and
(c) optionally, a multimerization moiety operably linked to the pMHC complex.

2. The multispecific molecule of embodiment 1, which comprises a single ICA targeting moiety.

3. The multispecific molecule of embodiment 1, which comprises two ICA targeting moieties.

4. The multispecific molecule of embodiment 3, wherein the two ICA targeting moieties bind to the same antigen, optionally wherein the two ICA targeting moieties are the same.

5. The multispecific molecule of embodiment 3, wherein the two ICA targeting moieties bind to different antigens.

6. The multispecific molecule of any one of embodiments 1 to 5, which comprises a single pMHC complex.

7. The multispecific molecule of any one of embodiments 1 to 5, which comprises two pMHC complexes.

8. The multispecific molecule of embodiment 7, wherein the two pMHC complexes are the same.

9. The multispecific molecule of any one of embodiments 1 to 8, which comprises a multimerization moiety operably linked to the pMHC complex.

10. The multispecific molecule of embodiment 9, wherein the multimerization moiety is an Fc domain.

11. The multispecific molecule of embodiment 10, wherein the Fc domain is an IgG1 Fc domain.

12. The multispecific molecule of embodiment 10, wherein the Fc domain is an IgG4 Fc domain.

13. The multispecific molecule of embodiment 10, wherein the Fc domain comprises an amino acid sequence having at least about 95% sequence identity to the amino acid sequence of any one of SEQ ID NOS: 10 to 28.

14. The multispecific molecule of embodiment 10, wherein the Fc domain comprises an amino acid sequence having at least about 95%, 96%, 97%, 98%, 99%, or 100% sequence identity to the amino acid sequence of SEQ ID NO:10.

15. The multispecific molecule of embodiment 10, wherein the Fc domain comprises an amino acid sequence having at least about 95%, 96%, 97%, 98%, 99%, or 100% sequence identity to the amino acid sequence of SEQ ID NO:11.

16. The multispecific molecule of embodiment 10, wherein the Fc domain comprises an amino acid sequence having at least about 95%, 96%, 97%, 98%, 99%, or 100% sequence identity to the amino acid sequence of SEQ ID NO: 12.

17. The multispecific molecule of embodiment 10, wherein the Fc domain comprises an amino acid sequence having at least about 95%, 96%, 97%, 98%, 99%, or 100% sequence identity to the amino acid sequence of SEQ ID NO: 13.

18. The multispecific molecule of embodiment 10, wherein the Fc domain comprises an amino acid sequence having at least about 95%, 96%, 97%, 98%, 99%, or 100% sequence identity to the amino acid sequence of SEQ ID NO:14.

19. The multispecific molecule of embodiment 10, wherein the Fc domain comprises an amino acid sequence having at least about 95%, 96%, 97%, 98%, 99%, or 100% sequence identity to the amino acid sequence of SEQ ID NO: 15.

20. The multispecific molecule of embodiment 10, wherein the Fc domain comprises an amino acid sequence having at least about 95%, 96%, 97%, 98%, 99%, or 100% sequence identity to the amino acid sequence of SEQ ID NO: 16.

21. The multispecific molecule of embodiment 10, wherein the Fc domain comprises an amino acid sequence having at least about 95%, 96%, 97%, 98%, 99%, or 100% sequence identity to the amino acid sequence of SEQ ID NO:17.

22. The multispecific molecule of embodiment 10, wherein the Fc domain comprises an amino acid sequence having at least about 95%, 96%, 97%, 98%, 99%, or 100% sequence identity to the amino acid sequence of SEQ ID NO:18.

23. The multispecific molecule of embodiment 10, wherein the Fc domain comprises an amino acid sequence having at least about 95%, 96%, 97%, 98%, 99%, or 100% sequence identity to the amino acid sequence of SEQ ID NO:19.

24. The multispecific molecule of embodiment 10, wherein the Fc domain comprises an amino acid sequence having at least about 95%, 96%, 97%, 98%, 99%, or 100% sequence identity to the amino acid sequence of SEQ ID NO:20.

25. The multispecific molecule of embodiment 10, wherein the Fc domain comprises an amino acid sequence having at least about 95%, 96%, 97%, 98%, 99%, or 100% sequence identity to the amino acid sequence of SEQ ID NO:21.

26. The multispecific molecule of embodiment 10, wherein the Fc domain comprises an amino acid sequence having at least about 95%, 96%, 97%, 98%, 99%, or 100% sequence identity to the amino acid sequence of SEQ ID NO:22.

27. The multispecific molecule of embodiment 10, wherein the Fc domain comprises an amino acid sequence having at least about 95%, 96%, 97%, 98%, 99%, or 100% sequence identity to the amino acid sequence of SEQ ID NO:23.

28. The multispecific molecule of embodiment 10, wherein the Fc domain comprises an amino acid sequence having at least about 95%, 96%, 97%, 98%, 99%, or 100% sequence identity to the amino acid sequence of SEQ ID NO:24.

29. The multispecific molecule of embodiment 10, wherein the Fc domain comprises an amino acid sequence having at least about 95%, 96%, 97%, 98%, 99%, or 100% sequence identity to the amino acid sequence of SEQ ID NO:25.

30. The multispecific molecule of embodiment 10, wherein the Fc domain comprises an amino acid sequence having at least about 95%, 96%, 97%, 98%, 99%, or 100% sequence identity to the amino acid sequence of SEQ ID NO:26.

31. The multispecific molecule of embodiment 10, wherein the Fc domain comprises an amino acid sequence having at least about 95%, 96%, 97%, 98%, 99%, or 100% sequence identity to the amino acid sequence of SEQ ID NO:27.

32. The multispecific molecule of embodiment 10, wherein the Fc domain comprises an amino acid sequence having at least about 95%, 96%, 97%, 98%, 99%, or 100% sequence identity to the amino acid sequence of SEQ ID NO:28.

33. The multispecific molecule of any one of embodiments 1 to 32, wherein the pMHC complex comprises:
(a) a peptide, optionally as set forth in Section 6.3.2; and
(b) an amino acid sequence having at least about 95%, 96%, 97%, 98%, 99%, or 100% sequence identity to the amino acid sequence of SEQ ID NO:252.

34. The multispecific molecule of embodiment 33, wherein the peptide is a peptide set forth in Table 1-A.

35. The multispecific molecule of embodiment 33, wherein the peptide is a peptide set forth in Table 1-B.

36. The multispecific molecule of embodiment 33, wherein the peptide is a peptide set forth in Table 1-C.

37. The multispecific molecule of embodiment 33, wherein the peptide is HPV 16E7 (11-19) (SEQ ID NO:242).

38. The multispecific molecule of embodiment 33, wherein the peptide is NYESO-1(157-165) (SEQ ID NO:244).

39. The multispecific molecule of embodiment 33, wherein the peptide is MAGEA4(230-239) (SEQ ID NO:246).

40. The multispecific molecule of embodiment 33, wherein the peptide is CMVpp65(465-503) (SEQ ID NO:248).

41. The multispecific molecule of any one of embodiments 1 to 33, wherein the pMHC complex comprises an amino acid sequence having at least about 95%, 96%, 97%, 98%, 99%, or 100% sequence identity to the amino acid sequence of SEQ ID NO:241, optionally wherein the peptide in the pMHC complex is identical to or has at most one or two amino acid substitutions to the peptide component of SEQ ID NO:241.

42. The multispecific molecule of any one of embodiments 1 to 33, wherein the pMHC complex comprises an amino acid sequence having at least about 95%, 96%, 97%, 98%, 99%, or 100% sequence identity to the amino acid sequence of SEQ ID NO:243, optionally wherein the peptide in the pMHC complex is identical to or has at most one or two amino acid substitutions to the peptide component of SEQ ID NO:243.

43. The multispecific molecule of any one of embodiments 1 to 33, wherein the pMHC complex comprises an amino acid sequence having at least about 95%, 96%, 97%, 98%, 99%, or 100% sequence identity to the amino acid sequence of SEQ ID NO:245, optionally wherein the peptide in the pMHC complex is identical to or has at most one or two amino acid substitutions to the peptide component of SEQ ID NO:245.

44. The multispecific molecule of any one of embodiments 1 to 33, wherein the pMHC complex comprises an amino acid sequence having at least about 95%, 96%, 97%, 98%, 99%, or 100% sequence identity to the amino acid sequence of SEQ ID NO:247, optionally wherein the peptide in the pMHC complex is identical to or has at most one or two amino acid substitutions to the peptide component of SEQ ID NO:247.

45. The multispecific molecule of any one of embodiments 1 to 44, wherein the multispecific molecule is a dimer.

46. The multispecific molecule of embodiment 45, wherein the multispecific molecule is a heterodimer.

47. The multispecific molecule of any one of embodiments 2 to 46, wherein the pMHC complex is N-terminal to the multimerization moiety.

48. The multispecific molecule of any one of embodiments 2 to 46, wherein the pMHC complex is C-terminal to the multimerization moiety.

49. The multispecific molecule of any one of embodiments 2 to 48, wherein the ICA targeting moiety is N-terminal to the multimerization moiety.

50. The multispecific molecule of any one of embodiments 2 to 48, wherein the ICA targeting moiety is C-terminal to the multimerization moiety.

51. The multispecific molecule of any one of embodiments 1 to 50, wherein the pMHC complex and the ICA targeting moiety are on a single polypeptide.

52. The multispecific molecule of any one of embodiments 1 to 51, wherein the pMHC complex and the ICA targeting moiety are on different polypeptides.

53. A multispecific molecule according to any one of embodiments 1 to 38, having the configuration of T Cell Activator 1 as set forth in Section 6.2.

54. A multispecific molecule according to any one of embodiments 1 to 38, having the configuration of T Cell Activator 2 as set forth in Section 6.2.

55. A multispecific molecule according to any one of embodiments 1 to 38, having the configuration of T Cell Activator 3 as set forth in Section 6.2.

56. A multispecific molecule according to any one of embodiments 1 to 38, having the configuration of T Cell Activator 4 as set forth in Section 6.2.

57. A multispecific molecule according to any one of embodiments 1 to 38, having the configuration of T Cell Activator 5 as set forth in Section 6.2.

58. A multispecific molecule according to any one of embodiments 1 to 38, having the configuration of T Cell Activator 6 as set forth in Section 6.2.

59. A multispecific molecule according to any one of embodiments 1 to 38, having the configuration of T Cell Activator 7 as set forth in Section 6.2.

60. A multispecific molecule according to any one of embodiments 1 to 38, having the configuration of T Cell Activator 8 as set forth in Section 6.2.

61. A multispecific molecule according to any one of embodiments 1 to 38, having the configuration of T Cell Activator 9 as set forth in Section 6.2.

62. A multispecific molecule according to any one of embodiments 1 to 38, having the configuration of T Cell Activator 10 as set forth in Section 6.2.

63. A multispecific molecule according to any one of embodiments 1 to 38, having

64. A multispecific molecule according to any one of embodiments 1 to 38, having the configuration of T Cell Activator 12 as set forth in Section 6.2.

65. A multispecific molecule according to any one of embodiments 1 to 38, having the configuration of T Cell Activator 13 as set forth in Section 6.2.

66. A multispecific molecule according to any one of embodiments 1 to 38, having the configuration of T Cell Activator 14 as set forth in Section 6.2.

67. A multispecific molecule according to any one of embodiments 1 to 38, having the configuration of T Cell Activator 15 as set forth in Section 6.2.

68. A multispecific molecule according to any one of embodiments 1 to 38, having the configuration of T Cell Activator 16 as set forth in Section 6.2.

69. A multispecific molecule according to any one of embodiments 1 to 38, having the configuration of T Cell Activator 17 as set forth in Section 6.2.

70. A multispecific molecule according to any one of embodiments 1 to 38, having the configuration of T Cell Activator 18 as set forth in Section 6.2.

71. A multispecific molecule according to any one of embodiments 1 to 38, having the configuration of T Cell Activator 19 as set forth in Section 6.2.

72. A multispecific molecule according to any one of embodiments 1 to 38, having the configuration of T Cell Activator 20 as set forth in Section 6.2.

73. A multispecific molecule according to any one of embodiments 1 to 38, having the configuration of T Cell Activator 21 as set forth in Section 6.2.

74. A multispecific molecule according to any one of embodiments 1 to 38, having the configuration of T Cell Activator 22 as set forth in Section 6.2.

75. A multispecific molecule according to any one of embodiments 1 to 38, having

76. A multispecific molecule according to any one of embodiments 1 to 38, having the configuration of T Cell Activator 24 as set forth in Section 6.2.

77. A multispecific molecule according to any one of embodiments 1 to 38, having the configuration of T Cell Activator 25 as set forth in Section 6.2.

78. A multispecific molecule according to any one of embodiments 1 to 38, having the configuration of T Cell Activator 26 as set forth in Section 6.2.

79. A multispecific molecule according to any one of embodiments 1 to 38, having the configuration of T Cell Activator 27 as set forth in Section 6.2.

80. A multispecific molecule according to any one of embodiments 1 to 38, having the configuration of T Cell Activator 28 as set forth in Section 6.2.

81. A multispecific molecule according to any one of embodiments 1 to 38, having the configuration of T Cell Activator 29 as set forth in Section 6.2.

82. A multispecific molecule according to any one of embodiments 1 to 38, having the configuration of T Cell Activator 30 as set forth in Section 6.2.

83. A multispecific molecule according to any one of embodiments 1 to 38, having the configuration of T Cell Activator 31 as set forth in Section 6.2.

84. A multispecific molecule according to any one of embodiments 1 to 38, having the configuration of T Cell Activator 32 as set forth in Section 6.2.

85. A multispecific molecule according to any one of embodiments 1 to 38, having the configuration of T Cell Activator 33 as set forth in Section 6.2.

86. A multispecific molecule according to any one of embodiments 1 to 38, having the configuration of T Cell Activator 34 as set forth in Section 6.2.

87. A multispecific molecule according to any one of embodiments 1 to 38, having

88. A multispecific molecule according to any one of embodiments 1 to 38, having the configuration of T Cell Activator 36 as set forth in Section 6.2.

89. A multispecific molecule according to any one of embodiments 1 to 38, having the configuration of T Cell Activator 37 as set forth in Section 6.2.

90. A multispecific molecule according to any one of embodiments 1 to 38, having the configuration of T Cell Activator 38 as set forth in Section 6.2.

91. A multispecific molecule according to any one of embodiments 1 to 38, having the configuration of T Cell Activator 39 as set forth in Section 6.2.

92. A multispecific molecule according to any one of embodiments 1 to 38, having the configuration of T Cell Activator 40 as set forth in Section 6.2.

93. A multispecific molecule according to any one of embodiments 1 to 38, having the configuration of T Cell Activator 41 as set forth in Section 6.2.

94. A multispecific molecule according to any one of embodiments 1 to 38, having the configuration of T Cell Activator 42 as set forth in Section 6.2.

95. A multispecific molecule according to any one of embodiments 1 to 38, having the configuration of T Cell Activator 43 as set forth in Section 6.2.

96. A multispecific molecule according to any one of embodiments 1 to 38, having the configuration of T Cell Activator 44 as set forth in Section 6.2.

97. A multispecific molecule according to any one of embodiments 1 to 38, having the configuration of T Cell Activator 45 as set forth in Section 6.2.

98. A multispecific molecule according to any one of embodiments 1 to 38, having the configuration of T Cell Activator 46 as set forth in Section 6.2.

99. A multispecific molecule according to any one of embodiments 1 to 38, having

100. A multispecific molecule according to any one of embodiments 1 to 38, having the configuration of T Cell Activator 48 as set forth in Section 6.2.

101. A multispecific molecule according to any one of embodiments 1 to 38, having the configuration of T Cell Activator 49 as set forth in Section 6.2.

102. A multispecific molecule according to any one of embodiments 1 to 38, having the configuration of T Cell Activator 50 as set forth in Section 6.2.

103. A multispecific molecule according to any one of embodiments 1 to 38, having the configuration of T Cell Activator 51 as set forth in Section 6.2.

104. A multispecific molecule according to any one of embodiments 1 to 38, having the configuration of T Cell Activator 52 as set forth in Section 6.2.

105. A multispecific molecule according to any one of embodiments 1 to 38, having the configuration of T Cell Activator 53 as set forth in Section 6.2.

106. A multispecific molecule according to any one of embodiments 1 to 38, having the configuration of T Cell Activator 54 as set forth in Section 6.2.

107. A multispecific molecule according to any one of embodiments 1 to 38, having the configuration of T Cell Activator 55 as set forth in Section 6.2.

108. A multispecific molecule according to any one of embodiments 1 to 38, having the configuration of T Cell Activator 56 as set forth in Section 6.2.

109. A multispecific molecule according to any one of embodiments 1 to 38, having the configuration of T Cell Activator 57 as set forth in Section 6.2.

110. A multispecific molecule according to any one of embodiments 1 to 38, having the configuration of T Cell Activator 58 as set forth in Section 6.2.

111. A multispecific molecule according to any one of embodiments 1 to 38, having

112. A multispecific molecule according to any one of embodiments 1 to 38, having the configuration of T Cell Activator 60 as set forth in Section 6.2.

113. A multispecific molecule according to any one of embodiments 1 to 38, having the configuration of T Cell Activator 61 as set forth in Section 6.2.

114. A multispecific molecule according to any one of embodiments 1 to 38, having the configuration of T Cell Activator 62 as set forth in Section 6.2.

115. A multispecific molecule according to any one of embodiments 1 to 38, having the configuration of T Cell Activator 63 as set forth in Section 6.2.

116. A multispecific molecule according to any one of embodiments 1 to 38, having the configuration of T Cell Activator 64 as set forth in Section 6.2.

117. A multispecific molecule according to any one of embodiments 1 to 38, having the configuration of T Cell Activator 65 as set forth in Section 6.2.

118. A multispecific molecule according to any one of embodiments 1 to 38, having the configuration of T Cell Activator 66 as set forth in Section 6.2.

119. A multispecific molecule according to any one of embodiments 1 to 38, having the configuration of T Cell Activator 67 as set forth in Section 6.2.

120. A multispecific molecule according to any one of embodiments 1 to 38, having the configuration of T Cell Activator 68 as set forth in Section 6.2.

121. A multispecific molecule according to any one of embodiments 1 to 38, having the configuration of T Cell Activator 69 as set forth in Section 6.2.

122. A multispecific molecule according to any one of embodiments 1 to 38, having the configuration of T Cell Activator 70 as set forth in Section 6.2.

123. A multispecific molecule according to any one of embodiments 1 to 38, having

124. A multispecific molecule according to any one of embodiments 1 to 38, having the configuration of T Cell Activator 72 as set forth in Section 6.2.

125. A multispecific molecule according to any one of embodiments 1 to 38, having the configuration of T Cell Activator 73 as set forth in Section 6.2.

126. A multispecific molecule according to any one of embodiments 1 to 38, having the configuration of T Cell Activator 74 as set forth in Section 6.2.

127. A multispecific molecule according to any one of embodiments 1 to 38, having the configuration of T Cell Activator 75 as set forth in Section 6.2.

128. A multispecific molecule according to any one of embodiments 1 to 38, having the configuration of T Cell Activator 76 as set forth in Section 6.2.

129. A multispecific molecule according to any one of embodiments 1 to 38, having the configuration of T Cell Activator 77 as set forth in Section 6.2.

130. A multispecific molecule according to any one of embodiments 1 to 38, having the configuration of T Cell Activator 78 as set forth in Section 6.2.

131. A multispecific molecule according to any one of embodiments 1 to 38, having the configuration of T Cell Activator 79 as set forth in Section 6.2.

132. A multispecific molecule according to any one of embodiments 1 to 38, having the configuration of T Cell Activator 80 as set forth in Section 6.2.

133. A multispecific molecule according to any one of embodiments 1 to 38, having the configuration of T Cell Activator 81 as set forth in Section 6.2.

134. A multispecific molecule according to any one of embodiments 1 to 38, having the configuration of T Cell Activator 82 as set forth in Section 6.2.

135. A multispecific molecule according to any one of embodiments 1 to 38, having

136. A multispecific molecule according to any one of embodiments 1 to 38, having the configuration of T Cell Activator 84 as set forth in Section 6.2.

137. A multispecific molecule according to any one of embodiments 1 to 38, having the configuration of T Cell Activator 85 as set forth in Section 6.2.

138. A multispecific molecule according to any one of embodiments 1 to 38, having the configuration of T Cell Activator 86 as set forth in Section 6.2.

139. A multispecific molecule according to any one of embodiments 1 to 38, having the configuration of T Cell Activator 87 as set forth in Section 6.2.

140. A multispecific molecule according to any one of embodiments 1 to 38, having the configuration of T Cell Activator 88 as set forth in Section 6.2.

141. A multispecific molecule according to any one of embodiments 1 to 38, having the configuration of T Cell Activator 89 as set forth in Section 6.2.

142. A multispecific molecule according to any one of embodiments 1 to 38, having the configuration of T Cell Activator 90 as set forth in Section 6.2.

143. A multispecific molecule according to any one of embodiments 1 to 38, having the configuration of T Cell Activator 91 as set forth in Section 6.2.

144. A multispecific molecule according to any one of embodiments 1 to 38, having the configuration of T Cell Activator 92 as set forth in Section 6.2.

145. A multispecific molecule according to any one of embodiments 1 to 38, having the configuration of T Cell Activator 93 as set forth in Section 6.2.

146. A multispecific molecule according to any one of embodiments 1 to 38, having the configuration of T Cell Activator 94 as set forth in Section 6.2.

147. A multispecific molecule according to any one of embodiments 1 to 38, having

148. A multispecific molecule according to any one of embodiments 1 to 38, having the configuration of T Cell Activator 96 as set forth in Section 6.2.

149. A multispecific molecule according to any one of embodiments 1 to 38, having the configuration of T Cell Activator 97 as set forth in Section 6.2.

150. A multispecific molecule according to any one of embodiments 1 to 38, having the configuration of T Cell Activator 98 as set forth in Section 6.2.

151. A multispecific molecule according to any one of embodiments 1 to 38, having the configuration of T Cell Activator 99 as set forth in Section 6.2.

152. A multispecific molecule according to any one of embodiments 1 to 38, having the configuration of T Cell Activator 100 as set forth in Section 6.2.

153. A multispecific molecule according to any one of embodiments 1 to 38, having the configuration of T Cell Activator 101 as set forth in Section 6.2.

154. A multispecific molecule according to any one of embodiments 1 to 38, having the configuration of T Cell Activator 102 as set forth in Section 6.2.

155. A multispecific molecule according to any one of embodiments 1 to 38, having the configuration of T Cell Activator 103 as set forth in Section 6.2.

156. A multispecific molecule according to any one of embodiments 1 to 38, having the configuration of T Cell Activator 104 as set forth in Section 6.2.

157. A multispecific molecule according to any one of embodiments 1 to 38, having the configuration of T Cell Activator 105 as set forth in Section 6.2.

158. A multispecific molecule according to any one of embodiments 1 to 38, having the configuration of T Cell Activator 106 as set forth in Section 6.2.

159. A multispecific molecule according to any one of embodiments 1 to 38, having

160. A multispecific molecule according to any one of embodiments 1 to 38, having the configuration of T Cell Activator 108 as set forth in Section 6.2.

161. A multispecific molecule according to any one of embodiments 1 to 38, having the configuration of T Cell Activator 109 as set forth in Section 6.2.

162. A multispecific molecule according to any one of embodiments 1 to 38, having the configuration of T Cell Activator 110 as set forth in Section 6.2.

163. A multispecific molecule according to any one of embodiments 1 to 38, having the configuration of T Cell Activator 111 as set forth in Section 6.2.

164. A multispecific molecule according to any one of embodiments 1 to 38, having the configuration of T Cell Activator 112 as set forth in Section 6.2.

165. A multispecific molecule according to any one of embodiments 1 to 38, having the configuration of T Cell Activator 113 as set forth in Section 6.2.

166. A multispecific molecule according to any one of embodiments 1 to 38, having the configuration of T Cell Activator 114 as set forth in Section 6.2.

167. A multispecific molecule according to any one of embodiments 1 to 38, having the configuration of T Cell Activator 115 as set forth in Section 6.2.

168. A multispecific molecule according to any one of embodiments 1 to 38, having the configuration of T Cell Activator 116 as set forth in Section 6.2.

169. A multispecific molecule according to any one of embodiments 1 to 38, having the configuration of T Cell Activator 117 as set forth in Section 6.2.

170. A multispecific molecule according to any one of embodiments 1 to 38, having the configuration of T Cell Activator 118 as set forth in Section 6.2.

171. A multispecific molecule according to any one of embodiments 1 to 38, having

172. A multispecific molecule according to any one of embodiments 1 to 38, having the configuration of T Cell Activator 120 as set forth in Section 6.2.

173. A multispecific molecule according to any one of embodiments 1 to 38, having the configuration of T Cell Activator 121 as set forth in Section 6.2.

174. A multispecific molecule according to any one of embodiments 1 to 38, having the configuration of T Cell Activator 122 as set forth in Section 6.2.

175. A multispecific molecule according to any one of embodiments 1 to 38, having the configuration of T Cell Activator 123 as set forth in Section 6.2.

176. A multispecific molecule according to any one of embodiments 1 to 38, having the configuration of T Cell Activator 124 as set forth in Section 6.2.

177. A multispecific molecule according to any one of embodiments 1 to 38, having the configuration of T Cell Activator 125 as set forth in Section 6.2.

178. A multispecific molecule according to any one of embodiments 1 to 38, having the configuration of T Cell Activator 126 as set forth in Section 6.2.

179. A multispecific molecule according to any one of embodiments 1 to 38, having the configuration of T Cell Activator 127 as set forth in Section 6.2.

180. A multispecific molecule according to any one of embodiments 1 to 38, having the configuration of T Cell Activator 128 as set forth in Section 6.2.

181. A multispecific molecule according to any one of embodiments 1 to 38, having the configuration of T Cell Activator 129 as set forth in Section 6.2.

182. A multispecific molecule according to any one of embodiments 1 to 38, having the configuration of T Cell Activator 130 as set forth in Section 6.2.

183. A multispecific molecule according to any one of embodiments 1 to 38, having

184. A multispecific molecule according to any one of embodiments 1 to 38, having the configuration of T Cell Activator 132 as set forth in Section 6.2.

185. A multispecific molecule according to any one of embodiments 1 to 38, having the configuration of T Cell Activator 133 as set forth in Section 6.2.

186. A multispecific molecule according to any one of embodiments 1 to 38, having the configuration of T Cell Activator 134 as set forth in Section 6.2.

187. A multispecific molecule according to any one of embodiments 1 to 38, having the configuration of T Cell Activator 135 as set forth in Section 6.2.

188. A multispecific molecule according to any one of embodiments 1 to 38, having the configuration of T Cell Activator 136 as set forth in Section 6.2.

189. A multispecific molecule according to any one of embodiments 1 to 38, having the configuration of T Cell Activator 137 as set forth in Section 6.2.

190. A multispecific molecule according to any one of embodiments 1 to 38, having the configuration of T Cell Activator 138 as set forth in Section 6.2.

191. A multispecific molecule according to any one of embodiments 1 to 38, having the configuration of T Cell Activator 139 as set forth in Section 6.2.

192. A multispecific molecule according to any one of embodiments 1 to 38, having the configuration of T Cell Activator 140 as set forth in Section 6.2.

193. A multispecific molecule according to any one of embodiments 1 to 38, having the configuration of T Cell Activator 141 as set forth in Section 6.2.

194. A multispecific molecule according to any one of embodiments 1 to 38, having the configuration of T Cell Activator 142 as set forth in Section 6.2.

195. A multispecific molecule according to any one of embodiments 1 to 38, having

196. A multispecific molecule according to any one of embodiments 1 to 38, having the configuration of T Cell Activator 144 as set forth in Section 6.2.

197. A multispecific molecule according to any one of embodiments 1 to 38, having the configuration of T Cell Activator 145 as set forth in Section 6.2.

198. A multispecific molecule according to any one of embodiments 1 to 38, having the configuration of T Cell Activator 146 as set forth in Section 6.2.

199. A multispecific molecule according to any one of embodiments 1 to 38, having the configuration of T Cell Activator 147 as set forth in Section 6.2.

200. A multispecific molecule according to any one of embodiments 1 to 38, having the configuration of T Cell Activator 148 as set forth in Section 6.2.

201. A multispecific molecule according to any one of embodiments 1 to 38, having the configuration of T Cell Activator 149 as set forth in Section 6.2.

202. A multispecific molecule according to any one of embodiments 1 to 38, having the configuration of T Cell Activator 150 as set forth in Section 6.2.

203. A multispecific molecule according to any one of embodiments 1 to 38, having the configuration of T Cell Activator 151 as set forth in Section 6.2.

204. A multispecific molecule according to any one of embodiments 1 to 38, having the configuration of T Cell Activator 152 as set forth in Section 6.2.

205. A multispecific molecule according to any one of embodiments 1 to 38, having the configuration of T Cell Activator 153 as set forth in Section 6.2.

206. A multispecific molecule according to any one of embodiments 1 to 38, having the configuration of T Cell Activator 154 as set forth in Section 6.2.

207. A multispecific molecule according to any one of embodiments 1 to 38, having

208. A multispecific molecule according to any one of embodiments 1 to 38, having the configuration of T Cell Activator 156 as set forth in Section 6.2.

209. A multispecific molecule according to any one of embodiments 1 to 38, having the configuration of T Cell Activator 157 as set forth in Section 6.2.

210. A multispecific molecule according to any one of embodiments 1 to 38, having the configuration of T Cell Activator 158 as set forth in Section 6.2.

211. A multispecific molecule according to any one of embodiments 1 to 38, having the configuration of T Cell Activator 159 as set forth in Section 6.2.

212. A multispecific molecule according to any one of embodiments 1 to 38, having the configuration of T Cell Activator 160 as set forth in Section 6.2.

213. A multispecific molecule according to any one of embodiments 1 to 38, having the configuration of T Cell Activator 161 as set forth in Section 6.2.

214. A multispecific molecule according to any one of embodiments 1 to 38, having the configuration of T Cell Activator 162 as set forth in Section 6.2.

215. A multispecific molecule according to any one of embodiments 1 to 38, having the configuration of T Cell Activator 163 as set forth in Section 6.2.

216. A multispecific molecule according to any one of embodiments 1 to 38, having the configuration of T Cell Activator 164 as set forth in Section 6.2.

217. A multispecific molecule according to any one of embodiments 1 to 38, having the configuration of T Cell Activator 165 as set forth in Section 6.2.

218. A multispecific molecule according to any one of embodiments 1 to 38, having the configuration of T Cell Activator 166 as set forth in Section 6.2.

219. A multispecific molecule according to any one of embodiments 1 to 38, having

220. A multispecific molecule according to any one of embodiments 1 to 38, having the configuration of T Cell Activator 168 as set forth in Section 6.2.

221. A multispecific molecule according to any one of embodiments 1 to 38, having the configuration of T Cell Activator 169 as set forth in Section 6.2.

222. A multispecific molecule according to any one of embodiments 1 to 38, having the configuration of T Cell Activator 170 as set forth in Section 6.2.

223. A multispecific molecule according to any one of embodiments 1 to 38, having the configuration of T Cell Activator 171 as set forth in Section 6.2.

224. A multispecific molecule according to any one of embodiments 1 to 38, having the configuration of T Cell Activator 172 as set forth in Section 6.2.

225. A multispecific molecule according to any one of embodiments 1 to 38, having the configuration of T Cell Activator 173 as set forth in Section 6.2.

226. A multispecific molecule according to any one of embodiments 1 to 38, having the configuration of T Cell Activator 174 as set forth in Section 6.2.

227. A multispecific molecule according to any one of embodiments 1 to 38, having the configuration of T Cell Activator 175 as set forth in Section 6.2.

228. A multispecific molecule according to any one of embodiments 1 to 38, having the configuration of T Cell Activator 176 as set forth in Section 6.2.

229. A multispecific molecule according to any one of embodiments 1 to 38, having the configuration of T Cell Activator 177 as set forth in Section 6.2.

230. A multispecific molecule according to any one of embodiments 1 to 38, having the configuration of T Cell Activator 178 as set forth in Section 6.2.

231. A multispecific molecule according to any one of embodiments 1 to 38, having

232. A multispecific molecule according to any one of embodiments 1 to 38, having the configuration of T Cell Activator 180 as set forth in Section 6.2.

233. A multispecific molecule according to any one of embodiments 1 to 38, having the configuration of T Cell Activator 181 as set forth in Section 6.2.

234. A multispecific molecule according to any one of embodiments 1 to 38, having the configuration of T Cell Activator 182 as set forth in Section 6.2.

235. A multispecific molecule according to any one of embodiments 1 to 38, having the configuration of T Cell Activator 183 as set forth in Section 6.2.

236. A multispecific molecule according to any one of embodiments 1 to 38, having the configuration of T Cell Activator 184 as set forth in Section 6.2.

237. A multispecific molecule according to any one of embodiments 1 to 38, having the configuration of T Cell Activator 185 as set forth in Section 6.2.

238. A multispecific molecule according to any one of embodiments 1 to 38, having the configuration of T Cell Activator 186 as set forth in Section 6.2.

239. A multispecific molecule according to any one of embodiments 1 to 38, having the configuration of T Cell Activator 187 as set forth in Section 6.2.

240. A multispecific molecule according to any one of embodiments 1 to 38, having the configuration of T Cell Activator 188 as set forth in Section 6.2.

241. A multispecific molecule according to any one of embodiments 1 to 38, having the configuration of T Cell Activator 189 as set forth in Section 6.2.

242. A multispecific molecule according to any one of embodiments 1 to 38, having the configuration of T Cell Activator 190 as set forth in Section 6.2.

243. A multispecific molecule according to any one of embodiments 1 to 38, having

244. A multispecific molecule according to any one of embodiments 1 to 38, having the configuration of T Cell Activator 192 as set forth in Section 6.2.

245. A multispecific molecule according to any one of embodiments 1 to 38, having the configuration of T Cell Activator 193 as set forth in Section 6.2.

246. A multispecific molecule according to any one of embodiments 1 to 38, having the configuration of T Cell Activator 194 as set forth in Section 6.2.

247. A multispecific molecule according to any one of embodiments 1 to 38, having the configuration of T Cell Activator 195 as set forth in Section 6.2.

248. A multispecific molecule according to any one of embodiments 1 to 38, having the configuration of T Cell Activator 196 as set forth in Section 6.2.

249. A multispecific molecule according to any one of embodiments 1 to 38, having the configuration of T Cell Activator 197 as set forth in Section 6.2.

250. A multispecific molecule according to any one of embodiments 1 to 38, having the configuration of T Cell Activator 198 as set forth in Section 6.2.

251. A multispecific molecule according to any one of embodiments 1 to 38, having the configuration of T Cell Activator 199 as set forth in Section 6.2.

252. A multispecific molecule according to any one of embodiments 1 to 38, having the configuration of T Cell Activator 200 as set forth in Section 6.2.

253. A multispecific molecule according to any one of embodiments 1 to 38, having the configuration of T Cell Activator 201 as set forth in Section 6.2.

254. A multispecific molecule according to any one of embodiments 1 to 38, having the configuration of T Cell Activator 202 as set forth in Section 6.2.

255. A multispecific molecule according to any one of embodiments 1 to 38, having

256. A multispecific molecule according to any one of embodiments 1 to 38, having the configuration of T Cell Activator 204 as set forth in Section 6.2.

257. A multispecific molecule according to any one of embodiments 1 to 38, having the configuration of T Cell Activator 205 as set forth in Section 6.2.

258. A multispecific molecule according to any one of embodiments 1 to 38, having the configuration of T Cell Activator 206 as set forth in Section 6.2.

259. A multispecific molecule according to any one of embodiments 1 to 38, having the configuration of T Cell Activator 207 as set forth in Section 6.2.

260. A multispecific molecule according to any one of embodiments 1 to 38, having the configuration of T Cell Activator 208 as set forth in Section 6.2.

261. A multispecific molecule according to any one of embodiments 1 to 38, having the configuration of T Cell Activator 209 as set forth in Section 6.2.

262. A multispecific molecule according to any one of embodiments 1 to 38, having the configuration of T Cell Activator 210 as set forth in Section 6.2.

263. A multispecific molecule according to any one of embodiments 1 to 38, having the configuration of T Cell Activator 211 as set forth in Section 6.2.

264. A multispecific molecule according to any one of embodiments 1 to 38, having the configuration of T Cell Activator 212 as set forth in Section 6.2.

265. A multispecific molecule according to any one of embodiments 1 to 38, having the configuration of T Cell Activator 213 as set forth in Section 6.2.

266. A multispecific molecule according to any one of embodiments 1 to 38, having the configuration of T Cell Activator 214 as set forth in Section 6.2.

267. A multispecific molecule according to any one of embodiments 1 to 38, having

268. A multispecific molecule according to any one of embodiments 1 to 38, having the configuration of T Cell Activator 216 as set forth in Section 6.2.

269. A multispecific molecule according to any one of embodiments 1 to 38, having the configuration of T Cell Activator 217 as set forth in Section 6.2.

270. A multispecific molecule according to any one of embodiments 1 to 38, having the configuration of T Cell Activator 218 as set forth in Section 6.2.

271. A multispecific molecule according to any one of embodiments 1 to 38, having the configuration of T Cell Activator 219 as set forth in Section 6.2.

272. A multispecific molecule according to any one of embodiments 1 to 38, having the configuration of T Cell Activator 220 as set forth in Section 6.2.

273. A multispecific molecule according to any one of embodiments 1 to 38, having the configuration of T Cell Activator 221 as set forth in Section 6.2.

274. A multispecific molecule according to any one of embodiments 1 to 38, having the configuration of T Cell Activator 222 as set forth in Section 6.2.

275. A multispecific molecule according to any one of embodiments 1 to 38, having the configuration of T Cell Activator 223 as set forth in Section 6.2.

276. A multispecific molecule according to any one of embodiments 1 to 38, having the configuration of T Cell Activator 224 as set forth in Section 6.2.

277. A multispecific molecule according to any one of embodiments 1 to 38, having the configuration of T Cell Activator 225 as set forth in Section 6.2.

278. A multispecific molecule according to any one of embodiments 1 to 38, having the configuration of T Cell Activator 226 as set forth in Section 6.2.

279. A multispecific molecule according to any one of embodiments 1 to 38, having

280. A multispecific molecule according to any one of embodiments 1 to 38, having the configuration of T Cell Activator 228 as set forth in Section 6.2.

281. A multispecific molecule according to any one of embodiments 1 to 38, having the configuration of T Cell Activator 229 as set forth in Section 6.2.

282. A multispecific molecule according to any one of embodiments 1 to 38, having the configuration of T Cell Activator 230 as set forth in Section 6.2.

283. A multispecific molecule according to any one of embodiments 1 to 38, having the configuration of T Cell Activator 231 as set forth in Section 6.2.

284. A multispecific molecule according to any one of embodiments 1 to 38, having the configuration of T Cell Activator 232 as set forth in Section 6.2.

285. A multispecific molecule according to any one of embodiments 1 to 38, having the configuration of T Cell Activator 233 as set forth in Section 6.2.

286. A multispecific molecule according to any one of embodiments 1 to 38, having the configuration of T Cell Activator 234 as set forth in Section 6.2.

287. A multispecific molecule according to any one of embodiments 1 to 38, having the configuration of T Cell Activator 235 as set forth in Section 6.2.

288. A multispecific molecule according to any one of embodiments 1 to 38, having the configuration of T Cell Activator 236 as set forth in Section 6.2.

289. A multispecific molecule according to any one of embodiments 1 to 38, having the configuration of T Cell Activator 237 as set forth in Section 6.2.

290. A multispecific molecule according to any one of embodiments 1 to 38, having the configuration of T Cell Activator 238 as set forth in Section 6.2.

291. A multispecific molecule according to any one of embodiments 1 to 38, having

292. A multispecific molecule according to any one of embodiments 1 to 38, having the configuration of T Cell Activator 240 as set forth in Section 6.2.

293. A multispecific molecule according to any one of embodiments 1 to 38, having the configuration of T Cell Activator 241 as set forth in Section 6.2.

294. A multispecific molecule according to any one of embodiments 1 to 38, having the configuration of T Cell Activator 242 as set forth in Section 6.2.

295. A multispecific molecule according to any one of embodiments 1 to 38, having the configuration of T Cell Activator 243 as set forth in Section 6.2.

296. A multispecific molecule according to any one of embodiments 1 to 38, having the configuration of T Cell Activator 244 as set forth in Section 6.2.

297. A multispecific molecule according to any one of embodiments 1 to 38, having the configuration of T Cell Activator 245 as set forth in Section 6.2.

298. A multispecific molecule according to any one of embodiments 1 to 38, having the configuration of T Cell Activator 246 as set forth in Section 6.2.

299. A multispecific molecule according to any one of embodiments 1 to 38, having the configuration of T Cell Activator 247 as set forth in Section 6.2.

300. A multispecific molecule according to any one of embodiments 1 to 38, having the configuration of T Cell Activator 248 as set forth in Section 6.2.

301. A multispecific molecule according to any one of embodiments 1 to 38, having the configuration of T Cell Activator 249 as set forth in Section 6.2.

302. A multispecific molecule according to any one of embodiments 1 to 38, having the configuration of T Cell Activator 250 as set forth in Section 6.2.

303. A multispecific molecule according to any one of embodiments 1 to 38, having

304. A multispecific molecule according to any one of embodiments 1 to 38, having the configuration of T Cell Activator 252 as set forth in Section 6.2.

305. A multispecific molecule according to any one of embodiments 1 to 38, having the configuration of T Cell Activator 253 as set forth in Section 6.2.

306. A multispecific molecule according to any one of embodiments 1 to 38, having the configuration of T Cell Activator 254 as set forth in Section 6.2.

307. A multispecific molecule according to any one of embodiments 1 to 38, having the configuration of T Cell Activator 255 as set forth in Section 6.2.

308. A multispecific molecule according to any one of embodiments 1 to 38, having the configuration of T Cell Activator 256 as set forth in Section 6.2.

309. A multispecific molecule according to any one of embodiments 1 to 38, having the configuration of T Cell Activator 257 as set forth in Section 6.2.

310. A multispecific molecule according to any one of embodiments 1 to 38, having the configuration of T Cell Activator 258 as set forth in Section 6.2.

311. A multispecific molecule according to any one of embodiments 1 to 38, having the configuration of T Cell Activator 259 as set forth in Section 6.2.

312. A multispecific molecule according to any one of embodiments 1 to 38, having the configuration of T Cell Activator 260 as set forth in Section 6.2.

313. A multispecific molecule according to any one of embodiments 1 to 38, having the configuration of T Cell Activator 261 as set forth in Section 6.2.

314. A multispecific molecule according to any one of embodiments 1 to 38, having the configuration of T Cell Activator 262 as set forth in Section 6.2.

315. A multispecific molecule according to any one of embodiments 1 to 38, having

316. A multispecific molecule according to any one of embodiments 1 to 38, having the configuration of T Cell Activator 264 as set forth in Section 6.2.

317. A multispecific molecule according to any one of embodiments 1 to 38, having the configuration of T Cell Activator 265 as set forth in Section 6.2.

318. A multispecific molecule according to any one of embodiments 1 to 38, having the configuration of T Cell Activator 266 as set forth in Section 6.2.

319. A multispecific molecule according to any one of embodiments 1 to 38, having the configuration of T Cell Activator 267 as set forth in Section 6.2.

320. A multispecific molecule according to any one of embodiments 1 to 38, having the configuration of T Cell Activator 268 as set forth in Section 6.2.

321. A multispecific molecule according to any one of embodiments 1 to 38, having the configuration of T Cell Activator 269 as set forth in Section 6.2.

322. A multispecific molecule according to any one of embodiments 1 to 38, having the configuration of T Cell Activator 270 as set forth in Section 6.2.

323. A multispecific molecule according to any one of embodiments 1 to 38, having the configuration of T Cell Activator 271 as set forth in Section 6.2.

324. A multispecific molecule according to any one of embodiments 1 to 38, having the configuration of T Cell Activator 272 as set forth in Section 6.2.

325. A multispecific molecule according to any one of embodiments 1 to 38, having the configuration of T Cell Activator 273 as set forth in Section 6.2.

326. A multispecific molecule according to any one of embodiments 1 to 38, having the configuration of T Cell Activator 274 as set forth in Section 6.2.

327. A multispecific molecule according to any one of embodiments 1 to 38, having

328. A multispecific molecule according to any one of embodiments 1 to 38, having the configuration of T Cell Activator 276 as set forth in Section 6.2.

329. A multispecific molecule according to any one of embodiments 1 to 38, having the configuration of T Cell Activator 277 as set forth in Section 6.2.

330. A multispecific molecule according to any one of embodiments 1 to 38, having the configuration of T Cell Activator 278 as set forth in Section 6.2.

331. A multispecific molecule according to any one of embodiments 1 to 38, having the configuration of T Cell Activator 279 as set forth in Section 6.2.

332. A multispecific molecule according to any one of embodiments 1 to 38, having the configuration of T Cell Activator 280 as set forth in Section 6.2.

333. A multispecific molecule according to any one of embodiments 1 to 38, having the configuration of T Cell Activator 281 as set forth in Section 6.2.

334. A multispecific molecule according to any one of embodiments 1 to 38, having the configuration of T Cell Activator 282 as set forth in Section 6.2.

335. A multispecific molecule according to any one of embodiments 1 to 38, having the configuration of T Cell Activator 283 as set forth in Section 6.2.

336. A multispecific molecule according to any one of embodiments 1 to 38, having the configuration of T Cell Activator 284 as set forth in Section 6.2.

337. A multispecific molecule according to any one of embodiments 1 to 38, having the configuration of T Cell Activator 285 as set forth in Section 6.2.

338. A multispecific molecule according to any one of embodiments 1 to 38, having the configuration of T Cell Activator 286 as set forth in Section 6.2.

339. A multispecific molecule according to any one of embodiments 1 to 38, having

340. A multispecific molecule according to any one of embodiments 1 to 38, having the configuration of T Cell Activator 288 as set forth in Section 6.2.

341. A multispecific molecule according to any one of embodiments 1 to 38, having the configuration of T Cell Activator 289 as set forth in Section 6.2.

342. A multispecific molecule according to any one of embodiments 1 to 38, having the configuration of T Cell Activator 290 as set forth in Section 6.2.

343. A multispecific molecule according to any one of embodiments 1 to 38, having the configuration of T Cell Activator 291 as set forth in Section 6.2.

344. A multispecific molecule according to any one of embodiments 1 to 38, having the configuration of T Cell Activator 292 as set forth in Section 6.2.

345. A multispecific molecule according to any one of embodiments 1 to 38, having the configuration of T Cell Activator 293 as set forth in Section 6.2.

346. A multispecific molecule according to any one of embodiments 1 to 38, having the configuration of T Cell Activator 294 as set forth in Section 6.2.

347. A multispecific molecule according to any one of embodiments 1 to 38, having the configuration of T Cell Activator 295 as set forth in Section 6.2.

348. A multispecific molecule according to any one of embodiments 1 to 38, having the configuration of T Cell Activator 296 as set forth in Section 6.2.

349. A multispecific molecule according to any one of embodiments 1 to 38, having the configuration of T Cell Activator 297 as set forth in Section 6.2.

350. A multispecific molecule according to any one of embodiments 1 to 38, having the configuration of T Cell Activator 298 as set forth in Section 6.2.

351. A multispecific molecule according to any one of embodiments 1 to 38, having

352. A multispecific molecule according to any one of embodiments 1 to 38, having the configuration of T Cell Activator 300 as set forth in Section 6.2.

353. A multispecific molecule according to any one of embodiments 1 to 38, having the configuration of T Cell Activator 301 as set forth in Section 6.2.

354. A multispecific molecule according to any one of embodiments 1 to 38, having the configuration of T Cell Activator 302 as set forth in Section 6.2.

355. A multispecific molecule according to any one of embodiments 1 to 38, having the configuration of T Cell Activator 303 as set forth in Section 6.2.

356. A multispecific molecule according to any one of embodiments 1 to 38, having the configuration of T Cell Activator 304 as set forth in Section 6.2.

357. A multispecific molecule according to any one of embodiments 1 to 38, having the configuration of T Cell Activator 305 as set forth in Section 6.2.

358. A multispecific molecule according to any one of embodiments 1 to 38, having the configuration of T Cell Activator 306 as set forth in Section 6.2.

359. A multispecific molecule according to any one of embodiments 1 to 38, having the configuration of T Cell Activator 307 as set forth in Section 6.2.

360. A multispecific molecule according to any one of embodiments 1 to 38, having the configuration of T Cell Activator 308 as set forth in Section 6.2.

361. A multispecific molecule according to any one of embodiments 1 to 38, having the configuration of T Cell Activator 309 as set forth in Section 6.2.

362. A multispecific molecule according to any one of embodiments 1 to 38, having the configuration of T Cell Activator 310 as set forth in Section 6.2.

363. A multispecific molecule according to any one of embodiments 1 to 38, having

364. A multispecific molecule according to any one of embodiments 1 to 38, having the configuration of T Cell Activator 312 as set forth in Section 6.2.

365. A multispecific molecule according to any one of embodiments 1 to 38, having the configuration of T Cell Activator 313 as set forth in Section 6.2.

366. A multispecific molecule according to any one of embodiments 1 to 38, having the configuration of T Cell Activator 314 as set forth in Section 6.2.

367. The multispecific molecule of any one of embodiments 2 to 366, which comprises a first linker connecting the pMHC complex to the multimerization moiety to which it is operably linked.

368. The multispecific molecule of any one of embodiments 2 to 367, which comprises one or more additional linkers, each connecting an ICA targeting moiety (when present) to the multimerization moiety to which it is operably linked.

369. The multispecific molecule of embodiment 367 or 368, wherein the first linker and/or one or more additional linker (or all additional linkers) are 7 or less amino acids in length.

370. The multispecific molecule of embodiment 367 or 368, wherein the first linker and/or one or more additional linker (or all additional linkers) are greater than 7 amino acids in length.

371. The multispecific molecule of any one of embodiments 1 to 370, wherein the ICA targeting moiety is a T cell antigen (TCA) targeting moiety.

372. The multispecific molecule of embodiment 371, wherein the TCA targeting moiety is a T cell receptor (TCR) targeting moiety.

373. The multispecific molecule of embodiment 372, wherein the TCR targeting moiety is a CD3 targeting moiety.

374. The multispecific molecule of embodiment 372, wherein the TCR targeting moiety is a TCRαβ targeting moiety.

375. The multispecific molecule of embodiment 372, wherein the TCR targeting moiety is a TCRγδ targeting moiety.

376. The multispecific molecule of any one of embodiments 372 to 375, wherein the TCR targeting moiety comprises a VH having at least 95%, at least 96%, at least 97%, at least 98%, at least 99%, or 100% identity to the amino acid sequence of SEQ ID NO:231 and a VL having at least 95%, at least 96%, at least 97%, at least 98%, at least 99%, or 100% identity to the amino acid sequence of SEQ ID NO:236.

377. The multispecific molecule of any one of embodiments 372 to 375, wherein the TCR targeting moiety comprises a VH having at least 95%, at least 96%, at least 97%, at least 98%, at least 99%, or 100% identity to the amino acid sequence of SEQ ID NO:232 and a VL having at least 95%, at least 96%, at least 97%, at least 98%, at least 99%, or 100% identity to the amino acid sequence of SEQ ID NO:236.

378. The multispecific molecule of any one of embodiments 372 to 375, wherein the TCR targeting moiety comprises a VH having at least 95%, at least 96%, at least 97%, at least 98%, at least 99%, or 100% identity to the amino acid sequence of SEQ ID NO:233 and a VL having at least 95%, at least 96%, at least 97%, at least 98%, at least 99%, or 100% identity to the amino acid sequence of SEQ ID NO:236.

379. The multispecific molecule of any one of embodiments 372 to 375, wherein the TCR targeting moiety comprises a VH having at least 95%, at least 96%, at least 97%, at least 98%, at least 99%, or 100% identity to the amino acid sequence of SEQ ID NO:234 and a VL having at least 95%, at least 96%, at least 97%, at least 98%, at least 99%, or 100% identity to the amino acid sequence of SEQ ID NO:236.

380. The multispecific molecule of any one of embodiments 372 to 375, wherein the TCR targeting moiety comprises an HCDR1, HCDR2, and HCDR3 of an anti-CD3 antibody of Table G-1.

381. The multispecific molecule of any one of embodiments 372 to 375 or 380, wherein the TCR targeting moiety comprises an LCDR1, LCDR2, and LCDR3 sequence of an anti-CD3 antibody of Table G-1.

382. The multispecific molecule of any one of embodiments 372 to 381, wherein the TCR targeting moiety has a K_{D} for the TCR of greater than 10⁻⁹M.

383. The multispecific molecule of any one of embodiments 372 to 381, wherein the TCR targeting moiety has a K_{D} for the TCR of greater than 10⁻⁸M.

384. The multispecific molecule of any one of embodiments 372 to 381, wherein the TCR targeting moiety has a K_{D} for the TCR of between 10⁻⁶M and 10⁻⁸M.

385. The multispecific molecule of embodiment 371, wherein the TCA targeting moiety is a CD28 targeting moiety.

386. The multispecific molecule of embodiment 385, wherein the CD28 targeting moiety comprises a VH having at least 95%, at least 96%, at least 97%, at least 98%, at least 99%, or 100% identity to the amino acid sequence of SEQ ID NO:237 and a VL having at least 95%, at least 96%, at least 97%, at least 98%, at least 99%, or 100% identity to the amino acid sequence of SEQ ID NO:236.

387. The multispecific molecule of embodiment 385, wherein the CD28 targeting moiety comprises an HCDR1, HCDR2, and HCDR3 of an anti-CD28 antibody of Table G-2.

388. The multispecific molecule of embodiment 385 or 387, wherein the CD28 targeting moiety comprises an LCDR1, LCDR2, and LCDR3 sequence of an anti-CD28 antibody of Table G-2.

389. The multispecific molecule of any one of embodiments 1 to 370, wherein the ICA targeting moiety is a B cell antigen (BCA) targeting moiety.

390. The multispecific molecule of embodiment 389, wherein the BCA targeting moiety is a CD19 targeting moiety.

391. The multispecific molecule of embodiment 390, wherein the BCA targeting moiety comprises an HCDR1, HCDR2, and HCDR3 of an anti-CD19 antibody of Table G-3.

392. The multispecific molecule of embodiment 390 or 391, wherein the BCA targeting moiety comprises an LCDR1, LCDR2, and LCDR3 sequence of an anti-CD19 antibody of Table G-3.

393. The multispecific molecule of embodiment 389, wherein the BCA targeting moiety is a CD20 targeting moiety.

394. The multispecific molecule of embodiment 393, wherein the CD28 targeting moiety comprises a VH having at least 95%, at least 96%, at least 97%, at least 98%, at least 99%, or 100% identity to the amino acid sequence of SEQ ID NO:238 and a VL having at least 95%, at least 96%, at least 97%, at least 98%, at least 99%, or 100% identity to the amino acid sequence of SEQ ID NO:239.

395. The multispecific molecule of embodiment 393, wherein the CD28 targeting moiety comprises a VH having at least 95%, at least 96%, at least 97%, at least 98%, at least 99%, or 100% identity to the amino acid sequence of SEQ ID NO:240 and a VL having at least 95%, at least 96%, at least 97%, at least 98%, at least 99%, or 100% identity to the amino acid sequence of SEQ ID NO:236.

396. The multispecific molecule of embodiment 393, wherein the BCA targeting moiety comprises an HCDR1, HCDR2, and HCDR3 of an anti-CD20 antibody of Table G-4.

397. The multispecific molecule of embodiment 393 or 396, wherein the BCA targeting moiety comprises an LCDR1, LCDR2, and LCDR3 sequence of an anti-CD20 antibody of Table G-4.

398. The multispecific molecule of embodiment 389, wherein the BCA targeting moiety is a CD22 targeting moiety.

399. The multispecific molecule of embodiment 398, wherein the BCA targeting moiety comprises an HCDR1, HCDR2, and HCDR3 of an anti-CD22 antibody of Table G-5.

400. The multispecific molecule of embodiment 398 or 399, wherein the BCA targeting moiety comprises an LCDR1, LCDR2, and LCDR3 sequence of an anti-CD22 antibody of Table G-5.

401. The multispecific molecule of any one of embodiments 1 to 400, further comprising an additional ICA targeting moiety.

402. The multispecific molecule of embodiment 401, wherein the additional ICA targeting moiety is an additional TCA targeting moiety.

403. The multispecific molecule of embodiment 402, wherein the additional TCA targeting moiety is a T cell receptor (TCR) targeting moiety.

404. The multispecific molecule of embodiment 403, wherein the TCR targeting moiety is a CD3 targeting moiety.

405. The multispecific molecule of embodiment 404, wherein the TCR targeting moiety is a TCRαβ targeting moiety.

406. The multispecific molecule of embodiment 404, wherein the TCR targeting moiety is a TCRγδ targeting moiety.

407. The multispecific molecule of any one of embodiments 403 to 406, wherein the TCR targeting moiety comprises a VH having at least 95%, at least 96%, at least 97%, at least 98%, at least 99%, or 100% identity to the amino acid sequence of SEQ ID NO:231 and a VL having at least 95%, at least 96%, at least 97%, at least 98%, at least 99%, or 100% identity to the amino acid sequence of SEQ ID NO:236.

408. The multispecific molecule of any one of embodiments 403 to 406, wherein the TCR targeting moiety comprises a VH having at least 95%, at least 96%, at least 97%, at least 98%, at least 99%, or 100% identity to the amino acid sequence of SEQ ID NO:232 and a VL having at least 95%, at least 96%, at least 97%, at least 98%, at least 99%, or 100% identity to the amino acid sequence of SEQ ID NO:236.

409. The multispecific molecule of any one of embodiments 403 to 406, wherein the TCR targeting moiety comprises a VH having at least 95%, at least 96%, at least 97%, at least 98%, at least 99%, or 100% identity to the amino acid sequence of SEQ ID NO:233 and a VL having at least 95%, at least 96%, at least 97%, at least 98%, at least 99%, or 100% identity to the amino acid sequence of SEQ ID NO:236.

410. The multispecific molecule of any one of embodiments 403 to 406, wherein the TCR targeting moiety comprises a VH having at least 95%, at least 96%, at least 97%, at least 98%, at least 99%, or 100% identity to the amino acid sequence of SEQ ID NO:234 and a VL having at least 95%, at least 96%, at least 97%, at least 98%, at least 99%, or 100% identity to the amino acid sequence of SEQ ID NO:236.

411. The multispecific molecule of any one of embodiments 403 to 406, wherein the TCR targeting moiety comprises an HCDR1, HCDR2, and HCDR3 of an anti-CD3 antibody of Table G-1.

412. The multispecific molecule of any one of embodiments 403 to 406 or 411, wherein the TCR targeting moiety comprises an LCDR1, LCDR2, and LCDR3 sequence of an anti-CD3 antibody of Table G-1.

413. The multispecific molecule of any one of embodiments 403 to 412, wherein the TCR targeting moiety has a K_{D} for the TCR of greater than 10⁻⁹M.

414. The multispecific molecule of any one of embodiments 403 to 412, wherein the TCR targeting moiety has a K_{D} for the TCR of greater than 10⁻⁸M.

415. The multispecific molecule of any one of embodiments 403 to 412, wherein the TCR targeting moiety has a K_{D} for the TCR of between 10⁻⁶M and 10⁻⁸M.

416. The multispecific molecule of embodiment 402, wherein the additional TCA targeting moiety is a CD28 targeting moiety.

417. The multispecific molecule of embodiment 416, wherein the CD28 targeting moiety comprises a VH having at least 95%, at least 96%, at least 97%, at least 98%, at least 99%, or 100% identity to the amino acid sequence of SEQ ID NO:237 and a VL having at least 95%, at least 96%, at least 97%, at least 98%, at least 99%, or 100% identity to the amino acid sequence of SEQ ID NO:236.

418. The multispecific molecule of embodiment 416, wherein the CD28 targeting moiety comprises an HCDR1, HCDR2, and HCDR3 of an anti-CD28 antibody of Table G-2.

419. The multispecific molecule of embodiment 416 or 418, wherein the CD28 targeting moiety comprises an LCDR1, LCDR2, and LCDR3 sequence of an anti-CD28 antibody of Table G-2.

420. The multispecific molecule of embodiment 401, wherein the additional ICA targeting moiety is an additional BCA targeting moiety.

421. The multispecific molecule of embodiment 420, wherein the additional BCA targeting moiety is a CD19 targeting moiety.

422. The multispecific molecule of embodiment 421, wherein the CD19 targeting moiety comprises an HCDR1, HCDR2, and HCDR3 of an anti-CD19 antibody of Table G-3.

423. The multispecific molecule of embodiment 421 or 422, wherein the CD19 targeting moiety comprises an LCDR1, LCDR2, and LCDR3 sequence of an anti-CD19 antibody of Table G-3.

424. The multispecific molecule of embodiment 420, wherein the additional BCA targeting moiety is a CD20 targeting moiety.

425. The multispecific molecule of embodiment 424, wherein the CD28 targeting moiety comprises a VH having at least 95%, at least 96%, at least 97%, at least 98%, at least 99%, or 100% identity to the amino acid sequence of SEQ ID NO:238 and a VL having at least 95%, at least 96%, at least 97%, at least 98%, at least 99%, or 100% identity to the amino acid sequence of SEQ ID NO:239.

426. The multispecific molecule of embodiment 424, wherein the CD28 targeting moiety comprises a VH having at least 95%, at least 96%, at least 97%, at least 98%, at least 99%, or 100% identity to the amino acid sequence of SEQ ID NO:240 and a VL having at least 95%, at least 96%, at least 97%, at least 98%, at least 99%, or 100% identity to the amino acid sequence of SEQ ID NO:236.

427. The multispecific molecule of embodiment 424, wherein the CD20 targeting moiety comprises an HCDR1, HCDR2, and HCDR3 of an anti-CD20 antibody of Table G-4.

428. The multispecific molecule of embodiment 424 or 425, wherein the CD20 targeting moiety comprises an LCDR1, LCDR2, and LCDR3 sequence of an anti-CD20 antibody of Table G-4.

429. The multispecific molecule of embodiment 420, wherein the additional BCA targeting moiety is a CD22 targeting moiety.

430. The multispecific molecule of embodiment 429, wherein the CD22 targeting moiety comprises an HCDR1, HCDR2, and HCDR3 of an anti-CD22 antibody of Table G-5.

431. The multispecific molecule of embodiment 429 or 430, wherein the CD22 targeting moiety comprises an LCDR1, LCDR2, and LCDR3 sequence of an anti-CD22 antibody of Table G-5.

432. The multispecific molecule of any one of embodiments 1 to 431, further comprising a tumor antigen targeting moiety.

433. The multispecific molecule of embodiment 432, wherein the tumor antigen targeting moiety is a tumor-associated antigen (TAA) targeting moiety.

434. The multispecific molecule of embodiment 433, wherein the TAA is CD20, EGFR, FITC, CD19, CD22, CD33, PSMA, GD2, EGFR variants, ROR1, c-Met, HER2, CEA, mesothelin, GM2, CD7, CD10, CD30, CD34, CD38, CD41, CD44, CD74, CD123 CD133, CD171, MUC16, MUC1, CS1 (CD319), IL-13Ra2, BCMA, Lewis Y, IgG kappa chain, folate receptor-alpha, PSCA, or EpCAM.

435. The multispecific molecule of embodiment 435, wherein the TAA is CD20.

436. The multispecific molecule of embodiment 435, wherein the TAA is BCMA.

437. The multispecific molecule of the embodiment 435, wherein the TAA is EGFR.

438. The multispecific molecule of embodiment 435, wherein the TAA is Muc16.

439. The multispecific molecule of embodiment 435, wherein the TAA is PSMA.

440. The multispecific molecule of any one of embodiments 1 to 439, wherein the MHC domain is a type I MHC domain.

441. The multispecific molecule of embodiment 440, wherein the pMHC complex further comprises a β2-microglobulin domain.

442. The multispecific molecule of embodiment 441, wherein the β2-microglobulin domain comprises an amino acid sequence having at least at least 95%, at least 96%, at least 97%, at least 98%, at least 99%, or 100% identity to the amino acid sequence of SEQ ID NO:250.

443. The multispecific molecule of embodiment 441, wherein the β2-microglobulin domain comprises the amino acid sequence of SEQ ID NO:250.

444. The multispecific molecule of any one of embodiments 441 to 443, wherein the pMHC complex further comprises a linker connecting the antigenic peptide and the β2-microglobulin domain.

445. The multispecific molecule of embodiment 442, wherein the pMHC complex further comprises an additional linker connecting the β2-microglobulin domain and the MHC domain.

446. The multispecific molecule of any one of embodiments 1 to 445, wherein the pMHC complex is a single polypeptide.

447. The multispecific molecule of embodiment 446, wherein the pMHC complex comprises, in N- to C-terminal orientation, an MHC peptide, a linker, a 32-microglobulin domain, a linker, and a type I MHC domain.

448. The multispecific molecule of any one of embodiments 1 to 447, wherein the multispecific molecule comprises a polypeptide comprising, in N- to C-terminal orientation, the pMHC complex and an Fc domain.

449. The multispecific molecule of any one of embodiments 1 to 448, wherein the multispecific molecule comprises a polypeptide comprising, in N- to C-terminal orientation, the pMHC complex, an Fc domain, and the ICA targeting moiety.

450. The multispecific molecule of any one of embodiments 1 to 449, further comprising an additional pMHC complex comprising an additional MHC domain and an additional antigenic peptide.

451. The multispecific molecule of embodiment 450, wherein the additional MHC domain and the additional antigenic peptide are the same as the MHC domain and the antigenic peptide.

452. The multispecific molecule of embodiment 450 or embodiment 451, wherein the additional pMHC complex is on a separate polypeptide from the pMHC complex.

453. A multispecific molecule comprising:
(a) a first polypeptide chain comprising:
   (i) a peptide-MHC (pMHC) complex comprising an MHC domain and an antigenic peptide; and
   (ii) a first Fc domain; and
(b) a second polypeptide chain comprising:
   (i) an immune cell antigen (ICA) targeting moiety or component thereof; and
   (iii) a second Fc domain that associates with the first Fc domain to form an Fc region.

454. The multispecific molecule of embodiment 453, wherein the second polypeptide chain comprises the ICA targeting moiety, wherein the ICA targeting moiety is an scFv.

455. The multispecific molecule of embodiment 453 or embodiment 454, wherein the second polypeptide comprises a first ICA targeting moiety component, wherein the multispecific molecule further comprises a third polypeptide chain comprising a second ICA targeting moiety component configured to associate with the first ICA targeting moiety component to form the ICA targeting moiety.

456. The multispecific molecule of embodiment 455, wherein the first ICA targeting moiety component is a VH and the second ICA targeting moiety component is a VL configured to associate with the VH.

457. The multispecific molecule of any one of embodiments 453 to 456, wherein the pMHC complex is N-terminal to the first Fc domain.

458. The multispecific molecule of any one of embodiments 453 to 456, wherein the pMHC complex is C-terminal to the first Fc domain.

459. The multispecific molecule of any one of embodiments 453 to 458, wherein the ICA targeting moiety or component thereof is N-terminal to the second Fc domain.

460. The multispecific molecule of any one of embodiments 453 to 458, wherein the ICA targeting moiety or component thereof is C-terminal to the second Fc domain.

461. The multispecific molecule of any one of embodiments 453 to 460, wherein the multispecific molecule further comprises a linker connecting the pMHC complex to the first Fc domain.

462. The multispecific molecule of embodiment 461, wherein the multispecific molecule further comprises a linker connecting the ICA targeting moiety to the second Fc domain.

463. The multispecific molecule of embodiment 461 or 462, wherein the linker is 7 or less amino acids in length.

464. The multispecific molecule of embodiment 463, wherein the linker is greater than 7 amino acids in length.

465. The multispecific molecule of any one of embodiments 453 to 464, wherein the ICA targeting moiety is a T cell antigen (TCA) targeting moiety.

466. The multispecific molecule of embodiment 465, wherein the TCA targeting moiety is a T cell receptor (TCR) targeting moiety.

467. The multispecific molecule of embodiment 466, wherein the TCR targeting moiety is a CD3 targeting moiety.

468. The multispecific molecule of embodiment 466, wherein the TCR targeting moiety is a TCRαβ targeting moiety.

469. The multispecific molecule of embodiment 466, wherein the TCR targeting moiety is a TCRγδ targeting moiety.

470. The multispecific molecule of any one of embodiments 466 to 469, wherein the TCR targeting moiety has a K_{D} for the TCR of greater than 10⁻⁹M.

471. The multispecific molecule of any one of embodiments 466 to 469, wherein the TCR targeting moiety has a K_{D} for the TCR of greater than 10⁻⁸M.

472. The multispecific molecule of any one of embodiments 466 to 469, wherein the TCR targeting moiety has a K_{D} for the TCR of between 10⁻⁶M and 10⁻⁸M.

473. The multispecific molecule of any one of embodiments 466 to 472, wherein the TCR targeting moiety comprises a VH having at least 95%, at least 96%, at least 97%, at least 98%, at least 99%, or 100% identity to the amino acid sequence of SEQ ID NO:231 and a VL having at least 95%, at least 96%, at least 97%, at least 98%, at least 99%, or 100% identity to the amino acid sequence of SEQ ID NO:236.

474. The multispecific molecule of any one of embodiments 466 to 472, wherein the TCR targeting moiety comprises a VH having at least 95%, at least 96%, at least 97%, at least 98%, at least 99%, or 100% identity to the amino acid sequence of SEQ ID NO:232 and a VL having at least 95%, at least 96%, at least 97%, at least 98%, at least 99%, or 100% identity to the amino acid sequence of SEQ ID NO:236.

475. The multispecific molecule of any one of embodiments 466 to 472, wherein the TCR targeting moiety comprises a VH having at least 95%, at least 96%, at least 97%, at least 98%, at least 99%, or 100% identity to the amino acid sequence of SEQ ID NO:233 and a VL having at least 95%, at least 96%, at least 97%, at least 98%, at least 99%, or 100% identity to the amino acid sequence of SEQ ID NO:236.

476. The multispecific molecule of any one of embodiments 466 to 472, wherein the TCR targeting moiety comprises a VH having at least 95%, at least 96%, at least 97%, at least 98%, at least 99%, or 100% identity to the amino acid sequence of SEQ ID NO:234 and a VL having at least 95%, at least 96%, at least 97%, at least 98%, at least 99%, or 100% identity to the amino acid sequence of SEQ ID NO:236.

477. The multispecific molecule of any one of embodiments 466 to 472, wherein the TCR targeting moiety comprises an HCDR1, HCDR2, and HCDR3 of an anti-CD3 antibody of Table G-1.

478. The multispecific molecule of any one of embodiments 466 to 473, wherein the TCR targeting moiety comprises an LCDR1, LCDR2, and LCDR3 sequence of an anti-CD3 antibody of Table G-1.

479. The multispecific molecule of embodiment 465, wherein the TCA targeting moiety is a CD28 targeting moiety.

480. The multispecific molecule of embodiment 479, wherein the CD28 targeting moiety comprises a VH having at least 95%, at least 96%, at least 97%, at least 98%, at least 99%, or 100% identity to the amino acid sequence of SEQ ID NO:237 and a VL having at least 95%, at least 96%, at least 97%, at least 98%, at least 99%, or 100% identity to the amino acid sequence of SEQ ID NO:236.

481. The multispecific molecule of embodiment 479, wherein the TCA targeting moiety comprises an HCDR1, HCDR2, and HCDR3 of an anti-CD28 antibody of Table G-2.

482. The multispecific molecule of embodiment 479 or 480, wherein the TCA targeting moiety comprises an LCDR1, LCDR2, and LCDR3 sequence of an anti-CD28 antibody of Table G-2.

483. The multispecific molecule of any one of embodiments 453 to 464, wherein the ICA targeting moiety is a B cell antigen (BCA) targeting moiety.

484. The multispecific molecule of embodiment 483, wherein the BCA targeting moiety is a CD19 targeting moiety.

485. The multispecific molecule of embodiment 484, wherein the BCA targeting moiety comprises an HCDR1, HCDR2, and HCDR3 of an anti-CD19 antibody of Table G-3.

486. The multispecific molecule of embodiment 484 or 485, wherein the BCA targeting moiety comprises an LCDR1, LCDR2, and LCDR3 sequence of an anti-CD19 antibody of Table G-3.

487. The multispecific molecule of embodiment 483, wherein the BCA targeting moiety is a CD20 targeting moiety.

488. The multispecific molecule of embodiment 487, wherein the CD28 targeting moiety comprises a VH having at least 95%, at least 96%, at least 97%, at least 98%, at least 99%, or 100% identity to the amino acid sequence of SEQ ID NO:238 and a VL having at least 95%, at least 96%, at least 97%, at least 98%, at least 99%, or 100% identity to the amino acid sequence of SEQ ID NO:239.

489. The multispecific molecule of embodiment 487, wherein the CD28 targeting moiety comprises a VH having at least 95%, at least 96%, at least 97%, at least 98%, at least 99%, or 100% identity to the amino acid sequence of SEQ ID NO:240 and a VL having at least 95%, at least 96%, at least 97%, at least 98%, at least 99%, or 100% identity to the amino acid sequence of SEQ ID NO:236.

490. The multispecific molecule of embodiment 487, wherein the BCA targeting moiety comprises an HCDR1, HCDR2, and HCDR3 of an anti-CD20 antibody of Table G-4.

491. The multispecific molecule of embodiment 487 or 488, wherein the BCA targeting moiety comprises an LCDR1, LCDR2, and LCDR3 sequence of an anti-CD20 antibody of Table G-4.

492. The multispecific molecule of embodiment 483, wherein the BCA targeting moiety is a CD22 targeting moiety.

493. The multispecific molecule of embodiment 492, wherein the BCA targeting moiety comprises an HCDR1, HCDR2, and HCDR3 of an anti-CD22 antibody of Table G-5.

494. The multispecific molecule of embodiment 492 or 493, wherein the BCA targeting moiety comprises an LCDR1, LCDR2, and LCDR3 sequence of an anti-CD22 antibody of Table G-5.

495. The multispecific molecule of any one of embodiments 453 to 494, further comprising an additional ICA targeting moiety.

496. The multispecific molecule of embodiment 495, wherein the additional ICA targeting moiety is an additional TCA targeting moiety.

497. The multispecific molecule of embodiment 496, wherein the additional TCA targeting moiety is a T cell receptor (TCR) targeting moiety.

498. The multispecific molecule of embodiment 497, wherein the TCR targeting moiety is a CD3 targeting moiety.

499. The multispecific molecule of embodiment 497, wherein the TCR targeting moiety is a TCRαβ targeting moiety.

500. The multispecific molecule of embodiment 497, wherein the TCR targeting moiety is a TCRγδ targeting moiety.

501. The multispecific molecule of any one of embodiments 497 to 500, wherein the TCR targeting moiety comprises a VH having at least 95%, at least 96%, at least 97%, at least 98%, at least 99%, or 100% identity to the amino acid sequence of SEQ ID NO:231 and a VL having at least 95%, at least 96%, at least 97%, at least 98%, at least 99%, or 100% identity to the amino acid sequence of SEQ ID NO:236.

502. The multispecific molecule of any one of embodiments 497 to 500, wherein the TCR targeting moiety comprises a VH having at least 95%, at least 96%, at least 97%, at least 98%, at least 99%, or 100% identity to the amino acid sequence of SEQ ID NO:232 and a VL having at least 95%, at least 96%, at least 97%, at least 98%, at least 99%, or 100% identity to the amino acid sequence of SEQ ID NO:236.

503. The multispecific molecule of any one of embodiments 497 to 500, wherein the TCR targeting moiety comprises a VH having at least 95%, at least 96%, at least 97%, at least 98%, at least 99%, or 100% identity to the amino acid sequence of SEQ ID NO:233 and a VL having at least 95%, at least 96%, at least 97%, at least 98%, at least 99%, or 100% identity to the amino acid sequence of SEQ ID NO:236.

504. The multispecific molecule of any one of embodiments 497 to 500, wherein the TCR targeting moiety comprises a VH having at least 95%, at least 96%, at least 97%, at least 98%, at least 99%, or 100% identity to the amino acid sequence of SEQ ID NO:234 and a VL having at least 95%, at least 96%, at least 97%, at least 98%, at least 99%, or 100% identity to the amino acid sequence of SEQ ID NO:236.

505. The multispecific molecule of any one of embodiments 497 to 500, wherein the TCR targeting moiety comprises an HCDR1, HCDR2, and HCDR3 of an anti-CD3 antibody of Table G-1.

506. The multispecific molecule of any one of embodiments 497 to 501, wherein the TCR targeting moiety comprises an LCDR1, LCDR2, and LCDR3 sequence of an anti-CD3 antibody of Table G-1.

507. The multispecific molecule of any one of embodiments 497 to 506, wherein the TCR targeting moiety has a K_{D} for the TCR of greater than 10⁻⁹M.

508. The multispecific molecule of any one of embodiments 497 to 506, wherein the TCR targeting moiety has a K_{D} for the TCR of greater than 10⁻⁸M.

509. The multispecific molecule of any one of embodiments 497 to 506, wherein the TCR targeting moiety has a K_{D} for the TCR of between 10⁻⁶M and 10⁻⁸M.

510. The multispecific molecule of embodiment 496, wherein the additional TCA targeting moiety is a CD28 targeting moiety.

511. The multispecific molecule of embodiment 510, wherein the CD28 targeting moiety comprises a VH having at least 95%, at least 96%, at least 97%, at least 98%, at least 99%, or 100% identity to the amino acid sequence of SEQ ID NO:237 and a VL having at least 95%, at least 96%, at least 97%, at least 98%, at least 99%, or 100% identity to the amino acid sequence of SEQ ID NO:236.

512. The multispecific molecule of embodiment 510, wherein the CD28 targeting moiety comprises an HCDR1, HCDR2, and HCDR3 of an anti-CD28 antibody of Table G-2.

513. The multispecific molecule of embodiment 510 or 511, wherein the CD28 targeting moiety comprises an LCDR1, LCDR2, and LCDR3 sequence of an anti-CD28 antibody of Table G-2.

514. The multispecific molecule of embodiment 495, wherein the additional ICA targeting moiety is an additional BCA targeting moiety.

515. The multispecific molecule of embodiment 514, wherein the additional BCA targeting moiety is a CD19 targeting moiety.

516. The multispecific molecule of embodiment 515, wherein the CD19 targeting moiety comprises an HCDR1, HCDR2, and HCDR3 of an anti-CD19 antibody of Table G-3.

517. The multispecific molecule of embodiment 515 or 516, wherein the CD19 targeting moiety comprises an LCDR1, LCDR2, and LCDR3 sequence of an anti-CD19 antibody of Table G-3.

518. The multispecific molecule of embodiment 514, wherein the additional BCA targeting moiety is a CD20 targeting moiety.

519. The multispecific molecule of embodiment 518, wherein the CD28 targeting moiety comprises a VH having at least 95%, at least 96%, at least 97%, at least 98%, at least 99%, or 100% identity to the amino acid sequence of SEQ ID NO:238 and a VL having at least 95%, at least 96%, at least 97%, at least 98%, at least 99%, or 100% identity to the amino acid sequence of SEQ ID NO:239.

520. The multispecific molecule of embodiment 518, wherein the CD28 targeting moiety comprises a VH having at least 95%, at least 96%, at least 97%, at least 98%, at least 99%, or 100% identity to the amino acid sequence of SEQ ID NO:240 and a VL having at least 95%, at least 96%, at least 97%, at least 98%, at least 99%, or 100% identity to the amino acid sequence of SEQ ID NO:236.

521. The multispecific molecule of embodiment 518, wherein the CD20 targeting moiety comprises an HCDR1, HCDR2, and HCDR3 of an anti-CD20 antibody of Table G-4.

522. The multispecific molecule of embodiment 518 or 521, wherein the CD20 targeting moiety comprises an LCDR1, LCDR2, and LCDR3 sequence of an anti-CD20 antibody of Table G-4.

523. The multispecific molecule of embodiment 514, wherein the additional BCA targeting moiety is a CD22 targeting moiety.

524. The multispecific molecule of embodiment 523, wherein the CD22 targeting moiety comprises an HCDR1, HCDR2, and HCDR3 of an anti-CD22 antibody of Table G-5.

525. The multispecific molecule of embodiment 523 or 524, wherein the CD22 targeting moiety comprises an LCDR1, LCDR2, and LCDR3 sequence of an anti-CD22 antibody of Table G-5.

526. The multispecific molecule of any one of embodiments 453 to 525, further comprising a tumor antigen targeting moiety.

527. The multispecific molecule of embodiment 526, wherein the tumor antigen targeting moiety is a tumor-associated antigen (TAA) targeting moiety.

528. The multispecific molecule of embodiment 527, wherein the TAA is CD20, EGFR, FITC, CD19, CD22, CD33, PSMA, GD2, EGFR variants, ROR1, c-Met, HER2, CEA, mesothelin, GM2, CD7, CD10, CD30, CD34, CD38, CD41, CD44, CD74, CD123 CD133, CD171, MUC16, MUC1, CS1 (CD319), IL-13Ra2, BCMA, Lewis Y, IgG kappa chain, folate receptor-alpha, PSCA, or EpCAM.

529. The multispecific molecule of embodiment 528, wherein the TAA is CD20.

530. The multispecific molecule of embodiment 528, wherein the TAA is BCMA.

531. The multispecific molecule of embodiment 528, wherein the TAA is EGFR.

532. The multispecific molecule of embodiment 528, wherein the TAA is Muc16.

533. The multispecific molecule of embodiment 528, wherein the TAA is PSMA.

534. The multispecific molecule of any one of embodiments 453 to 533, wherein the MHC domain is a type I MHC domain.

535. The multispecific molecule of any one of embodiments 453 to 534, wherein the pMHC complex further comprises a β2-microglobulin domain (e.g., as described in Section 6.3.3).

536. The multispecific molecule of embodiment 535, wherein the β2-microglobulin domain comprises an amino acid sequence having at least at least 95%, at least 96%, at least 97%, at least 98%, at least 99%, or 100% identity to the amino acid sequence of SEQ ID NO:250.

537. The multispecific molecule of embodiment 535, wherein the β2-microglobulin domain comprises the amino acid sequence of SEQ ID NO:250.

538. The multispecific molecule of any one of embodiments 535 to 537, wherein the pMHC complex further comprises a linker connecting the antigenic peptide and the β2-microglobulin domain.

539. The multispecific molecule of embodiment 536, wherein the linker is 7 or less amino acids in length.

540. The multispecific molecule of embodiment 539, wherein the linker is greater than 7 amino acids in length.

541. The multispecific molecule of any one of embodiments 536 to 540, wherein the pMHC complex further comprises an additional linker connecting the β2-microglobulin domain and the MHC domain.

542. The multispecific molecule of any one of embodiments 453 to 541, wherein:
(a) the first polypeptide chain comprises, in N- to C-terminal orientation:
   (i) the pMHC complex; and
   (ii) the first Fc domain; and
(b) the second polypeptide chain comprises, in N- to C-terminal orientation:
   (i) the ICA targeting moiety or component thereof; and
   (ii) the second Fc domain.

543. The multispecific molecule of any one of embodiments 453 to 541, wherein the second polypeptide further comprises an additional pMHC complex comprising an additional MHC domain.

544. The multispecific molecule of embodiment 543, wherein:
(a) the first polypeptide chain comprises, in N- to C-terminal orientation:
   (i) the pMHC complex; and
   (ii) the first Fc domain; and
(b) the second polypeptide chain comprises, in N- to C-terminal orientation:
   (i) the additional pMHC complex;
   (ii) the second Fc domain; and
   (iii) the ICA targeting moiety or component thereof.

545. The multispecific molecule of any one of the embodiments 453 to 544, wherein the first Fc domain is as defined by any one of embodiments 11 to 32.

546. The multispecific molecule of any one of the embodiments 453 to 544, wherein the second Fc domain is as defined by any one of embodiments 11 to 32.

547. The multispecific molecule of any one of the embodiments 453 to 546, wherein the pMHC complex is as defined by any one of embodiments 33 to 44.

548. The multispecific molecule of any one of the embodiments 1 to 547, wherein the multispecific molecule is a dimer.

549. The multispecific molecule of embodiment 548, wherein the multispecific molecule is a homodimer.

550. The multispecific molecule of embodiment 548, wherein the multispecific molecule is a heterodimer.

551. The multispecific molecule of any one of the embodiments 1 to 550, wherein the antigenic peptide is an antigenic determinant of cancer cells.

552. The multispecific molecule of embodiment 551, wherein the antigenic peptide is LCMV derived peptide gp33-41, APF (126-134), BALF(276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAG-A3 (112-120), MAGE-A4 (230-239), MAGE-A4 (286-294), NYESO1 (157-165, C165A), NYESO1 (157-165, C165V), p54 WT (264-272), PAP-3 (136-143), PSMA (4-12), PSMA (135-145), Survivin (96-014), Tyrosinase (369-377, 371D), WT1 (137-145), or WT1 (126-134).

553. The multispecific molecule of any one of the embodiments 1 to 550, wherein the antigenic peptide is a peptide set forth in Table 1-A.

554. The multispecific molecule of any one of the embodiments 1 to 550, wherein the antigenic peptide is a peptide set forth in Table 1-B.

555. The multispecific molecule of any one of the embodiments 1 to 550, wherein the antigenic peptide is a peptide set forth in Table 1-C.

556. The multispecific molecule of any one of the embodiments 1 to 550, wherein the antigenic peptide is HPV 16E7 (11-19) (SEQ ID NO:242).

557. The multispecific molecule of any one of the embodiments 1 to 550, wherein the antigenic peptide is NYESO-1(157-165) (SEQ ID NO:244).

558. The multispecific molecule of any one of the embodiments 1 to 550, wherein the antigenic peptide is MAGEA4(230-239) (SEQ ID NO:246).

559. The multispecific molecule of any one of the embodiments 1 to 550, wherein the antigenic peptide is CMVpp65(465-503) (SEQ ID NO:248).

560. A multispecific molecule comprising:
(a) a peptide-MHC (pMHC) complex comprising an MHC domain and an antigenic peptide (e.g., as defined by any one of embodiments 11 to 32);
(b) means for binding an immune cell antigen; and
(c) optionally, a multimerization moiety operably linked to the pMHC complex (e.g., as defined by any one of embodiments 33 to 44).

561. A multispecific molecule comprising:
(a) a peptide-MHC (pMHC) complex comprising an MHC domain and an antigenic peptide (e.g., as defined by any one of embodiments 11 to 32);
(b) means for binding a T cell antigen; and
(c) optionally, a multimerization moiety operably linked to the pMHC complex (e.g., as defined by any one of embodiments 33 to 44).

562. A multispecific molecule comprising:
(a) a peptide-MHC (pMHC) complex comprising an MHC domain and an antigenic peptide (e.g., as defined by any one of embodiments 11 to 32);
(b) means for binding human CD3; and
(c) optionally, a multimerization moiety operably linked to the pMHC complex (e.g., as defined by any one of embodiments 33 to 44).

563. A multispecific molecule comprising:
(a) a peptide-MHC (pMHC) complex comprising an MHC domain and an antigenic peptide (e.g., as defined by any one of embodiments 11 to 32);
(b) means for binding a B cell antigen; and
(c) optionally, a multimerization moiety operably linked to the pMHC complex (e.g., as defined by any one of embodiments 33 to 44).

564. A multispecific molecule comprising:
(a) a peptide-MHC (pMHC) complex comprising an MHC domain and an antigenic peptide (e.g., as defined by any one of embodiments 11 to 32);
(b) means for binding human CD19; and
(c) optionally, a multimerization moiety operably linked to the pMHC complex (e.g., as defined by any one of embodiments 33 to 44).

565. A multispecific molecule comprising:
(a) a peptide-MHC (pMHC) complex comprising an MHC domain and an antigenic peptide (e.g., as defined by any one of embodiments 11 to 32);
(b) means for binding human CD20; and
(c) optionally, a multimerization moiety operably linked to the pMHC complex (e.g., as defined by any one of embodiments 33 to 44).

566. A multispecific molecule comprising:
(a) a peptide-MHC (pMHC) complex comprising an MHC domain and an antigenic peptide (e.g., as defined by any one of embodiments 11 to 32);
(b) means for binding human CD22; and
(c) optionally, a multimerization moiety operably linked to the pMHC complex (e.g., as defined by any one of embodiments 33 to 44).

567. A nucleic acid or plurality of nucleic acids encoding the multispecific molecule of any one of embodiments 1 to 566.

568. A host cell engineered to express the multispecific molecule of any one of embodiments 1 to 566.

569. A cell transfected with one or more expression vectors comprising one or more nucleic acid sequences encoding the multispecific molecule of any one of embodiments 1 to 566 under the control of one or more promoters.

570. A method of producing the multispecific molecule of any one of embodiments 1 to 566, comprising culturing the host cell of embodiment 568 or 569 and recovering the multispecific molecule expressed thereby.

571. A method of activating T cell receptor signaling in a T cell or a population of T cells comprising administering to the T cell or population of T cells the multispecific molecule of any one of embodiments 1 to 566.

572. The method of embodiment 571, wherein the administration is in vitro.

573. The method of embodiment 571, wherein the administration is in vivo.

574. The method of any of embodiments 571 to 573, wherein the pMHC complex binds to a TCR on a T cell and the TCA targeting moiety binds to a T cell antigen on the same T cell.

575. A pharmaceutical composition comprising (a) the multispecific molecule of any one of embodiments 1 to 566 or one or more nucleic acids encoding the multispecific molecule of any one of embodiments 1 to 566 and (b) an excipient.

576. The pharmaceutical composition of embodiment 575, further comprising a multispecific antigen binding molecule (e.g., as described in Section 6.6) comprising (a) a first antigen-binding domain that specifically binds a tumor antigen; and (b) a second antigen-binding domain that specifically binds a T cell antigen (or a nucleic acid encoding such multispecific antigen binding molecule).

577. The pharmaceutical composition of embodiment 576, wherein the multispecific antigen binding molecule is a multispecific antigen binding molecule set forth in Table K-2.

578. The pharmaceutical composition of embodiment 576, wherein the second antigen-binding domain is a TCR binding domain.

579. The pharmaceutical composition of embodiment 576 or embodiment 577, wherein the second antigen-binding domain is a CD3 binding domain.

580. The pharmaceutical composition of embodiment 576, wherein the second antigen-binding domain is a CD28 binding domain.

581. A method of treating cancer comprising administering to a subject in need thereof a multispecific molecule comprising (a) a peptide-MHC (pMHC) complex comprising an MHC domain and an antigenic peptide; and (b) one or more immune cell antigen (ICA) targeting moieties (e.g., as described in Section 6.4).

582. A method of inhibiting growth of a tumor cell in a subject, comprising administering to the subject a multispecific molecule comprising (a) a peptide-MHC (pMHC) complex comprising an MHC domain and an antigenic peptide; and (b) one or more immune cell antigen (ICA) targeting moieties (e.g., as described in Section 6.4).

583. A method of stimulating proliferation of cancer antigen specific T cells, comprising administering to a subject having cancer a multispecific molecule comprising (a) a peptide-MHC (pMHC) complex comprising an MHC domain and an antigenic peptide; and (b) one or more immune cell antigen (ICA) targeting moieties (e.g., as described in Section 6.4).

584. The method of any one of embodiments 581 to 583, wherein the multispecific molecule is the multispecific molecule of any one of embodiments 1 to 566.

585. The method of embodiment 584, wherein the multispecific molecule is administered by administration of the pharmaceutical composition of any one of embodiments 575 to 580.

586. The method of any one of embodiments 581 to 585, wherein administration of the multispecific molecule reduces the viability of cancer cells expressing a protein comprising the amino acid sequence of the antigenic peptide in the subject.

587. The method of any one of embodiments 581 to 586, wherein administration of the multispecific molecule suppresses metastasis of cancer cells expressing a protein comprising the amino acid sequence of the antigenic peptide in the subject.

588. The method of any one of embodiments 581 to 587, further comprising administering to the subject a multispecific antigen binding molecule (e.g., as described in Section 6.6) comprising (a) a first antigen-binding domain that specifically binds a tumor antigen or a B cell antigen; and (b) a second antigen-binding domain that specifically binds a T cell antigen.

589. The method of embodiment 588, wherein the second antigen-binding domain is a TCR binding domain.

590. The method of embodiment 588, wherein the second antigen-binding domain is a CD3 binding domain.

591. The method of embodiment 588, wherein the second antigen-binding domain is a CD28 binding domain.

592. The method of any one of embodiments 581 to 591, wherein the multispecific antigen binding molecule is administered prior to administration of the multispecific molecule.

593. The method of any one of embodiments 581 to 591, wherein the multispecific antigen binding molecule is administered concurrently with the multispecific molecule.

594. The method of any one of embodiments 581 to 591, wherein the multispecific antigen binding molecule is administered after administration of the multispecific molecule.

595. The method of any one of embodiments 588 to 594, wherein the first antigen-binding domain is a tumor antigen targeting moiety.

596. The method of embodiment 595, wherein the first antigen-binding domain comprises an HCDR1, HCDR2, and HCDR3 of an antibody set forth in Table K-2.

597. The method of embodiment 595 or 596, wherein the first antigen-binding domain comprises an LCDR1, LCDR2, and LCDR3 of an antibody set forth in Table K-2.

598. The method of any one of embodiments 595 to 597, wherein the tumor antigen is CD20, EGFR, FITC, CD19, CD22, CD33, PSMA, GD2, EGFR variants, ROR1, c-Met, HER2, CEA, mesothelin, GM2, CD7, CD10, CD30, CD34, CD38, CD41, CD44, CD74, CD123 CD133, CD171, MUC16, MUC1, CS1 (CD319), IL-13Ra2, BCMA, Lewis Y, IgG kappa chain, folate receptor-alpha, PSCA, or EpCAM.

599. The method of embodiment 598, wherein the tumor antigen is CD20.

600. The method of embodiment 598, wherein the tumor antigen is BCMA.

601. The method of embodiment 598, wherein the tumor antigen is EGFR.

602. The method of embodiment 598, wherein the tumor antigen is Muc16.

603. The method of embodiment 598, wherein the tumor antigen is PSMA.

604. The method of any one of embodiments 588 to 594, wherein the first antigen-binding domain is a B cell antigen targeting moiety.

605. The method of embodiment 604, wherein the B cell antigen is CD19.

606. The method of embodiment 604, wherein the B cell antigen is CD20.

607. The method of embodiment 604, wherein the B cell antigen is CD22.

608. The method of any one of embodiments 588 to 607, wherein the T cell antigen is CD3.

609. The method of embodiment 608, wherein the second antigen-binding domain comprises an HCDR1, HCDR2, and HCDR3 of an anti-CD3 antibody of Table G-1.

610. The method of embodiment 608 or embodiment 609, wherein the second antigen-binding domain comprises an LCDR1, LCDR2, and LCDR3 sequence of an anti-CD3 antibody of Table G-1.

611. The method of any one of embodiments 588 to 607, wherein the T cell antigen is CD28.

612. The multispecific molecule of embodiment 611, wherein the CD28 targeting moiety comprises a VH having at least 95%, at least 96%, at least 97%, at least 98%, at least 99%, or 100% identity to the amino acid sequence of SEQ ID NO:237 and a VL having at least 95%, at least 96%, at least 97%, at least 98%, at least 99%, or 100% identity to the amino acid sequence of SEQ ID NO:236.

613. The method of embodiment 611, wherein the second antigen-binding domain comprises an HCDR1, HCDR2, and HCDR3 of an anti-CD28 antibody of Table G-2.

614. The method of embodiment 611 or embodiment 612, wherein the second antigen-binding domain comprises an LCDR1, LCDR2, and LCDR3 sequence of an anti-CD28 antibody of Table G-2.

615. The method of any one of embodiments 588 to 610, wherein the multispecific antigen binding molecule is a CD3×CD20 bispecific antigen binding molecule.

616. The method of any one of embodiments 588 to 610, wherein the multispecific antigen binding molecule is a CD3×CD19 bispecific antigen binding molecule.

617. The method of any one of embodiments 588 to 610, wherein the multispecific antigen binding molecule is a CD3×CD22 bispecific antigen binding molecule.

618. The method of any one of embodiments 588 to 610, wherein the multispecific antigen binding molecule is a CD3×BCMA bispecific antigen binding molecule.

619. The method of any one of embodiments 588 to 618, further comprising administering to the subject an additional therapeutic.

620. The method of embodiment 619, wherein the additional therapeutic is a chemotherapeutic, immunotherapeutic, or cytokine therapeutic.

621. The method of embodiment 619 or embodiment 620, wherein the additional therapeutic is a checkpoint inhibitor.

622. A method for preventing a disease or condition, the method comprising administering to a subject a multispecific molecule comprising (a) a peptide-MHC (pMHC) complex comprising an MHC domain and an antigenic peptide; and (b) one or more immune cell antigen (ICA) targeting moieties (e.g., as described in Section 6.4).

623. The method of embodiment 622, wherein the disease or condition is cancer.

624. The method of embodiment 623, wherein the antigenic peptide is a tumor antigen or portion thereof.

625. The method of any one of embodiments 622 to 624, further comprising administering to the subject one or more adjuvants.

626. A method of stimulating proliferation of antigen specific T cells, comprising administering to a subject a multispecific molecule comprising (a) a peptide-MHC (pMHC) complex comprising an MHC domain and an antigenic peptide; and (b) one or more immune cell antigen (ICA) targeting moieties (e.g., as described in Section 6.4).

627. The method of embodiment 626, wherein the subject does not have a disease or condition associated with the antigenic peptide.

628. The method of any one of embodiments 622 to 624, wherein the multispecific molecule is the multispecific molecule of any one of embodiments 1 to 566.

629. The method of embodiment 628, wherein the multispecific molecule is administered by administration of the pharmaceutical composition of any one of embodiments 575 to 580.

630. A method comprising administering to a subject the multispecific molecule of any one of embodiments 1 to 566, one or more nucleic acids encoding the multispecific molecule of any one of embodiments 1 to 566, or a pharmaceutical composition according to any one of embodiments 575 to 580.

631. The method of embodiment 630, wherein administration elicits an immune response against a cancerous cell.

632. The method of embodiment 630 or embodiment 631, wherein the subject has cancer and the method treats the cancer.

633. The method of embodiment 630 or embodiment 631, wherein the subject does not have cancer and the administration prevents cancer or delays onset of cancer.

634. The method of embodiment 633, wherein the subject is predisposed to cancer.

635. The method of embodiment 634, wherein the subject has a genetic marker for cancer.

636. The method of embodiment 634 or embodiment 635, wherein the subject has a viral infection that increases risk of cancer (e.g., HPV, HIV, etc.).

637. The method of any one of embodiments 634 to 636, wherein the subject has been treated for, and is in remission from, cancer.

### 8. EXAMPLES

### 8.1. Materials and Methods

### 8.1.1. Design and Production of T Cell Activator Constructs

Monovalent T cell activator constructs were designed to comprise a pMHC complex and a TCR targeting moiety connected to the N-terminus of an Fc domain. Exemplary monovalent T cell activator constructs are presented in FIGS. 1A and 1B. Bivalent T cell activator constructs were designed to comprise two pMHC complexes connected to the Fc domain at the N-terminus and a TCR targeting moiety connected to the Fc domain at the C-terminus. Exemplary bivalent T cell activator constructs are presented in FIGS. 1C and 1D.

In order to assess the effect of linker length, T cell activator constructs were designed to comprise either a short linker (i.e., a linker at most or exactly 7 amino acids in length) or a long linker (i.e., a linker more than 7 amino acids in length) between the pMHC complex and the Fc domain. Furthermore, to assess the effect of T cell-stimulating Fab domains, constructs were designed to comprise either an anti-CD3 Fab, an anti-CD20 Fab, an anti-CD19 Fab, or an anti-CD28 Fab. Anti-CD3 Fab domain of a construct was selected from a set of four anti-CD3 antibodies with distinct CD3 binding properties as set forth in Table E-1.

TABLE E-1

| | CD3 Epitope - Binding | | |
|---|---|---|---|
| Anti-CD3 Fab | Strength | FACS EC50 [M] | Biacore KD [M] |
| 7195P | CD3 ε/δ - Very Strong | 6.9E−10 | 3.83E−11 |
| 7221G | CD3 ε/δ - Moderate | 1.3E−08 | 2.49E−08 |
| 7221G20 | CD3 ε/δ - Weak | 5.4E−07 | 3.20E−07 |
| 7225G5 | CD3 ε/δ - Very Weak | Below | Below |
| | | Determination Limit | Determination Limit |
| L2K | CD3 ε/δ - N/A | N/A | N/A |
| A2J | CD3 ε/δ - N/A | N/A | N/A |
| 6G12 | CD3 ε/δ - N/A | N/A | N/A |
| 1A4 | CD3 ε/δ and ε/γ - N/A | N/A | 6.34E−10 |
| OKT3 | CD3 ε/δ and ε/γ - N/A | N/A | 9.62E−09 |
| 7208P | CD3 ε/δ - N/A | N/A | 4.51E−09 |

All constructs were expressed in Expi293F™ cells by transient transfection (Thermo Fisher Scientific). Proteins in Expi293F supernatant were purified using the ProteinMaker system (Protein BioSolutions, Gaithersburg, MD) with HiTrap Protein G HP columns (GE Healthcare). After single step elution, the muteins were neutralized, dialyzed into a final buffer of phosphate buffered saline (PBS) with 5% glycerol, aliquoted and stored at −80° C.

T cell activator and control constructs utilized in the examples are presented in Table E-2. All constructs evaluated in the examples below contain IgG4 Fc domains unless otherwise indicated.

TABLE E-2
Constructs

| Construct | Polypeptide Chains |
|---|---|
| pMHC × 7195P | Chain 1: HPVE7(11-19) pMHC - Short linker - Fc_{knob} |
| (with HPVE7(11-19) pMHC | Chain 2: Anti-CD3 Fab (7195P) - Fcₕₒₗₑ |
| and Short linker) | |
| pMHC × 7195P | Chain 1: HPVE7(11-19) pMHC - long linker - Fc_{knob} |
| (with HPVE7(11-19) pMHC | Chain 2: Anti-CD3 Fab (7195P) - Fcₕₒₗₑ |
| and long linker) | |
| pMHC × 7221G | Chain 1: HPVE7(11-19) pMHC - Short linker - Fc_{knob} |
| (with HPVE7(11-19) pMHC | Chain 2: Anti-CD3 Fab (7221G) - Fcₕₒₗₑ |
| and Short linker) | |
| pMHC × 7221G | Chain 1: HPVE7(11-19) pMHC - long linker - Fc_{knob} |
| (with HPVE7(11-19) pMHC | Chain 2: Anti-CD3 Fab (7221G) - Fcₕₒₗₑ |
| and long linker) | |
| pMHC × 7221G20 | Chain 1: HPVE7(11-19) pMHC - Short linker - Fc_{knob} |
| (with HPVE7(11-19) pMHC | Chain 2: Anti-CD3 Fab (7221G20) - Fcₕₒₗₑ |
| and Short linker) | |
| pMHC × 7221G20 | Chain 1: HPVE7(11-19) pMHC - long linker - Fc_{knob} |
| (with HPVE7(11-19) pMHC | Chain 2: Anti-CD3 Fab (7221G20) - Fcₕₒₗₑ |
| and long linker) | |
| pMHC × 7221G5 | Chain 1: HPVE7(11-19) pMHC - Short linker - Fc_{knob} |
| (with HPVE7(11-19) pMHC | Chain 2: Anti-CD3 Fab (7221G5) - Fcₕₒₗₑ |
| and Short linker) | |
| pMHC × 7221G5 | Chain 1: HPVE7(11-19) pMHC - long linker - Fc_{knob} |
| (with HPVE7(11-19) pMHC | Chain 2: Anti-CD3 Fab (7221G5) - Fcₕₒₗₑ |
| and long linker) | |
| pMHC × L2K | Chain 1: HPVE7(11-19) pMHC - short linker - Fcknob |
| (with HPVE7(11-19) pMHC | Chain 2: Anti-CD3 Fab (L2K) - Fchole |
| and Short linker) | |
| pMHC × L2K | Chain 1: HPVE7(11-19) pMHC - long linker - Fcknob |
| (with HPVE7(11-19) pMHC | Chain 2: Anti-CD3 Fab (L2K) - Fchole |
| and long linker) | |
| pMHC × A2J | Chain 1: HPVE7(11-19) pMHC - short linker - Fcknob |
| (with HPVE7(11-19) pMHC | Chain 2: Anti-CD3 Fab (A2J) - Fchole |
| and Short linker) | |
| pMHC × A2J | Chain 1: HPVE7(11-19) pMHC - long linker - Fcknob |
| (with HPVE7(11-19) pMHC | Chain 2: Anti-CD3 Fab (A2J) - Fchole |
| and long linker) | |
| pMHC × 6G12 | Chain 1: HPVE7(11-19) pMHC - short linker - Fcknob |
| (with HPVE7(11-19) pMHC | Chain 2: Anti-CD3 Fab (G612) - Fchole |
| and Short linker) | |
| pMHC × 6G12 | Chain 1: HPVE7(11-19) pMHC - long linker - Fcknob |
| (with HPVE7(11-19) pMHC | Chain 2: Anti-CD3 Fab (G612) - Fchole |
| and long linker) | |
| pMHC × 1A4 | Chain 1: HPVE7(11-19) pMHC - short linker - Fcknob |
| (with HPVE7(11-19) pMHC | Chain 2: Anti-CD3 Fab (1A4) - Fchole |
| and Short linker) | |
| pMHC × 1A4 | Chain 1: HPVE7(11-19) pMHC - long linker - Fcknob |
| (with HPVE7(11-19) pMHC | Chain 2: Anti-CD3 Fab (1A4) - Fchole |
| and long linker) | |
| pMHC × OKT3 | Chain 1: HPVE7(11-19) pMHC - short linker - Fcknob |
| (with HPVE7(11-19) pMHC | Chain 2: Anti-CD3 Fab (OKT3) - Fchole |
| and Short linker) | |
| pMHC × OKT3 | Chain 1: HPVE7(11-19) pMHC - long linker - Fcknob |
| (with HPVE7(11-19) pMHC | Chain 2: Anti-CD3 Fab (OKT3) - Fchole |
| and long linker) | |
| pMHC × 7208P | Chain 1: HPVE7(11-19) pMHC - short linker - Fcknob |
| (with HPVE7(11-19) pMHC | Chain 2: Anti-CD3 Fab (7208P) - Fchole |
| and Short linker) | |
| pMHC × 7208P | Chain 1: HPVE7(11-19) pMHC - long linker - Fcknob |
| (with HPVE7(11-19) pMHC | Chain 2: Anti-CD3 Fab (7208P) - Fchole |
| and long linker) | |
| pMHC₂ × 7195P | Chain 1: HPVE7(11-19) pMHC - Short linker - Fc_{knob} - anti- |
| (with HPVE7(11-19) pMHC | CD3 Fab (7195P) |
| and Short linker) | Chain 2: HPVE7(11-19) pMHC - Short linker - Fcₕₒₗₑ |
| pMHC₂ × 7195P | Chain 1: HPVE7(11-19) pMHC - long linker - Fc_{knob} - anti- |
| (with HPVE7(11-19) pMHC | CD3 Fab (7195P) |
| and long linker) | Chain 2: HPVE7(11-19) pMHC - long linker - Fcₕₒₗₑ |
| pMHC₂ × 7221G | Chain 1: HPVE7(11-19) pMHC - Short linker - Fc_{knob} - anti- |
| (with HPVE7(11-19) pMHC | CD3 Fab (7221G) |
| and Short linker) | Chain 2: HPVE7(11-19) pMHC - Short linker - Fcₕₒₗₑ |
| pMHC₂ × 7221G | Chain 1: HPVE7(11-19) pMHC - long linker - Fc_{knob} - anti- |
| (with HPVE7(11-19) pMHC | CD3 Fab (7221G) |
| and long linker) | Chain 2: HPVE7(11-19) pMHC - long linker - Fcₕₒₗₑ |
| pMHC₂ × 7221G20 | Chain 1: HPVE7(11-19) pMHC - Short linker - Fc_{knob} - anti- |
| (with HPVE7(11-19) pMHC | CD3 Fab (7221G20) |
| and Short linker) | Chain 2: HPVE7(11-19) pMHC - Short linker - Fcₕₒₗₑ |
| pMHC2 × 7221G20 | Chain 1: HPVE7(11-19) pMHC - long linker - Fc_{knob} - anti- |
| (with HPVE7(11-19) pMHC | CD3 Fab (7221G20) |
| and long linker) | Chain 2: HPVE7(11-19) pMHC - long linker - Fcₕₒₗₑ |
| pMHC2 × 7221G5 | Chain 1: HPVE7(11-19) pMHC - Short linker - Fc_{knob} - anti- |
| (with HPVE7(11-19) pMHC | CD3 Fab (7221G5) |
| and Short linker) | Chain 2: HPVE7(11-19) pMHC - Short linker - Fcₕₒₗₑ |
| pMHC2 × 7221G5 | Chain 1: HPVE7(11-19) pMHC - long linker - Fc_{knob} - anti- |
| (with HPVE7(11-19) pMHC | CD3 Fab (7221G5) |
| and long linker) | Chain 2: HPVE7(11-19) pMHC - long linker - Fcₕₒₗₑ |
| pMHC × 7195P | Chain 1: HPVE7(11-19) pMHC - Short linker - Fc_{knob} |
| (with MAGE-A4 (230-239) | Chain 2: Anti-CD3 Fab (7195P) - Fcₕₒₗₑ |
| pMHC and Short linker) | |
| pMHC × 7195P | Chain 1: MAGE-A4 (230-239) pMHC - long linker - Fc_{knob} |
| (with MAGE-A4 (230-239) | Chain 2: Anti-CD3 Fab (7195P) - Fcₕₒₗₑ |
| pMHC and long linker) | |
| pMHC × 7221G | Chain 1: MAGE-A4 (230-239) pMHC - Short linker - Fc_{knob} |
| (with MAGE-A4 (230-239) | Chain 2: Anti-CD3 Fab (7221G) - Fcₕₒₗₑ |
| pMHC and Short linker) | |
| pMHC × 7221G | Chain 1: MAGE-A4 (230-239) pMHC - long linker - Fc_{knob} |
| (with MAGE-A4 (230-239) | Chain 2: Anti-CD3 Fab (7221G) - Fcₕₒₗₑ |
| pMHC and long linker) | |
| pMHC × 7221G20 | Chain 1: MAGE-A4 (230-239) pMHC - Short linker - Fc_{knob} |
| (with MAGE-A4 (230-239) | Chain 2: Anti-CD3 Fab (7221G20) - Fcₕₒₗₑ |
| pMHC and Short linker) | |
| pMHC × 7221G20 | Chain 1: MAGE-A4 (230-239) pMHC - long linker - Fc_{knob} |
| (with MAGE-A4 (230-239) | Chain 2: Anti-CD3 Fab (7221G20) - Fcₕₒₗₑ |
| pMHC and long linker) | |
| pMHC × 7221G5 | Chain 1: MAGE-A4 (230-239) pMHC - Short linker - Fc_{knob} |
| (with MAGE-A4 (230-239) | Chain 2: Anti-CD3 Fab (7221G5) - Fcₕₒₗₑ |
| pMHC and Short linker) | |
| pMHC × 7221G5 | Chain 1: MAGE-A4 (230-239) pMHC - long linker - Fc_{knob} |
| (with MAGE-A4 (230-239) | Chain 2: Anti-CD3 Fab (7221G5) - Fcₕₒₗₑ |
| pMHC and long linker) | |
| pMHC × 7195P | Chain 1: NY-ESO-1 (157-165) pMHC - short linker - Fc_{knob} |
| (with NY-ESO-1 (157-165) | Chain 2: Anti-CD3 Fab (7195P) - Fcₕₒₗₑ |
| pMHC and short linker) | |
| pMHC × 7195P | Chain 1: NY-ESO-1 (157-165) pMHC - long linker - Fc_{knob} |
| (with NY-ESO-1 (157-165) | Chain 2: Anti-CD3 Fab (7195P) - Fcₕₒₗₑ |
| pMHC and long linker) | |
| pMHC × 7221G | Chain 1: NY-ESO-1 (157-165) pMHC - Short linker - Fc_{knob} |
| (with NY-ESO-1 (157-165) | Chain 2: Anti-CD3 Fab (7221G) - Fcₕₒₗₑ |
| pMHC and Short linker) | |
| pMHC × 7221G | Chain 1: NY-ESO-1 (157-165) pMHC - long linker - Fc_{knob} |
| (with NY-ESO-1 (157-165) | Chain 2: Anti-CD3 Fab (7221G) - Fcₕₒₗₑ |
| pMHC and long linker) | |
| pMHC × 7221G20 | Chain 1: NY-ESO-1 (157-165) pMHC - Short linker - Fc_{knob} |
| (with NY-ESO-1 (157-165) | Chain 2: Anti-CD3 Fab (7221G20) - Fcₕₒₗₑ |
| pMHC and Short linker) | |
| pMHC × 7221G20 | Chain 1: NY-ESO-1 (157-165) pMHC - long linker - Fc_{knob} |
| (with NY-ESO-1 (157-165) | Chain 2: Anti-CD3 Fab (7221G20) - Fcₕₒₗₑ |
| pMHC and long linker) | |
| pMHC × 7221G5 | Chain 1: NY-ESO-1 (157-165) pMHC - Short linker - Fc_{knob} |
| (with NY-ESO-1 (157-165) | Chain 2: Anti-CD3 Fab (7221G5) - Fcₕₒₗₑ |
| pMHC and Short linker) | |
| pMHC × 7221G5 | Chain 1: NY-ESO-1 (157-165) pMHC - long linker - Fc_{knob} |
| (with NY-ESO-1 (157-165) | Chain 2: Anti-CD3 Fab (7221G5) - Fcₕₒₗₑ |
| pMHC and long linker) | |
| pMHC × 7221G20 | Chain 1: CMVpp65(465-503) pMHC - Short linker - Fc_{knob} |
| (with CMVpp65(465-503) | Chain 2: anti-CD3 (7221G20) - Fcₕₒₗₑ |
| pMHC and Short linker) | |
| pMHC × 7221G20 | Chain 1: CMVpp65(465-503) pMHC - Long linker - Fc_{knob} |
| (with CMVpp65(465-503) | Chain 2: anti-CD3 (7221G20) - Fcₕₒₗₑ |
| pMHC and Long linker) | |
| pMHC × BetV1 | Chain 1: HPVE7(11-19) pMHC - Short linker - Fc_{knob} |
| (with HPVE7(11-19) pMHC | Chain 2: Anti-BetV1 Fab - Fcₕₒₗₑ |
| and Short linker) | |
| pMHC × BetV1 | Chain 1: HPVE7(11-19) pMHC - long linker - Fc_{knob} |
| (with HPVE7(11-19) pMHC | Chain 2: Anti-BetV1 Fab - Fcₕₒₗₑ |
| and long linker) | |
| pMHC × BetV1 | Chain 1: NY-ESO-1 (157-165) pMHC - Short linker - Fc_{knob} |
| (with NY-ESO-1 (157-165) | Chain 2: Anti-BetV1 Fab - Fcₕₒₗₑ |
| pMHC and Short linker) | |
| pMHC × BetV1 | Chain 1: NY-ESO-1 (157-165) pMHC - long linker - Fc_{knob} |
| (with NY-ESO-1 (157-165) | Chain 2: Anti- BetV1 Fab - Fcₕₒₗₑ |
| pMHC and long linker) | |
| pMHC × BetV1 | Chain 1: MAGE-A4 (230-239) pMHC - Short linker - Fc_{knob} |
| (with MAGE-A4 (230-239) | Chain 2: Anti-BetV1 Fab - Fcₕₒₗₑ |
| pMHC and Short linker) | |
| pMHC × BetV1 | Chain 1: MAGE-A4 (230-239) pMHC - long linker - Fc_{knob} |
| (with MAGE-A4 (230-239) | Chain 2: Anti- BetV1 Fab - Fcₕₒₗₑ |
| pMHC and long linker) | |
| pMHC₂ × BetV1 | Chain 1: HPVE7(11-19) pMHC - Short linker - Fc_{knob} - anti- |
| (with HPVE7(11-19) pMHC | BetV1 Fab |
| and Short linker) | Chain 2: HPVE7(11-19) pMHC - Short linker - Fcₕₒₗₑ |
| pMHC₂ × BetV1 | Chain 1: HPVE7(11-19) pMHC - long linker - Fc_{knob} - anti- |
| (with HPVE7(11-19) pMHC | BetV1 Fab |
| and long linker) | Chain 2: HPVE7(11-19) pMHC - long linker - Fcₕₒₗₑ |
| pMHC × pMHC | Chain 1: HPVE7(11-19) pMHC - Short linker - Fc_{knob} |
| (with HPVE7(11-19) pMHC | Chain 2: HPVE7(11-19) pMHC - Fcₕₒₗₑ |
| and Short linker) | |
| pMHC × pMHC | Chain 1: HPVE7(11-19) pMHC - long linker - Fc_{knob} |
| (with HPVE7(11-19) pMHC | Chain 2: HPVE7(11-19) pMHC - Fcₕₒₗₑ |
| and long linker) | |
| pMHC × pMHC | Chain 1: NY-ESO-1 (157-165) pMHC - Short linker - Fc_{knob} |
| (with NY-ESO-1 (157-165) | Chain 2: NY-ESO-1 (157-165) pMHC - Fcₕₒₗₑ |
| pMHC and Short linker) | |
| pMHC × pMHC | Chain 1: NY-ESO-1 (157-165) pMHC - long linker - Fc_{knob} |
| (with NY-ESO-1 (157-165) | Chain 2: NY-ESO-1 (157-165) pMHC - Fcₕₒₗₑ |
| pMHC and long linker) | |
| pMHC × pMHC | Chain 1: MAGE-A4 (230-239) pMHC - Short linker - Fc_{knob} |
| (with MAGE-A4 (230-239) | Chain 2: MAGE-A4 (230-239) pMHC - Fcₕₒₗₑ |
| pMHC and Short linker) | |
| pMHC × pMHC | Chain 1: MAGE-A4 (230-239) pMHC - long linker - Fc_{knob} |
| (with MAGE-A4 (230-239) | Chain 2: MAGE-A4 (230-239) pMHC - Fcₕₒₗₑ |
| pMHC and long linker) | |
| pMHC × CD28 | Chain 1: HPVE7(11-19) pMHC - Short linker - Fc_{knob} |
| (with HPVE7(11-19) pMHC | Chain 2: anti-CD28 Fab - Fcₕₒₗₑ |
| and Short linker) | |
| pMHC × CD28 | Chain 1: HPVE7(11-19) pMHC - long linker - Fc_{knob} |
| (with HPVE7(11-19) pMHC | Chain 2: anti-CD28 Fab - Fcₕₒₗₑ |
| and long linker) | |
| pMHC × CD28 | Chain 1: NY-ESO-1 (157-165) pMHC - Short linker - Fc_{knob} |
| (with NY-ESO-1 (157-165) | Chain 2: anti-CD28 Fab - Fcₕₒₗₑ |
| pMHC and Short linker) | |
| pMHC × CD28 | Chain 1: NY-ESO-1 (157-165) pMHC - long linker - Fc_{knob} |
| (with NY-ESO-1 (157-165) | Chain 2: anti-CD28 Fab - Fcₕₒₗₑ |
| pMHC and long linker) | |
| pMHC × CD28 | Chain 1: MAGE-A4 (230-239) pMHC - Short linker - Fc_{knob} |
| (with MAGE-A4 (230-239) | Chain 2: anti-CD28 Fab - Fcₕₒₗₑ |
| pMHC and Short linker) | |
| pMHC × CD28 | Chain 1: MAGE-A4 (230-239) pMHC - long linker - Fc_{knob} |
| (with MAGE-A4 (230-239) | Chain 2: anti-CD28 Fab - Fcₕₒₗₑ |
| pMHC and long linker) | |
| pMHC × CD20 (VAC3B9-10) | Chain 1: HPVE7(11-19) pMHC - Short linker - Fc_{knob} |
| (with HPVE7(11-19) pMHC | Chain 2: anti-CD20 Fab (VAC3B89) - Fcₕₒₗₑ |
| and Short linker) | |
| pMHC × CD20 (VAC3B89) | Chain 1: HPVE7(11-19) pMHC - long linker - Fc_{knob} |
| (with HPVE7(11-19) pMHC | Chain 2: anti-CD20 Fab (VAC3B89) - Fcₕₒₗₑ |
| and long linker) | |
| pMHC × CD20 (14303) | Chain 1: HPVE7(11-19) pMHC - Short linker - Fc_{knob} |
| (with HPVE7(11-19) pMHC | Chain 2: anti-CD20 Fab (14303) - Fcₕₒₗₑ |
| and Short linker) | |
| pMHC × CD20 (14303) | Chain 1: HPVE7(11-19) pMHC - long linker - Fc_{knob} |
| (with HPVE7(11-19) pMHC | Chain 2: anti-CD20 Fab (14303) - Fcₕₒₗₑ |
| and long linker) | |
| pMHC × CD20 (VAC3B89) | Chain 1: CMVpp65(465-503) pMHC - Short linker - Fc_{knob} |
| (with CMVpp65(465-503) | Chain 2: anti-CD20 (VAC3B89) - Fcₕₒₗₑ |
| pMHC and Short linker) | |
| pMHC × CD20 (VAC3B89) | Chain 1: CMVpp65(465-503) pMHC - Long linker - Fc_{knob} |
| (with CMVpp65(465-503) | Chain 2: anti-CD20 (VAC3B89) - Fcₕₒₗₑ |
| pMHC and Long linker) | |
| pMHC × CD20 (VAC3B89) | Chain 1: MAGE-A4(230-239) pMHC - Short linker - Fc_{knob} |
| (with MAGE-A4 (230-239) | Chain 2: anti-CD20 (VAC3B89) - Fcₕₒₗₑ |
| pMHC and Short linker) | |
| pMHC × CD20 (VAC3B89) | Chain 1: MAGE-A4(230-239) pMHC - Long linker - Fc_{knob} |
| (with MAGE-A4 (230-239) | Chain 2: anti-CD20 (VAC3B89) - Fcₕₒₗₑ |
| pMHC and Long linker) | |
| pMHC × CD19 (34678) | Chain 1: HPVE7(11-19) pMHC - Short linker - Fc_{knob} |
| (with HPVE7(11-19) pMHC | Chain 2: anti-CD19 Fab (34678) - Fcₕₒₗₑ |
| and Short linker) | |
| pMHC × CD19 (34678) | Chain 1: HPVE7(11-19) pMHC - Long linker - Fc_{knob} |
| (with HPVE7(11-19) pMHC | Chain 2: anti-CD19 Fab (34678) - Fcₕₒₗₑ |
| and Long linker) | |
| pMHC × CD19 (34668) | Chain 1: HPVE7(11-19) pMHC - Short linker - Fc_{knob} |
| (with HPVE7(11-19) pMHC | Chain 2: anti-CD19 Fab (34668) - Fcₕₒₗₑ |
| and Short linker) | |
| pMHC × CD19 (34668) | Chain 1: HPVE7(11-19) pMHC - Long linker - Fc_{knob} |
| (with HPVE7(11-19) pMHC | Chain 2: anti-CD19 Fab (34668) - Fcₕₒₗₑ |
| and Long linker) | |

Certain amino acid sequences of targeting moieties and pMHC complexes of the T cell activators set forth in Table E-2 are presented in Table E-3.

TABLE E-3
Sequences

| Identifier | Sequence | SEQ ID NO |
|---|---|---|
| Anti-CD3 (7195) | EVQLVESGGGLVQPGRSLRLSCAASGFTFADYTMHWV | 231 |
| VH sequence | RQAPGKGLEWVSDISWNSGSIAYADSVKGRFTISRDNA | |
| | KNSLYLQMNSLRTEDTAFYYCAKDSRGYGHYKYLGLDV | |
| | WGQGTTVTVSS | |
| Anti-CD3 | EVQLVESGGGLVQPGRSLRLSCAASGFTFDDYSMHWV | 232 |
| (7221G) VH | RQAPGKGLEWVSGISWNSGSKGYADSVKGRFTISRDN | |
| sequence | AKNSLYLQMNSLRAEDTALYYCAKYGSGYGKFYHYGLD | |
| | VWGQGTTVTVSS | |
| Anti-CD3 | EVQLVESGGGLVQPGRSLRLSCAASGFTFDDYSMHWV | 233 |
| (7221G5) VH | RQAPGKGLEWVSGISWNSGSIGYADSVKGRFTISRDNA | |
| sequence | KNSLYLQMNSLRAEDTALYYCAKDGSGYGKFYYYGMD | |
| | VWGQGTTVTVSS | |
| Anti-CD3 | EVQLVESGGGLVQPGRSLRLSCAASGFTFDDYSMHWV | 234 |
| (7221G20) VH | RQAPGKGLEWVSGISWNSGSIGYADSVKGRFTISRDNA | |
| sequence | KNSLYLQMNSLRAEDTALYYCAKYGSGYGKFYYYGMDV | |
| | WGQGTTVTVSS | |
| Anti-BetV1 VH | EVQLVQSGAEVKKPGESLKISCSGSGYRFTNYWIAWVR | 235 |
| sequence | QMPGKGLEWMGLIHPDDSDIRYSPSFQGQVTFSVDKSI | |
| | NTAYLQWSSLKASDTAMYYCTRQDGILWSHNAWFDPW | |
| | GQGTLVTVSS | |
| ULC1-39 Light | DIQMTQSPSSLSASVGDRVTITCRASQSISSYLNWYQQK | 236 |
| chain sequence | PGKAPKLLIYAASSLQSGVPSRFSGSGSGTDFTLTISSLQ | |
| | PEDFATYYCQQSYSTPPITFGQGTRLEIK | |
| Anti-CD28 VH | EVQLVESGGGLVQPGGSLRLSCAASGFTFSRNNMHWV | 237 |
| sequence | RQAPGKGLEYVSGISSNGGRTYYADSVKGRFTISRDNS | |
| | KNTLYLQMGGLRAADMAVYFCTRDDELLSFDYWGQGT | |
| | LVTVSS | |
| Anti-CD20 VH | EVQLVESGGGLVQPGRSLRLSCVASGFTFNDYAMHWV | 238 |
| (VAC3B9-10 VH) | RQAPGKGLEWVSVISWNSDSIGYADSVKGRFTISRDNA | |
| sequence | KNSLYLQMHSLRAEDTALYYCAKDNHYGSGSYYYYQYG | |
| | MDVWGQGTTVTVSS | |
| Anti-CD20 | EIVMTQSPATLSVSPGERATLSCRASQSVSSNLAWYQQ | 239 |
| (VAC3B9-10) | KPGQAPRLLIYGASTRATGIPARFSGSGSGTEFTLTISSL | |
| light chain | QSEDFAVYYCQHYINWPLTFGGGTKVEIK | |
| sequence | | |
| Anti-CD20 | QVHLVESGGGVVQPGRSLRLSCSVSGLTFNSYGMHWV | 240 |
| (14303) VH | RQAPGKGLEWVTMISYDGSDKYYLDSVKGRFTISRDNS | |
| sequence | KNILYLQINSLRPEDTAVYYCVNWDNWRGNFDYWGQG | |
| | TLVTVSS | |
| HPV 16E7 | YMLDLQPETGCGGSGGGGSGGGGSIQRTPKIQVYSRH | 241 |
| (11-19) pMHC | PAENGKSNFLNCYVSGFHPSDIEVDLLKNGERIEKVEHS | |
| (HPV 16E7 | DLSFSKDWSFYLLYYTEFTPTEKDEYACRVNHVTLSQPK | |
| (11-19) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |
| HPV 16E7 | YMLDLQPET | 242 |
| (11-19) peptide | | |
| sequence | | |
| NYESO-1 | SLLMWITQCGCGGSGGGGSGGGGSIQRTPKIQVYSRH | 243 |
| (157-165) pMHC | PAENGKSNFLNCYVSGFHPSDIEVDLLKNGERIEKVEHS | |
| (NYESO-1 | DLSFSKDWSFYLLYYTEFTPTEKDEYACRVNHVTLSQPK | |
| (157-165) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |
| NYESO-1 | SLLMWITQC | 244 |
| (157-165) peptide | | |
| sequence | | |
| MAGEA4 | GVYDGREHTVGCGGSGGGGSGGGGSIQRTPKIQVYSR | 245 |
| (230-239) pMHC | HPAENGKSNFLNCYVSGFHPSDIEVDLLKNGERIEKVEH | |
| (MAGEA4 | SDLSFSKDWSFYLLYYTEFTPTEKDEYACRVNHVTLSQP | |
| (230-239) peptide- | KIVKWDRDMGGGGSGGGGSGGGGSGGGGSGSHSMR | |
| linker-hB2M- | YFFTSVSRPGRGEPRFIAVGYVDDTQFVRFDSDAASQR | |
| linker-HLA-A2) | MEPRAPWIEQEGPEYWDGETRKVKAHSQTHRVDLGTL | |
| | RGCYNQSEAGSHTVQRMYGCDVGSDWRFLRGYHQYA | |
| | YDGKDYIALKEDLRSWTAADMAAQTTKHKWEAAHVAEQ | |
| | LRAYLEGTCVEWLRRYLENGKETLQRTDAPKTHMTHHA | |
| | VSDHEATLRCWALSFYPAEITLTWQRDGEDQTQDTELV | |
| | ETRPAGDGTFQKWAAVVVPSGQEQRYTCHVQHEGLPK | |
| | PLTLRWEP | |
| MAGEA4 | GVYDGREHTV | 246 |
| (230-239) peptide | | |
| sequence | | |
| CMVpp65 | NLVPMVATVGCGGSGGGGSGGGGSIQRTPKIQVYSRH | 247 |
| (465-503) pMHC | PAENGKSNFLNCYVSGFHPSDIEVDLLKNGERIEKVEHS | |
| (CMVpp65 | DLSFSKDWSFYLLYYTEFTPTEKDEYACRVNHVTLSQPK | |
| (465-503) peptide- | IVKWDRDMGGGGSGGGGSGGGGSGGGGSGSHSMRY | |
| linker-hB2M- | FFTSVSRPGRGEPRFIAVGYVDDTQFVRFDSDAASQRM | |
| linker-HLA-A2) | EPRAPWIEQEGPEYWDGETRKVKAHSQTHRVDLGTLR | |
| | GCYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAV | |
| | SDHEATLRCWALSFYPAEITLTWQRDGEDQTQDTELVE | |
| | TRPAGDGTFQKWAAVVVPSGQEQRYTCHVQHEGLPKP | |
| | LTLRWEP | |
| CMVpp65(465- | NLVPMVATV | 248 |
| 503) | | |

### 8.1.2. Jurkat NFAT Luciferase Reporter Assay

TCR activation was assessed using a Jurkat NFAT luciferase reporter assay. A Jurkat cell line with a stably incorporated NFAT-responsive luciferase reporter gene were maintained in appropriate conditions.

T cell activator and control constructs were diluted 1:3 following an 8-point dilution range. For assessments of soluble constructs, serially diluted constructs were added to cells and plates were incubated for 4 hours at 37° C. and 5% CO₂, and the luciferase activity was detected using the ONE-Glo™ (Promega) system. For assessments of plate-bound constructs, serially diluted constructs were bound to anti-Fc coated plates. In both types of assessments, emitted light was captured in relative light units (RLU) on a multilabel plate reader Envision (PerkinElmer). All serial dilutions were tested in duplicates.

### 8.1.3. Expansion of CMVpp65-Specific T Cells

Frozen PBMC cells from a healthy HLA-A02:01+ and CMV+ donor were thawed on Day 1 and rested overnight. The cells were labeled with cell trace violet on Day 2 and plated at a density of 300,000 cells/well. CMV pMHC×7221G20 was added at 5, 1, 0.2, or 0.04 nM, CMV pMHC×CD20 was added at 1.8, 0.6, 0.2, 0.06, or 0.02 nM, and IL-7 and IL-15 were added at 5 ng/ml to each well. Cells were allowed to expand for 5 days. At day 7, tetramer+ and CMVpp65+ T cell expansion was measured with a flow cytometer.

### 8.1.4. Primary TCR-T Cell IFNγ Release Assay

Frozen TCR-T cells were thawed and rested for two days at 37° C. in the presence of 100 U/mL IL2. On Day 3, 96-well flat bottom plates were blocked with media. TCR-T cells were added to blocked wells at 50,000 cells/well. T cell activator and control constructs were diluted and added to each well. Cytokine levels were detected after 4 hours of incubation.

### 8.1.5. In Vitro Tumor Killing Assay

On Day 1, rested HPV TCR-T cells were primed with HPVE7(11-19) pMHC-comprising T cell activators at 37° C. for 16 hours. The next day, CaSki HPV tumor cells were loaded with Calcein AM viability dye. TCR-T cells and CaSki tumor cells were incubated at 37° C. for 2 hours. Tumor cell death was monitored.

### 8.2. Example 1: Jurkat Reporter Cell Activation by Monovalent T Cell Activator Constructs

Monovalent HPVE7(11-19) pMHC-comprising T cell activator constructs were designed as described in Section 8.1.1. Activation of T cells via monovalent HPVE7(11-19) pMHC-comprising T cell activator constructs was assessed via Jurkat NFAT luciferase reporter assay as described in Section 8.1.2.

In a plate-bound format, monovalent T cell activator constructs pMHC×7195P, pMHC×7221G, pMHC×7221G20, and pMHC×7221G5, which comprise an HPVE7(11-19) pMHC complex linked to the Fc domain via a short linker and an anti-CD3 Fab moiety displayed comparable levels of luminescence in HPVE7(11-19) TCR709 Jurkat reporter cells and HPVE6(29-38) negative control cells (FIGS. 2A and 2B). Similar results were observed also for constructs with long linkers (FIGS. 2C and 2D), indicating that the T cell activation was driven by the CD3 arm in the plate bound format and is not antigen specific. The monovalent construct pMHC×CD28, comprising an HPVE7(11-19) pMHC complex and an anti-CD28 Fab moiety, displayed a markedly higher level of HPVE7(11-19) TCR709 Jurkat reporter cell activation than HPVE6(29-38) negative control cell activation, indicating antigen-specific activation by pMHC×CD28 (FIGS. 2A-2D).

When monovalent HPVE7(11-19) pMHC-comprising T cell activator constructs were assessed in a soluble format, monovalent constructs with moderately weak anti-CD3 Fab moieties, pMHC×7221G20 and pMHC×7221G, displayed the highest levels of HPVE7(11-19) TCR709 Jurkat reporter cell activation, followed by the other two anti-CD3 Fab moiety-comprising constructs, pMHC×7195P and pMHC×7221G5 (FIG. 3A). These constructs were associated with relatively low levels of luminescence in HPVE6(29-38) negative control cells (FIG. 3B), indicating that T cell activation by soluble constructs comprising anti-CD3 moieties was antigen specific. Similarly, constructs with long linkers comprising anti-CD3 moieties also displayed high levels of activation of HPVE7(11-19) TCR709 Jurkat reporter cells (FIG. 3C) but not HPVE6(29-38) negative control cell activation (FIG. 3D). More specifically, the highest level of reporter cell activation was observed with pMHC×7221G20, followed by pMHC×7221G and pMHC×7221G5, suggesting that soluble constructs comprising moderate to weak anti-CD3 Fab moieties were better at activating T cells.

### 8.3. Example 2: Jurkat Reporter Cell Activation by Bivalent T Cell Activator Constructs

Bivalent HPVE7(11-19) pMHC-comprising T cell activator constructs were designed as described in Section 8.1.1. Activation of T cells via monovalent T cell activator constructs was assessed via Jurkat NFAT luciferase reporter assay as described in Section 8.1.2.

In a plate-bound format, bivalent HPVE7(11-19) pMHC-comprising T cell activator constructs pMHC₂×7221G20 and pMHC₂×7221G5, which comprise weak and very weak anti-CD3 Fab moieties, respectively, were associated with the highest levels of antigen-specific activation of HPVE7(11-19) TCR709 Jurkat reporter cells (FIGS. 4A and 4B). However, constructs with moderate to strong anti-CD3 Fab moieties displayed high levels of luminescence in both HPVE7(11-19) TCR709 Jurkat reporter cells and HPVE6(29-38) negative control cells (FIGS. 4A and 4B), indicating that T cell activation by these constructs was nonspecific. Activation of HPVE7(11-19) TCR709 Jurkat reporter cells and HPVE6(29-38) negative control cells by plate-bound long linker-linked bivalent T cell activator constructs resembled those by their short linker-linked counterparts (FIGS. 4C and 4D), suggesting that the linker length had little impact on T cell activation.

When bivalent HPVE7(11-19) pMHC-comprising T cell activator constructs were assessed in a soluble format, none of the constructs displayed specific activation of TCR709 Jurkat reporter cells (FIGS. 5A-5D).

When pre-clustered with anti-Fc, bivalent HPVE7(11-19) pMHC-comprising T cell activator constructs with strong anti-CD3 Fab moieties, pMHC₂×7195P and pMHC₂×7221G, were associated with increased luminescence in both experimental and negative control Jurkat cells, although more so for constructs with short linkers (FIGS. 6A and 6B) than for constructs with long linkers (FIGS. 6C and 6D).

### 8.4. Example 3: IFNγ Release from Primary TCR-T Cells Treated with T Cell Activator Constructs

Monovalent MAGE-A4 (230-239) pMHC-comprising T cell activator constructs and HPVE7(11-19) pMHC-comprising T cell activator constructs were designed and produced as described in Section 8.1.1. T cell activator construct-mediated cytokine release from T cells was measured via TCR-T cell IFNγ release assay as described in Section 8.1.4.

In one set of assessments, IFNγ release was evaluated using MAGE-A4 PN45545 TCR-T cells treated with monovalent MAGE-A4 (230-239) pMHC-comprising T cell activator constructs. HPVE6 TCR-T cells treated with monovalent MAGE-A4 (230-239) pMHC-comprising T cell activator constructs were used as negative controls. Generally, both short and long linker-linked pMHC-comprising T cell activator constructs with anti-CD3 Fab moieties were associated with more substantial IFNγ release from MAGE-A4 PN45545 TCR-T cells than constructs with anti-CD28 Fab moieties (FIGS. 7A and 7C). Negative control cells treated with T cell activator constructs comprising anti-CD3 Fab moieties also displayed IFNγ release, but only at the highest concentrations that were evaluated (FIGS. 7B and 7D).

In another set of assessments, IFNγ release was evaluated using HPV16E7 TCR709 TCR-T cells treated with monovalent HPVE7(11-19) pMHC-comprising T cell activator constructs. NY-ESO-1 1G4 TCR-T cells treated with monovalent HPVE7(11-19) pMHC-comprising T cell activator constructs were used as negative controls. Similar to the observations with MAGE-A4 PN45545 TCR-T cells, constructs with short linkers comprising anti-CD3 Fab moieties were associated with more substantial levels of IFNγ release from HPV16E7 TCR709 TCR-T cells than constructs with anti-CD28 Fab moieties (FIG. 7E), whereas IFNγ release from negative control cells was very low even at high concentrations (FIG. 7F). MAGE-A4 PN45545 TCR-T cells treated with constructs with long linkers had the highest levels of IFNγ release if they were treated with those comprising moderate to very weak anti-CD3 Fab moieties, and moderate levels of IFNγ release if they were treated with constructs comprising a strong anti-CD3 Fab moiety or an anti-CD28 moiety (FIG. 7G). Again, IFNγ release from negative control cells was very low even at high concentrations (FIG. 7H), suggesting that the IFNγ release in HPV16E7 TCR709 TCR-T cells was antigen-specific.

### 8.5. Example 4: T Cell Activators Comprising pMHC Moieties Enhance CaSki Tumor Cell Killing by TCR-T Cells

Monovalent HPVE7(11-19) pMHC-comprising T cell activator constructs were designed and produced as described in Section 8.1.1 and in vitro tumor cell killing activity of TCR-T cells treated with pMHC-comprising T cell activator constructs was assessed as described in Section 8.1.5.

Using HPV7 TCR-T cells for priming, three HPVE7(11-19) pMHC-comprising T cell activator constructs were evaluated: pMHC×7221G, which as a moderately strong anti-CD3 Fab moiety; pMHC×7221G20, which has a weak anti-CD3 Fab moiety; and pMHC×7221G5, which has a very weak anti-CD3 Fab moiety (FIG. 8A). NY-ESO-1 TCR-T cells primed with the three HPVE7(11-19) pMHC-comprising T cell activator constructs and unprimed cells were used as controls. The highest levels of tumor cell death were observed with HPV7 TCR-T cells primed with constructs comprising very weak or weak anti-CD3 Fab domains. Priming HPV7 TCR-T cells did not enhance tumor cell death (FIG. 8B).

### 8.6. Example 5: Cis-Binding of T Cell Activators Prevents Nonspecific T Cell Activation

Monovalent NY-ESO-1(157-165) pMHC-comprising T cell activator constructs and HPVE7(11-19) pMHC-comprising T cell activator constructs were designed and produced as described in Section 8.1.1 and applied to mixed Jurkat cells comprising non-reporter 1G4-expressing TCR-T cells that are NY-ESO-1 pMHC-specific and luciferase reporter HPV7 TCR-T cells that are HPVE7(11-19) pMHC-specific. Activation of HPV7 TCR-T cells via monovalent NY-ESO-1(157-165) pMHC-comprising T cell activator constructs was assessed via Jurkat NFAT luciferase reporter assay as described in Section 8.1.2.

Application of NY-ESO-1(157-165) pMHC-comprising T cell activator constructs resulted in low levels of increase in luminescence at high concentrations of constructs, regardless of which linker was used (FIGS. 9A and 9B). In contrast, application of HPVE7(11-19) pMHC-comprising T cell activator constructs resulted in higher levels of luminescence (FIGS. 9C and 9D). Taken together, these results indicated that low levels of luciferase activity in mixed Jurkat cells treated with NY-ESO-1(157-165) pMHC-comprising T cell activator constructs was due to cis binding of these constructs to specifically to non-reporter 1G4 TCR-T cells.

### 8.7. Example 6: T Cell Activator Constructs Induce Antigen-Specific T Cell Expansion

Monovalent pMHC-comprising T cell activator constructs, CMVpp65 pMHC×7221G20 and MAGE-A4 pMHC×7221G20, comprising either long or short linkers, were designed and produced as described in Section 8.1.1. CMVpp65 T cell expansion upon treatment with CMVpp65 pMHC×7221G20 or MAGE-A4 pMHC×7221G20 was assessed in CMV PBMCs as described in Section 8.1.3.

As a control, pulsing CMV PBMCs with CMVpp65 peptide resulted in minimal overlap between CMVpp65+ T cells and CMVpp65-CD8+ T cells (FIG. 10A). Treatment with 0.2 nM CMVpp65 pMHC×7221G20 T cell activator comprising a long linker enabled CMVpp65+ T cells to proliferate specifically, whereas CMVpp65-CD8+ population overlapped with unexpanded population of cells (FIG. 10B), indicating that the CD8+ Tetramer-population did not proliferate with this treatment. Increasing the concentration of the CMVpp65 pMHC×7221G20 T cell activator comprising a long linker from 0.2 nM to 5 nM increased CMVpp65-CD8+ T cell proliferation (FIG. 10C). Negative control treatments with 0.2 nM MAGE-A4 pMHC×7221G20 with a long linker displayed negligible proliferation of the CD8+ Tetramer-population as indicated by the overlap of Tetramer+ and Tetramer-peaks with no significant divisions (FIGS. 10D and 10F) although the peaks corresponding to Tetramer+ and Tetramer-cells overlapped more extensively when cells were treated with 5 nM MAGE-A4 pMHC×7221G20 with a long linker (FIGS. 10E and 10F).

In another set of assessments, CMV PBMCs were treated with 5 nM CMVpp65 pMHC×7221G20 or MAGE-A4 pMHC×7221G20 with short linkers, the results of which were similar to those observed with assessments with T cell activators comprising long linkers (FIGS. 11A-11F).

Relative to untreated CMV PBMCs (FIGS. 12A and 12B), CMV PBMCs pulsed with CMVpp65 displayed steadfast expansion of CD8+ tetramer+ cells and TCRα/β+ tetramer+ cells (FIGS. 12C and 12D). Treatment with CMVpp65 pMHC×7221G20 comprising a long linker was also associated with expansion of CD8+ tetramer+ cells and TCRα/β+ tetramer+ cells at 0.2 nM (FIGS. 12E and 12F) and 5 nM (FIGS. 12G and 12H). Treatment with MAGE-A4 pMHC×7221G20 did not cause expansion of CD8+ tetramer+ cells and TCRα/β+ tetramer+ cells at either concentration (FIGS. 121-12L), indicating that the expansion upon treatment with CMVpp65 pMHC×7221G20 comprising a long inker was antigen-specific. Similarly, treatment with CMVpp65 pMHC×7221G20 comprising a short linker was associated with expansion of CD8+ tetramer+ cells and TCRα/β+ tetramer+ cells at 0.2 nM (FIGS. 12M and 12N) and 5 nM (FIGS. 12O and 12P), whereas treatment with MAGE-A4 pMHC×7221G20 did not cause expansion of CD8+ tetramer+ cells and TCRα/β+ tetramer+ cells at either concentration (FIGS. 12Q-12T).

### 8.8. Example 7: Effect of CD22×CD28 Costimulation on Antigen-Specific T Cell Activation with Anchored T Cell Activators

Anchored monovalent HPVE7(11-19) pMHC-comprising T cell activator constructs comprising either CD20 or CD19 arms were designed as described in Section 8.1.1. Activation of T cells by these T cell activator constructs with or without costimulation with a constant concentration of REGN5837 (CD22×CD28 bispecific antibody) was assessed via Jurkat NFAT luciferase reporter assay as described in Section 8.1.2., using a 1:1 mixture of HPV16E7 (11-19) TCR709 TCR-T cells: Raji dKO (CD80 CD86 KO) cells. Control experiments were conducted using 1:1 mixture of HPV16E6 (29-38) TCR Jurkat cells: Raji dKO (CD80 CD86 KO) cells.

Anchored monovalent T cell activators comprising CD20 arms displayed antigen-specific T cell activation. Similar constructs comprising CD19 arms did not activate HPV16E7(11-19) TCR709 Jurkat T cells at the concentrations tested (solid lines in FIGS. 13A and 13B). Costimulation with 31.5 μg/mL REGN5837 had synergistic effects, enhancing antigen specific T cell activation by CD20-comprising T cell activators (dashed lines in FIG. 13A). Costimulation with 62.5 μg/mL REGN5837 also had synergistic effects, again characterized by an enhancement of antigen specific T cell activation by CD20-comprising T cell activators (FIGS. 13C and 13D). No synergistic effects were observed when REGN5837 concentration was increased to 125 μg/mL (FIGS. 13E and 13F).

### 8.9. Example 8: Effect of CD22×CD28 Costimulation on Antigen-Specific T Cell Activation with Anchored pMHC×CD20 T Cell Activators

Anchored monovalent HPVE7(11-19) pMHC-comprising T cell activator constructs comprising a CD20 arm, wherein a pMHC complex is connected to the hinge region of the Fc domain via a short or a long linker, were designed as described in Section 8.1.1. Activation of T cells by the T cell activator constructs with or without costimulation with various concentrations of the CD22×CD28 bispecific antibody, REGN5837, was assessed via Jurkat NFAT luciferase reporter assay as described in Section 8.1.2., using a 1:1 (50,000 cells:50,000 cells) mixture of HPV16E7 (11-19) TCR TCR-T cells:Raji dKO (CD80 CD86 KO) cells. Control experiments were conducted using 1:1 (50,000 cells:50,000 cells) mixture of HPV16E6 (29-38) TCR Jurkat cells:Raji dKO (CD80 CD86 KO) cells.

The anchored monovalent pMHC×CD20 T cell activator construct comprising a short linker displayed activation of HPV16E7(11-19) TCR Jurkat T cells but not of HPVE6(29-38) TCR Jurkat T cells (“Negative Control”). Costimulation with REGN5837 had synergistic effects even at the lowest concentration tested (0.06 μg/mL), enhancing antigen specific T cell activation (FIGS. 14A and 14B). Similarly, anchored monovalent pMHC×CD20 T cell activator construct comprising a long linker also displayed antigen-specific T cell activation of HPV16E7(11-19) TCR Jurkat T cells, and each evaluated concentration of REGN5837 enhanced antigen specific T cell activation (FIGS. 14C and 14D).

### 8.10. Example 9: Effect of Fc Domain and Linker Length on Jurkat Reporter Cell Activation by T Cell Activators

T cell activator constructs were designed as described in Section 8.1.1. Each construct contained either an IgG1 Fc domain or an IgG4 Fc domain, an anti-CD3 Fab moiety, and an HPVE7(11-19) pMHC complex connected to the hinge region of the Fc domain with either a short linker (i.e., a linker at most or exactly 7 amino acids in length) or a long linker (i.e., a linker more than 7 amino acids in length). Activation of T cells via T cell activator constructs was assessed via Jurkat NFAT luciferase reporter assay as described in Section 8.1.2. Results obtained from constructs having a short linker are shown in FIGS. 15A-15D (IgG1-containing constructs shown in FIGS. 15A and 15B; IgG4-containing constructs shown in FIGS. 15C and 15D). Results obtained from constructs having a long linker are shown in FIGS. 16A-16D (IgG1-containing constructs shown in FIGS. 16A and 16B; IgG4-containing constructs shown in FIGS. 16C and 16D).

IgG1-containing T cell activator constructs with short linkers displayed dose-dependent increase in luminescence in HPVE7(11-19) TCR709 Jurkat reporter cells (FIG. 15A). Treatment of HPVE6(29-38) negative control cells also showed T cell activation (FIG. 15B), suggesting that at least some of the luminescence observed with HPVE7(11-19) TCR709 Jurkat reporter cells was non-specific. The luminescence observed with IgG4-containing T cell activator constructs with short linkers in HPVE7(11-19) TCR709 Jurkat reporter cells was less pronounced than the luminescence observed with IgG4-comprising constructs (FIG. 15C). However, IgG4-comprising T cell activator constructs with short linkers were associated with little to no luminescence in HPVE6(29-38) negative control cells (FIG. 15D).

Results obtained with IgG1-containing T cell activator constructs with long linkers resembled those obtained with their short linker counterparts (FIG. 16A in view of FIG. 15A and FIG. 16B in view of FIG. 15B). The luminescence observed with IgG4-containing T cell activator constructs with long linkers in HPVE7(11-19) TCR709 Jurkat reporter cells was relatively weaker than the luminescence associated with their short linker counterparts (FIG. 16C in view of FIG. 15C), whereas it was comparable in HPVE6(29-38) negative control cells.

Taken together, these results suggest that constructs with IgG4 Fc domains and short linkers between the pMHC complex and the hinge region of the Fc domain were associated with lower yet more specific activation of T cells than their IgG1-containing counterparts.

### 8.11. Example 10: Effect of Linker Length on Jurkat Reporter Cell Activation by T Cell Activators

T cell activator constructs were designed as described in Section 8.1.1. Each construct contained 7221G20 or a comparator anti-CD3 moiety selected from those listed in Table E-1 and an HPVE7(11-19) pMHC complex connected to the hinge region of the Fc domain with either a short linker (i.e., a linker at most or exactly 7 amino acids in length) or a long linker (i.e., a linker more than 7 amino acids in length). Activation of T cells via T cell activator constructs was assessed via Jurkat NFAT luciferase reporter assay as described in Section 8.1.2. Results obtained from constructs having a short linker are shown in FIGS. 17A and 17B. Results obtained from constructs having a long linker are shown in FIGS. 17C and 17D.

T cell activator constructs with short linkers displayed various levels of luminescence in HPVE7(11-19) TCR709 Jurkat reporter cells (FIG. 17A). The levels of luminescence of each construct in control cells mirrored those in reporter cells (FIG. 17B). Generally, the constructs with long linkers were associated with weaker HPVE7(11-19) TCR709 Jurkat reporter cell activation than their short linker counterparts (FIG. 17C). Results obtained with constructs comprising long linkers were similar to those obtained with their short linker counterparts in control cells (FIG. 17D).

### 8.12. Example 11: pMHC×CD20 T Cell Activator Constructs Induce Antigen-Specific T Cell Expansion

Monovalent pMHC-comprising T cell activator constructs, CMVpp65 pMHC×CD20 (VAC3B9) and MAGE-A4 pMHC×CD20 (VAC3B9), comprising either long or short linkers, were designed as depicted in FIG. 1A and produced as described in Section 8.1.1. CMVpp65 T cell expansion upon treatment with T cell activator constructs was assessed in CMV PBMCs with a CMVpp65+ T cell:B cell ratio of 1:58 as described in Section 8.1.3.

Pulsing CMV PBMCs with CMVpp65 peptide resulted in minimal overlap between CMVpp65+ T cells and CMVpp65-CD8+ T cells (FIG. 18A). Treatment with 0.02 nM to 1.8 nM CMVpp65 pMHC×CD20 T cell activator comprising a long linker stimulated CMVpp65+ T cell proliferation, whereas CMVpp65-CD8+ population overlapped with unexpanded population of cells, indicating that the CD8+ Tetramer-population did not proliferate with this treatment (FIGS. 18B, 18C, and 18F). Negative control treatments with 0.2 nM MAGE-A4 pMHC×CD20 with a long linker displayed no proliferation of the CD8+ Tetramer+ population as indicated by the overlap of Tetramer+ and Tetramer-peaks with no significant divisions (FIGS. 18D and 18E). Higher concentrations of CMVpp65 pMHC×CD20 T cell activator construct resulted in higher rates of B cell killing, in which percent reduction in B cells relative to mock control rose from 23% at 0.02 nM CMVpp65 pMHC×anti-CD20 to 54% at 1.8 nM CMVpp65 pMHC×anti-CD20 (FIG. 18G).

Next, CMV PBMCs were treated with between 0.02 nM and 1.8 nM CMVpp65 pMHC×CD20 or MAGE-A4 pMHC×CD20 with short linkers, the results of which were similar to those observed with assessments with T cell activators comprising long linkers (FIGS. 19A-19G).

In the next set of assessments, treatment with CMVpp65 pMHC×CD20 comprising a long linker was associated with expansion of CD8+ tetramer+ cells at 0.02 nM (FIG. 20A) and 1.8 nM (FIG. 20B). CMV PBMCs pulsed with CMVpp65+IFNα displayed steadfast expansion of CD8+ tetramer+ cells (FIG. 20C). Treatment with MAGE-A4 pMHC×CD20 did not cause expansion of CD8+ tetramer+ cells at either concentration (FIGS. 20D and 20E), indicating that the expansion upon treatment with CMVpp65 pMHC×CD20 comprising a long inker was antigen specific. Similarly, treatment with CMVpp65 pMHC×7221G20 comprising a short linker was associated with expansion of CD8+ tetramer+ cells at 0.02 nM and 1.8 nM (FIGS. 20F and 20G), whereas treatment with MAGE-A4 pMHC×CD20 did not cause expansion of CD8+ tetramer+ cells at either concentration (FIGS. 20I and 20J).

### 8.13. Example 12: Effect of T Cell:B Cell Ratio on Antigen Specific T Cell Proliferation

Monovalent pMHC-comprising T cell activator constructs, CMVpp65 pMHC×CD20 and MAGE-A4 pMHC×CD20, comprising either long or short linkers, were designed as depicted in FIG. 1A and produced as described in Section 8.1.1. CMVpp65 T cell expansion upon treatment with T cell activator constructs was assessed in nondepleted CMV PBMCs with a CMVpp65+ T cell:B cell ratio of 1:5, and in B cell depleted CMV PBMCs, isolated Pan T cells, and CMV PBMCs with a CMVpp65+ T cell:B cell ratio of 1:58 as described in Section 8.1.3.

Nondepleted PBMCs with a CMVpp65+ T cell:B cell ratio of 1:5 were pulsed with CMVpp65 peptide, which resulted in only a small degree of overlap between CMVpp65+ T cells and CMVpp65-CD8+ T cells (FIG. 21A). Treatment with 0.02 nM CMVpp65 pMHC×CD20 T cell activator comprising a long linker stimulated CMVpp65+ T cell proliferation (FIG. 21B) and similar results were obtained with 0.02 nM CMVpp65 pMHC×CD20 T cell activator comprising a short linker (FIG. 21C). Treatment with MAGE-A4 pMHC×CD20 did not cause expansion of CD8+ tetramer+ cells for MAGE-A4 pMHC×CD20 comprising either a long linker or a short linker (FIGS. 21D and 21E, respectively).

Treatment of B cell depleted CMV PBMCs with CMVpp65 pMHC×CD20 T cell activator comprising either a long linker or a short linker did not result in antigen specific T cell expansion (FIGS. 21F-21H). Similarly, no antigen specific T cell expansion was observed in isolated Pan T cells treated with CMVpp65 pMHC×CD20 T cell activator comprising a long linker or a short linker (FIGS. 211-21K).

Next, antigen-specific expansion of T cells was assessed using PBMCs with a high B cell population whose T cell:B cell ratio was 1:58. As was the case for the PBMCs with a CMVpp65+ T cell:B cell ratio of 1:5, pulsing with CMVpp65 peptide resulted in minimal overlap between CMVpp65+ T cells and CMVpp65-CD8+ T cells (FIG. 22A). Treatment of PBMCs with a T cell:B cell ratio of 1:58 with 0.02 nM CMVpp65 pMHC×CD20 comprising a long linker resulted in a greater antigen specific expansion of CMVpp65+ T cells relative to the expansion observed with the PBMCs with a 1:5 T cell:B cell ratio (FIGS. 22B and 21B, respectively). Similarly, treatment of these high B cell population PBMCs with 0.02 nM CMVpp65 pMHC×CD20 comprising a short linker was associated with a greater antigen specific expansion of CMVpp65+ T cells relative to the expansion observed with the PBMCs with a T cell:B cell ratio of 1:5 (FIGS. 22C and 21C, respectively). Treatment with MAGE-A4 pMHC×CD20 comprising either a long linker or a short linker did not cause antigen specific expansion of CD8+ tetramer+ cells (FIGS. 22D and 22E, respectively).

Assessment of B cell death associated with CMVpp65 pMHC×CD20 treatment at 0.02 nM showed that B cell killing was low when the B cell population in CMV PBMCs was high (FIG. 22F). When PBMCs with a T cell:B cell ratio of 1:58 were treated with 0.02 nM parental CD3×CD20 antibody (REGN1979), the B cell population was diminished (FIG. 22G). Treatment with the parental antibody resulted in a large degree of overlap between CMVpp65+ T cells and CMVpp65-CD8+ T cells (FIG. 22H) but no such overlap was observed when the cells were pulsed with CMVpp65 peptide (FIG. 221), suggesting that the parental antibody treatment-induced overlap between CMVpp65+ T cells and CMVpp65-CD8+ T cells and killing of B cells was associated with CD8+ T cell proliferation.

### 8.14. Example 13: T Cell Expansion Efficacy of Different T Cell Activators

CMVpp65 T cell expansion upon treatment with T cell activator constructs CMVpp65 pMHC×CD3 and MAGE-A4 pMHC×CD3 was assessed in CMV PBMCs with a CMVpp65+ T cell:B cell ratio of 1:58 as described in Section 8.1.3 and compared to those obtained with CMVpp65 pMHC×CD20 and MAGE-A4 pMHC×CD20.

There was minimal overlap between CMVpp65+ T cells and CMVpp65-CD8+ T cells when PBMCs were pulsed with the CMVpp65 peptide (FIG. 23A). Treatment with CMVpp65 pMHC×CD20 with a long linker was associated with a greater expansion of CMVpp65 T cells (FIG. 23B) than with CMVpp65 pMHC×CD3 with a long linker (FIG. 23F). Similarly, treatment with CMVpp65 pMHC×CD20 with a short linker was associated with a greater expansion of CMVpp65 T cells (FIG. 23C) than with CMVpp65 pMHC×CD3 with a short linker (FIG. 23G). Treatment with MAGE-A4 pMHC×CD20 constructs was not associated with antigen specific expansion of CD8+ tetramer+ cells (FIGS. 23D and 23E). These results suggested that CMVpp65 pMHC×CD20 T cell activator constructs evaluated in this Example were more efficacious at antigen specific expansion of T cells than their CMVpp65 pMHC×CD3 T cell activator counterparts.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A multispecific molecule comprising:
(a) a peptide-MHC (pMHC) complex comprising an MHC domain and an antigenic peptide;
(b) an immune cell antigen (ICA) targeting moiety; and
(c) optionally, a multimerization moiety operably linked to the pMHC complex.

2. The multispecific molecule of claim 1, which comprises a single ICA targeting moiety.

3. The multispecific molecule of claim 1, which comprises two ICA targeting moieties.

4. The multispecific molecule of claim 3, wherein the two ICA targeting moieties bind to the same antigen, optionally wherein the two ICA targeting moieties are the same.

5. The multispecific molecule of claim 3, wherein the two ICA targeting moieties bind to different antigens.

6. The multispecific molecule of any one of claims 1 to 5, which comprises a single pMHC complex.

7. The multispecific molecule of any one of claims 1 to 5, which comprises two pMHC complexes.

8. The multispecific molecule of claim 7, wherein the two pMHC complexes are the same.

9. The multispecific molecule of any one of claims 1 to 8, which comprises a multimerization moiety operably linked to the pMHC complex, wherein the multimerization moiety is an Fc domain.

10. The multispecific molecule of any one of claims 1 to 9, wherein the multispecific molecule is a heterodimer.

11. The multispecific molecule of any one of claims 2 to 10, wherein the pMHC complex is N-terminal to the multimerization moiety.

12. The multispecific molecule of any one of claims 2 to 10, wherein the pMHC complex is C-terminal to the multimerization moiety.

13. The multispecific molecule of any one of claims 2 to 12, wherein the ICA targeting moiety is N-terminal to the multimerization moiety.

14. The multispecific molecule of any one of claims 2 to 12, wherein the ICA targeting moiety is C-terminal to the multimerization moiety.

15. The multispecific molecule of any one of claims 1 to 14, wherein the pMHC complex and the ICA targeting moiety are on a single polypeptide.

16. The multispecific molecule of any one of claims 1 to 14, wherein the pMHC complex and the ICA targeting moiety are on different polypeptides.

17. The multispecific molecule of any one of claims 1 to 16, wherein the ICA targeting moiety is a T cell antigen (TCA) targeting moiety.

18. The multispecific molecule of claim 17, wherein the TCA targeting moiety is a CD3 targeting moiety.

19. The multispecific molecule of claim 17, wherein the TCA targeting moiety is a CD28 targeting moiety.

20. The multispecific molecule of any one of claims 1 to 16, wherein the ICA targeting moiety is a B cell antigen (BCA) targeting moiety.

21. The multispecific molecule of claim 20, which lacks a TCA targeting moiety.

22. The multispecific molecule of claim 20 or 21, wherein the BCA targeting moiety is a CD19 targeting moiety.

23. The multispecific molecule of claim 20 or 21, wherein the BCA targeting moiety is a CD20 targeting moiety.

24. The multispecific molecule of claim 20 or 21, wherein the BCA targeting moiety is a CD22 targeting moiety.

25. The multispecific molecule of any one of claims 1 to 24, further comprising a tumor antigen targeting moiety.

26. The multispecific molecule of claim 25, wherein the tumor antigen targeting moiety is a tumor-associated antigen (TAA) targeting moiety.

27. The multispecific molecule of any one of claims 1 to 26, wherein the MHC domain is a type I MHC domain.

28. The multispecific molecule of claim 27, wherein the pMHC complex further comprises a 32-microglobulin domain.

29. The multispecific molecule of any one of claims 1 to 28, wherein the pMHC complex is a single polypeptide.

30. The multispecific molecule of any one of claims 1 to 29, wherein the multispecific molecule comprises a polypeptide comprising, in N- to C-terminal orientation, the pMHC complex and an Fc domain.

31. A multispecific molecule comprising:
(a) a first polypeptide chain comprising:
(i) a peptide-MHC (pMHC) complex comprising an MHC domain and an antigenic peptide; and
(ii) a first Fc domain; and
(b) a second polypeptide chain comprising:
(i) a VH; and
(ii) a second Fc domain that associates with the first Fc domain to form an Fc region; and
(c) a third polypeptide comprising:
(i) a VL that associates with the VH to form an immune cell antigen (ICA) targeting moiety.

32. The multispecific molecule of claim 31, wherein the pMHC complex is N-terminal to the first Fc domain.

33. The multispecific molecule of claim 31, wherein the pMHC complex is C-terminal to the first Fc domain.

34. The multispecific molecule of any one of claims 31 to 33, wherein the VH is N-terminal to the second Fc domain.

35. The multispecific molecule of any one of claims 31 to 33, wherein the VH is C-terminal to the second Fc domain.

36. The multispecific molecule of any one of claims 31 to 35, wherein the ICA targeting moiety is a T cell antigen (TCA) targeting moiety.

37. The multispecific molecule of claim 36, wherein the TCA targeting moiety is a CD3 targeting moiety.

38. The multispecific molecule of claim 36, wherein the TCA targeting moiety is a CD28 targeting moiety.

39. The multispecific molecule of any one of claims 31 to 35, wherein the ICA targeting moiety is a B cell antigen (BCA) targeting moiety.

40. The multispecific molecule of claim 39, which lacks a TCA targeting moiety.

41. The multispecific molecule of claim 39 or 40, wherein the BCA targeting moiety is a CD19 targeting moiety.

42. The multispecific molecule of claim 39 or 40, wherein the BCA targeting moiety is a CD20 targeting moiety.

43. The multispecific molecule of claim 39 or 40, wherein the BCA targeting moiety is a CD22 targeting moiety.

44. The multispecific molecule of any one of claims 31 to 43, further comprising a tumor antigen targeting moiety.

45. The multispecific molecule of claim 44, wherein the tumor antigen targeting moiety is a tumor-associated antigen (TAA) targeting moiety.

46. The multispecific molecule of any one of claims 31 to 45, wherein the MHC domain is a type I MHC domain.

47. The multispecific molecule of any one of claims 31 to 46, wherein the pMHC complex further comprises a β2-microglobulin domain.

48. The multispecific molecule of claim any one of claims 1 to 47, wherein the antigenic peptide is an antigenic peptide set forth in Table 2-C.

49. A nucleic acid or plurality of nucleic acids encoding the multispecific molecule of any one of claims 1 to 48.

50. A cell transfected with one or more expression vectors comprising one or more nucleic acid sequences encoding the multispecific molecule of any one of claims 1 to 48 under the control of one or more promoters.

51. A method of producing the multispecific molecule of any one of claims 1 to 48, comprising culturing the host cell of claim 50 and recovering the multispecific molecule expressed thereby.

52. A method of activating T cell receptor signaling in a T cell or a population of T cells comprising administering to the T cell or population of T cells the multispecific molecule of any one of claims 1 to 48.

53. A pharmaceutical composition comprising (a) the multispecific molecule of any one of claims 1 to 48 or one or more nucleic acids encoding the multispecific molecule of any one of claims 1 to 48 and (b) an excipient.

54. A method of treating cancer comprising administering to a subject in need thereof the multispecific molecule is the multispecific molecule of any one of claims 1 to 48 or the nucleic acid(s) of claim 49.

55. A method of inhibiting growth of a tumor cell in a subject, comprising administering to the subject the multispecific molecule is the multispecific molecule of any one of claims 1 to 48 or the nucleic acid(s) of claim 49.

56. A method of stimulating proliferation of cancer antigen specific T cells, comprising administering to a subject the multispecific molecule is the multispecific molecule of any one of claims 1 to 48 or the nucleic acid(s) of claim 49.

57. The method of any one of claims 54 to 56, wherein administration of the multispecific molecule reduces the viability of cancer cells expressing a protein comprising the amino acid sequence of the antigenic peptide in the subject.

58. The method of any one of claims 54 to 56, further comprising administering to the subject a multispecific antigen binding molecule comprising (a) a first antigen-binding domain that specifically binds a tumor antigen; and (b) a second antigen-binding domain that specifically binds a T cell antigen.

59. The method of claim 58, wherein the second antigen-binding domain is a CD28 binding domain.

60. A method of stimulating proliferation of antigen specific T cells, comprising administering to a subject the multispecific molecule is the multispecific molecule of any one of claims 1 to 48 or the nucleic acid(s) of claim 49.

61. A method comprising administering to a subject the multispecific molecule of any one of claims 1 to 48, one or more nucleic acids encoding the multispecific molecule of any one of claims 1 to 48, or a pharmaceutical composition according to claim 53.
